(19) United States
(12) Patent Application Publication
(10) Pub. No.: US 2021/0290676 A1
(43) Pub. Date: Sep. 23, 2021

(54) IMPROVING THE EFFICACY AND SAFETY OF ADOPTIVE CELLULAR THERAPIES

(71) Applicant: University of Southern California, Los Angeles, CA (US)

(72) Inventor: Preet M. Chaudhary, Toluca Lake, CA (US)

(21) Appl. No.: 17/264,788

(22) PCT Filed: Jul. 30, 2019
(86) PCT No.: PCT/US2019/044255

§ 371 Date: Jan. 29, 2021

Related U.S. Application Data
(60) Provisional application No. 62/712,102, filed on Jul. 30, 2018.

(51) Int. Cl. A61K 35/17; A61K 31/496; C07K 14/705; C07K 14/725

(52) U.S. Cl. CPC A61K 35/17; A61K 31/496; C07K 14/70578; C07K 2319/33; C07K 2317/76; C07K 2319/03; C07K 14/7051

(57) ABSTRACT

The disclosure relates generally to the generation and use of immune effector cells (e.g., T cells, NK cells) engineered to express an immune receptor (e.g., Chimeric Antigen Receptor, Synthetic Immune Receptor, T Cell Receptor etc.) to treat a disease associated with expression of a target antigen.

### CROSS REFERENCE TO RELATED APPLICATIONS

This application claims priority of U.S. Provisional Application No. 62/712,102, filed Jul. 30, 2018, the disclosures of which are incorporated herein by reference in their entirety.

### SEQUENCE LISTING

The instant application contains a Sequence Listing which has been submitted electronically in ASCII format and is hereby incorporated by reference in its entirety. Said ASCII copy entitled “SEQ-1-3059_ST25”, created on Jul. 30, 2019, and is 4,053,956 bytes in size.

### FIELD OF THE INVENTION

The disclosure relates generally to the use of immune effector cells (e.g., T cells, NK cells) engineered to express a Chimeric Antigen Receptor (CAR) to treat a disease associated with expression of a tumor antigen.

### BACKGROUND

Adoptive cell transfer (ACT) therapy with immune-cells, especially with T-cells transduced with Chimeric Antigen Receptors (CARs) and recombinant TCRs, has shown promise in hematologic malignancies and solid tumor trials. However, there is high rate of failure in the manufacturing of cell therapy products, resulting in poor yield, poor proliferation, expansion and tissue (e.g., tumor) penetration of the product upon administration, poor efficacy and increased cost.

There is also a medical need for cell therapies (e.g., immune cell therapies, e.g., CAR-T, SIR-T, Ab-TCR-T, TFP-T, TILs or TCR-T) with improved efficacy and reduced toxicity. The efficacy of adoptive immune therapy with T cells expressing conventional and next generation chimeric antigen receptors, (e.g., SIR, zSIR, AbTCR, and TFP) and native (e.g., Tumor Infiltrating Lymphocytes) or engineered T cell receptors (e.g., NY-ESO-1 TCR), correlates well with the expansion and proliferation of the T cells when infused into the patient. Thus, patients showing robust in vivo expansion of the adoptively transferred T cells have better chance of showing disease response as compared to the patients in whom the adoptively transferred T cells either fail to expand or do so poorly. Failure of expansion of infused T cells is a bigger problem in adoptive immunotherapy of solid tumors as compared to hematologic malignancies and may play a role in the overall inferior response of solid tumors to adoptive immune therapies. Strategies to improve the in vivo expansion of adoptively transferred T cells include administration of lymphodepleting chemotherapy. For example, fludarabine and cyclophosphamide are commonly administered to the patients prior to infusion of adoptively transferred T cells. However, administration of lymphodepleting chemotherapy is associated with significant toxicities (e.g., pancytopenia, fever, risk of infection, neurotoxicity) and death. Additionally, adoptively transferred T cells fail to expand despite the administration of lymphodelpeting chemotherapy. A related problem with adoptively transferred immune cells is excessive stimulation and proliferation, which is associated with toxicities such as cytokine release syndrome, neurotoxicity, including death. Additionally, over-stimulation and proliferation of adoptively transferred T cells have been associated with early T cell exhaustion and disease relapse.

Accordingly, the need exists to improve the yield, expansion, activation, proliferation, expansion, diversity, tissue (e.g., tumor) penetrance, persistence, efficacy and safety of cell therapy products, e.g., immune cell therapy products, e.g., CAR-T, SIR-T, TFP-T, Ab-TCR-T and TCR-T products etc. In particular, there is a need to for new methods to improve the manufacturing of cell therapy products to improve yield, reduce manufacturing time, improve in vivo expansion and efficacy and reduce cost. In addition, the need exists for a method in which the expansion, activation and proliferation of adoptively transferred immune cells can be controlled after infusion to a subject. Finally, the need exists to improve the penetration of adoptively transferred cells to the sites of disease, e.g., tumor.

### SUMMARY

The disclosure relates to a method of improving the yield, expansion, activation, proliferation, expansion, diversity, tissue (e.g., tumor) penetrance, persistence, efficacy and safety of cell therapy products, e.g., immune cell therapy products, e.g., CAR-T, SIR-T, TFP-T, Ab-TCR-T and TCR-T products etc. The disclosure related to improving the yield, expansion, activation, proliferation, expansion, diversity, tissue (e.g., tumor) penetrance, persistence and efficacy of immune cell therapies, such as engineered CAR-T, TCR-T, SIR-T, Ab-TCR-T cell therapies or NK cell therapies, by using mobilized immune cells for the manufacturing of cell therapy products. In various embodiments, the method involves mobilizing immune cells prior to collecton of immune cells (e.g. leukopheresis) from the donor. In various embodiments, the immune cells are mobilized by administrating to the donor from whom the immune cells are harvested a CXCR antagonist (e.g., Mozibil or Plerixafor), a cytokine (e.g., G-CSF, GM-CSF or sargramostim, Neulasta or Pegfilgastrim), a beta2 adrenergic agonist (e.g., epinephrine), a tyrosine kinase inhibitor (e.g., dasatinib), chemotherapy drug(s) (e.g., cyclophosphamide, doxorubicin) or a combination of the above agents prior to the collection of immune cells. In various embodiments, the immune cells are mobilized by making the subject (i.e. donor) exercise. In some embodiments, the immune cells are mobilized from an autologous donor. In other embodiments, the immune cells are mobilized from an allogeneic donor.

The disclosure also pertains, at least in part, to methods for improving the expansion and/or activation (e.g., in vitro and in vivo expansion and/or activation) of immune cells, e.g., immune T cells, e.g., CAR-T cells or TCR-T cells or TILs or NK cells or CAR-NK cells or macrophage cells or macrophage-CAR cells, for the purpose of adoptive cellular therapy. Some embodiments described herein provide for expansion and/or activation of immune cells by exposing them to a bispecific or multi-specific engager that contains at least one antigen binding domain capable of engaging the immune cells and at least one antigen binding domain capable of engaging an antigen presenting cell (APC) or antigen presenting substrate (APS), e.g. antigen-conjugated beads.

The disclosure also pertains, at least in part, to methods for improving the expansion and/or activation (e.g., in vitro and in vivo expansion and/or activation) of immune cells, e.g., immune T cells, e.g., CAR-T cells CAR-NK cells or macrophage-CAR cells, for the purpose of adoptive cellular therapy. Some embodiments described herein provide for expansion and/or activation of immune cells by culturing with cells and/or cell lines derived from Mantle cell lymphoma. Exemplary cell lines derived from Mantle cell lymphoma include, but are not limited to, REC-1, MINO, JEKO-1, and GRANTA-519. In a preferred embodiment, the immune cells, e.g., T cells, express a CAR targeting an antigen (e.g., CD19, CD20, CD22 and BCMA) expressed on B and plasma cell lineage cells, and are expanded/activated by co-culturing with REC-1 cells.

The disclosure also relates to methods for preventing or ameliorating toxicity caused by or due to a therapy, e.g., cell therapy or immune therapy, e.g., CAR-T therapy or Blinatumomab therapy, by interfering with and/or blocking the activity of TRAIL (TNFSF10). In some embodiments, the therapy involves a bispecific or multispecific engager that activates immne cells, e.g., T cells. Exemplary bispecific engager that activate immune cells include Blinatumomab, CD3×CD123 DART and BCMA×CD3 BiTE. In some embodiments, the therapy is a cell therapy in which the cells generally express recombinant receptors such as chimeric receptors, e.g., chimeric antigen receptors (CARs) or other transgenic receptors such as T cell receptors (TCRs).

In some embodiments, the activity of TRAIL is interfered or blocked by administering a TRAIL/TNFSF10 antagonist. In some embodiments, the TRAIL antagonist is an antibody, an antibody fragment, a small molecule, a peptide or a nucleic acid. In some embodiments, the TRAIL antagonist is an agent that binds to TRAIL (TNFSF10) or its receptors Death Receptor 5 (DR5 or TNFRSF10B), or Death Receptor 4 (DR4 or TNFRSF10A), such as an antibody, an antibody fragment or a non-immunoglobulin antigen binding scaffold such as a DARPIN, an affibody, an affilin, an adnectin, an affitin, an obodies, a repebody, a fynomer, an alphabody, an avimer, an atrimer, a centyrin, a pronectin, an anticalin, a kunitz domain, an Armadillo repeat protein or a fragment thereof.

In some embodiments, the TRAIL antagonist is a soluble TRAIL receptor (e.g., DR5-SP-ECD-hIgFc; SEQ ID NO: 2428, DR4-SP-ECD-hIgFc; SEQ ID NO: 2441, DcR1-SP-ECD-hIgFc; SEQ ID NO: 2448; or DcR2-SP-ECD-hIgFc; SEQ ID NO; 2455). In some embodiment, the TRAIL antagonist is a nucleic acid targeting TRAIL (e.g, a shRNA targeting TRAIL or a gRNA targeting TRAIL) or its receptor DR5 (e.g, a shRNA targeting DR5 or a gRNA targeting DR5) and DR4 (e.g, a shRNA targeting DR4 or a gRNA targeting DR4). The target sequence for gRNAs targeting TRAIL (SEQ ID NO: 2060-2109), DR4 (SEQ ID NO: 2111-2160) and DR5 (SEQ ID NO: 2162-2211) are provided in the disclosure. The target sequence for shRNAs targeting TRAIL (SEQ ID NO: 2213-2216), DR4 (SEQ ID NO: 2217-2220) and DR5 (SEQ ID NO: 2221-2224) are provided in the disclosure. The sequence of shRNA constructs targeting TRAIL (SEQ ID NO: 2226-2230), DR4 (SEQ ID NO: 2231-2233) and DR5 (SEQ ID NO: 2234-2237) are also provided in the disclosure.

In some embodiments, the TRAIL antagonist is a small molecule compound that interferes with signal transduction pathways activated by binding of TRAIL to its receptors DR5 or DR4. In some embodiments, the TRAIL antagonist is an agent that alters the expression of TRAIL or its receptors. In some embodiments, the TRAIL antagonist is an agent that down-regulates the expression of TRAIL or its receptors DR5 and/or DR4. As an example, a TRAIL antagonist may interfere with the expression of TRAIL, DR5 and/or DR4 at the transcription, post-transcription, translation, or post-transplation levels. In some embodiments, the TRAIL antagonist is included in the manufacturing of cell therapy products. In other embodiments, the cell therapy product is exposed to TRAIL antagonist prior to administration to a subject. In other embodiments, the TRAIL antagonist is incorporated into the cell therapy product. In exemplary embodiments, a cell therapy products (e.g., CAR-T cell product, TCR-T product or TIL), may be genetically engineered to express a) a soluble TRAIL receptor (e.g., DR5-SP-ECD-hIgFc; SEQ ID NO: 2428, DR4-SP-ECD-hIgFc; SEQ ID NO: 2441, DcR1-SP-ECD-hIgFc; SEQ ID NO: 2448; or DcR2-SP-ECD-hIgFc; SEQ ID NO; 2455); b) a scFV targeting TRAIL, DR5 and/or DR5; c) an shRNA targeting TRAIL, DR5 or DR4; d) a gRNA targeting TRAIL, DR5 or DR4; e) ectopically over-express a DcR1 or DcR2; f) ectopically overexpress a dominant negative mutant of DR5 (e.g., SEQ ID NO: 2370, 2391) or DR4 (e.g., SEQ ID NO: 2275 and 2385); or g) ectopically overexpress a fusion protein containing an extracellular TRAIL-binding domain fused to the transmembrane and cytosolic domain of CD27, CD28, 41BB, OX40, GITR or BCMA (e.g., SEQ ID NO: 2429 to 2440).

The TRAIL antagonist can be administered to the subject prior to, concurrent with or following the administration of cell and/or immune therapy product, e.g., CAR-T cell or Blinatumomab.

In some embodiments, the TRAIL antagonist is administered to prevent the of toxicity of cell or immune therapy products. In other embodiments, the TRAIL antagonist is administered pre-emptively at the early signs of toxicity of cell or immune therapy products. In other embodiments, the TRAIL antagonist is administered after the appearance of signs and symptoms toxicity of cell therapy or immune therapy products. In other embodiments, the TRAIL antagonist is administered to treat the signs and symptoms toxicity of cell- or immune-therapy products. In yet other embodiments, in addition to prevention and treatment of toxicity of cell- or immune-therapy products, the TRAIL antagonist is used to improve their efficacy, e.g., to improve the survival, proliferation and expansion of cell therapy products, to prevent exhaustion, and to improve their long-term persistence and anti-tumor activity in vivo.

The disclosure also relates to methods for prevention and treatment of immune disorders, such as Rheumatoid Arthritis, Systemic-onset juvenile idiopathic arthritis, Still's disease, Macrophage activation syndrome, Hemophagocytic lymphohistiocytosis (HLH), systemic lupus erythematosus (SLE), Kawasaki disease, and inflammatory bowel disease by administration to the subject a TRAIL antagonist, e.g., a TRAIL antagonist described herein, e.g., an agent that interferes with and/or blocks the activity of TRAIL (TNFSF10) or its receptors DR5. In particular, the present disclosure relates to methods for prevention and treatment of immune disorders caused by macrophage activation. In some embodiments, the present disclosure relates to methods for prevention and treatment of immune disorders caused by macrophage activation induced by T cells.

The disclosure also provides compositions and methods that results in the ectopic and/or over-expression of the wild-type or mutant form of one or more genes from the group of CD27 (TNFRSF7, Gene ID: 939; SEQ ID NO: 2254), CD28 (Gene ID: 940; SEQ ID NO: 2255), 41BB (TNFRSF9, CD137; Gene ID: 3604; SEQ ID NO: 2256), OX40 (TNFRSF4, Gene ID: 7293; SEQ ID NO: 2257), DcR2 (TNFRSF10D, Gene ID: 8793; SEQ ID NO: 2251), DcR1 (TNFRSF10C, Gene ID: 8794; SEQ ID NO: 2250), BCMA (TNFRSF17, Gene ID: 608; SEQ ID NO: 2259), and GITR (TNFRSF18; Gene ID: 8784; SEQ ID NO: 2258) and uses of such compositions and methods for modifying the phenotype, differentiation state and functional activities of immune effector cells (e.g., gene-modified antigen-specific T cells, such as CART cells, TCR modified T cells and SIR-T cells).

The disclosure provides compositions and methods that disrupt or downregulate the expression or inhibit the activity of one or more genes from the group of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL (TNFSF10) and Death Receptor 5 (DR5 or TNFRSF10B) and uses of such compositions and methods for modifying the phenotype, differentiation state, functional activities and toxicities of immune effector cells (e.g., gene-modified antigen-specific T cells, such as CART cells).

The disclosure also provides compositions and methods that results in the expression of a mutant form of one or more genes from the group BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, JAK1, JAK3, STAT5b, STAT3, IL2RG, CARD11, BRAF, TRAIL (TNFSF10), Death Receptor 5 (DR5 or TNFRSF10B), or Death Receptor 4 (DR4 or TNFRSF10A) and uses of such compositions and methods for modifying the phenotype, differentiation state, functional activities and toxicities of immune effector cells (e.g., gene-modified antigen-specific T cells, such as CAR-T cells, TCR modified T cells and SIR-T cells).

In particular, the disclosure provides methods and compositions for bolstering the therapeutic efficacy of cell therapy products, e.g, chimeric antigen receptor (CAR) T cells and TCR-modified T cells. While not to be bound by the theory, inhibition of activity or disruption of one or both alleles of one or more of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL (TNFSF10) and Death Receptor 5 (DR5 or TNFRSF10B) genes leads to change in immune cells (e.g., gene-modified antigen-specific T cells, such as CART cells, TCR modified T cells and SIR-T cells) phenotype, differentiation, proliferation, function and/or toxicity.

In particular, the disclosure provides methods and compositions for bolstering the therapeutic efficacy of gene-modified antigen-specific T cells (e.g., chimeric antigen receptor (CAR) T cells and TCR-modified T cells and SIR-T cells etc.) by treatment with chemical inhibitors of EZH2, MLL2, MLL3, MLL4, BRD9, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and/or DR5 when used alone or in combination.

Accordingly, the disclosure provides a cell (e.g., a population of cells, such as population of immune effector cells) engineered to express an antigen specific receptor, such as chimeric antigen receptor (CAR), TCR receptor fusion protein (TFPs), a synthetic immune receptor (SIR), an Antibody-TCR, a natural TCR or an artificial TCR, wherein the antigen specific receptor comprises an antigen-binding domain, a transmembrane domain, and an optional intracellular signaling domain, and wherein expression and/or function of one or more of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1 and HDAC2, JAK1, JAK3, STAT5b, STAT3, IL2RG, CARD11, BRAF, TRAIL and/or DR5 in said cell has been reduced, eliminated or altered.

In some embodiments, the immune cells are collected from a donor who has been administered a CXCR4 antagonist (e.g., Plerixafor), a cytokine (e.g., G-CSF, GM-CSF or sargramostim, Neulasta or Pegfilgastrim), a beta2 agonist (e.g., epinephrine), a tyrosine kinase inhibitor (e.g., dasatinib), chemotherapy drug(s) (e.g. cyclophosphamide, doxorubicin etc) either singly or in combination, prior to the collection of immune cells.

In some embodiments, the donor is an autologous donor while in other embodiments, the donor is an allogeneic donor. In one aspect, the cells of the disclosure are human cells. In one aspect, the cells (e.g., engineered immune effector cells, e.g., CART cells) of the disclosure comprise an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and DR5.

In some embodiments, the cells of the disclosure comprise an immune receptor (e.g., a CAR, SIR, TFP, Ab-TCR or TCR) and an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and/or DR5 wherein said inhibitor is (1) a gene editing system targeted to one or more sites within the gene encoding EZH2, MLL2, MLL3, MLL4, BRD9, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and/or DR5 or its regulatory elements, e.g., BRD9, or its regulatory elements; (2) a gene editing system targeted to one or more sites within the RNA (e.g., mRNA) encoding EZH2, MLL2, MLL3, MLL4, BRD9, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and/or DR5 or its regulatory elements, e.g., BRD9, or its 5′ or 3′ untranslated region or its promoter; 3) nucleic acid encoding one or more components of said gene editing system; or (4) combinations thereof.

In some embodiments, the cells of the disclosure comprise an immune receptor and an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and/or DR5 wherein said inhibitor is a gene editing system targeted to one or more sites within the gene or the RNA encoding BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and/or DR5 or its regulatory elements, e.g., BRD9, or its regulatory elements, and wherein the gene editing system is selected from the group consisting of: a CRISPR/Cas9 system, a zinc finger nuclease system, a TALEN system and a meganuclease system.

In some embodiments, the cells of the disclosure comprise a immune receptor (e.g., a CAR, SIR, TFP, Ab-TCR or TCR), and an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, wherein said inhibitor is a gene editing system targeted to one or more sites within the gene/RNA encoding BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, or its regulatory elements, e.g., BRD9, or its regulatory elements, and wherein the gene editing system is a CRISPR/Cas system comprising a gRNA molecule comprising a targeting sequence which hybridize to a target sequence of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5. In some embodiments, the targeting sequence is a targeting sequence listed in Tables 4a and 4b. In some embodiments, the gRNA molecule has a sequence listed in Table 8.

In some embodiments, the cells of the disclosure comprise an immune receptor (e.g., a CAR, SIR, TFP, Ab-TCR or TCR), and an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, wherein said inhibitor is an siRNA or shRNA specific for BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, or nucleic acid encoding said siRNA or shRNA. In some embodiments, the siRNA or shRNA comprises a sequence complementary to a sequence of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, mRNA, e.g., comprises a target sequence of shRNA listed in Table 5.

In some embodiments, the cells of the disclosure comprise an immune receptor (e.g., a CAR, SIR, TFP, Ab-TCR or TCR), and an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, wherein said inhibitor is a small molecule. In some embodiments, the small molecule is a molecule listed in Table 6.

In some embodiments, the cells of the disclosure comprise an immune receptor (e.g., a CAR, SIR, TFP, Ab-TCR or TCR), and an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, wherein the inhibitor is a protein, e.g., is a dominant negative binding partner of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, or nucleic acid encoding said dominant negative binding partner of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5.

In some embodiments, the cells of the disclosure comprise an immune receptor (e.g., a CAR, SIR, TFP, Ab-TCR or TCR), and an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, wherein the inhibitor is a protein, e.g., is a dominant negative (e.g., catalytically inactive) BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, or nucleic acid encoding said dominant negative BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5. Exemplary dominant negative mutants of DR5 are provided in SEQ ID NO: 2260 and 2318 to 2330.

In one aspect, the disclosure provides a method of altering the phenotype, differentiation state and/or the therapeutic efficacy of an immune cell, e.g., an immune receptor-expressing cell, e.g., a cell of any of the previous claims, e.g., a CAR19-expressing cell, comprising a step of decreasing the level or activity of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, in said cell. In some embodiments, said step comprises contacting said cells with an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5. In some embodiments, a BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, inhibitor of the disclosure is selected from the group consisting of: (1) a gene editing system targeted to one or more sites within the gene/RNA encoding BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, or its corresponding regulatory elements; (2) a nucleic acid (e.g., an siRNA or shRNA) that inhibits expression of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5; (3) a protein (e.g., a dominant negative, e.g., catalytically inactive) BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, or a binding partner of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5; (4) a small molecule that inhibits expression and/or function of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5; (5) a nucleic acid encoding any of (1)-(3); and (6) any combination of (1)-(5).

In one aspect, the disclosure provides a method of altering the phenotype, differentiation state and/or the therapeutic efficacy of an immune cell, e.g., immune receptor-expressing cell, e.g., a cell of any of the previous claims, e.g., a CAR19-expressing cell, comprising a step of decreasing the level or activity of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 in said cell. In some embodiments, said step comprises contacting said cells with an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5. In some embodiments, said contacting occurs ex vivo. In some embodiments, said contacting occurs in vivo. In some embodiments, said contacting occurs in vivo prior to delivery of nucleic acid encoding an immune receptor into the cell. In some embodiments, said contacting occurs in vivo after the cells have been administered to a subject in need thereof.

In one aspect, the disclosure provides a method of increasing the diversity of immune cells, e.g., immune receptor-expressing cells, e.g., cells of any of the previous claims, e.g., CAR19-expressing cells, comprising a step of decreasing the level or activity of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, in said cells. In some embodiments, said step comprises contacting said cells with an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5. In some embodiments, said contacting occurs ex vivo. In some embodiments, said contacting occurs in vivo. In some embodiments, said contacting occurs in vivo prior to delivery of nucleic acid encoding an immune receptor into the cell. In some embodiments, said contacting occurs in vivo after the cells have been administered to a subject in need thereof.

In one aspects, the disclosure provides a method of treating a subject in need thereof, comprising administering to said subject an effective amount of the cells as described herein, e.g., an immune effector cell (e.g., T cell or NK cell) comprising an immune receptor, and, optionally, administering to said subject an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5. In some embodiments, the subject receives a pre-treatment with an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 prior to the initiation of immune- or cell-therapy. In some embodiments, the subject receives concurrent treatment with an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 and the immune- and/or cell-therapy. In some embodiments, the subject receives treatment with an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 following the administration of immune- and/or cell-therapy.

In some embodiments, the subject has a disease associated with expression of a tumor antigen, e.g., a proliferative disease, a precancerous condition, a cancer, and a non-cancer related indication associated with expression of the tumor antigen.

The disclosure provides uses of the compositions and/or methods described here for treatment of cancer.

The disclosure provides inhibitors of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 and mutants of JAK1, JAK3, STAT5b, STAT3, IL2RG, CARD11, and/or BRAF, and a wild-type and mutant of CD28 (Gene ID: 940), 41BB (TNFRSF9, CD137; Gene ID: 3604), OX40 (TNFRSF4, Gene ID: 7293), DcR2 (TNFRSF10D, Gene ID: 8793), DcR1 (TNFRSF10C, Gene ID: 8794), BCMA (TNFRSF17, Gene ID: 608), and GITR (TNFRSF18; Gene ID: 8784) for use in the treatment of a subject, wherein said subject has received, is receiving, or is about to receive immune- and/or cell-therapy.

The disclosure further provides a method of manufacturing immune cells, e.g., immune effector cells, comprising introducing nucleic acid encoding an immune receptor into immune effector cells such that said nucleic acid (or immune receptor-encoding portion thereof) integrates into the genome of the cells within a BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 gene (e.g., within an intron or exon of a BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 gene), such that BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, expression and/or function is reduced or eliminated.

The disclosure further provides a method of manufacturing immune cells, e.g., immune effector-cells for adoptive cellular therapy, comprising contacting said immune cells ex vivo with a BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, inhibitor either singly or in combination. In some embodiments, the inhibitor is a BRD9 inhibitor. In some embodiments, the inhibitor is a TRAIL inhibitor.

The disclosure further provides a method of manufacturing immune cells, e.g., immune effector cells, e.g., for adoptive cellular therapy, comprising administrating to the donor from whom the immune cells are harvested a CXCR antagonist, such as Mozibil (Plerixafor), a cytokine (e.g., G-CSF or GM-CSF), a beta2 agonist (e.g., epinephrine), a tyrosine kinase inhibitor (e.g, a Src kinase inhibitor, e.g., Dasatinib), chemotherapy drugs (e.g., cyclophosphamide, doxorubicin etc.) either alone or in combination prior to the collection (i.e. leukophersis) of immune cells for manufacturing. The disclosure further provides a method of manufacturing immune cells for adoptive cellular therapy, comprising subjecting the donor from whom the immune cells are harvested to exercise so as to increase the heart rate to about 30% (e.g., 40%, 50%, 60%, 80%, 100%, 125%, 150%) higher than the donor's heart rate at rest.

The disclosure further provides a vector comprising sequence encoding an immune receptor and sequence encoding an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5. In some embodiments, the inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 is a (1) a gene editing system targeted to one or more sites within the gene/RNA encoding BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, or its corresponding regulatory elements; (2) a nucleic acid (e.g., an siRNA or shRNA) that inhibits expression of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5; (3) a protein (e.g., a dominant negative, e.g., catalytically inactive) BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, or a binding partner of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5; and (4) a nucleic acid encoding any of (1)-(3), or combinations thereof. In some embodiments, the sequence encoding an immune receptor and the sequence encoding an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 are separated by a 2A site.

The disclosure further provides a gene editing system that is specific for a sequence of the BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 gene or its regulatory elements, e.g., a BRD9, BCOR, PRDM1, TRAIL gene or its regulatory elements. In some embodiments, the gene editing system is specific for a sequence of a BRD9 gene or its RNA. In some embodiments, the gene editing system is (1) a CRISPR/Cas gene editing system, (2) a zinc finger nuclease system, a TALEN system and a meganuclease system. In some embodiments, the gene editing system is a CRISPR/Cas gene editing system. In some embodiments, the gene editing system comprises: a gRNA molecule comprising a targeting sequence specific to BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 gene or its regulatory elements, and a Cas9 protein; a gRNA molecule comprising a targeting sequence specific to BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 gene or its regulatory elements, and a nucleic acid encoding a Cas9 protein; a nucleic acid encoding a gRNA molecule comprising a targeting sequence specific to BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 gene or its regulatory elements, and a Cas9 protein; or a nucleic acid encoding a gRNA molecule comprising a targeting sequence specific to BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 gene or its regulatory elements, and a nucleic acid encoding a Cas9 protein. In some embodiments, the gene editing system further comprises a template DNA. In some embodiments, the template DNA comprises nucleic acid sequence encoding an immune receptor, e.g., an immune receptor as described herein.

In some embodiments, the present disclosure also provides a method of treatment or prevention of disease in a subject with an immune cell therapy comprising the steps of i) checking the expression of the ligands of costimulatory receptors in the disease-causing or disease-associated cells, e.g., cancer cells or stromal cells; ii) identifying the ligands of the costimulatory receptors that are expressed in the disease-causing or disease-associated cells, e.g., cancer cells or stromal cells; iii) enhancing the expression in the immune cells of one or more of the costimulatory receptors whose ligands are expressed in the disease-causing or disease-associated cells; iv) administering to the subject the immune cells from step (iii) to prevent or treat a disease. In some embodiments, the expression of the ligands in the disease-causing or disease-associated cells or tissues is examined at the mRNA level. Methods to examine the expression of a gene at the mRNA are known in the art and include, but are not limited, to RT-PCR, northern blot, RNA-Seq, and gene expression microarrays etc. In some embodiments, the expression of the ligands in the disease-causing or disease-associated cells or tissues is examined at the protein level. Methods to examine the expression of a protein are known in the art and include, but are not limited, to immunohistochemistry, flow cytometry, tissue micro-arrays, immunoblotting and mass spectroscopy etc. In some embodiments, the ligand consists of one or more of, but is not limited to, TRAIL, CD70, CD80, CD86, 41BBL, OX40L, GITRL and TNFSF13B/TALL-1/BAFF. In some embodiments, the costimulatory receptor consists of or comprises of one or more of, but is not limited to, a receptor containing the extracellular ligand binding domain of DcR1, DcR2, DR4, DR5, CD27, CD28, 41BB, OX40, GITR and BCMA. In some embodiments, the costimulatory receptor consists of or comprises of one or more of, but is not limited to DcR1, DcR2, CD27, CD28, 41BB, OX40, GITR and BCMA. In some embodiments, the costimulatory receptor consists of or comprises of one or more of, but is not limited to, a receptor containing the extracellular ligand binding domain of DcR1, DcR2, DR4, DR5, CD27, CD28, 41BB, OX40, GITR and BCMA fused via a transmembrane domain to the cytosolic domain of a costimulatory receptor, e.g., CD27, CD28, 41BB, OX40, GITR and BCMA. Table 1 provides the SEQ ID NO of several exemplary fusion proteins containing the extracellular domain of DR5, DR4, DcR1 and DcR2 fused to the cytosolic domain of CD27, CD28, 41BB, OX40, GITR and BCMA. In some embodiments, the immune cell is a T cell, e.g., a CAR-T cell or a TCR-T cell or a TIL. In an exemplary embodiment, the CAR-T cells express a CAR targeting CD19 and coexpress CD27, where the disease-causing and/or disease-associated cells express CD19 and CD70. In another exemplary embodiment, the CAR-T cells express a CAR targeting Mesothelin and coexpress DcR1, where the disease-causing and/or disease-associated cells express Mesothelin and TRAIL. In another exemplary embodiment, the CAR-T cells express a CAR targeting Her2 and coexpress 41BB, where the disease-causing and/or disease-associated cells express Her2 and 41BBL. In another exemplary embodiment, the T cells express a TCR targeting NYESO-1/HLA-A2 complex and coexpress OX40, where the disease-causing and/or disease-associated cells express NYESO-1/HLA-A2 and OX40L. In an exemplary embodiment, the NK-T cells express a CAR targeting CD19 and coexpress CD27, where the disease-causing and/or disease-associated cells express CD19 and CD70.

The disclosure further provides a composition for the ex vivo manufacture of immune cells, e.g., immune effector cells, such as an immune receptor expressing cells (e.g., CAR-T, SIR-T, TCR-T cells etc) comprising a BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and/or DR5 inhibitor, e.g., a BRD9, CBFb, and/or YY1 inhibitor. The inhibitors can be used singly or in combination.

The disclosure further provides a composition for the ex vivo manufacture of a diverse pool of immune effector cells, such as an immune receptor expressing cells (e.g., CAR-T, SIR-T, TCR-T cells etc) comprising a BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and/or DR5 inhibitor, e.g., a BRD9, CBFb, and/or YY1 inhibitor. The inhibitors can be used singly or in combination.

Table 6 presents the targets, names and Cas number of various exemplary chemical inhibitors targeting different genes that can be used in the methods of the disclosure.

The disclosure further provides a population of cells comprising one or more cells described herein, wherein the population of cells comprises a higher percentage of stem like T cells or Tscm cells (e.g., CD45RA+CD62L+CCR7+CD27+CD95+ T cells) than a population of cells which does not comprise one or more cells in which expression and/or function of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and/or DR5 in said cell has been reduced or eliminated.

The present disclosure also pertains, at least in part, to methods for improving the expansion and/or activation (e.g., in vitro and in vivo expansion and/or activation) of immune cells, e.g., immune effector T cells, e.g., CAR-T cells or TCR-T cells or TILs, for the purpose of adoptive cellular therapy. Some embodiments described herein provide for expansion and/or activation of immune cells by exposing them to a bispecific or multi-specific engager that contains at least one antigen binding domain, e.g., first antigen binding domain, capable of engaging the immune cells and at least one antigen binding domain, e.g., a second antigen binding domain, capable of engaging an antigen presenting cell (APC) or antigen presenting substrate (APS), e.g. antigen-conjugated beads. In some embodiment, the APC is a hematopoietic cell. In some embodiments, the bispecific engager is a bispecific antibody, e.g., Blinatumomab. In some embodiments, the method further involves exposing the immune cells to an agonist, such as an antibody (e.g., Utomilumab) or a ligand (e.g., 41BBL), capable of activating a costimulatory receptor (e.g., CD28, 41BB, CD27 etc.) on immune cells, e.g., T cells or NK cells.

In some embodiment, the bispecific or multispecific engager comprises at least one binding domain capable of binding to and activating the T cell receptor (TCR) complex of T cells. In some embodiment, the bispecific or multispecific engager comprises at least one binding domain capable of binding to and activating the CD3 subunit of the TCR complex. In some embodiments, the bispecific or multispecific engager comprises at least one binding domain capable of binding to and activating the CD3-epsilon subunit of the TCR complex. In some embodiments, the bispecific or multispecific engager comprises at least one binding domain capable of binding to and activating a receptor on the T cells that provides co-stimulation; i.e., a co-stimulatory receptor. Exemplary co-stimulatory receptor that can be bound by the bispecific engager include CD28, CD137 (4-1BB), CD134 (OX40), Dap10, CD27, CD2, CD5, ICAM-1, LFA-1, TNFR-I, TNFR-II, Fas, CD30 and CD40. In some embodiment, the bispecific or multispecific engager in the presence of APC or APS activates the signaling through the TCR complex. In some embodiment, the bispecific or multispecific engager in the presence of APC or APS activates T cells via signaling through a co-stimulatory receptor.

In some embodiment, the bispecific or multispecific engager comprises at least one binding domain capable of binding to the hematopoietic cells. In some embodiment, the bispecific or multispecific engager comprises at least one binding domain capable of binding to the lymphoid-lineage hematopoietic cells. In some embodiment, the bispecific or multispecific engager comprises at least one binding domain capable of binding to the B-lymphoid-lineage hematopoietic cells. Exemplary B-lineage lymphoid cells bound by the bispecific or multispecific engager include immature B cells, mature B cells and plasma cells and combination thereof. In some embodiment, the bispecific or multispecific engager comprises at least one binding domain, e.g., second binding domain, capable of binding to an antigen expressed on B-lymphoid-lineage hematopoietic cells. In some embodiments, the at least one binding domain of the bispecific or multispecific engager binds to CD3e (or CD3ε) and at least one binding domain, e.g., the second antigen binding domain, binds to CD19. In some embodiments, the bispecific engager is Blinatumomab. In some embodiments, the at least one binding domain of the bispecific or multispecific engager binds to CD3e (or CD3ε) and at least one binding domain, e.g., the second antigen binding domain, binds to CD22. In some embodiments, the at least one binding domain of the bispecific or multispecific engager binds to CD3e (or CD3ε) and at least one binding domain, e.g., the second antigen binding domain, binds to CD20/MS4A1. In some embodiments, the at least one binding domain of the bispecific or multispecific engager binds to CD3e (or CD3ε) and at least one binding domain, e.g., the second antigen binding domain, binds to CD23. In some embodiments, the at least one binding domain of the bispecific or multispecific engager binds to CD3e (or CD3ε) and at least one binding domain, e.g., the second antigen binding domain, binds to BCMA. In some embodiments, the bispecific engager is BI 836909 (AMG 420). In some embodiments, the at least one binding domain of the bispecific or multispecific engager binds to CD3e (or CD3ε) and at least one binding domain, e.g., the second antigen binding domain, binds to CS1/SLAMF7. In some embodiments, the at least one binding domain of the bispecific or multispecific engager binds to CD3e (or CD3ε) and at least one binding domain, e.g., the second antigen binding domain, binds to CD138. In some embodiments, the at least one binding domain of the bispecific or multispecific engager binds to CD3e (or CD3ε) and at least one binding domain, e.g., the second antigen binding domain, binds to CD123. In some embodiments, the at least one binding domain of the bispecific or multispecific engager binds to CD3e (or CD3ε) and at least one binding domain, e.g., the second antigen binding domain, binds to MPL.

In some embodiment, the bispecific or multispecific engager comprises at one binding domain capable of binding to an activating receptor (e.g., CD3) and/or costimulatory receptor (e.g., CD28, 41BB etc) on T cells and at least one binding domain capable of binding to the non-hematopoietic cells (e.g, breast cells, lung cells, colon cells, skin cells etc.) or cell lines (e.g., MCF7, H460, SW480 etc.).

In some embodiment, the bispecific or multispecific engager comprises at least one binding domain capable of binding to and activating a NK cell receptor and at least one binding domain capable of binding to an antigen expressed on APC or APS.

In some embodiments, the activation and expansion of T cells involves exposing them to a bispecific or multispecific engager (e.g., antibody) that binds to a T cell activating- or costimulatory receptor (e.g., TCR or CD28) in the presence of a cell expressing a cognate ligand (e.g. an antigen) bound by the second antigen binding domain of the bispecific or multispecific engager (e.g., antibody).

In some embodiments, the activation and expansion of T cells involves exposing them to a bispecific or multispecific engager (e.g., antibody) that binds to a T cell activating- or costimulatory receptor (e.g., TCR or CD28) in the presence of a solid substrate expressing a cognate ligand (e.g. an antigen or anti-idiotype antibody) bound by the second antigen binding domain of bispecific or multispecific engager (e.g., antibody).

In some embodiments, the method involves activation/expansion of immune cells (e.g., T cells) by exposing them to two different bispecific or multi-specific engagers where the at least one antigen binding domain of the first bispecific or multispecific engager binds to and activates an activating cell receptor (e.g., T cell receptor or CD3 receptor complex) and at least one antigen binding domain of the second bispecific or multispecific engager binds to and activates a costimulatory receptor (e.g., 41BB or CD28) and at least one antigen binding domain of the two bispecific or multispecific engagers binds to at least one antigen (e.g., CD19, CD22, CD20/MS4A1 and/or BCMA, etc.) expressed on APC, e.g., hematopoietic cells (e.g., B cells, plasma cells or lymphoma cell lines etc.) or non-hematopoietic cells (e.g., breast cells, lung cancer cell line etc.) or APS (e.g, CD19-ectodomain coated beads or CD19 anti-ideotype antibody coated beads).

In some embodiments, the population of immune cells used in the methods described herein is acquired, e.g., obtained, from a blood sample from a subject (e.g., a cancer patient). In one embodiment, the population of immune cells is obtained by apheresis. In one embodiment, the population of immune cells is obtained by apheresis from a subject who has been made to exercise or administered a CXCR antagonist (e.g., Mozibil or Plerixafor), a cytokine (e.g., G-CSF, GM-CSF or sargramostim, Neulasta or Pegfilgastrim), a beta2 adrenergic agonist (e.g., epinephrine), a tyrosine kinase inhibitor (e.g., dasatinib), chemotherapy drug(s) (e.g., cyclophosphamide, doxorubicin) or a combination of the above agents prior to the collection of immune cells.

In some embodiments, the immune cell population includes immune effector cells, e.g., as described herein. Exemplary immune effector cells include T cells, e.g., alpha/beta T cells and gamma/delta T cells, natural killer T (NKT) cells, or a combination thereof.

In some embodiments, the immune cell population includes peripheral blood mononucleated cells (PBMCs), or cord blood cells, tissue resident lymphocytes, tumor infilterating lymphocytes, bone marrow resident mono-nuclear cells or a combination thereof.

In certain embodiments, the immune cell population includes primary T cells or subsets of lymphocytes, including, for example, anergized T cells, naive T cells, T-regulatory cells, Th-17 cells, stem T cells, tissue-resident T cells, tumor infilterating T cells or a combination thereof.

In certain embodiments, the immune cell population includes T cells that have been engineered to express a natural or a synthetic receptor targeting a specific antigen. An exemplary natural receptor includes a T cell receptor (TCR) targeting NY-ESO1 or WTI. Exemplary synthetic receptors include a CAR or a next generation CARs (e.g., K13-CAR, SIR, zSIR, Ab-TCR, TFP etc.) or a recombinant TCR (rTCR).

The disclosure further relates to novel nucleic acid constructs and their incorporation into immune cells. Particularly, the disclosure relates to the discovery that immune cells, preferably T cells, e.g., primary human T cells, which are optionally engineered to express at least one immune receptor (e.g., CAR, SIR, AbTCR, TFP, TCR etc) and one or more signaling molecules, e.g., JAK1, JAK3, STAT5b, STAT3, IL2RG, CARD11, and/or BRAF or mutated forms thereof, possess properties which render these cells well suited for use in human or animal therapy. In some embodiments, the nucleic acid construct or constructs encoding the immune receptor and the signaling molecule or variant thereof may be on the same or different vectors. In some embodiments, the signaling molecule or variant thereof is expressed in the immune effector cell that expresses an endogenous immune receptor (e.g., TCR). In some embodiments, the signaling molecule or variant thereof is expressed in the immune effector cell that expresses an exogenous (or engineered) immune receptor (e.g., CAR, SIR, AbTCR, TFP or cTCR etc.). In some embodiments, the signaling molecule or variant thereof is expressed in the immune effector cell transiently. In some embodiments, the signaling molecule or variant thereof is expressed in the immune effector cell stably.

TABLE 1

| | | | | SEQ ID- | SEQ ID- |
|---|---|---|---|---|---|
| NAME | SEQ ID-DNA | SEQ ID-PRT | NAME | DNA | PRT |
| JAK3 | 360 | 1790 | GITR-SP | 2285 | 2395 |
| JAK3-M511I | 361 | 1791 | GITR-SP-ECD | 2286 | 2396 |
| JAK3-A573V | 362 | 1792 | GITR-SP-ECD-TM | 2287 | 2397 |
| JAK3-R657W | 363 | 1793 | GITR-ECD | 2288 | 2398 |
| JAK3-Q507P | 364 | 1794 | GITR-TM | 2289 | 2399 |
| JAK3-G491S | 365 | 1795 | GITR-CP | 2290 | 2400 |
| JAK3-V674A | 366 | 1796 | OX40-SP | 2291 | 2401 |
| JAK3-V678L | 367 | 1797 | OX40-SP-ECD | 2292 | 2402 |
| Stat5b | 368 | 1798 | OX40-SP-ECD-TM | 2293 | 2403 |
| Stat5b-T628S | 369 | 1799 | OX40-ECD | 2294 | 2404 |
| Stat5b-N642H | 370 | 1800 | OX40-TM | 2295 | 2405 |
| Stat5b-Y665F | 371 | 1801 | OX40-CP | 2296 | 2406 |
| Stat5b-Q706L | 372 | 1802 | CD27-SP | 2297 | 2407 |
| Stat5b-R659C | 373 | 1803 | CD27-SP-ECD | 2298 | 2408 |
| JAK1 | 374 | 1804 | CD27-SP-ECD-TM | 2299 | 2409 |
| JAK1-V658F | 375 | 1805 | CD27-ECD | 2300 | 2410 |
| JAK1-S7031 | 376 | 1806 | CD27-TM | 2301 | 2411 |
| CARD11 | 377 | 1807 | CD27-CP | 2302 | 2412 |
| CARD11-S615F | 378 | 1808 | 41BB-SP | 2303 | 2413 |
| CARD11-E626K | 379 | 1809 | 41BB-SP-ECD | 2304 | 2414 |
| STAT3 | 380 | 1810 | 41BB-SP-ECD-TM | 2305 | 2415 |
| STAT3-D661Y | 381 | 1811 | 41BB-ECD | 2306 | 2416 |
| STAT3-Y640F | 382 | 1812 | 41BB-TM | 2307 | 2417 |
| STAT3-A702T | 383 | 1813 | 41BB-CP | 2308 | 2418 |
| STAT3-S614R | 384 | 1814 | CD28-SP | 2309 | 2419 |
| STAT3-G618R | 385 | 1815 | CD28-SP-ECD | 2310 | 2420 |
| IL2RG | 386 | 1816 | CD28-SP-ECD-TM | 2311 | 2421 |
| IL2RG-K315E | 387 | 1817 | CD28-ECD | 2312 | 2422 |
| BRAF | 388 | 1818 | CD28-TM | 2313 | 2423 |
| BRAF-V600E | 389 | 1819 | CD28-CP | 2314 | 2424 |
| DcR1-(TNFRSF10C) | 2250 | 2360 | BCMA-ECD | 2315 | 2425 |
| DcR2-(TNFRSF10D) | 2251 | 2361 | BCMA-TM | 2316 | 2426 |
| CD27 | 2254 | 2364 | BCMA-CP | 2317 | 2427 |
| CD28 | 2255 | 2365 | DcR2-SP | 2267 | 2377 |
| 41BB | 2256 | 2366 | DcR2-SP-ECD | 2268 | 2378 |
| OX40 | 2257 | 2367 | DcR2-SP-ECD-TM | 2269 | 2379 |
| GITR-(TNFRSF18) | 2258 | 2368 | DcR2-ECD | 2270 | 2380 |
| BCMA | 2259 | 2369 | DcR2-TM | 2271 | 2381 |
| DR5-L363N | 2260 | 2370 | DcR2-CP | 2272 | 2382 |
| DcR1-SP | 2262 | 2372 | DR4-SP | 2273 | 2383 |
| DcRl-SP-ECD | 2263 | 2373 | DR4-SP-ECD | 2274 | 2384 |
| DcRl-SP-ECD-TM | 2264 | 2374 | DR4-SP-ECD-TM | 2275 | 2385 |
| DcR1-ECD | 2265 | 2375 | DR4-ECD | 2276 | 2386 |
| DcR1-TM | 2266 | 2376 | DR4-TM | 2277 | 2387 |
| DR4-CP | 2278 | 2388 | FADD-DN | 2353 | 2463 |
| DR5-SP | 2279 | 2389 | Caspase-8-D73A | 2354 | 2464 |
| DR5-SP-ECD | 2280 | 2390 | Caspase-8-D73A- | 2355 | 2465 |
| | | | L74A | | |
| DR5-SP-ECD-TM | 2281 | 2391 | Caspase-8-D73A- | 2356 | 2466 |
| | | | L74A-L75A | | |
| DR5-ECD | 2282 | 2392 | DR5-CP | 2284 | 2394 |
| DR5-TM | 2283 | 2393 | DcRl-SP-ECD- | 2338 | 2448 |
| | | | hIgFc | | |
| DR5-SP-ECD-hIgFc | 2318 | 2428 | DcRl-SP-ECD- | 2339 | 2449 |
| | | | CD27-TM-CP | | |
| DR5-SP-ECD-TM-CD27- | 2319 | 2429 | DcRl-SP-ECD- | 2340 | 2450 |
| CP | | | CD28-TM-CP | | |
| DR5-SP-ECD-TM-CD28- | 2320 | 2430 | DcRl-SP-ECD- | 2341 | 2451 |
| CP | | | 41BB-TM-CP | | |
| DR5-SP-ECD-TM-41BB- | 2321 | 2431 | DcRl-SP-ECD- | 2342 | 2452 |
| CP | | | OX40-TM-CP | | |
| DR5-SP-ECD-TM- | 2322 | 2432 | DcRl-SP-ECD- | 2343 | 2453 |
| OX40-CP | | | GITR-TM-CP | | |
| DR5-SP-ECD-TM-GITR- | 2323 | 2433 | DcRl-SP-ECD- | 2344 | 2454 |
| CP | | | BCMA-TM-CP | | |
| DR5-SP-ECD-TM- | 2324 | 2434 | DcR2-SP-ECD- | 2345 | 2455 |
| BCMA-CP | | | hIgFc | | |
| DR5-SP-ECD-CD27-TM- | 2325 | 2435 | DcR2-SP-ECD- | 2346 | 2456 |
| CP | | | CD27-TM-CP | | |
| DR5-SP-ECD-CD28-TM- | 2326 | 2436 | DcR2-SP-ECD- | 2347 | 2457 |
| CP | | | CD28-TM-CP | | |
| DR5-SP-ECD-41BB-TM- | 2327 | 2437 | DcR2-SP-ECD- | 2348 | 2458 |
| CP | | | 41BB-TM-CP | | |
| DR5-SP-ECD-OX40- | 2328 | 2438 | DcR2-SP-ECD- | 2349 | 2459 |
| TM-CP | | | OX40-TM-CP | | |
| DR5-SP-ECD-GITR-TM- | 2329 | 2439 | DcR2-SP-ECD- | 2350 | 2460 |
| CP | | | GITR-TM-CP | | |
| DR5-SP-ECD-BCMA- | 2330 | 2440 | DcR2-SP-ECD- | 2351 | 2461 |
| TM-CP | | | BCMA-TM-CP | | |
| DR4-SP-ECD-hIgFc | 2331 | 2441 | DR4-SP-ECD- | 2334 | 2444 |
| | | | 41BB-TM-CP | | |
| DR4-SP-ECD-CD27-TM- | 2332 | 2442 | DR4-SP-ECD- | 2335 | 2445 |
| CP | | | OX40-TM-CP | | |
| DR4-SP-ECD-CD28-TM- | 2333 | 2443 | DR4-SP-ECD- | 2336 | 2446 |
| CP | | | GITR-TM-CP | | |
| | | | DR4-SP-ECD- | 2337 | 2447 |
| | | | BCMA-TM-CP | | |

TABLE 2

| | DNA | PRT |
|---|---|---|
| NAME | SEQ ID | SEQ ID |
| BRD9-transcript | 391 | 1821 |
| variant 1 | | |
| BRD9-variant 2 | 392 | 1822 |
| BRD9-transcript | 393 | 1823 |
| variant2-Q479H | | |
| EZH2 | 394 | 1824 |
| FBXW10 | 395 | 1825 |
| FBXW10-D318N | 396 | 1826 |
| CHEK2 | 397 | 1827 |
| BCOR | 398 | 1828 |
| BCOR-V896L | 399 | 1829 |
| BCOR-A165P | 400 | 1830 |
| ASXL1 | 401 | 1831 |
| PHF6 | 402 | 1832 |
| SF3B1 | 403 | 1833 |

### BRIEF DESCRIPTION OF THE DRAWINGS

FIG. 1 depicts Matador Cytotoxicity assay with different CD19 CAR-T cell products generated using Perixafor mobilized blood cells on RAJI cells stably expressing LucPPe.

FIG. 2 depicts Matador Cytotoxicity assay with different CD19 CAR-T cell products generated using Perixafor mobilized blood cells on Nalm6 cells stably expressing LucPPe.

FIG. 3 depicts the effect of TRAIL antibody on PMA-induced IL1α production.

FIG. 4 depicts TRAIL antibody blocks IL1α production by THP1 cells when co-cultured with CD19 CAR-T cells in the presence of CD19-expressing BV173 cells.

FIG. 5 depicts TRAIL antibody blocks IL1α production by PMA-differentiated THP1 cells when co-cultured with CD19 CAR-T cells in the presence of CD19-expressing BV173 cells.

### DETAILED DESCRIPTION

Unless defined otherwise, all technical and scientific terms used herein have the same meaning as commonly understood by one of ordinary skill in the art to which the disclosure pertains.

The term “a”, “an” and “the” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some aspects, the set of polypeptides are contiguous with each other. In some embodiments, the set of polypeptides include a dimerization switch that, upon the presence of a dimerization molecule, can couple the polypeptides to one another, e.g., can couple an antigen binding domain to an intracellular signaling domain. In one aspect, the stimulatory molecule is the zeta chain associated with the T cell receptor complex. In one aspect, the cytoplasmic signaling domain further comprises one or more functional signaling domains derived from at least one costimulatory molecule as defined below. In one aspect, the costimulatory molecule is chosen from the costimulatory molecules described herein, e.g., 41BB or 4-1BB (i.e., CD137), CD27, GITR, OX40, BCMA and/or CD28. In one aspect, the CAR comprises a chimeric fusion protein comprising an extracellular antigen binding domain, a transmembrane domain and an intracellular signaling domain comprising a functional signaling domain derived from a stimulatory molecule. In one aspect, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In one aspect, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In one aspect, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In one aspect, the CAR comprises an optional leader sequence at the amino-terminus (N-ter) of the CAR fusion protein. In one aspect, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane. As used herein, the term “CAR” or “CARs” also encompasses newer approaches to conferring antigen specificity onto cells, including but not limited to Antibody-TCR chimeric molecules or AbTCR (WO 2017/070608 A1), TCR receptor fusion proteins or TFP (WO 2016/187349 A1), chimeric T cell receptors or cTCR and Synthetic Immune Recptors (PCT/US17/64379).

A CAR that comprises an antigen binding domain (e.g., a scFv, or TCR) that targets a specific tumor maker X, such as those described herein, is also referred to as XCAR. For example, a CAR that comprises an antigen binding domain that targets CD19 is referred to as CD19CAR.

The term “signaling domain” refers to the functional portion of a protein which acts by transmitting information within the cell to regulate cellular activity via defined signaling pathways by generating second messengers or functioning as effectors by responding to such messengers.

The term “costimulatory ligand” or “ligand of a costimulatory receptor” refers to a protein or polypeptide that binds to a co-stimulatory receptor, e.g., a costimulatory receptor expressed herein, e.g., DcR1, DcR2, CD27, CD28, 41BB, OX40, GITR, BCMA. Exemplary costimulatory ligands include CD70, CD80, CD86, 41BBL, OX40L, GITRL, BAFF and TRAIL. Binding of the costimulatory ligand to its cognate receptor on immune cells may lead to initiation of a signal transduction pathway that promotes the proliferation, activation, cytokine secretion and/or differentiation of the immune cells. Some costimulatory ligands, such as TRAIL, may bind to more than one receptor. Thus, TRAIL binds to at least four receptors, DcR1, DcR2, DR4 and DR5. Binding of TRAIL to DcR2 is known to activate NF-κB pathway without inducing cell death. In contrast, binding of TRAIL to DR4 and DR5 is known to activate NF-κB as well as cell death depending on cellular context.

The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an antibody, that retains the ability to specifically interact with (e.g., by binding, steric hinderance, stabilizing/destabilizing, spatial distribution) an epitope of an antigen. Examples of antibody fragments include, but are not limited to, Fab, Fab′, F(ab′h, Fv fragments, scFv antibody fragments, disulfide-linked Fvs (sdFv), a Fd fragment consisting of the VH and CHI domains, linear antibodies, single domain antibodies such as sdAb (either VL or VH), camelid VHH domains, multi-specific antibodies formed from antibody fragments such as a bivalent fragment comprising two Fab fragments linked by a disulfide brudge at the hinge region, and an isolated CDR or other epitope binding fragments of an antibody.

The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

As used herein, the term “binding domain” or “antibody molecule” refers to a protein, e.g., an immunoglobulin chain or fragment thereof, comprising at least one immunoglobulin variable domain sequence. The term “binding domain” or “antibody molecule” encompasses antibodies and antibody fragments. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (K) and lambda (A) light chains refer to the two major antibody light chain isotypes.

The term “recombinant antibody” refers to an antibody which is generated using recombinant DNA technology, such as, for example, an antibody expressed by a bacteriophage or yeast expression system.

The term “antigen” or “Ag” refers to a molecule that provokes an immune response.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place. The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The term “autologous” refers to any material derived from the same individual to whom it is later to be re-introduced into the individual.

The term “allogeneic” refers to any material derived from a different animal of the same species as the individual to whom the material is introduced

The term “cancer” refers to a disease characterized by the uncontrolled growth of aberrant cells.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connotate or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The phrase “disease associated with expression of a tumor antigen as described herein” includes, but is not limited to, a disease associated with expression of a tumor antigen as described herein or condition associated with cells which express a tumor antigen as described herein including, e.g., proliferative diseases such as a cancer or malignancy or a precancerous condition such as a myelodysplasia, a myelodysplastic syndrome or a preleukemia; or a noncancer related indication associated with cells which express a tumor antigen as described herein. Non-cancer related indications associated with expression of a tumor antigen as described herein include, but are not limited to, e.g., autoimmune disease, (e.g., lupus), inflammatory disorders (allergy and asthma) and transplantation. In some embodiments, the tumor antigen-expressing cells express, or at any time expressed, mRNA encoding the tumor antigen. In an embodiment, the tumor antigen-expressing cells produce the tumor antigen protein (e.g., wild-type or mutant), and the tumor antigen protein may be present at normal levels or reduced levels. In an embodiment, the tumor antigen-expressing cells produced detectable levels of a tumor antigen protein at one point, and subsequently produced substantially no detectable tumor antigen protein.

The term “conservative sequence modifications” refers to amino acid modifications that do not significantly affect or alter the binding characteristics of the antibody or antibody fragment containing the amino acid sequence.

The term “stimulation,” refers to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules.

The term “stimulatory molecule,” refers to a molecule expressed by aan immune cell (e.g., T cell, NK cell, B cell) that provides the cytoplasmic signaling sequence(s) that regulate activation of the immune cell in a stimulatory way for at least some aspect of the immune cell signaling pathway. In one aspect, the signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. In a specific CAR of the disclosure, the intracellular signaling domain in any one or more CARS of the disclosure comprises an intracellular signaling sequence, e.g., a primary signaling sequence of CD3-zeta. In a specific CAR of the disclosure, the primary signaling sequence of CD3-zeta is the sequence provided as SEQ ID NO (DNA): 431 and SEQ ID NO (PRT): 1858, or the equivalent residues from a non-human species, e.g., mouse, rodent, monkey, ape and the like. In a specific CAR of the disclosure, the primary signaling sequence of CD3-zeta is the sequence as provided in SEQ ID NO (DNA): 432 and SEQ ID NO (PRT): 1859, or the equivalent residues from a non-human species, e.g., mouse, rodent, monkey, ape and the like.

The term “antigen presenting cell” or “APC” refers to any cell such as an accessory cell (e.g., a B-cell, a dendritic cell, and the like) or cell line (e.g., cancer cell line, e.g., breast cancer cell line, e.g, MCF7 cell line) that presents an antigen that can be recognized by an immune cell. An APC may display a foreign antigen complexed with major histocompatibility complexes (MHC's) on its surface. T-cells may recognize these complexes using their T-cell receptors (TCRs). APCs process antigens and present them to T-cells. An APC may present an antigen independent of MHC. As an example, a B lymphocyte or a B cell line (e.g., REC-1) that express CD19 may serve as an APC for T cells expressing a CAR, e.g., FMC63-BBz CAR (SEQ ID NO: 2822) directed against CD19. An APC may present an antigen that is recognized by an immune cell in the presence of a bispecific or multispecific engager. As an example, a B lymphocyte or a B cell line (e.g., REC-1) that express CD19 may serve as an APC for T cells in the presence of a CD3×CD19 bispecific antibody, e.g., Blinatumomab. An APC may be a normal cell, an immortalized cell or a cancer cell. An APC may be a cell line. The SEQ ID NOs of several antigens that can be expressed on the surface of cells to serve as antigen presenting cells for the methods of the disclosure are presented in Table 7D.

The term “antigen presenting substrate” or “APS” refers to any substrate such as a beads, microbeads, agarose bead, magnetic bead, membrane, plate, bubble, nano-particles that presents an antigen that can be recognized by an immune cell. An APS may display a foreign antigen complexed with major histocompatibility complexes (MHC's) on its surface. T-cells may recognize these complexes using their T-cell receptors (TCRs). An APS may present an antigen independent of MHC. As an example, a bead or a plate coated with CD19 ectodomain may serve as an APS for T cells expressing a CAR, e.g., FMC63-BBz CAR (SEQ ID NO: 2822) directed against CD19. An APS may present an antigen that is recognized by an immune cell in the presence of a bispecific or multispecific engager. As an example, a beads or plate coated with CD19 ectodomain (ECD) may serve as an APS for T cells in the presence of a CD3×CD19 bispecific antibody, e.g., Blinatumomab. The SEQ ID NOs of several antigens that can be conjugated to substrates (e.g. beads or membrane) to serve as antigen presenting substrates for the methods of the disclosure are presented in Table 7D.

An “intracellular signaling domain,” as the term is used herein, refers to an intracellular portion of a molecule. The intracellular signaling domain generates a signal that promotes an immune effector function of the CAR containing cell, e.g., a CAR-T cell.

The term a “costimulatory molecule” refers to a cognate binding partner on a T cell that specifically binds with a costimulatory ligand, thereby mediating a costimulatory response by the T cell, such as, but not limited to, proliferation.

A “costimulatory intracellular signaling domain” can be the intracellular portion of a costimulatory molecule. The intracellular signaling domain can comprise the entire intracellular portion, or the entire native intracellular signaling domain, of the molecule from which it is derived, or a functional fragment or derivative thereof.

A “CXCR4 signaling inhibitor” is an exogenous factor, such as a pharmaceutical compound or molecule, that inhibits or prevents the activation of CXCR4 by its ligand C—X—C motif ligand 12 (CXCL12) and thereby blocks or inhibits CXCR4 signaling in cells.

Suitable CXCR4 signaling inhibitors may be identified using standard in vitro or ex vivo CXCL12/CXCR4 ligation assays, such as chemotaxis or increased free intracellular Ca 2+. For example, the absence of rapid, transient increases in free intracellular Ca 2+ when CXCR4 on a cell surface is exposed to CXCL12 may be indicative of the presence of a CXCR4 signaling inhibitor.

Preferred examples of a CXCR4 signaling inhibitor includes, but is not limited to, a CXCR4 antagonist and/or a CXCL12 antagonist.

In the disclosure, a “CXCR4 antagonist” is defined as a molecule that inhibits CXCR4 signaling by binding to or interacting with CXCR4 to prevent or inhibit the binding and/or activation of CXCR4 by CXCL12, thereby inhibiting CXCR4 signaling. Preferred examples of a CXCR4 antagonist, include, but are not limited to an anti-CXCR4 antibody, examples of which are well known in the art. For example, preferred anti-CXCR4 antibodies include, but are not limited to BMS-936564/MDX-1338 (Kuhne et al (2013) Clin Cancer Res 19(2) 357-366).

Additionally, CXCR4 antagonists include peptides, such as LY2510924 (Eli Lilly) or small organic compounds, such as 1,1′-[1,4-phenylenebis(methylene)]bis [1,4,8,11-tetraazacyclotetradecane] (AMD3100; Plerixafor), N, N-dipropyl-N-[4-({[(1H-imidazol-2-yl)methyl)benzyl][(1-methyl-1H-imidazol-2-yl) methyl]amino]methyl)benzyl]-N-methylbutane-1, 4-diamine tri(2R, 3R)-tartrate (KRH-3955), ([5-(4-methyl-1-piperazinyl)-2-({methyl [(8S)-5,6,7,8-tetrahydro-8-quinolinyl]amino}methyl)imidazo[1,2-a]pyridin-3-yl]methanol) (GSK812397; Jenkinson et al., Antimicrob. Agents Chemother. 2010, 54(2):817), and N′-(1H-benzimidazol-2-ylmethyl)-N′-(5,6,7,8-tetrahydroquinolin-8-yl)butane-1,4-diamine (AMD11070; Moyle et al Clin. Infect. Dis. 48:798-805)).

In the disclosure, a “CXCL12 antagonist” is defined as a molecule that inhibits CXCR4 signaling by binding to or inhibiting CXCL12 from binding and/or activating CXCR4, thereby inhibiting CXCR4 signaling. CXCL12 may, for example, be produced by stromal cells in the cancerous tumor that express fibroblast activation protein (FAP). Preferred examples of a CXCL12 antagonist, include, but are not limited to an anti-CXCL12 antibody, which are well known in the art. Examples of such anti-CXCL12 antibodies, include, but are not limited to an anti-CXCL12 antibody from R&D Systems (MAB310) or SDF-1 antibody. Other examples of CXCL12 antagonists include, but are not limited to, NOX-A12.

Other suitable CXCR4 and CXCL12 antagonists include non-antibody specific binding molecules, such as adnectins, affibodies, avimers, anticalins, tetranectins, DARPins, mTCRs, engineered Kunitz-type inhibitors, nucleic acid aptamers and spiegelmers, peptide aptamers and cyclic and bicyclic peptides (Ruigrok et al Biochem. J. (2011) 436, 1-13; Gebauer et al Curr Opin Chem Biol. (2009)(3):245-55). Suitable specific binding molecules for use as CXCR4 and CXCL12 antagonists may be generated using standard techniques.

CXCR4 signaling is mediated by activation ofphosphoinositide 3-kinases. Other suitable CXCR4 signaling inhibitors include PI 3-kinase inhibitors, for example inhibitors of p110 delta or p110 gamma isoforms of PI3K.

The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like. In one aspect, the “4-1BB costimulatory domain” is the sequence provided as SEQ ID NO: 1857 or the equivalent residues from a non-human species, e.g., mouse, rodent, monkey, ape and the like.

“Immune cell” as that term is used herein, refers to a cell that is involved in an immune response. Examples of immune effector cells include T cells, e.g., alpha/beta T cells and gamma/delta T cells, B cells, natural killer (NK) cells, natural killer T(NKT) cells, mast cells, monocytes, macrophage, and myeloic-derived phagocytes.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T(NKT) cells, mast cells, monocytes, macrophage, and myeloic-derived phagocytes.

“Immune effector function or immune effector response or immune response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

“Immune therapy” as the term is used herein, refers to a form of therapy that involves the use of immune cells or products of immune cells. Examples of immune therapy include bispecific T cell engagers (e.g., Blinatumomab), DARTs, PD-1 inhibitors, PDL-1 inhibitors, CAR-T, CAR-NK, macrophage-CAR, TCR-T and TIL therapies as well as vaccination approaches.

“Cell therapy”, “Adoptive cell therapy”, “Cellular Therapy” or “Immune cell therapy” as the term is used herein, refers to a form of therapy that involves the use of cells, e.g., immune cells, in the prevention and/or treatment of a disease. Examples of cell therapy include allogeneic stem cell transplant, CAR-T, CAR-NK, macrophage-CAR, TCR-T and TIL therapies as well as T cell vaccination approaches. A form of cell therapy is immune cell therapy, e.g., CAR-T cell therapy. As bispecific T cell engagers also engage T cells, they are also considered a form of immune cell therapy.

The terms “Cell therapy product”, “Cellular Therapy product” or “Immune cell therapy product” as used herein, refer to a product used for the purpose of cell therapy.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Unless otherwise specified, a “nucleotide sequence encoding an amino acid sequence” includes all nucleotide sequences that are degenerate versions of each other and that encode the same amino acid sequence.

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed.

The term “lentivirus” refers to a genus of the Retroviridae family.

The term “lentiviral vector” refers to a vector derived from at least a portion of a lentivirus genome, including especially a self-inactivating lentiviral vector.

The term “homologous” or “identity” refers to the subunit sequence identity between two polymeric molecules, e.g., between two nucleic acid molecules, such as, two DNA molecules or two RNA molecules, or between two polypeptide molecules.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state.

The term “operably linked” or “transcriptional control” refers to functional linkage between a regulatory sequence and a heterologous nucleic acid sequence resulting in expression of the latter.

The term “parenteral” administration of an immunogenic composition includes, e.g., subcutaneous (s.c.), intravenous (i.v.), intramuscular (i.m.), or intrastemal injection, intratumoral, or infusion techniques.

The term “mobilized” cell e.g., a mobilized immune cell, a mobilized immune effector cell or mobilized T cell refers to a cell that has been mobilized from its normal location. For example, administration of CXCR antagonist can be used to mobilize immune cells from bone marrow, lymph organs, tissues and tumors into peripheral circulation from where they can be collected by leukapheresis and used for immune cell therapy applications, e.g., manufacturing of CAR-T cells or TCR-T cells or TILs, described in this disclosure. Other exemplary mobilization agents that can be used to mobilize cells include cytokines (e.g., G-CSF, GM-CSF or sargramostim, Neulasta or Pegfilgastrim etc.), chemotherapy drugs (e.g cyclophosphamide). Tyrosine kinase inhibitors (e.g., Dasatinib), beta2 adrenergic agonists (e.g., epinephrine) and exercise.

The term “mobilization agent” refers to an agent that can be used to mobilize a cell from its normal location. Exemplary mobilization agents that can be used to mobilize cells include cytokines (e.g., G-CSF, GM-CSF or sargramostim, Neulasta or Pegfilgastrim etc.), chemotherapy drugs (e.g. cyclophosphamide). Tyrosine kinase inhibitors (e.g., Dasatinib), beta2 adrenergic agonists (e.g., epinephrine) and exercise.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

The terms “peptide,” “polypeptide,” and “protein” are used interchangeably, and refer to a compound comprised of amino acid residues covalently linked by peptide bonds.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell.

The term “tumor-supporting antigen” or “cancer-supporting antigen” interchangeably refer to a molecule (typically a protein, carbohydrate or lipid) that is expressed on the surface of a cell that is, itself, not cancerous, but supports the cancer cells, e.g., by promoting their growth or survival e.g., resistance to immune cells.

The term “flexible polypeptide linker” or “linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)n, where n is a positive integer equal to or greater than 1. For example, n=1, n=2, n=3, n=4, n=5 and n=6, n=7, n=8, n=9 and n=10. In one embodiment, the flexible polypeptide linkers include, but are not limited to, or (Gly4 Ser)3 (SEQ ID NO (DNA):411 and SEQ ID NO (PRT):1838). In another embodiment, the linkers include multiple repeats of (Gly2Ser), (GlySer) or (Gly3Ser). Also included within the scope of the disclosure are linkers described in WO2012/138475, incorporated herein by reference).

As used herein, a 5′ cap (also termed an RNA cap, an RNA 7-methylguanosine cap or an RNA m⁷G cap) is a modified guanine nucleotide that has been added to the “front” or 5′ end of a eukaryotic messenger RNA shortly after the start of transcription.

As used herein, “in vitro transcribed RNA” refers to RNA, preferably mRNA, that has been synthesized in vitro.

As used herein, a “poly(A)” is a series of adenosines attached by polyadenylation to the mRNA. In the preferred embodiment of a construct for transient expression, the polyA is between 50 and 5000 (SEQ ID NO: 341-343), preferably greater than 64, more preferably greater than 100, most preferably greater than 300 or 400. poly(A) sequences can be modified chemically or enzymatically to modulate mRNA functionality such as localization, stability or efficiency of translation.

The term “prophylaxis” as used herein means the prevention of or protective treatment for a disease or disease state.

As used herein, “polyadenylation” refers to the covalent linkage of a polyadenylyl moiety, or its modified variant, to a messenger RNA molecule.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as a CAR of the disclosure).

The term “therapeutic” as used herein means a treatment. A therapeutic effect is obtained by reduction, suppression, remission, or eradication of a disease state.

As used herein, the terms “Regulatory T cells” or “TREGs” refer to T cells which have a role in regulating or suppressing other cells in the immune system. Tregs control the immune response to self and foreign particles (antigens) and help prevent autoimmune disease.

In the context of the disclosure, “tumor antigen” or “hyperproliferative disorder antigen” or “antigen associated with a hyperproliferative disorder” refers to antigens that are common to specific hyperproliferative disorders. In certain aspects, the hyperproliferative disorder antigens of the disclosure are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer, ovarian cancer, pancreatic cancer, and the like.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a binding partner (e.g., a tumor antigen) protein present in a sample, but which antibody or ligand does not substantially recognize or bind other molecules in the sample.

“Membrane anchor” or “membrane tethering domain”, as that termis used herein, refers to a polypeptide or moiety, e.g., a myristoyl group, sufficient to anchor an extracellular or intracellular domain to the plasma membrane.

“Switch domain,” as that term is used herein, e.g., when referring to an RCAR, refers to an entity, typically a polypeptide-based entity, that, in the presence of a dimerization molecule, associates with another switch domain. “Dimerization molecule,” as that term is used herein, e.g., when referring to an RCAR, refers to a molecule that promotes the association of a first switch domain with a second switch domain.

“Refractory” as used herein refers to a disease, e.g., cancer, that does not respond to a treatment.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

“Tet” as the term is used herein, refers to the family of genes, and the proteins encoded by said genes, of the ten-eleven translocation methlcytosine dioxygenase family. Tet includes, for example, Tet1, Tet2 and Tet3.

“Tet2” as the term is used herein, refers to gene, tet methylcytosine dioxygenase 2, and the protein encoded by said gene, the tet2 methylcytosine dioxygenase, which catalyzes the conversion of methylcytosine to 5-hydroxymethylcytosine.

TABLE 3
Nucleic Acid Reference SEQ of the exemplary
genes of the disclosure.

| | |
|---|---|
| BRD9 | |
| | NM_001009877 |
| | NM_001317951 |
| | NM_023924 |
| | NR_134296 |
| EZH2 | |
| | NM_001203247 |
| | NM_001203248 |
| | NM_001203249 |
| | NM_004456 |
| | NM_152998 |
| ASXL1 | |
| | NM_001164603 |
| | NM_015338 |
| KDM6A | |
| | NM_001291415 |
| | NM_001291416 |
| | NM_001291417 |
| | NM_001291418 |
| | NM_001291421 |
| | NM_021140 |
| | NR_111960 |
| PHF6 | |
| | NM_001015877 |
| | NM_032335 |
| | NM_032458 |
| SF3B1 | |
| | NM_001005526 |
| | NM_001308824 |
| | NM_012433 |
| CBFB | |
| | NM_001755 |
| | NM_022845 |
| RUNX1 | |
| | NM_001001890 |
| | NM_001122607 |
| | NM_001754 |
| PRDM1 | |
| | NM_001198 |
| | NM_182907 |
| HDAC2 | |
| | NM_001527 |
| | NR_033441 |
| | NR_073443 |
| EHMT2 | |
| | NM_001289413 |
| | NM_001289413 |
| | NM_001289413 |
| | NM_001289413 |
| | NM_001289413 |
| | NM_001289413 |
| | NM_001289413 |
| | NM_001318833 |
| | NM_001318833 |
| | NM_001318833 |
| | NM_001318833 |
| | NM_001318833 |
| | NM_001318833 |
| | NM_001318833 |
| | NM_006709 |
| | NM_006709 |
| | NM_006709 |
| | NM_006709 |
| | NM_006709 |
| | NM_006709 |
| | NM_006709 |
| | NM_025256 |
| | NM_025256 |
| | NM_025256 |
| | NM_025256 |
| | NM_025256 |
| | NM_025256 |
| | NM_025256 |
| SMARCA4 | |
| | NM_001128844 |
| | NM_001128845 |
| | NM_001128846 |
| | NM_001128847 |
| | NM_001128848 |
| | NM_001128849 |
| | NM_003072 |
| CREBBP | |
| | NM_001079846 |
| | NM_004380 |
| KMT2D | |
| | NM_003482.3 |
| KMT2C | |
| | NM_170606.2 |
| KMT2B | |
| | NM_014727.2 |
| Tet2 | |
| | NM_001127208 |
| | NM_017628 |
| Stat5b | |
| | NM_012448.3 |
| Stat3 | |
| | NM_003150.3 |
| | NM_139276.2 |
| | NM_213662.1 |
| JAK3 | |
| | NM_000215.3 |
| JAK1 | |
| | NM_001320923.1 |
| | NM_001321852.1 |
| | NM_001321853.1 |
| | NM_001321854.1 |
| | NM_001321855.1 |
| | NM_001321856.1 |
| | NM_001321857.1 |
| | NM_002227.3 |
| BRAF | |
| | NM_001354609 |
| | NM_004333 |
| | NR_148928 |
| CARD11 | |
| | NM_001324281 |
| | NM_032415 |
| FBXW10 | |
| | NM_001267585.1 |
| | NM_001267586.1 |
| IL2RG | |
| | NM_000206.2 |
| BCOR | |
| | NM_001123383.1 |
| | NM_001123384.1 |
| | NM_001123385.1 |
| | NM_017745.5 |
| CHEK2 | |
| | NM_001005735 |
| | NM_001257387 |
| | NM_001349956 |
| | NM_007194 |
| | NM_145862 |
| YY1 | |
| | NM_003403.4 |
| ATM | |
| | NM_000051 |
| | NM_001351834 |
| | NM_001351835 |
| | NM_001351836 |
| TRAIL | |
| (TNFSF10) | |
| | NM_001190942.1 |
| | NM_001190943.1 |
| | NM_003810.3 |
| DR5 | |
| (TNFRSF10B) | |
| | NM_003842.4 |
| | NM_147187.2 |
| DR4 | |
| (TNFRSF10A) | |
| | NM_003844.3 |

“X inhibitor” or “Inhibitor X” as the terms are used herein, refer to molecule, or group of molecules (e.g., a system) that reduces or eliminates the function and/or expression of corresponding “X” gene and/or protein where “X” represents BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL or DR5. In embodiments, “X” inhibitor is a molecule that inhibits the expression of “X”, e.g., reduces or eliminates expression of “X”. In embodiments, the “X” inhibitor is a molecule that inhibits the function of “X”. An example of “X” inhibitor that inhibits the expression of “X” is a gene editing system, e.g., as described herein, that is targeted to nucleic acid within the “X” gene, or its regulatory elements, such that modification of the nucleic acid at or near the gene editing system binding site(s) is modified to reduce or eliminate expression of “X”. Another example of a “X” inhibitor that inhibits the expression of “X” is a nucleic acid molecule, e.g., RNA molecule, e.g., a short hairpin RNA (shRNA) or short interfering RNA (siRNA), capable of hybridizing with “X” mRNA and causing a reduction or elimination of “X” translation. “X” inhibitors also include nucleic acids encoding molecules which inhibit “X” expression (e.g., nucleic acid encoding an anti-“X” shRNA or siRNA, or nucleic acid encoding one or more, e.g., all, components of an anti-“X” gene editing system). An example of a molecule that inhibits the function of “X” is a molecule, e.g., a protein or small molecule which inhibits one or more activities of “X”. An example is a small molecule inhibitor of “X”. Another example is a dominant negative “X” protein. Another example is a dominant negative version of a “X” binding partner. Another example is a molecule, e.g., a small molecule, which inhibits a “X” binding partner. “X” inhibitors also include nucleic acids encoding inhibitors of “X” function.

“BRD9 inhibitor” as the term is used herein, refers to a molecule, or group of molecules (e.g., a system) that reduces or eliminates the function and/or expression of BRD9. In embodiments, a BRD9 inhibitor is a molecule that inhibits the expression of BRD9, e.g., reduces or eliminates expression of BRD9. In embodiments, the BRD9 inhibitor is a molecule that inhibits the function of BRD9. An example of BRD9 inhibitor that inhibits the expression of BRD9 is a gene editing system, e.g., as described herein, that is targeted to nucleic acid within the BRD9 gene, or its regulatory elements, such that modification of the nucleic acid at or near the gene editing system binding site(s) is modified to reduce or eliminate expression of BRD9. Another example of a BRD9 inhibitor that inhibits the expression of BRD9 is a nucleic acid molecule, e.g., RNA molecule, e.g., a short hairpin RNA (shRNA) or short interfering RNA (siRNA), capable of hybridizing with BRD9 mRNA and causing a reduction or elimination of BRD9 translation. BRD9 inhibitors also include nucleic acids encoding molecules which inhibit BRD9 expression (e.g., nucleic acid encoding an anti-BRD9 shRNA or siRNA, or nucleic acid encoding one or more, e.g., all, components of an anti-BRD9 gene editing system). An example of a molecule that inhibits the function of BRD9 is a molecule, e.g., a protein or small molecule which inhibits one or more activities of BRD9. An example is a small molecule inhibitor of BRD9. Another example is a dominant negative BRD9 protein. Another example is a dominant negative version of a BRD9 binding partner. Another example is a molecule, e.g., a small molecule, which inhibits a BRD9 binding partner. BRD9 inhibitors also include nucleic acids encoding inhibitors of BRD9 function.

“Tet inhibitor” or “Tet[x] inhibitor” (e.g., “Tet1 inhibitor,” “Tet2 inhibitor”, or “Tet3 inhibitor”) as the terms are used herein, refers to a molecule, or group of molecules (e.g., a system) that reduces or eliminates the function and/or expression of the corresponding Tet, e.g., Tet1, Tet2 and/or Tet3, e.g., Tet2.

“MLL2 inhibitor” as the terms are used herein, refers to a molecule, or group of molecules (e.g., a system) that reduces or eliminates the function and/or expression of MLL2. “MLL3 inhibitor” as the terms are used herein, refers to a molecule, or group of molecules (e.g., a system) that reduces or eliminates the function and/or expression of MLL3. “MLL4 inhibitor” as the terms are used herein, refers to a molecule, or group of molecules (e.g., a system) that reduces or eliminates the function and/or expression of MLL4. “EZH2 inhibitor” as the terms are used herein, refers to a molecule, or group of molecules (e.g., a system) that reduces or eliminates the function and/or expression of EZH2.

“TRAIL inhibitor” or “TRAIL antagonist” as the terms are used herein, refers to a molecule, or group of molecules (e.g., a system) that reduces or eliminates the function and/or expression of TRAIL and/or its receptors DR5 ande/or DR4. In embodiments, a TRAIL inhibitor is a molecule that inhibits the expression of TRAIL and DR5, e.g., reduces or eliminates expression of TRAIL and/or DR5. In embodiments, the TRAIL inhibitor is a molecule that inhibits the function of TRAIL and/or DR5. An example of TRAIL inhibitor that inhibits the expression of TRAIL is a gene editing system, e.g., as described herein, that is targeted to nucleic acid within the TRAIL gene, or its regulatory elements, such that modification of the nucleic acid at or near the gene editing system binding site(s) is modified to reduce or eliminate expression of TRAIL. Another example of TRAIL inhibitor that inhibits the expression of TRAIL is a nucleic acid molecule, e.g., RNA molecule, e.g., a short hairpin RNA (shRNA) or short interfering RNA (siRNA), capable ofhybridizing with TRAIL mRNA and causing a reduction or elimination TRAIL translation. TRAIL inhibitors also include nucleic acids encoding molecules which inhibit TRAIL expression (e.g., nucleic acid encoding an anti-TRAIL, e.g., TRAIL shRNA or siRNA, or nucleic acid encoding one or more, e.g., all, components of an anti-TRAIL, e.g., TRAIL gene editing system). An example of a molecule that inhibits the function of TRAIL is a molecule, e.g., a protein or small molecule which inhibits one or more activities of TRAIL. An example is a small molecule inhibitor of TRAIL. Another example is an antibody, antibody fragment, a non-immunoglobulin binding scaffold that binds to TRAIL and/or DR5 and prevents signaling through DR5. Another example is a dominant negative TRAIL protein. Another example is a dominant negative mutant of TRAIL receptor DR5. Another example is a soluble or decoy form of TRAIL receptors DR4 (e.g., SEQ ID NO: 2441), DR5 (SEQ ID NO: 2428), DcR1 (SEQ ID NO: 2448) and DcR2 (SEQ ID NO: 2455). Another example is a mutant form of DR5 that lacks its cytoplasmic signaling domain or in which the signaling domain of DR5 is replaced by the signaling domain of a costimulatory receptor, such CD27, CD28, 41BB, GITR, BCMA, or OX40 (e.g., SEQ ID NO: 2429-2440). Additional exemplary TRAIL antagonists include DcR1 (SEQ ID NO:2360), DcR2 (SEQ ID NO: 2361) and fusion receptors containing the TRAIL-binding ectodomains of DcR1, DcR2, DR4 and DR5(SEQ ID NO: 2429-2461). Another example of a TRAIL inhibitor is a molecule, e.g., a small molecule, which inhibits a TRAIL binding partner, e.g., a TRAIL receptor, e.g., DR5. Another example of a TRAIL inhibitor is a molecule, e.g., a small molecule, which inhibits signal transduction downstream of a TRAIL receptor, e.g., DR5. An exemplary small molecule that inhibits signaling downstream of a TRAIL receptor is an NF-κB inhibitor, e.g., IKK-2 Inhibitor IV (CAS 507475-17-4), IKK2 Inhibitor VI (CAS 354811-10-2), LY2409881 or Bortezomib. Other TRAIL inhibitors, including NF-κB inhibitors, are known in the art and can be used in alternate embodiment of the disclosure. TRAIL signaling is known to activate a number of other signaling pathways, e.g., JNK pathways, and inhibitors of these pathways can be used in alternate embodiment of the disclosure to improve the efficacy and safety of immune effector cell therapies. Other examples of TRAIL inhibitor include dominant negative mutants of FADD (e.g., FADD death domain; SEQ ID NO (DNA): 2453, SEQ ID NO (PRT): 2463) and Caspase 8 (e.g., Caspase 8 D73A [SEQ ID NO (DNA): 2354, SEQ ID NO (PRT): 2464], Caspase 8 D73A/L74A; SEQ ID NO (DNA): 2355 and SEQ ID NO (PRT): 2365; Caspase 8 D73A/L74A/L75A; SEQ ID NO (DNA): 2356 and SEQ ID NO (PRT): 2366. TRAIL inhibitors also include nucleic acids encoding inhibitors of TRAIL function, e.g., binding of TRAIL to its receptor, e.g., DR5 and/or signal transduction downstream of a TRAIL receptor. TRAIL inhibitors also include nucleic acids encoding molecules which inhibit TRAIL signaling (e.g., nucleic acid encoding an anti-FADD or anti-Caspase 8, e.g., FADD shRNA or siRNA, or Caspase 8 shRNA or siRNA or nucleic acid encoding one or more, e.g., all, components of an anti-FADD or Caspase 8, e.g., FADD gene editing system or Caspase 8 gene editing system).

The inhibitor/antagonists of other molecules of the disclosure can be described similarly.

A “system” as the term is used herein in connection with gene editing refers to a group of molecules, e.g., one or more molecules, which together act to effect a desired function.

A “gene editing system” as the term is used herein, refers to a system, e.g., one or more molecules that direct and effect an alteration, e.g., a deletion, of one or more nucleic acids at or near a site of genomic DNA targeted by said system. Gene editing systems are known in the art, and are described more fully below.

“Binding partner” as the term is used herein in the context of a “X”, e.g., Tet1, Tet2 and/or Tet3, e.g., Tet2 binding partner, refers to a molecule, e.g., a protein, which interacts, e.g., binds to “X”, e.g., Tet, e.g., Tet1, Tet2 and/or Tet3, e.g., Tet2 protein. Without being bound by theory, it is believed that Tet, e.g., Tet1, Tet2 and/or Tet3, e.g., Tet2 binds to one or more HDAC proteins. Such HDAC proteins are considered examples of Tet, e.g., Tet1, Tet2 and/or Tet3, e.g., Tet2 binding partners. Binding partners of other proteins of the disclosure are known in the art.

A “dominant negative” gene product or protein is one that interferes with the function of another gene product or protein. In one embodiment, a dominant negative DR5 is a mutant of DR5 that is incapable of transmitting a signal, e.g., a death inducing signal.

Manufacturing cell therapy products (e.g., CAR-T or TCR-T or cellular vaccines) genrerally begins with the collection of lymphocytes from a subject via leukapheresis and T-cell selection, followed by activation, modification, expansion and cryopreservation of the final product. While this process has worked well in proof-of-concept clinical trials, several challenges exist that impede the scaling-up and scaling-out of viable cellular therapies. The term “subject” is intended to include living organisms in which an immune response can be elicited (e.g., mammals). Examples of subjects include humans, monkeys, chimpanzees, dogs, cats, mice, rats, and transgenic species thereof. T cells can be obtained from a number of sources, including peripheral blood mononuclear cells, bone marrow, lymph node tissue, cord blood, tissue resident lymphocytes, tumor-resident lymphocytes, thymus tissue, tissue from a site of infection, ascites, pleural effusion, spleen tissue, and tumors.

The disclosure relates to a method of improving the the yield, expansion, activation, proliferation, expansion, diversity, tissue (e.g., tumor) penetrance, persistence, efficacy and safety of cell therapy products, e.g., immune cell therapy products, e.g., CAR-T, SIR-T, TFP-T, Ab-TCR-T and TCR-T products etc. In one embodiment, the disclosure related to improving the yield, expansion, activation, proliferation, expansion, diversity, tissue (e.g., tumor) penetrance, persistence and efficacy of immune cell therapies, such as engineered CAR-T, TCR-T, SIR-T, Ab-TCR-T cell therapies or NK cell therapies, by using mobilized immune cells for the manufacturing of cell therapy products. In various embodiments, the cells (e.g., immune cells, e.g., T cells or NK cells or macrophage/monocytes or dendritic cells) are mobilized by administrating to the donor from whom the cells are harvested a CXCR antagonist (e.g., Mozibil or Plerixafor), a cytokine (e.g., G-CSF, GM-CSF or sargramostim, Neulasta or Pegfilgastrim), a beta2 adrenergic agonist (e.g., epinephrine), a tyrosine kinase inhibitor (e.g., dasatinib), chemotherapy drug(s) (e.g., cyclophosphamide, doxorubicin) or a combination of the above agents prior to the collection of cells (e.g., immune cells, e.g., T cells or NK cells). In various embodiments, the cells (e.g., immune cells, e.g., T cells or NK cells or macrophage/monocytes or dendritic cells) are mobilized by making the subject (i.e. donor) exercise.

In one aspect, the mobilized cells (e.g., immune cells, e.g., T cells or NK cells) are obtained from the subject using leukapheresis. In one aspect, the mobilized cells (e.g., immune cells, e.g., T cells or NK cells) are obtained from the subject using bone marrow harvest.

In one aspect, the mobilized cells (e.g., immune cells, e.g., T cells or NK cells) are obtained from a subject who is candidate for receiving the cell therapy product.

In one aspect, the cells (e.g., immune cells, e.g., T cells or NK cells) are obtained from a subject who is a healthy donor. In one aspect, the mobilized cells (e.g., immune cells, e.g., T cells or NK cells) are obtained from a subject who has a disease (e.g., a cancer, infection or auto-immune disease).

In one aspect, the mobilized cells (e.g., immune cells, e.g., T cells or NK cells) are obtained from a subject who has received prior chemotherapy, e.g., anti-cancer drugs. In one aspect, the mobilized cells (e.g., immune cells, e.g., T cells or NK cells) are obtained from a subject who has insufficient number of circulating (e.g., immune cells, e.g., T cells or NK cells or macrophage/monocytes or dendritic cells) cells prior to administration of the mobilizing agent.

In one aspect, the mobilized cells (e.g., immune cells, e.g., T cells or NK cells) are obtained from a subject who has less than about 1000×10⁶/L (e.g., less than 600×10⁶/L, 400×10⁶/L, 200×10⁶/L or 100×10⁶/L) CD3+ cells in peripheral blood prior to the administration of the mobilizing agent. In one aspect, the mobilized cells (e.g., immune cells, e.g., T cells or NK cells) are obtained from a subject who has less than about 800×10⁶/L (e.g., less than 400×10⁶/L, 200×10⁶/L, 100×10⁶/L, 50×10⁶/L) CD4+ cells in peripheral blood prior to the administration of the mobilizing agent.

In one aspect, the mobilized cells (e.g., immune cells, e.g., T cells or NK cells) are obtained from a subject who has less than about 500×10⁶/L (e.g., less than 400×10⁶/L, 200×10⁶/L, 100×10⁶/L, or 50×10⁶/L) CD8+ cells in peripheral blood prior to the administration of the mobilizing agent. In one aspect, the mobilized cells (e.g., immune cells, e.g., T cells or NK cells) are obtained from a subject who has less than about 50×10⁶/L (e.g., less than 40×10⁶/L, 20×10⁶/L, 10×10⁶/L, or 5×10⁶/L) CD3+/CD16+ cells in peripheral blood prior to the administration of the mobilizing agent.

In one aspect, the mobilized cells (e.g., immune cells, e.g., T cells or NK cells) are obtained from a subject who has less than about 50×10⁶/L (e.g., less than 40×10⁶/L, 20×10⁶/L, 10×10⁶/L, or 5×10⁶/L) CD3+/CD56+ cells in peripheral blood prior to the administration of the mobilizing agent. In one aspect, the mobilized cells (e.g., immune cells, e.g., T cells or NK cells) are obtained from a subject who has poor quality of peripheral blood cells (e.g., immune cells, e.g., T cells or NK cells etc.) prior to administration of the mobilizing agent.

In one aspect, the mobilized cells (e.g., immune cells, e.g., T cells or NK cells) are obtained from a subject who has poor diversity of peripheral blood cells (e.g., immune cells, e.g., T cells or NK cells) prior to administration of the mobilizing agent.

In one aspect, the mobilized cells (e.g., immune cells, e.g., T cells or NK cells) are obtained from a subject who has poor polyfuncationality (e.g., antigen induced production of IL2, TNFα and IFNγ etc.) of peripheral blood cells (e.g., immune cells, e.g., T cells or NK cells) prior to administration of the mobilizing agent.

In one aspect, the mobilized cells (e.g., immune cells, e.g., T cells or NK cells) are obtained from a subject who has or is expected to have a poor yield of leukapheresed product without the administration of the mobilizing agent. In one aspect, the mobilized cells (e.g., immune cells, e.g., T cells or NK cells) are obtained from a subject who has or is expected to have less than about 50×10⁹ (e.g., less than 40×10⁹, 20×10⁹, 10×10⁹ etc.) white blood cells (WBC) in the leukapheresed product without the administration of the mobilizing agent.

In one embodiment, the method involves obtaining cells (e.g., immune cells, e.g., T cells or NK cells) for the manufacturing and use of a cellular therapy product from a subject (i.e. an autologous or an allogeneic donor) who has received a mobilizing agent, (e.g., a CXCR4 antagonist, e.g., Plerixafor) prior to the collection of cells.

In a further aspect of the disclosure, the method involves obtaining cells (e.g., immune cells, e.g., T cells or NK cells) for the manufacturing and use of a cellular therapy product from a subject (i.e. an autologous or an allogeneic donor) who has received a mobilizing agent, (e.g., a CXCR4 antagonist, e.g., Plerixafor) to aid in mobilization of immune cells (e.g., T cells) into peripheral circulation.

In further aspect of the disclosure, the method involves obtaining cells (e.g., immune cells, e.g., T cells or NK cells) for the manufacturing and use of a cellular therapy product from a subject (i.e. an autologous or an allogeneic donor) who has received a mobilizing agent, (e.g., a CXCR4 antagonist, e.g., Plerixafor) to aid in mobilization of specific subset of immune cells into peripheral circulation.

In one aspect, the cells (e.g., immune cells, e.g., T cells or NK cells) that have been mobilized by a mobilizing agent (e.g., a CXCR4 antagonist, e.g., Plerixafor) are obtained from a subject who is candidate for receiving the cell therapy product. In one aspect, the cells (e.g., immune cells, e.g., T cells or NK cells) that have been mobilized by a mobilizing agent (e.g., a CXCR4 antagonist, e.g., Plerixafor) are obtained from a subject who is a healthy donor. In one aspect, the cells (e.g., immune cells, e.g., T cells or NK cells) that have been mobilized by a mobilizing agent (e.g., a CXCR4 antagonist, e.g., Plerixafor) are obtained from a subject who has a disease (e.g., a cancer, infection or auto-immune disease).

In one aspect, the cells (e.g., immune cells, e.g., T cells or NK cells) that have been mobilized by a mobilizing agent (e.g., a CXCR4 antagonist, e.g., Plerixafor) are obtained from a subject who has previously received treatment for a disease (e.g., cancer, immune or infection disease). In one aspect, the cells (e.g., immune cells, e.g., T cells or NK cells) that have been mobilized by a mobilizing agent (e.g., a CXCR4 antagonist, e.g., Plerixafor) are obtained from a subject who has previously received treatment for a cancer.

In one aspect, the cells (e.g., immune cells, e.g., T cells or NK cells) that have been mobilized by a mobilizing agent (e.g., a CXCR4 antagonist, e.g., Plerixafor) are obtained from a subject who has previously received treatment for a blood cancer (e.g., plasma cell disorder, myeloma, lymphoma, acute lymphocytic leukemia, chronic lymphocytic leukemia, MDS etc.). In one aspect, the cells (e.g., immune cells, e.g., T cells or NK cells) that have been mobilized by a mobilizing agent (e.g., a CXCR4 antagonist, e.g., Plerixafor) are obtained from a subject who has previously received treatment for a solid tumor (e.g., lung cancer, breast cancer, gastrointestinal cancer, liver cancer etc.). In one aspect, the cells (e.g., immune cells, e.g., T cells or NK cells) that have been mobilized by a mobilizing agent (e.g., a CXCR4 antagonist, e.g., Plerixafor) are obtained from a subject who has previously received treatment for an immune disorder. In one aspect, the cells (e.g., immune cells, e.g., T cells or NK cells) that have been mobilized by a mobilizing agent (e.g., a CXCR4 antagonist, e.g., Plerixafor) are obtained from a subject who has previously received treatment for an infection (e.g., HIV-1/AIDS).

In one aspect, the cells (e.g., immune cells, e.g., T cells or NK cells) that have been mobilized by a mobilizing agent (e.g., a CXCR4 antagonist, e.g., Plerixafor) are obtained from a subject who has previously received a chemotherapy drug, e.g., an anti-cancer drug, e.g., alkylating agent, e.g., melphalan, cyclophosphamide etc. Other anticancer drugs are known in the art and include etoposide, doxorubicin, ARA-C, fludarabine, vincristine, vinblastine etc. In one aspect, the cells (e.g., immune cells, e.g., T cells or NK cells) that have been mobilized by a mobilizing agent (e.g., a CXCR4 antagonist, e.g., Plerixafor) are obtained from a subject who has previously received an immune modulatory drug (IMiDs; e.g., lenalidomide). In one aspect, the cells (e.g., immune cells, e.g., T cells or NK cells) that have been mobilized by a mobilizing agent (e.g., a CXCR4 antagonist, e.g., Plerixafor) are obtained from a subject who has previously received a steroid (e.g., dexamethasone). In one aspect, the cells (e.g., immune cells, e.g., T cells or NK cells) that have been mobilized by a mobilizing agent (e.g., a CXCR4 antagonist, e.g., Plerixafor) are obtained from a subject who has previously received an antibody (e.g., rituximab, CAMPATH etc). In one aspect, the cells (e.g., immune cells, e.g., T cells or NK cells) that have been mobilized by a mobilizing agent (e.g., a CXCR4 antagonist, e.g., Plerixafor) are obtained from a subject who has previously received an antibody drug conjugate (e.g., ADCETRIS). In one aspect, the cells (e.g., immune cells, e.g., T cells or NK cells) that have been mobilized by a mobilizing agent (e.g., a CXCR4 antagonist, e.g., Plerixafor) are obtained from a subject who has previously received a bispecific antibody (e.g., Blinatumomab). In one aspect, the cells (e.g., immune cells, e.g., T cells or NK cells) that have been mobilized by a mobilizing agent (e.g., a CXCR4 antagonist, e.g., Plerixafor) are obtained from a subject who has previously received a targeted therapy (e.g. a tyrosine kinase inhibitor, e.g., Ibrutinib). In one aspect, the cells (e.g., immune cells, e.g., T cells or NK cells) that have been mobilized by a mobilizing agent (e.g., a CXCR4 antagonist, e.g., Plerixafor) are obtained from a subject who has previously received radiation therapy. In one aspect, the cells (e.g., immune cells, e.g., T cells or NK cells) that have been mobilized by a mobilizing agent (e.g., a CXCR4 antagonist, e.g., Plerixafor) are obtained from a subject who has previously received a radiolabeled antibody.

In one aspect, the cells (e.g., immune cells, e.g., T cells or NK cells) that have been mobilized by a mobilizing agent (e.g., a CXCR4 antagonist, e.g., Plerixafor) are obtained from a subject who has insufficient number of circulating cells (e.g., immune cells, e.g., T cells or NK cells) prior to administration of the mobilizing agent. In one aspect, the CXCR4 antagonist-mobilized cells (e.g., immune cells, e.g., T cells or NK cells) are obtained from a subject who has less than about 1000×10⁶/L (e.g., less than 600×10⁶/L, 400×10⁶/L, 200×10⁶/L or 100×10⁶/L) CD3+ cells in peripheral blood prior to the administration of the mobilizing agent. In one aspect, the CXCR4 antagonist-mobilized cells (e.g., immune cells, e.g., T cells or NK cells) are obtained from a subject who has less than about 800×10⁶/L (e.g., less than 400×10⁶/L, 200×10⁶/L, 100×10⁶/L, 50×10⁶/L) CD4+ cells in peripheral blood prior to the administration of the mobilizing agent. In one aspect, the CXCR4 antagonist-mobilized cells (e.g., immune cells, e.g., T cells or NK cells) are obtained from a subject who has less than about 500×10⁶/L (e.g., less than 400×10⁶/L, 200×10⁶/L, 100×10⁶/L, or 50×10⁶/L) CD8+ cells in peripheral blood prior to the administration of the mobilizing agent. In one aspect, the CXCR4 antagonist-mobilized cells (e.g., immune cells, e.g., T cells or NK cells) are obtained from a subject who has less than about 50×10⁶/L (e.g., less than 40×10⁶/L, 20×10⁶/L, 10×10⁶/L, or 5×10⁶/L) CD3+/CD16+ cells in peripheral blood prior to the administration of the mobilizing agent. In one aspect, the CXCR4 antagonist-mobilized cells (e.g., immune cells, e.g., T cells or NK cells) are obtained from a subject who has less than about 50×10⁶/L (e.g., less than 40×10⁶/L, 20×10⁶/L, 10×10⁶/L, or 5×10⁶/L) CD3+/CD56+ cells in peripheral blood prior to the administration of the mobilizing agent. In one aspect, the CXCR4 antagonist-mobilized cells (e.g., immune cells, e.g., T cells or NK cells) are obtained from a subject who has poor quality of peripheral blood cells (e.g., immune cells, e.g., T cells or NK cells etc.) prior to administration of the mobilizing agent. In one aspect, the CXCR4 antagonist-mobilized cells (e.g., immune cells, e.g., T cells or NK cells) are obtained from a subject who has poor diversity of peripheral blood cells (e.g., immune cells, e.g., T cells or NK cells) prior to administration of the mobilizing agent. Diversity of blood cells can be measured using measurement of clonality by next generation sequencing and/or by multicolor flow cytometry.

In one aspect, the CXCR4 antagonist-mobilized cells (e.g., immune cells, e.g., T cells or NK cells) are obtained from a subject who has poor polyfuncationality (e.g., antigen induced production of IL2, TNFα and IFNγ etc.) of peripheral blood cells (e.g., immune cells, e.g., T cells or NK cells) prior to administration of the mobilizing agent.

In one aspect, the CXCR4 antagonist-mobilized cells (e.g., immune cells, e.g., T cells or NK cells) are obtained from a subject who has or is expected to have a poor yield of leukapheresed product without the administration of the mobilizing agent. In one aspect, the CXCR4 antagonist-mobilized cells (e.g., immune cells, e.g., T cells or NK cells) are obtained from a subject who has or is expected to have less than about 50×10⁹ (e.g., less than 40×10⁹, 20×10⁹, 10×10⁹ etc.) white blood cells (WBC) in the leukapheresed product without the administration of the mobilizing agent.

In an embodiment, a CXCR4 antagonist is administered to the subject (i.e., donor) at a dose of 0.24 mg/kg subcutaneosly daily prior to and optionally on the day of collection (e.g., leukapheresis) of cells. In some embodiments, a CXCR4 antagnoist is administered to the subject (i.e., donor) at a dose of about 0.20 mg/kg to 5 mg/kg (e.g., 0.01 mg/kg/, 0.05 mg/kg, 0.1 mg/kg, 0.15 mg/kg, 0.2 mg/kg, 0.25 mg/kg, 0.3 mg/kg, 0.4 mg/kg, 0.5 mg/kg, 0.75 mg/kg, 1 mg/kg, 2 mg/kg or 5 mg/kg), e.g., subcutaneously prior to and optionally on the day of collection of the cells. In some embodiments, a CXCR4 antagnoist is administered to the subject (i.e., donor) via alternate routes of administration (e.g., intravenous, intramuscular, intraperiotenal, transdermal, and oral etc). In some embodiments, a CXCR4 antagnoist is administered to the subject (i.e., donor) daily for about 1 to 10 days (e.g, 1 day, 2 days, 3 days, 4 days, 5 days, 6 days, 7 days, 10 days) prior to collection of the cells. In some embodiments, the cells (e.g, immune cells) are collected from the subject (i.e., donor) between about 15 min to 96 h (e.g., 15 min, 2 h, 6 h, 12 h, 24 h, 36 h, or 48 h) after the administration of the last dose of a CXCR4 antagnoist.

In an embodiment, the CXCR4 antagonist is Mozibil or Plerixafor. In an embodiment, Perixafor is administered to the subject (i.e., donor) at a dose of 0.24 mg/kg subcutaneosly daily prior to and optinoally on the day of collection (e.g., leukapheresis) of cells. In some embodiments, Perixafor is administered to the subject (i.e., donor) at a dose of about 0.20 mg/kg to 5 mg/kg (e.g., 0.01 mg/kg/, 0.05 mg/kg, 0.1 mg/kg, 0.15 mg/kg, 0.2 mg/kg, 0.25 mg/kg, 0.3 mg/kg, 0.4 mg/kg, 0.5 mg/kg, 0.75 mg/kg, 1 mg/kg, 2 mg/kg or 5 mg/kg), e.g., subcutaneously prior to and optionally on the day of collection of the cells. In some embodiments, Perixafor is administered to the subject (i.e., donor) at a dose of about 0.20 mg/kg to 5 mg/kg (e.g., 0.01 mg/kg, 0.05 mg/kg, 0.1 mg/kg, 0.15 mg/kg, 0.2 mg/kg, 0.25 mg/kg, 0.3 mg/kg, 0.4 mg/kg, 0.5 mg/kg, 0.75 mg/kg, 1 mg/kg, 2 mg/kg or 5 mg/kg), e.g., intravenously prior to and optionally on the day of collection of cells. In some embodiments, Perixafor is administered to the subject (i.e., donor) at a dose of about 0.20 mg/kg to 5 mg/kg (e.g., 0.01 mg/kg, 0.05 mg/kg, 0.1 mg/kg, 0.15 mg/kg, 0.2 mg/kg, 0.25 mg/kg, 0.3 mg/kg, 0.4 mg/kg, 0.5 mg/kg, 0.75 mg/kg, 1 mg/kg, 2 mg/kg or 5 mg/kg), e.g., intramuscularly prior to and optionally on the day of collection of cells. In some embodiments, Perixafor is administered to the subject (i.e., donor) daily for about 1 to 10 days (e.g, 1 day, 2 days, 3 days, 4 days, 5 days, 6 days, 7 days, 10 days) prior to collection of the cells. In some embodiments, the cells (e.g, immune cells) are collected from the subject (i.e., donor) between about 15 min to 96 h (e.g., 15 min, 1 h, 2 h, 6 h, 12 h, 24 h, 36 h, 48 h, or 96 h) after the administration of the last dose of Perixafor.

In further embodiments, the CXCR4 antagonist is BMS-936564/MDX-1338, LY2510924, 1,1′-[1,4-phenylenebis(methylene)]bis [1,4,8,11-tetraazacyclotetradecane] (AMD3100; Plerixafor), N, N-dipropyl-N-[4-({[(1H-imidazol-2-yl)methyl)benzyl][(1-methyl-1H-imidazol-2-yl) methyl]amino]methyl)benzyl]-N-methylbutane-1, 4-diamine tri(2R, 3R)-tartrate (KRH-3955), ([5-(4-methyl-1-piperazinyl)-2-({methyl[(8S)-5,6,7,8-tetrahydro-8-quinolinyl]amino}methyl)imidazo[1,2-a]pyridin-3-yl]methanol) (GSK812397), or N-(1H-benzimidazol-2-ylmethyl)-N′-(5,6,7,8-tetrahydroquinolin-8-yl)butane-1,4-diamine (AMD11070). In an embodiment, a CXCR4 antagonist is BL-8040. Additional CXCR4 antagonists are known in the art, such as T140 analogs, NUCC-388 and CXCR4 Antagonist III (calbiochem), and can be used in alternate embodiments of the disclosure.

In further embodiments, the CXCR4 antagonist, is a CXCL12 antagonist. In further preferred embodiments, the CXCL12 antagonist is an anti-CXCL12 antibody. One example of an anti-CXCL12 antibody includes, but is not limited to an anti-SDF-1 antibody. Examples of such a CXCL12 antagonist, can be, but are not limited to RNA oligonucleotide NOX-A12 or Tannic acid or any other chemical that blocks the interaction of CXCL12 with CXCR4.

A CXCR4 antagonist as described herein may be administered by continuous intravenous infusion in an amount sufficient to maintain the serum concentration at a level that yields >90% inhibition of CXCL12 binding by CXCR4 (see for example Hendrix et al J Acquir Immune Defic Syndr. 2004 Oct. 1; 37(2):1253-62). Other CXCR4 signal inhibitors described herein can also be used in this same manner.

Administration in vivo can be effected in one dose, continuously or intermittently (e.g., in divided doses at appropriate intervals). Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the physician. For example, the dose and duration of treatment with a CXCR4 antagonist and other mobilizing agents of the disclosure can adjusted by the physician based on peripheral blood lymphocyte count.

In a further aspect of the disclosure, cells (e.g., immune cells, e.g., T cells or NK cells) are obtained by leukophersis from a subject (i.e. an autologous or an allogeneic donor) who has received a cytokine to aid in mobilization of specific subset of immune cells into peripheral circulation. In one embodiment, the cytokine is G-CSF (filgrastim, Amgen). In some embodiments, G-CSF is administered at a dose of about 1-30 μg/kg (e.g., 1 μg/kg, 2 μg/kg, 5 μg/kg, 10 μg/kg or 30 μg/kg) subcutaneously daily for about 1-10 days. In some embodiments, G-CSF is administered at a dose of about 1-30 μg/kg (e.g., 1 μg/kg, 2 μg/kg, 5 μg/kg, or 30 μg/kg) intravenously daily for about 1-10 days. In some embodiments, the cells (e.g., immune cells, e.g., T cells or NK cells) are collected from the subject (i.e., donor) between about 2 h to 96 h after the administration of the last dose of G-CSF.

In a further aspect of the disclosure, cells (e.g., immune cells, e.g., T cells or NK cells) are obtained by leukophersis from a subject (i.e. an autologous or an allogeneic donor) who has received a cytokine to aid in mobilization of specific subset of cells (e.g., immune cells, e.g., T cells or NK cells) into peripheral circulation. In one embodiment, the cytokine is Pegfilgrastim (Neulasta, Amgen). In some embodiments, Pegfilgrastim is administered at a dose of about 6 mg (e.g., 6 mg, 8 mg, 10 mg, 12 mg, or 16 mg) by subcutaneous injection. In some embodiments, the cells (e.g., immune cells, e.g., T cells or NK cells) are collected from the subject (i.e., donor) between about 4 dyas after the administration of dose of Pegfilgrastim.

In a further aspect of the disclosure, cells (e.g., immune cells, e.g., T cells or NK cells) are obtained by leukophersis from a subject (i.e. an autologous or an allogeneic donor) who has received GM-CSF to aid in mobilization of specific subset of cells (e.g., immune cells, e.g., T cells or NK cells) into peripheral circulation. In some embodiments, GM-CSF (Leukine) is administered at a dose of about 250 μg/m²/day IV over 24 hr or subcutaneously daily for about 1-10 days. In some embodiments, the cells (e.g., immune cells, e.g., T cells or NK cells) are collected from the subject (i.e., donor) between about 2 h to 96 h after the administration of the last dose of GM-CSF.

In a further aspect of the disclosure, cells (e.g., immune cells, e.g., T cells or NK cells) are obtained by leukophersis from a subject (i.e. a donor) who has received a combination of a CXCR4 antagonist (e.g, Perixafor) and a cytokine (e.g., G-CSF, GM-CSF or Pegfilgrastim) to aid in mobilization of specific subset of cells (e.g., immune cells, e.g., T cells or NK cells) into peripheral circulation. In an exemplary embodiment, the subject receives G-CSF at dose of 10 μg/kg subcutaneously daily for 5 days followed by Perixafor at dose of 0.25 mg/kg subcutanelously on day 5. Leukapheresis products are collected approximately 2 hours after the dose of Perixafor using a CS3000-Plus blood cell separator (Baxter Healthcare). In an exemplary embodiment, the subject receives a dose of Pegfilgastrim at a dose of 12 mg by subcutaneous injection followed 4 days later by Perixafor at dose of 0.25 mg/kg subcutanelously. Leukapheresis products are collected approximately 2 hours after the dose of Perixafor using a CS3000-Plus blood cell separator (Baxter Healthcare) and used for manufacturing of cell therapy product (e.g., generation of CAR-T cells).

In a further aspect of the disclosure, cells (e.g., immune cells, e.g., T cells or NK cells) are obtained by leukophersis from a subject (i.e. a donor) after chemo-mobilization, e.g., from a subject who has received chemotherapy followed by, during the recovery of counts from chemotherapy, a CXCR4 antagonist (e.g, Perixafor) and/or a cytokine (e.g., G-CSF or GM-CSF) to aid in mobilization of specific subset of cells (e.g., immune cells, e.g., T cells or NK cells) into peripheral circulation. A number of chemo-mobilization regimens are known in the art and can be used in the methods of the disclosure. Exemplary chemo-mobilization regimens include high-dose cyclophosphamide (HDC, 4 gm/m²) or cyclophosphamide (4 gm/m²) plus etoposide (600 mg/m²) (HDCE).

In a further aspect of the disclosure, cells (e.g., immune cells, e.g., T cells or NK cells) are obtained by leukophersis from a subject (i.e. a donor) who has received a beta2 adrenergic receptor agonist to aid in mobilization of specific subset of cells (e.g., immune cells, e.g., T cells or NK cells) into peripheral circulation. In an exemplary embodiment, the beta2 adrenergic agonist is epinephrine. In an exemplary embodiment, the subject receives epinephrine by intravenous infusion at a dose of about 0.005 mg/kg/min to 0.02 mg/kg/min for about 30 minutes to 2 hours. Leukapheresis products are collected after 30 min to 2 hr of epinephrine infusion.

In a further aspect of the disclosure, cells (e.g., immune cells, e.g., T cells or NK cells) are obtained by leukophersis from a subject (i.e. a donor) following exercise. In an exemplary embodiment, the subject carries out moderate to severe exercise on a treadmill for about 10 minutes to 1 hours and leukapheresis products are collected after 10 min to 1 hr of moderate to severe exerceise. In an exemplary embodiment, the cells (e.g., immune cells, e.g., T cells or NK cells) are obtained by leukophersis from a subject (i.e. a donor) following exercise that results in heart rate that is more than 50-100% higher than the heart rate at rest.

In a further aspect of the disclosure, cells (e.g., immune cells, e.g., T cells or NK cells) are obtained by leukophersis from a subject (i.e. a donor) after a dose of Src kinase inhibitor. An exemplary Src kinase inhibitor is Dasatinb. In an exemplary embodiment, the subject receives Dasatinb at a dose of about 40-140 mg orally and the cells (e.g., immune cells, e.g., T cells or NK cells) are obtained by leukophersis from the subject between about 1-5 hour after the dose of Dasatinib. In an embodiment, Dasatinib is administered to the subject for about 7 days (e.g., 6 days, 4 days, 2 days etc.) prior to leukapheresis.

In one embodiment, the immune effector cells are obtained from a subject that has received a low, immune enhancing dose of an mTOR inhibitor. In an embodiment, the population of immune effector cells, e.g., T cells, to be engineered to express a CAR/TCR, are harvested after a sufficient time, or after sufficient dosing of the low, immune enhancing, dose of an mTOR inhibitor, such that the level of PD1 negative immune effector cells, e.g., T cells, or the ratio of PD1 negative immune effector cells, e.g., T cells/PDl positive immune effector cells, e.g., T cells, in the subject or harvested from the subject has been, at least transiently, increased.

The mobilized cells (e.g., immune cells, e.g., T cells or NK cells) of the disclosure can be used for a number of cell therapy applications. In an embodiment, the mobilized cells are administered to a subject without manipulation or genetic modifications. In an embodiment, T regulatory cells (CD3+, CD4+, CD25^{high}, CD127^{low}, FoxP3+) are removed from the mobilized cells by depletion of CD25-positive T cells. In an embodiment, the mobilized cells are depleted of regulatory T cells (TREGs) by removal of CD25^{hi}-expressing T cells using methods known in the art, such as immunomagnetic labeling with CD25 antibody labeled magnetic beads and magnet assisted cell sorting.

In an embodiment, the mobilized cells, with or without the removal of TREGs, are genetically modified prior to administration to a subject. In an embodiment, the mobilized cells (e.g., immune cells, e.g., T cells or NK cells or macrophage/monocytes or dendritic cells) are genetically modified to express a natural or a non-natural (e.g., synthetic) immune receptor. Exemplary immune receptors that can be expressed in the mobilized cells of the disclosure include a natural TCR, a recombinant TCR, a chimeric antigen receptor (CAR), including next generation CAR (e.g., SIR, TFP, Ab-TCR, SuperCAR, TAC etc), a synthetic notch receptor and the like. In an embodiment, the mobilized cells, with or without the removal of TREGs, are used for generation of a cellular vaccine.

In an embodiment, the mobilized cells are genetically modified to express an immune receptor that recognizes one or more of antigens selected from the group of CD5, CD19; CD123; CD22; CD30; CD171; CS1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fins Like Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20/MS4A1; Folate receptor alpha (FRa or FRI); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CA1X); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B 1); CCCTC-Binding Factor (Zinc Finger Protein)-Like (BORIS or Brother of the Regulator ofimprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLECI2A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); immunoglobulin lambda-like polypeptide 1 (IGLL1), MPL, Biotin, c-MYC epitope Tag, CD34, LAMP1 TROP2, GFRalpha4, CDH17, CDH6, NYBRI, CDH19, CD200R, Slea (CA19.9; Sialyl Lewis Antigen); Fucosyl-GM1, PTK7, gpNMB, CDH1-CD324, DLL3, CD276/B7H3, IL11Ra, IL13Ra2, CD179b-IGL11, TCRgamma-delta, NKG2D, CD32 (FCGR2A), Tn ag, Tim1-/HVCR1, CSF2RA (GM-CSFR-alpha), TGFbetaR2, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Gonadotropin Hormone receptor (CGHR or GR), CCR4, GD3, SLAMF6, SLAMF4, HIV1envelope glycoprotein, HTLV1-Tax, CMV pp65, EBV-EBNA3c, KSHV K8.1, KSHV-gH, influenza A hemagglutinin (HA), GAD, PDL1, Guanylyl cyclase C (GCC), auto antibody to desmoglein 3 (Dsg3), auto antibody to desmoglein 1 (Dsg1), HLA, HLA-A, HLA-A2, HLA-B, HLA-C, HLA-DP, HLA-DM, HLA-DOA, HLA-DOB, HLA-DQ, HLA-DR, HLA-G, IgE, CD99, Ras G12V, Tissue Factor 1 (TF1), AFP, GPRC5D, Claudin18.2 (CLD18A2 or CLDN18A.2), CLDN6, P-glycoprotein, STEAPI, Livl, Nectin-4, Cripto, MPL, gpA33, BST1/CD157, low conductance chloride channel, CLDN6, MMP16, UPK1B, BMPR1B, Ly6E, STEAPI, WISP1, SLC34A2 and the antigen recognized by TNT antibody.

Methods to express immune receptors (e.g., TCR, CAR, SIR, TFP, Ab-TCR etc) to generate cell therapy products are described in this disclosure and are also known in the art.

In an embodiment, the mobilized cells (e.g., immune cells, e.g., T cells or NK cells) of the disclosure are genetically modified so as to modify the expression of one or more of genes. In an exemplary embodiment, the mobilized cells of the disclosure are genetically modified to reduce or eliminate the expression of Tet2 gene. In an exemplary embodiment, the mobilized cells of the disclosure are genetically modified to reduce or eliminate the expression of TCR and HLA class I genes. In an exemplary embodiment, the mobilized cells of the disclosure are genetically modified to reduce or eliminate the expression of TCR-alpha (TRAC) gene and/or P2 macroglobulin gene. In an exemplary embodiment, the mobilized cells of the disclosure are genetically modified to alter the expression of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL (TNFSF10) and Death Receptor 5 (DR5 or TNFRSF10B). In an exemplary embodiment, the mobilized cells of the disclosure are genetically modified to increase the expression of a gene (e.g., vFLIP-K13, NEMO, cJun, JAK3, STAT5 etc.). Methods to alter the expression of a gene to generate cell therapy products are described in this disclosure and are known in the art.

In an embodiment, the mobilized cells of the disclosure are used for the purpose of generating an immune response. In an embodiment, the mobilized cells of the disclosure are used for the purpose of generating a T cell immune response.

In an embodiment, the mobilized cells of the disclosure are further processed and purified into different subtypes prior to generation of cell therapy products. In an embodiment, the mobilized cells of the disclosure are depleted of TREGs (Regulatory T cells) by depletion of CD25^{hi} cells prior to generation of cell therapy products (e.g., CAR-T or TCR-T cells). Methods to collect, process, select or deplete different cell subsets for the generation of cell therapy products are described in this disclosure and are also known in the art.

In an embodiment, the mobilized cells of the disclosure are activated and/or expanded in vitro for the generation of cell therapy products. In an embodiment, the mobilized cells of the disclosure are activated and/or expanded in vitro for about 21 days (e.g., 1 day, 2 days, 3 days, 5 days, 7 days, 9 days, 12 days, 14 days, 21 days etc.) for the generation of cell therapy product. In some embodiments, mobilized cells of the disclosure are activated and/or expanded in vitro for more than 1 day (e.g., more than 1 day, 2 days, 3 days, 5 days, 7 days, 9 days, 12 days, 14 days, 21 days etc.) for the generation of cell therapy product. In an embodiment, the mobilized cells of the disclosure are activated and/or expanded in vitro prior to administration to the subject. In an embodiment, the mobilized cells of the disclosure are activated and/or expanded in vitro for about 21 days (e.g., 1 day, 2 days, 3 days, 5 days, 7 days, 9 days, 12 days, 14 days, 21 days etc.) prior to administration to the patient. In some embodiments, mobilized cells of the disclosure are activated and/or expanded in vitro for more than 1 day (e.g., more than 1 day, 2 days, 3 days, 5 days, 7 days, 9 days, 12 days, 14 days, 21 days etc.) prior to administration to the patient. In an embodiment, the mobilized cells of the disclosure are cryopreserved prior to administration to a subject. Methods to activate and expand cells for the generation of cell therapy products are described in this disclosure and are also known in the art. Methods to cryopreserve and administer cell therapy products are also described in this disclosure and known in the art.

In an embodiment, the mobilized cells of the disclosure are activated and/or expanded in vitro under culture conditions that include a CD3 antibody, a CD28 antibody and IL2. In an exemplary embodiment, the mobilized T cells are cultured in XVIVO medium (Lonza) supplanted with 10 ng/ml CD3 antibody, 10 ng/ml CD28 antibody and 30-100 IU recombinant human-IL2. In an exemplary embodiment, CD3/CD28 beads and 100 IU recombinant human-IL2 can be used. In an exemplary embodiment, cells are cultured at 37° C., in a 5% CO₂ humidified incubator. In an embodiment, the mobilized cells of the disclosure are activated and/or expanded in vitro under culture conditions that include cytokines such as IL-7, IL-15, IL-21, IL12F, or a combination thereof.

In an embodiment, the mobilized cells of the disclosure are activated and/or expanded in vitro under culture conditions that promote the generation of central memory T cells.

In an embodiment, the mobilized cells of the disclosure are activated and/or expanded in vitro under culture conditions that suppress the generation of TREGs. In an embodiment, the mobilized cells of the disclosure are activated and/or expanded in vitro under culture conditions that promote the generation of TREGs. In an embodiment, the mobilized cells of the disclosure are activated and/or expanded in vitro under culture conditions that supress the generation of suppressor T cells. In an embodiment, the mobilized cells of the disclosure are activated and/or expanded in vitro under culture conditions that promote the generation of suppressor T cells. Methods to activate and/or expand T cells to promote or suppress the generation of TREGs and suppressor T cells are known in the art.

In an embodiment, the mobilized cells of the disclosure are used to generate a cell therapy product expressing a native or a synthetic T cell receptor. In an embodiment, the mobilized cells of the disclosure are used to generate a cell therapy product expressing a chimeric antigen receptor, including a next generation CAR (e.g., a SIR, TFP, Ab-TCR, TAC, SuperCAR, etc.). In an embodiment, the mobilized cells of the disclosure are used to generate a cell therapy product expressing a native or a synthetic NK cell receptor. In an embodiment, the mobilized cells of the disclosure are used to generate a cell therapy product expressing a native or a synthetic macrophage receptor. In an embodiment, the mobilized cells of the disclosure are used to generate a vaccine, e.g., a T cell vaccine. Methods to generate cell therapy products expressing different receptors are described in this disclosure and are also known in the art.

In an embodiment, the cell therapy product generated from the mobilized cells of the disclosure are used for autologous administration; i.e., the cell therapy product is administered to the same subject from whom the mobilized cells were harvested. In an embodiment, the cell therapy product generated from the mobilized cells of the disclosure are used for allogeneic administration; i.e., the cell therapy product is administered to a different subject from than the one from whom the mobilized cells were harvested. Methods to use cell therapy products for autologous or allogeneic use are described in this disclosure and are also known in the art.

In an embodiment, the cell therapy product generated from the mobilized cells of the disclosure are used for the prevention and treatment of various diseases, (e.g., cancer, immune, degenerative and infectious diseases). In an embodiment, the cell therapy product generated from the mobilized cells of the disclosure are used for the prevention and treatment of various diseases in combination with other agents (e.g., chemotherapy drugs; antibodies, cytokines etc.).

In some embodiment, the use of a mobilization agent (e.g., CXCR4 antagonist, G-CSF, GM-CSF or Dasatinib) results in higher yield of the cell therapy product (e.g., CAR-T, SIR-T or TCR-T). In an embodiment, the use of a mobilization agent (e.g., CXCR4 antagonist, G-CSF, GM-CSF or Dasatinib) results in about 20% (e.g., 20%, 50%, 75%, 100% etc.) higher number of cells in the manufactured cell therapy product as compared to the cells (e.g., CAR-T, SIR-T or TCR-T cells) in cell therapy product manufactured from apheresis product collected without the use of the mobilization agent.

In an embodiment, the use of a mobilization agent (e.g., CXCR4 antagonist, G-CSF, GM-CSF or Dasatinib) results in shorter manufacturing time of cell therapy (e.g., CAR-T, SIR-T or TCR-T) product. In an embodiment, the use of a mobilization agent (e.g., CXCR4 antagonist, G-CSF, GM-CSF or Dasatinib) results in about 10% (e.g., 20%, 50%, 75%, 100% etc.) shorter manufacturing time for cell therapy product as compared to the manufacturing time for cell therapy product manufactured from apheresis product collected without the use of the mobilization agent.

In an embodiment, the use of a mobilization agent (e.g., CXCR4 antagonist, G-CSF, GM-CSF or Dasatinib) results in fewer failures in the manufacturing of cell therapy (e.g., CAR-T, SIR-T or TCR-T) product. In an embodiment, the use of a mobilization agent (e.g., CXCR4 antagonist, G-CSF, GM-CSF or Dasatinib) results in about 10% (e.g., 20%, 50%, 75%, 100% etc.) reduction in the manufacturing failure for cell therapy product as compared to the manufacturing failure for cell therapy product manufactured from apheresis product collected without the use of the mobilization agent.

In an embodiment, the use of a mobilization agent (e.g., CXCR4 antagonist, G-CSF, GM-CSF or Dasatinib) results in lower cost of manufacturing of cell therapy (e.g., CAR-T, SIR-T or TCR-T) product. In an embodiment, the use of a mobilization agent (e.g., CXCR4 antagonist, G-CSF, GM-CSF or Dasatinib) results in about 10% (e.g., 20%, 50%, 75%, 100% etc.) reduction in the manufacturing cost for cell therapy product as compared to the manufacturing failure for cell therapy product manufactured from apheresis product collected without the use of the mobilization agent.

In an embodiment, the use of a mobilization agent (e.g., CXCR4 antagonist, G-CSF, GM-CSF or Dasatinib) results in improved diversity of cell therapy e.g., CAR-T, SIR-T or TCR-T) product. In an embodiment, the use of a mobilization agent (e.g., CXCR4 antagonist, G-CSF, GM-CSF or Dasatinib) results in about 10% (e.g., 20%, 50%, 75%, 100% etc.) higher diversity of cell therapy product as compared to the cell therapy product manufactured from apheresis product collected without the use of the mobilization agent. Diversity of a cell therapy product can be measured by methods known in the art such as clonal analysis, multi-color flow cytometry and the like.

In an embodiment, the cell therapy product generated from a population of cells that had been collected (e.g. apheresis product) following the administration of mobilization agents (e.g., CXCR4 antagonist, G-CSF, GM-CSF or Dasatinib etc) show no significant loss of cytotoxicity activity or improved cytotoxicity against their target cells as compared to a cell therapy product generated from a population of cells that had been collected (e.g. apheresis product) without the administration of mobilization agents. In an embodiment, the use of a mobilization agent (e.g., CXCR4 antagonist, G-CSF, GM-CSF or Dasatinib) results in no greater than 50% (e.g., 20%, 25%, 45% etc.) loss of cytotoxicity of cell therapy product as compared to the cell therapy product manufactured from apheresis product collected without the use of the mobilization agent. The cytotoxicity of a cell therapy product can be measured using methods known in the art, such as the Matador cytotoxicity assay or radioactive chromium release assay.

In an embodiment, the cell therapy product generated from a population of cells that had been collected (e.g. apheresis product) following the administration of mobilization agents (e.g., CXCR4 antagonist, G-CSF, GM-CSF or Dasatinib etc) show improved or no significant loss of target antigen-induced cytokine production (TNFα, IL2, IFNγ) as compared to a cell therapy product generated from a population of cells that had been collected (e.g. apheresis product) without the administration of mobilization agents. In an embodiment, the use of a mobilization agent (e.g., CXCR4 antagonist, G-CSF, GM-CSF or Dasatinib) results in no more than 50% (e.g., 20%, 25%, 50%, etc.) loss of target antigen induced cytokine production of cell therapy product as compared to the cell therapy product manufactured from apheresis product collected without the use of the mobilization agent. Cytokine production by the cell therapy product is measured using methods known in the art, such as ELISA and Flow cytometry etc.

In an embodiment, the cell therapy product generated from a population of cells that had been collected (e.g. apheresis product) following the administration of mobilization agents (e.g., CXCR4 antagonist, G-CSF, GM-CSF or Dasatinib etc) show improved or no significant loss of in vivo efficacy as compared to a cell therapy product generated from a population of cells that had been collected (e.g. apheresis product) without the administration of mobilization agents. In an embodiment, the use of a mobilization agent (e.g., CXCR4 antagonist, G-CSF, GM-CSF or Dasatinib) results in no more than 50% (e.g., 20%, 25%, 50%, etc.) loss of in vivo efficacy of cell therapy product as compared to the cell therapy product manufactured from apheresis product collected without the use of the mobilization agent. The vivo efficacy of a cell therapy product is measured using methods known in the art, such as xenograft studies in immunodeficient mice.

In an embodiment, the cell therapy product generated from a population of cells that had been collected (e.g. apheresis product) following the administration of mobilization agents (e.g., CXCR4 antagonist, G-CSF, GM-CSF or Dasatinib etc) show improved or no significant loss of stem like T cells as compared to a cell therapy product generated from a population of cells that had been collected (e.g. apheresis product) without the administration of mobilization agents. In an embodiment, the use of a mobilization agent (e.g., CXCR4 antagonist, G-CSF, GM-CSF or Dasatinib) results in no more than 50% (e.g., no more than 20%, 25%, 50%, etc.) loss of stem like T cells in the cell therapy product as compared to the cell therapy product manufactured from apheresis product collected without the use of the mobilization agent. The stem like T cell population in the cell therapy product is measured by Flow cytometry using markers (e.g., CD62L+, CD7+, Pgp+) known in the art.

In an embodiment, the cell therapy product generated from a population of cells that had been collected (e.g. apheresis product) following the administration of mobilization agents (e.g., CXCR4 antagonist, G-CSF, GM-CSF or Dasatinib etc) show improved or no significant loss of naïve T cells as compared to a cell therapy product generated from a population of cells that had been collected (e.g. apheresis product) without the administration of mobilization agents. In an embodiment, the use of a mobilization agent (e.g., CXCR4 antagonist, G-CSF, GM-CSF or Dasatinib) results in no more than 50% (e.g., no more than 20%, 25%, 50%, etc.) loss of naive T cells in the cell therapy product as compared to the cell therapy product manufactured from apheresis product collected without the use of the mobilization agent. The naive T cell population in the cell therapy product is measured by Flow cytometry using markers known in the art.

In an embodiment, the cell therapy product generated from a population of cells that had been collected (e.g. apheresis product) following the administration of mobilization agents (e.g., CXCR4 antagonist, G-CSF, GM-CSF or Dasatinib etc) show improved or no significant loss of central memory T cells as compared to a cell therapy product generated from a population of cells that had been collected (e.g. apheresis product) without the administration of mobilization agents. In an embodiment, the use of a mobilization agent (e.g., CXCR4 antagonist, G-CSF, GM-CSF or Dasatinib) results in no more than 50% (e.g., no more than 20%, 25%, 50%, etc.) loss of central memory T cells in the cell therapy product as compared to the cell therapy product manufactured from apheresis product collected without the use of the mobilization agent. The central memory T cell population in the cell therapy product is measured by Flow cytometry using markers known in the art.

In an embodiment, the cell therapy product generated from a population of cells that had been collected (e.g. apheresis product) following the administration of mobilization agents (e.g., CXCR4 antagonist, G-CSF, GM-CSF or Dasatinib etc) show improved or no significant loss of effector T cells as compared to a cell therapy product generated from a population of cells that had been collected (e.g. apheresis product) without the administration of mobilization agents. In an embodiment, the use of a mobilization agent (e.g., CXCR4 antagonist, G-CSF, GM-CSF or Dasatinib) results in no more than 50% (e.g., no more than 20%, 25%, 50%, etc.) loss of effector T cells in the cell therapy product as compared to the cell therapy product manufactured from apheresis product collected without the use of the mobilization agent. The effector T cell population in the cell therapy product is measured by Flow cytometry using markers known in the art.

In an embodiment, the cell therapy product generated from a population of cells that had been collected (e.g. apheresis product) following the administration of mobilization agents (e.g., CXCR4 antagonist, G-CSF, GM-CSF or Dasatinib etc) show no major enrichment of regulatory T cells (e.g. TREGs) as compared to a cell therapy product generated from a population of cells that had been collected (e.g. apheresis product) without the administration of mobilization agents. In an embodiment, the use of a mobilization agent (e.g., CXCR4 antagonist, G-CSF, GM-CSF or Dasatinib) results in no more than 50% (e.g., no more than 20%, 25%, 50%, etc.) increase in the proportion of regulatory T cells (TREGs) in the cell therapy product as compared to the cell therapy product manufactured from apheresis product collected without the use of the mobilization agent. The TREGs population in the cell therapy product is measured by Flow cytometry using markers known in the art.

In an embodiment, the use of a mobilization agent (e.g., CXCR4 antagonist, G-CSF, GM-CSF or Dasatinib) results in no significant loss and/or improved polyfuncationality (e.g., antigen induced IL2, TNFα, IFNγ production) of cell therapy e.g., CAR-T, SIR-T or TCR-T) product. In an embodiment, the use of a mobilization agent (e.g., CXCR4 antagonist, G-CSF, GM-CSF or Dasatinib) results in about 10% (e.g., 20%, 50%, 75%, 100% etc.) higher polyfunctionality of cell therapy product as compared to the cell therapy product manufactured from apheresis product collected without the use of the mobilization agent.

In an embodiment, the use of a mobilization agent (e.g., CXCR4 antagonist, G-CSF, GM-CSF or Dasatinib) results in no significant loss and/or improved in vivo expansion of cell therapy e.g., CAR-T, SIR-T or TCR-T) product. In an embodiment, the use of a mobilization agent (e.g., CXCR4 antagonist, G-CSF, GM-CSF or Dasatinib) results in about 10% (e.g., 20%, 50%, 75%, 100% etc.) higher expansion of cell therapy product following administration to the subject as compared to the cell therapy product manufactured from apheresis product collected without the use of the mobilization agent.

In an embodiment, the use of a mobilization agent (e.g., CXCR4 antagonist, G-CSF, GM-CSF or Dasatinib) results in no significant loss and/or improved persistence of cell therapy e.g., CAR-T, SIR-T or TCR-T) product. In an embodiment, the use of a mobilization agent (e.g., CXCR4 antagonist, G-CSF, GM-CSF or Dasatinib) results in about 10% (e.g., 20%, 50%, 75%, 100% etc.) greater persistence of cell therapy product following administration to the subject as compared to the cell therapy product manufactured from apheresis product collected without the use of the mobilization agent.

In an embodiment, the use of a mobilization agent (e.g., CXCR4 antagonist, G-CSF, GM-CSF or Dasatinib) results in improved or no significant loss of tissue (e.g. tumor) penetration of cell therapy e.g., CAR-T, SIR-T or TCR-T) product. In an embodiment, the use of a mobilization agent (e.g., CXCR4 antagonist, G-CSF, GM-CSF or Dasatinib) results in about 10% (e.g., 20%, 50%, 75%, 100% etc.) greater tissue (e.g. tumor) penetration of cell therapy product following administration to the subject as compared to the cell therapy product manufactured from apheresis product collected without the use of the mobilization agent.

In an embodiment, the use of a mobilization agent (e.g., CXCR4 antagonist, G-CSF, GM-CSF or Dasatinib) results in improved or no significant loss of anti-tumor efficacy of cell therapy e.g., CAR-T, SIR-T or TCR-T) product. In an embodiment, the use of a mobilization agent (e.g., CXCR4 antagonist, G-CSF, GM-CSF or Dasatinib) results in about 10% (e.g., 20%, 50%, 75%, 100% etc.) greater anti-tumor efficacy of cell therapy product following administration to the subject as compared to the cell therapy product manufactured from apheresis product collected without the use of the mobilization agent.

In an embodiment, the use of a mobilization agent (e.g., CXCR4 antagonist, G-CSF, GM-CSF or Dasatinib) results no significant increase in the toxicity (e.g., cytokine release syndrome, neurotoxicity etc.) of cell therapy e.g., CAR-T, SIR-T or TCR-T) product.

In certain aspects of the present disclosure, immune effector cells, e.g., T cells, can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations.

In one aspect, T cells are isolated from peripheral blood lymphocytes by lysing the red blood cells and depleting the monocytes, for example, by centrifugation through a PERCOLL™ gradient or by counterflow centrifugal elutriation.

In one embodiment, the mobilized cells are further enriched for cells that expresses P-glycoprotein ((P-gp or Pgp; MDR1, ABCB1, CD243). In one embodiment, the mobilized cells are enriched for cells that stains dull with dyes that are substrates of P-glycoprotein mediated efflux as described in application no. PCT/US2017/042248, which is incorporated herein by reference. In some embodiments, cells which lack expression of p-gp or p-gp activity are removed from the population.

The methods described herein can include more than one selection step, e.g., more than one depletion step.

The methods described herein can further include removing cells from the population which express a tumor antigen, e.g., CD19, CD30, CD38, CD123, CD20/MS4A1, CD14 or CD11b. Also provided are methods that include removing cells from the population which express a check point inhibitor.

The methods described herein can further include removing regulatory T cells (TREGs) from the mobilized cells. Methods for removal of TREGs are known in the art and include removal of CD25 expressing T cells.

In one embodiment, a T cell population can be selected that expresses one or more of IFN-γ, TNFα, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

T cells for stimulation can also be frozen after a washing step.

Also contemplated in the context of the disclosure is the collection of blood samples or apheresis product from a subject at a time period prior to when the expanded cells as described herein might be needed. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use.

In certain aspects, the T cells may be expanded, frozen, and used at a later time. In certain aspects, samples are collected from a patient shortly after diagnosis of a particular disease as described herein but prior to any treatments.

The disclosure also provides BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and DR5 inhibitors and methods of use therefore. In particular, the disclosure provides immune effector cells, e.g., CAR- and TCR-expressing T cells, comprising BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and DR5 inhibitors, and use of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and DR5 inhibitors in connection with immune effector cellular therapy product, e.g., CAR-T cells or TCR-T cells. BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and DR5 inhibitors of the disclosure, together with their methods of use, are described in more detail below.

In an embodiment the immune effector cell therapy product is a CAR-T cell or a TCR-T cell or a BiTE, wherein the CAR-T/TCR-T or BiTE is targeted to, but not limited to, one or more of antigens selected from the group of CD5, CD19; CD123; CD22; CD30; CD171; CS1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fins Like Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20/MS4A1; Folate receptor alpha (FRa or FRI); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CA1X); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B 1); CCCTC-Binding Factor (Zinc Finger Protein)-Like (BORIS or Brother of the Regulator ofimprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); immunoglobulin lambda-like polypeptide 1 (IGLL1), MPL, Biotin, c-MYC epitope Tag, CD34, LAMP1 TROP2, GFRalpha4, CDH17, CDH6, NYBRI, CDH19, CD200R, Slea (CA19.9; Sialyl Lewis Antigen); Fucosyl-GM1, PTK7, gpNMB, CDH1-CD324, DLL3, CD276/B7H3, IL11Ra, IL13Ra2, CD179b-IGL11, TCRgamma-delta, NKG2D, CD32 (FCGR2A), Tn ag, Timi-/HVCRi, CSF2RA (GM-CSFR-alpha), TGFbetaR2, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Gonadotropin Hormone receptor (CGHR or GR), CCR4, GD3, SLAMF6, SLAMF4, HIV1envelope glycoprotein, HTLV1-Tax, CMV pp65, EBV-EBNA3c, KSHV K8.1, KSHV-gH, influenza A hemagglutinin (HA), GAD, PDL1, Guanylyl cyclase C (GCC), auto antibody to desmoglein 3 (Dsg3), auto antibody to desmoglein 1 (Dsg1), HLA, HLA-A, HLA-A2, HLA-B, HLA-C, HLA-DP, HLA-DM, HLA-DOA, HLA-DOB, HLA-DQ, HLA-DR, HLA-G, IgE, CD99, Ras G12V, Tissue Factor 1 (TF1), AFP, GPRC5D, Claudin18.2 (CLD18A2 or CLDN18A.2), CLDN6, P-glycoprotein, STEAPI, Livl, Nectin-4, Cripto, MPL, gpA33, BST1/CD157, low conductance chloride channel, CLDN6, MMP16, UPK1B, BMPR1B, Ly6E, STEAPI, WISP1, SLC34A2 and the antigen recognized by TNT antibody.

The disclosure provides inhibitors of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and DR5, and methods for enhancing immune effector cell functions, e.g., CAR-expressing cell functions, by using such compositions and/or other means as described herein. Any inhibitors of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and DR5 known in the art can be used as a BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and DR5 inhibitor according to the disclosure. In some embodiments, the inhibitors of TRAIL and DR5 are also used to prevent, ameliorate or treat the side effects, e.g., CRS and neurotoxicity, of immune therapy (e.g., Blinatumomab) and immune effector cell therapy products (e.g., CAR-T). Examples of inhibitors of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and DR5 are described below.

According to the disclosure, gene editing systems can be used as inhibitors of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and DR5. Also contemplated by the disclosure are the uses of nucleic acid encoding one or more components of a BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and DR5 gene editing system. A number of gene editing systems, such as CRISPR/Cas9, Zn finger nucleases, Talons etc are known in the art and can be used in the methods of the disclosure.

The CRISPR/Cas system can be used to modify, e.g., delete one or more nucleic acids of a gene, or a gene regulatory element, or introduce a premature stop which thus decreases expression of a functional gene, e.g., BRD9. The CRISPR/Cas system can alternatively be used like RNA interference, turning off a gene in a reversible fashion. In a mammalian cell, for example, the RNA can guide the Cas protein to the BRD9 promoter, sterically blocking RNA polymerases.

An exemplary gRNA molecule of the disclosure comprises, e.g., consists of a first nucleic acid having the sequence (where the “n′”s refer to the residues of the targeting sequence (e.g., as described herein, e.g., in Table 8), and may consist of 15-25 nucleotides, e.g., consist of 20 nucleotides): nnnnnnnnnnnnnnnnnnnnGUUUUAGAGCUAUGCUGUUUUG (SEQ ID NO: 2041); and a second nucleic acid sequence having the sequence: AACUUACCAAGGAACAGCAUAGCAAGUUAAAAUAAGGCUAGUCCGUUAUCAAC UUGAAAAAGUGGCACCGAGUCGGUGC, optionally with 1, 2, 3, 4, 5, 6, or 7 (e.g., 4 or 7, e.g., 7) additional U nucleotides at the 3′ end (SEQ ID NO: 2042).

The second nucleic acid molecule may alternatively consist of a fragment of the sequence above, wherein such fragment is capable of hybridizing to the first nucleic acid. An example of such second nucleic acid molecule is: AACAGCAUAGCAAGUUAAAAUAAGGCUAGUCCGUUAUCAACUUGAAAAAG UGGCACCGAGUCGGUGC, optionally with 1, 2, 3, 4, 5, 6, or 7 (e.g., 4 or 7, e.g., 7) additional U nucleotides at the 3′ end (SEQ ID NO: 2054).

Another exemplary gRNA molecule of the disclosure comprises, e.g., consists of a first nucleic acid having the sequence (where the “n′”s refer to the residues of the targeting sequence (e.g., as described herein, e.g., in Table 8), and may consist of 15-25 nucleotides, e.g., consist of 20 nucleotides): nnnnnnnnnnnnnnnnnnnGUUUUAGAGCUAGAAAUAGCAAGUUAAAA UAAGGCUAGUCCGUUAUCAACUUGAAAAAGUGGCACCGAGUCGGUGC (SEQ ID NO: 2055), optionally with 1, 2, 3, 4, 5, 6, or 7 (e.g., 4 or 7, e.g., 4) additional U nucleotides at the 3′ end.

Alternatively, gRNAs containing the target-specific sequence for guiding Cas9 protein to genomic location of a gene can be obtained from commercial vendors, such as Integrated DNA Technologies (IDT). The crRNAs or crRNA XT, which form a gRNA duplex with tracrRNA, or sgRNAs, which are single RNA molecules comprised of both crRNA and tracrRNA sequences can be ordered pre-designed or custom-designed using user-defined protospacer designs at the IDT website (www.idtdna.com). The crRNA or sgRNAs can be used to edit a gene using the instructions of the manufacturer and/or methods known in the art.

In embodiments, the gRNA comprises a targeting sequence which is fully complementarity to 15-25 nucleotides, e.g., 20 nucleotides of the targeted gene, e.g., BRD9. In embodiments, the 15-25 nucleotides, e.g., 20 nucleotides, of the targeted gene are disposed immediately 5′ to a protospacer adjacent motif (PAM) sequence recognized by the Cas protein of the CRISPR/Cas system (e.g., where the system comprises a S. pyogenes Cas9 protein, the PAM sequence comprises NGG, where N can be any of A, T, G or C). Examples of gRNA targeting sequences (including the PAM sequences) which are useful in the various embodiments of the disclosure to inhibit target BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, are provided below in Table 4b.

In embodiments, the targeting sequence of the gRNA comprises, e.g., consists of, a RNA sequence complementary to a sequence listed in Table 4a. In embodiments, the gRNA comprises a sequence listed in Table 8.

In one embodiment, foreign DNA can be introduced into the cell along with the CRISPR/Cas system, e.g., DNA encoding a CAR, e.g., as described herein; depending on the sequences of the foreign DNA and chromosomal sequence, this process can be used to integrate the DNA encoding the CAR, e.g., as described herein, at or near the site targeted by the CRISPR/Cas system. As shown herein, in the examples, but without being bound by theory, such integration may lead to the expression of the CAR as well as disruption of the BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, gene. Such foreign DNA molecule is referred to herein as “template DNA.” In embodiments, the template DNA further comprises homology arms 5′ to, 3′ to, or both 5′ and 3′ to the nucleic acid of the template DNA which encodes the molecule or molecules of interest (e.g., which encodes a CAR described herein), wherein said homology arms are complementary to genomic DNA sequence flanking the target sequence.

In an embodiment, the CRISPR/Cas system of the disclosure comprises Cas9, e.g., S. pyogenes Cas9, and a gRNA comprising a targeting sequence which hybridizes to a sequence of the target gene, e.g. BRD9 gene. In an embodiment, the CRISPR/Cas system comprises nucleic acid encoding a BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 gRNA and nucleic acid encoding a Cas protein, e.g., Cas9, e.g., S. pyogenes Cas9. In an embodiment, the CRISPR/Cas system comprises a gRNA against the target gene and nucleic acid encoding a Cas protein, e.g., Cas9, e.g., S. pyogenes Cas9.

Examples of genomic target sequences for for which gRNAs comprising complementary targeting sequences can be generated for use in the disclosure are listed in the Table 4a below. In embodiments, the inhibitor is nucleic acid encoding a gRNA molecule specific for BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, DR4 and DR5, wherein the nucleic acid comprises the sequence of a Target Sequence from the Table 4a, e.g., under the control of a U6- or H1-promoter. For example, the target sequence from Table 4a can be cloned in the pLenti-CRISPR-v2 vector (SEQ ID NO: 359) available from Addgene (Plasmid #52961) and following the instructions provided by the distributor. The lentiviral vectors encoding the gRNAs can be prepared and used according to the instructions provided by the distributor and methods described in Sanjana N E, Shalem O, Zhang F. Nat Methods. 2014 August; 11(8):783-4.

The pLenti-CRISPR-v2 vector vector also coexpresses the S. pyogenes Cas9 and a puromycin resistance gene. Alternatively, these gRNAs are expressed from one vector and the S. pyogenes Cas9 is expressed from another vector. Methods to express more than one gRNA from the same vector are known in the art (Kabadi A M V et al, Nucleic Acids Research, 2014, Vol. 42, No. 19 e147, 2014) and can be used in alternate embodiment of the disclosure.

TABLE 4a
gRNA target sequence within the indicated targeted genes.

| | Target | gRNA | Target context |
|---|---|---|---|
| TARGET GENE | Gene ID | SEQ ID NO | SEQ ID NO: |
| BRD9 | 65980 | 1 to 4 | 106-109 |
| EZH2 | 2146 | 5 to 8 | 110-113 |
| MLL2 | 8085 | 9 to 12 | 114-117 |
| MLL3 | 58508 | 13-16 | 118-121 |
| MLL4 (KMT2B) | 9757 | 17-20 | 122-125 |
| BRD8 | 10902 | 21-24 | 126-129 |
| CHEK2 | 11200 | 25-28 | 130-133 |
| TET1 | 80312 | 29-32 | 134-137 |
| TET2 | 54790 | 33-36 | 138-141 |
| TET3 | 200424 | 37-40 | 142-145 |
| ATM | 472 | 41-44 | 146-149 |
| FAT1 | 2195 | 45-48 | 150-153 |
| FBXW10 | 10517 | 49-52 | 154-157 |
| BCOR | 54880 | 53-56 | 158-161 |
| ASXL1 | 171023 | 57-60 | 162-165 |
| PHF6 | 84295 | 61-64 | 166-169 |
| KDM6A | 7403 | 65-68 | 170-173 |
| SF3B1 | 23451 | 69-72 | 174-177 |
| YY1 | 7528 | 73-76 | 178-181 |
| CBFB | 865 | 77-80 | 182-185 |
| Runx1 | 861 | 81-84 | 186-189 |
| PRDM1 (BLIMP1) | 639 | 85-88 | 190-193 |
| HDAC2 | 3066 | 89-92 | 194-197 |
| EHMT2 (G9A) | 10919 | 93-96 | 198-201 |
| SMARCA4 | 6597 | 97-100 | 202-205 |
| CREBBP | 1387 | 101-104 | 206-209 |
| TNFSF10 | 8743 | 1775-1778 | |
| TNFRSF10A | 8797 | 1779-1782 | |
| TNFRSF10B | 8795 | 1783-1786 | |

TABLE 4b
Target sequence with PAM sequence for
different gRNAs targeting different genes

| | SEQ ID NO | | SEQ ID NO |
|---|---|---|---|
| | of gRNA | | of gRNA |
| TARGET | with PAM | TARGET | with PAM |
| GENE | SEQUENCE | GENE | SEQUENCE |
| BRD9 | 500-549 | KDM6A | 1163-1212 |
| EZH2 | 551-600 | PHF6 | 1214-1263 |
| MLL2 | 602-651 | SF3B1 | 1265-1314 |
| (KMT2D) | | | |
| MLL3 | 653-702 | YY1 | 1316-1365 |
| (KMT2C) | | | |
| MLL4 | 704-753 | CBFB | 1367-1416 |
| (KMT2B) | | | |
| CHEK2 | 908-957 | RUNX1 | 1418-1467 |
| TET1 | 755-804 | PRDM1 | 1469-1518 |
| TET2 | 806-855 | HDAC2 | 1520-1569 |
| TET3 | 857-906 | EHMT2 | 1571-1620 |
| ASXL1 | 959-1008 | SMARCA4 | 1622-1671 |
| ATM | 1010-1059 | CREBBP | 1673-1722 |
| FBXW10 | 1061-1110 | BCOR | 1724-1773 |
| FAT1 | 1112-1161 | TNFSF10 | 2060-2109 |
| TNFRSF10A | 2111-2160 | TNFRSF10B | 2162-2211 |

In addition to Cas9/CRISP, other gene editing methods, such as TALENS and Zn finger nucleases, can be used in alternate embodiment of the disclosure.

A BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 TALEN can be used inside a cell to produce a double-stranded break (DSB). A mutation can be introduced at the break site if the repair mechanisms improperly repair the break via non-homologous end joining.

TALENs specific to sequences a gene, e.g., BRD9, can be constructed using any method known in the art, including various schemes using modular components. Zhang et al. (2011) Nature Biotech. 29: 149-53; Geibler et al. (2011) PLoS ONE 6: e19509; U.S. Pat. Nos. 8,420,782; 8,470,973, the contents of which are hereby incorproated by reference in their entirety.

Like a TALEN, a ZFN can create a double-stranded break in the DNA, which can create a frame-shift mutation if improperly repaired, leading to a decrease in the expression and amount of a gene, e.g., BRD9, in a cell. ZFNs can also be used with homologous recombination to mutate a gene or to introduce nucleic acid encoding a CAR at a site at or near the targeted sequence. As discussed above, the nucleic acid encoding a CAR may be introduced as part of a template DNA. In embodiments, the template DNA further comprises homology arms 5′ to, 3′ to, or both 5′ and 3′ to the nucleic acid of the template DNA which encodes the molecule or molecules of interest (e.g., which encodes a CAR described herein), wherein said homology arms are complementary to genomic DNA sequence flanking the target sequence.

ZFNs specific to sequences in the BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 gene can be constructed using any method known in the art. Without being bound by theory, it is believed that use of gene editing systems (e.g., CRISPR/Cas gene editing systems) which target a gene may allow one to inhibit one or more functions of the targeted gene, for example, causing an editing event which results in expression of a truncated gene. Again, without being bound by theory, such truncated gene/protein may preserve one or more functions of the targeted gene/protein, while inhibiting one or more other functions. Gene editing systems which target a late exon or intron of a gene may be particularly preferred in this regard. In an aspect, the gene editing system of the disclosure targets a late exon or intron of the targeted gene, e.g., BRD9. In an aspect, the gene editing system inhibitor of the disclosure targets an exon or intron downstream of exon 8 when applicable.

Without being bound by theory, it may also be preferable in other embodiments to target an early exon or intron of targeted gene, e.g., BRD9 gene, for example, to introduce a premature stop codon in the targeted gene which results in no expression of the gene product, or expression of a completely non-functional gene product. Gene editing systems which target an early exon or intron of the targeted gene, e.g., BRD9 gene, may be particularly preferred in this regard. In an aspect, the gene editing system BRD9 inhibitor, e.g., BRD9 inhibitor of the disclosure targets an early exon or intron of the BRD9 gene. In an aspect, the gene editing system of the disclosure targets an exon or intron upstream of exon 4 of the targeted gene. In embodiments, the gene editing system inhibitor, e.g., a BRD9 inhibitor, targets exon 1, exon 2, or exon 3, e.g., exon 3, of the targeted gene, e.g., BRD9 gene.

Without being bound by theory, it may also be preferable in other embodiments to target a sequence of the gene that is specific to one or more isoforms of the above genes but does not affect one or more other isoforms of the above genes. In embodiments, it may be preferable to specifically target isoforms of the above genes which contain a catalytic and/or signaling domain.

According to the disclosure, double stranded RNA (“dsRNA”), e.g., siRNA or shRNA can be used as inhibitors of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5. Also contemplated by the disclosure are the uses of nucleic acid encoding said dsRNA BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, inhibitors.

Examples of nucleic acid sequences that encode shRNA sequences are provided in Table 5. The Target Sequence refers to the sequence within the BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 genomic DNA (or surrounding DNA). In embodiments, the BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 inhibitor is an siRNA or shRNA specific for a Target sequence listed below, or specific for its mRNA complement. In embodiments, the inhibitor is a shRNA encoded by the Nucleic Acid encoding the BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 shRNA of the Table 5.

In embodiments, the inhibitor is nucleic acid comprising BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 shRNA of the Table 5 below, e.g., which is under the control of a U6 or Hl promoter such that a BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, shRNA is produced. In embodiments, the disclosure provides a siRNA or shRNA comprising sequence which is the RNA analog (i.e., all T nucleic acid residues replaced with U nucleic acid residues) of the Target sequence of shRNA, e.g., the Target sequence of shRNA of any of the shRNAs of Table 5.

TABLE 5
Sequence of shRNAs targeting different genes

| | | shRNA target | Nucleic Acid |
|---|---|---|---|
| TARGET | Target | Sequence | encoding shRNA |
| GENE | Gene ID | SEQ ID NO: | SEQ ID NO: |
| BRD9 | 65980 | 230-232 | 293-295 |
| EZH2 | 2146 | 211-212 | 274-275 |
| MLL3 | 58508 | 223-225 | 286-288 |
| MLL4 | 9757 | 235-237 | 298-300 |
| (KMT2B) | | | |
| CHEK2 | 11200 | 219-220 | 282-283 |
| TET2 | 54790 | 215-216 | 278-279 |
| ATM | 472 | 213-214 | 276-277 |
| FAT1 | 2195 | 240-241 | 303-304 |
| FBXW10 | 10517 | 217-218 | 280-281 |
| BCOR | 54880 | 221-222 | 284-285 |
| YY1 | 7528 | 242-246 | 305-309 |
| CBFB | 865 | 247-249 | 310-312 |
| Runx1 | 861 | 250-252 | 313-315 |
| PRDM1 | 639 | 253-255 | 316-318 |
| (BLIMP1) | | | |
| HDAC2 | 3066 | 256-260 | 319-323 |
| EHMT2 | 10919 | 261-265 | 324-328 |
| (G9A) | | | |
| SMARCA4 | 6597 | 266-268 | 329-331 |
| CREBBP | 1387 | 269-272 | 332-335 |
| TNFSF10 | 8743 | 2213-2216 | 2226-2229 |
| TNFRSF10A | 8797 | 2217-2220 | 2230-2233 |
| TNFRSF10B | 8795 | 2221-2224 | 2234-2237 |

Additional dsRNA inhibitor of the above genes, e.g., shRNA and siRNA molecules can be designed and tested using methods known in the art and as described herein.

In embodiments, it may be preferable to specifically target isoforms of the genes, e.g., BRD9, which contain an active domain, e.g a catalytic domain or signaling domain.

Table 6 provides names and Cas # of several other chemical inhibitors targeting that can be used in the present disclosure to generate a diverse population of immune effector cells for the purpose of cellular therapy and to modify the effect of BiTEs and other immune therapy agents.

TABLE 6
Chemical inhibitors of different genes/proteins that can be
used in the methods of the disclosure.

| Class | Name | Cas # |
|---|---|---|
| BRD9 inhibitor | BI-7273 | 1883429-21-7 |
| BRD9 inhibitor | I-BRD9 | 1714146-59-4 |
| BRD7/BRD9 inhibitor | BI-9564 | 1883429-22-8 |
| BRD7/BRD9 inhibitor | LP99 | 1808951-93-0 |
| EZH2 inhibitor | Tazemetostat | 1403254-99-8 |
| | (EPZ-6438) | |
| EZH2 inhibitor | 3-Deazaneplanocin A | 102052-95-9 |
| EZH2 inhibitor | GSK503 | 1346572-63-1 |
| EZH2 inhibitor | GSK126 | 1346574-57-9 |
| EZH2 inhibitor | CPI-169 | 1450655-76-1 |
| EZH2 inhibitor | UNC1999 | 1431612-23-5 |
| EZH2 inhibitor | CPI-1205 | 1621862-70-1 |
| EZH2 inhibitor | El1 | 1418308-27-6 |
| EZH2 inhibitor | EPZ011989 | 1598383-40-4 |
| EZH2 inhibitor | GSK343 | 1346704-33-3 |
| EZH2 inhibitor | CPI-360 | 1802175-06-9 |
| EZH2 inhibitor | EPZ005687 | 1396772-26-1 |
| PCR2 inhibitor | EED226 | 2083627-02-3 |
| CBFβ-Runx1 Inhibitor | Ro5-3335 | 30195-30-3 |
| G9a inhibitor | UNC0642 | 1481677-78-4 |
| G9a inhibitor | BIX01294 | 1808255-64-2 |
| | (hydrochloride | |
| | hydrate) | |
| G9a inhibitor | UNC0321 | 1238673-32-9 |
| | (trifluoroacetate salt) | |
| G9a/GLP inhibitor | UNC0646 | 1320288-17-2 |
| G9a/GLP inhibitor | A-366 | 1527503-11-2 |
| G9a/GLP inhibitor | SGC707 | 1687736-54-4 |
| HDAC1/2/3 inhibitor | BRD6688 | 1404562-17-9 |
| HDAC 2/3 inhibitor | MI-192 | 1415340-63-4 |
| YY-1 (Proteasome Inhibitor) | NPI-0052 | 437742-34-2 |
| SMARCA2 (BRM), | PFI-3 | 1819363-80-8 |
| SMARCA4 (BRG1) and | | |
| polybromo-1 (PB1) Inhibitor | | |
| Inhibitors of CREBBP and | SGC-CBP30 | 1613695-14-9 |
| EP300 bromodomains | | |
| RasGAP, ERK1 and Tet2 | SC-1 (Pluripotin) | 839707-37-8 |
| Inhibitor | | |
| Tet2 | Meclofenamate | 6385-02-0 |
| Core Binding Factor-β | CBFβ Inhibitor | 493028-20-9 |
| Inhibitor | | |

According to the present disclosure, dominant negative mutants can be used as inhibitors of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5. In embodiments, the dominant negative mutants of the above genes lack catalytic or signaling function. An example of a dominant negative BRD9 is a protein comprising or consisting of SEQ ID NO: 1823 with the mutation Q479H, according to the numbering of SEQ ID NO: 1822. An example of a dominant negative BRD9 is a protein comprising or consisting of SEQ ID NO: 1823 with the mutation Q479H, according to the numbering of SEQ ID NO: 1821. An example of a dominant negative BRD9 is a protein comprising or consisting of SEQ ID NO: 1820 with the mutation N216A, according to the numbering of SEQ ID NO: 1822. In embodiments, the dominant negative BRD9 may include combinations of any of the aforementioned mutations. Examples of a dominant negative BCOR are proteins comprising or consisting of SEQ ID NO:1829 and 1830 with the mutations V896L and A165P, according to the numbering of SEQ ID NO:1828. In embodiments, the dominant negative BCOR may include combinations of any of the aforementioned mutations. An example of a dominant negative FBXW10 is a protein comprising or consisting of SEQ ID NO: 1826 with the mutation FBXW10-D318N, according to the numbering of SEQ ID NO:1825. An example of dominant negative DR5 is DR5-L363N which is represented by SEQ ID NO:2370.

In embodiments, an inhibitor of the disclosure is a dominant negative binding partner of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, e.g., a dominant negative BRD9-binding partner or a dominant negative DR5 binding partner. In other embodiments, an inhibitor of the disclosure comprises nucleic acid encoding a dominant negative binding partner, e.g., a dominant negative BRD9-binding protein or a dominant negative DR5-binding protein. An example of a DR5 binding protein that can act in a dominant negative manner is a deletion mutant of FADD (Fas Associated Death Domain) containing its amino acids 80-208 that encodes the death domain of FADD but lacks its death effector domain (SEQ ID NO: 2463). Other examples are dominant negative mutants of Caspase 8, e.g., Caspase8 D73A (SEQ ID NO: 2464).

As described herein, the disclosure provides vectors, e.g., as described herein, which encode inhibitors, such as the gene editing systems, shRNA or siRNA inhibitors or dominant negative inhibitors of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, e.g., BRD9 (e.g., as described herein).

In embodiments further comprising, for example, a CAR or a TCR, the nucleic acid may further comprise sequence encoding a CAR or TCR, e.g., as described herein. In some embodiments, the disclosure provides a vector comprising a nucleic acid sequence encoding an inhibitor described herein and comprising a nucleic acid sequence encoding a CAR or a TCR molecule described herein. In embodiments, nucleic acid sequences are disposed on separate vectors. In other embodiments, the two or more nucleic acid sequences are encoded by a single nucleic molecule in the same frame and as a single polypeptide chain. In this aspect, the two or more CARs can, e.g., be separated by one or more peptide cleavage sites. (e.g., an auto-cleavage site or a substrate for an intracellular protease). Examples of peptide cleavage sites include F2A (SEQ ID NO: 1845), T2A (SEQ ID NO: 1846 and 1847) and P2A (SEQ ID NO: 1848). These peptide cleavage sites are referred to collectively herein as “2A sites.”

In embodiments, the vector comprises nucleic acid sequence encoding a CAR described herein and nucleic acid sequence encoding a shRNA described herein. In embodiments, the vector comprises nucleic acid sequence encoding a CAR or a TCR described herein and nucleic acid sequence encoding a genome editing system described herein.

The disclosure provides methods of increasing the therapeutic efficacy of a CAR- or a TCR-expressing cell, e.g., a cell expressing a CAR or TCR as described herein, e.g., a CAR19-expressing cell (e.g., CTL019), comprising a step of reducing or eliminating the function or expression of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5. In embodiments, the method comprises contacting said cells with a BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 inhibitor as described herein.

The disclosure provides methods of increasing the safety of a CAR- or a TCR-expressing cell, e.g., a cell expressing a CAR or TCR as described herein, e.g., a CAR19-expressing cell (e.g., CTL019), comprising a step of reducing or eliminating the function or expression of TRAIL and/or DR5. In embodiments, the method comprises contacting said cells with a TRAIL and/or DR5 inhibitor as described herein.

The disclosure further provides methods of manufacturing a CAR and/or TCR-expressing cell, e.g., a CAR- and/or TCR-expressing cell having improved function (e.g., having improved efficacy, e.g., tumor targeting, or proliferation) comprising the step of reducing or eliminating the expression or function of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 in said cell. In embodiments, the method comprises contacting said cells with a BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 inhibitor as described herein. In embodiments, the contacting is done ex vivo. In embodiments, the contacting is done in vivo. In embodiments, the contacting is done prior to simultaneously with or after said cells are modified to express a CAR or TCR, e.g., a CAR or TCR as described herein.

The disclosure further provides methods of manufacturing a CAR and/or TCR-expressing cell, e.g., a CAR- and/or TCR-expressing cell having improved safety (e.g., having less propensity to cause Cytokine Release Syndrome or neurotoxicity) comprising the step of reducing or eliminating the expression or function of TRAIL, and/or DR5 as described herein. In embodiments, the method comprises contacting said cells with a TRAIL, and/or DR5 inhibitor as described herein. In embodiments, the contacting is done ex vivo. In embodiments, the contacting is done in vivo. In embodiments, the contacting is done prior to simultaneously with or after said cells are modified to express a CAR or TCR, e.g., a CAR or TCR as described herein.

In embodiments, the disclosure provides a method for inhibiting a function or expression of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 in a CAR/TCR-expressing cell, e.g., a cell expressing a CAR/TCR as described herein, e.g., a CAR19-expressing cell, the method comprising a step of reducing or eliminating the function or expression of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5. In embodiments, the method comprises contacting said cells with a BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 inhibitor as described herein.

In one embodiment, the disclosure provides a method, e.g., a method described above, comprises introducing nucleic acid encoding a CAR and/or TCR into a cell, e.g., an immune effector cell, e.g., a T cell, at a site within the BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 gene, or the regulatory elements of the BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 gens such that expression of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 is disrupted. Integration at a site within the BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 gene may be accomplished, for example, using a BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 gene-targeting gene editing system as described above.

In one embodiment, the disclosure provides a method, e.g., a method described above, comprising a step of introducing into the cell a gene editing system, e.g., a CRISPR/Cas gene editing system which targets BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, e.g., a CRISPR/Cas system comprising a gRNA which has a targeting sequence complementary to a target sequence of the BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 gene. In embodiments, the CRISPR/Cas system is introduced into said cell as a ribonuclear protein complex of gRNA and Cas enzyme, e.g., is introduced via electroporation. In one embodiment, the method comprises introducing nucleic acid encoding one or more of the components of the CRISPR/Cas system into said cell. In one embodiment, said nucleic acid is disposed on the vector encoding a CAR and/or TCR, e.g., a CAR and/or TCR as described herein.

In one embodiment, the disclosure provides a method, e.g., a method described above, comprising a step of introducing into the cell an inhibitory dsRNA, e.g., a shRNA or siRNA, which targets BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, e.g., BRD9. In one embodiment, the method comprises introducing into said cell nucleic acid encoding an inhibitory dsRNA, e.g., a shRNA or siRNA, which targets BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, e.g., BRD9. In one embodiment, said nucleic acid is disposed on the vector encoding a CAR and/or TCR, e.g., a CAR and/or TCR as described herein.

In other embodiments, population of immune effector cells, e.g., T cells, which have, or will be engineered to express a CAR/TCR, can be treated ex vivo by contact with an amount of an BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL (TNFSF10) and/or Death Receptor 5 (DR5 or TNFRSF10B) inhibitor that improve their efficacy and reduce their side-effects (e.g., CRS) when administered to a subject.

In one embodiment, a T cell population is deficient in one or more of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL (TNFSF10) and/or Death Receptor 5 (DR5 or TNFRSF10B). T cells that are deficient in BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL (TNFSF10) and/or Death Receptor 5 (DR5 or TNFRSF10B) include cells that do not express the BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL (TNFSF10) and/or Death Receptor 5 (DR5 or TNFRSF10B) RNA or protein, or have reduced or inhibited BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL (TNFSF10) and/or Death Receptor 5 (DR5 or TNFRSF10B) activity. BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL (TNFSF10) and/or Death Receptor 5 (DR5 or TNFRSF10B)-deficient cells can be generated by genetic approaches, e.g., administering RNA-interfering agents, e.g., siRNA, shRNA, miRNA, to reduce or prevent BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL (TNFSF10) and/or Death Receptor 5 (DR5 or TNFRSF10B) expression. Alternatively, BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL (TNFSF10) and/or Death Receptor 5 (DR5 or TNFRSF10B)-deficient cells can be generated by treatment with BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL (TNFSF10) and/or Death Receptor 5 (DR5 or TNFRSF10B) inhibitors described herein.

The disclosure further provides signaling molecules, such as JAK1, JAK3, STAT5b, STAT3, IL2RG, CARD11 and BRAF and their mutants and methods of use therefore in immune and cell therapy. In particular, the disclosure provides immune effector cellular therapy products, e.g., CAR- and TCR-expressing T cells, comprising JAK1, JAK3, STAT5b, STAT3, IL2RG, CARD11 and BRAF and their mutants, and use of JAK1, JAK3, STAT5b, STAT3, IL2RG, CARD11 and BRAF and their mutants in connection with immune effector cell therapy products, e.g., CAR-T and TCR-T cells. JAK1, JAK3, STAT5b, STAT3, IL2RG, CARD11 and BRAF and their mutants of the disclosure, together with their methods of use, are described in more detail below. CARs, CAR T and TCR-T cells, and methods of use are further described below.

In some embodiments, the signaling molecule may elicit one or more of the following effects in an immune cell containing said construct or constructs:

a) enhances the proliferation of such immune effector cell

b) Modulate (increase or decrease) cytokine secretion by immune effector cell

c) Decrease the dependence on exogenous cytokines for survival and proliferation

d) enhance cytotoxicity of immune effector cell

e) enhance the survival of immune effector cell

f) block apoptosis of immune cells

g) Delay senescence of immune cells

h) Delay exhaustion of immune cells

i) Enhance the persistence of immune cells in vivo when administered to patients

j) Enhance the efficacy of immune cells in vivo when administered to patients

In some embodiments, the signaling molecule may be selected from JAK1, JAK3, STAT5b, STAT3, IL2RG, CARD11, BRAF, CD27 (TNFRSF7, Gene ID: 939), CD28 (Gene ID: 940), 41BB (TNFRSF9, CD137; Gene ID: 3604), OX40 (TNFRSF4, Gene ID: 7293), DcR2 (TNFRSF10D, Gene ID: 8793), DcR1 (TNFRSF10C, Gene ID: 8794), BCMA (TNFRSF17, Gene ID: 608), and GITR (TNFRSF18; Gene ID: 8784) or a mutated form of any one of the foregoing.

In some embodiments, the signaling molecule may comprise a TRAIL binding domain, e.g., ectodomain (extracellular domain) of DR5 (SEQ ID NO: 2392); ectodomain of DR4 (SEQ ID NO; 2386), ectodomain of DcR1 (SEQ ID NO: 2375) or ectodomain of DcR1 (SEQ ID NO: 2380). In certain embodiments, the protein comprising the TRAIL binding domain may be joined via a transmembrane domain to an intracellular signaling domain. In some embodiments, the intracellular signaling domains comprise of signaling domains of CD27, CD28, 41BB, BCMA, GITR and OX40. Table 1 lists several exemplary cytoplasmic (CP) signaling domains and several exemplary fusion proteins containing the extracellular TRAIL binding domains and cytoplasmic signaling domains.

In some embodiments, the signaling molecule may be JAK1(SEQ ID NO: 374 and SEQ ID NO:1804), or a homologue or orthologue thereof.

In some embodiments, the signaling molecule may be a mutated form of JAK1, or a homologue or orthologue thereof, optionally wherein the mutation:
i) results in altered signaling as compared to wild-type JAK1(e.g., phosphorylation of STATs)
ii) results in constitutive active signaling
   Exemplary amino acids that can be mutated in JAK1 include one or more of the amino acids V658 and/or S703
   Exemplary amino acids substitutions in JAK1 that result in altered signaling include V658F (SEQ ID NO: 375) and S7031 (SEQ ID NO: 376)
iii) results in nucleic acid sequence that encodes an amino acid sequence having at least 80%, at least 85%, at least 90%, at least 95%, at least 98%, at least 99%, or 100% identity to the amino acid sequence of SEQ ID NO: 1804, 1805 or 1806;

In some embodiments, the signaling molecule may be a mutated form of JAK3, or a homologue or orthologue thereof, optionally wherein the mutation
i) results in altered signaling as compared to wild-type JAK3 (e.g., phosphorylation of STATs)
ii) results in constitutive active signaling
   Exemplary amino acids that can be mutated in JAK3 (DNA SEQ ID NO: 360 and protein SEQ ID NO: 1790) to result in altered signaling include one or more of M511, A573, R657, Q507, G491, V674 and V678.
   Exemplary amino acids substitutions in JAK3 (DNA SEQ ID NO: 360 and protein SEQ ID NO: 1790) that result in altered signaling include one or more of M511, A573, R657, Q507, G491, V674, V678M511I, A573V, R657W, Q507P, G491S, V674A, V678L
iii) nucleic acid sequence that encodes an amino acid sequence having at least 80%, at least 85%, at least 90%, at least 95%, at least 98%, at least 99%, or 100% identity to the amino acid sequence of SEQ ID NO:1790-1797.
   In some embodiments, the signaling molecule may be Stat5b (NM 012448.3; SEQ ID NO: 368), or a homologue or orthologue thereof.

In some embodiments, the signaling molecule may be a mutated form of Stat5b, or a homologue or orthologue thereof, optionally wherein the mutation:
i) results in altered signaling as compared to wild-type Stat5b
ii) results in constitutive active signaling
   Exemplary amino acids that can be mutated in Stat5b (DNA SEQ ID NO: 368 and protein SEQ ID NO: 1798) to result in altered signaling include one or more of T628, N642, Y665, Q706, R659, 1704, E579.
   Exemplary amino acids substitutions in Stat5b (DNA SEQ ID NO: 368 and protein SEQ ID NO: 1798) that result in altered signaling include one or more of T628S (SEQ ID NO: 1799), N642H (SEQ ID NO: 1800), Y665F (SEQ ID NO: 1801), Q706L (SEQ ID NO: 1802), R659C (SEQ ID NO: 1803), 1704L and E579K.
iii) nucleic acid sequence that encodes an amino acid sequence having at least 80%, at least 85%, at least 90%, at least 95%, at least 98%, at least 99%, or 100% identity to the amino acid sequence of SEQ ID NO: 1798-1803.

In some embodiments, the signaling molecule may be Stat3, or a homologue or orthologue thereof.

In some embodiments, the signaling molecule may be a mutated form of Stat3, or a homologue or orthologue thereof, optionally wherein the mutation:
i) results in altered signaling as compared to wild-type Stat3
ii) results in constitutive active signaling
   Exemplary amino acids that can be mutated in Stat3 (DNA SEQ ID NO: 380 and protein SEQ ID NO: 1810) to result in altered signaling include one or more of D661, Y640, A702, S614 and G618.
   Exemplary amino acids substitutions in Stat3 (DNA SEQ ID NO: 380 and protein SEQ ID NO: 1810) that result in altered signaling include one or more of D661Y (SEQ ID NO: 1811), Y640F (SEQ ID NO: 1812), A702T (SEQ ID NO: 1813), S614R (SEQ ID NO: 1814) and G618R (SEQ ID NO: 1815).
iii) nucleic acid sequence that encodes an amino acid sequence having at least 80%, at least 85%, at least 90%, at least 95%, at least 98%, at least 99%, or 100% identity to the amino acid sequence of SEQ ID NO:1810-1815.

In some embodiments, the signaling molecule may be BRAF (e.g., NM_004333.4), or a homologue or orthologue thereof.

In some embodiments, the signaling molecule may be a mutated form of BRAF, or a homologue or orthologue thereof, optionally wherein the mutation:
i) results in altered signaling as compared to wild-type BRAF (provide examples)
ii) results in constitutive active signaling
   Exemplary amino acids that can be mutated in BRAF (DNA SEQ ID NO: 388 and protein SEQ ID NO: 1818) to result in altered signaling include one or more of V600, K601 and E586.
   Exemplary amino acids substitutions in BRAF (DNA SEQ ID NO: 388) and protein SEQ ID NO: 1818) that result in altered signaling include one or more of V600E (SEQ ID NO: 1819), V600D, V600K, V600R, V600 any, K601E, K601 any, E586K.
iii) nucleic acid sequence that encodes an amino acid sequence having at least 80%, at least 85%, at least 90%, at least 95%, at least 98%, at least 99%, or 100% identity to the amino acid sequence of SEQ ID NO: 1818-1819.

In some embodiments, the signaling molecule may be CARD11 (e.g.,), or a homologue or orthologue thereof.

In some embodiments, the signaling molecule may be a mutated form of CARD11, or a homologue or orthologue thereof, optionally wherein the mutation:
i) results in altered signaling as compared to wild-type CARD11
ii) results in constitutive active signaling
   Exemplary amino acids that can be mutated in CARD11 (DNA SEQ ID NO: 377 and protein SEQ ID NO: 1807) to result in altered signaling include one or more of S615 and E626.
   Exemplary amino acids substitutions in CARD11 (DNA SEQ ID NO: 377 and protein SEQ ID NO: 1807) to result in altered signaling include one or more of S615F (SEQ ID NO: 1808) and E626K (SEQ ID NO: 1809).
iii) nucleic acid sequence that encodes an amino acid sequence having at least 80%, at least 85%, at least 90%, at least 95%, at least 98%, at least 99%, or 100% identity to the amino acid sequence of SEQ ID NO: 1807-1809, or amino acid sequence having at least 80%, at least 85%, at least 90%, at least 95%, at least 98%, at least 99%, or 100% identity to the amino acid sequence by SEQ ID NO: 1807-1809;
   wherein the foregoing mutations are indicated with respect to the human sequences, but are intended to further include corresponding or analogous mutations to the homologous or orthologous residues or domains of a homologue or orthologue, e.g., mouse JAK1, JAK3, STAT5b, BRAF or CARD11.

In some embodiments, the T cells ectopically express or over-express wild-type or mutant form of one or more genes from the group of JAK1(SEQ ID NO: 371-374), JAK3 (SEQ ID NO: 360-367), STAT5b (SEQ ID NO:368-373), STAT3 (SEQ ID NO: 380-385), IL2RG (SEQ ID NO: 386-387), CARD11 (SEQ ID NO: 377-379), BRAF (SEQ ID NO: 388-389), CD27 (TNFRSF7, Gene ID: 939), CD28 (Gene ID: 940), 41BB (TNFRSF9, CD137; Gene ID: 3604), OX40 (TNFRSF4, Gene ID: 7293), DcR2 (TNFRSF10D, Gene ID: 8793), DcR1 (TNFRSF10C, Gene ID: 8794), BCMA (TNFRSF17, Gene ID: 608), and GITR (TNFRSF18; Gene ID: 8784). T cells ectopically expressing the wild-type and mutant forms of the above genes can be generated using methods described herein, e.g., using lentiviral mediated gene transfer or transfection of DNA or RNA encoding the corresponding gene.

The disclosure further provides a vector comprising sequence encoding the wild type or constitutive active mutant form of JAK1, JAK3, STAT5b, STAT3, IL2RG, CARD11, BRAF, CD28, 41BB, OX40, DcR2, DcR1, BCMA, and/or GITR genes or a TRAIL antagonist.

In some embodiments, the expression of the nucleic acid encoding the wild type or constitutive active mutant form of JAK1, JAK3, STAT5b, STAT3, IL2RG, CARD11, BRAF, CD28, 41BB, OX40, DcR2, DcR1, BCMA, and/or GITR genes or a TRAIL antagonist may be regulated by constitutive or inducible promoters.

The disclosure further provides a vector comprising sequence encoding an immune receptor and sequence encoding a wild-type or constitutive active mutant of JAK1, JAK3, STAT5b, STAT3, IL2RG, CARD11, and/or BRAF and/or a wild-type and/or mutant of CD28, 41BB, OX40, DcR2, DcR1, BCMA, and GITR genes.

In one embodiment, the sequence encoding an immune receptor and sequence encoding a wild-type or constitutive active mutant of JAK1, JAK3, STAT5b, STAT3, IL2RG, CARD11, and/or BRAF and/or a wild-type and/or mutant of CD28, 41BB, OX40, DcR2, DcR1, BCMA, and GITR genes are separated by a 2A sequence. Several exemplary nucleic acids sequences encoding a CAR/TCR, a 2A sequence and a constitutive active mutant of JAK1, JAK3, STAT5b, STAT3, IL2RG, CARD11 and BRAF are represented by SEQ ID Nos; 484 to 499.

These nucleic acid sequences can be cloned in a suitable vector, e.g., vector described herein, for expression in immune cells. Vectors encoding other CARs/TCRs and wild type and mutants of JAK1, JAK3, STAT5b, STAT3, IL2RG, CARD11, and/or BRAF and/or a wild-type and/or mutant of CD28, 41BB, OX40, DcR2, DcR1, BCMA, and GITR genes can be constructed similarly.

The disclosure further provides a vector comprising a sequence encoding an immune receptor and sequence encoding a TRAIL antagonist. Several exemplary nucleic acids sequences encoding a CAR/TCR, a 2A sequence and a TRAIL antagonist are represented by SEQ ID Nos; 2853- to 2863, 2865-2875, 2877-2887, 2889-2899, 2901-2911, 2913-2923, 2925-2935. The disclosure further provides a vector comprising a sequence encoding a TRAIL antagonist that can be used to genetically modify an immune cell, e.g., an immune effector cell.

The disclosure further provides a vector comprising a sequence encoding an immune receptor and sequence encoding a mutant form of TRAIL receptors DR5, DR4, DcR1 and DcR2. The disclosure further provides a vector comprising a sequence encoding a fusion protein of TRAIL receptors DR5 (SEQ ID NO: 2318-2330), DR4 (SEQ ID NO: 2331-2337), DcR1 (SEQ ID NO: 2338-2344) and DcR2 (SEQ ID NO: 2345-2351) that can be used to genetically modify an immune cell, e.g., an effector cell, so as to block or alter the activity of physiological TRAIL receptors.

In some embodiments, the nucleic acid encoding the CAR/TCR and the nucleic acid encoding the a wild-type or constitutive active mutant form of JAK1, JAK3, STAT5b, STAT3, IL2RG, CARD11, BRAF, CD28, 41BB, OX40, DcR2, DcR1, BCMA, and/or GITR genes or a TRAIL antagonist may be on the same vector.

In some embodiments, the nucleic acid encoding the CAR/TCR and the nucleic acid encoding the wild type or constitutive active mutant form of JAK1, JAK3, STAT5b, STAT3, IL2RG, CARD11, BRAF, CD28, 41BB, OX40, DcR2, DcR1, BCMA, and/or GITR genes or a TRAIL antagonist may be on different vectors.

In some embodiments, the expression of the nucleic acid encoding the CAR/TCR and the nucleic acid encoding the wild type or constitutive active mutant form of JAK1, JAK3, STAT5b, STAT3, IL2RG, CARD11, BRAF, CD28, 41BB, OX40, DcR2, DcR1, BCMA, and/or GITR genes or a TRAIL antagonist may be regulated by different constitutive or inducible promoters.

In some embodiments, the expression of the nucleic acid encoding the CAR/TCR and the nucleic acid encoding the wild-type or constitutive active mutant form of JAK1, JAK3, STAT5b, STAT3, IL2RG, CARD11, BRAF, CD28, 41BB, OX40, DcR2, DcR1, BCMA, and/or GITR genes or a TRAIL antagonist may be regulated by the same constitutive or inducible promoter.

In some embodiments, the present disclosure provides cells expressing an immune receptor (e.g., a CAR, SIR, TFP, Ab-TCR or TCR) and ectopically expressing or over-expressing wild-type or mutant form of one or more genes from the group of JAK1, JAK3, STAT5b, STAT3, IL2RG, CARD11, and/or BRAF and/or a wild-type and/or mutant of CD28, 41BB, OX40, DcR2, DcR1, BCMA, and GITR.

In some embodiments, the cells of the disclosure comprise an immune receptor (e.g., a CAR, SIR, TFP, Ab-TCR or TCR), and a mutant form of one or more genes from the group of JAK1, JAK3, STAT5b, STAT3, IL2RG, CARD11, and/or BRAF. In some embodiments, the cells of the disclosure comprise an immune receptor (e.g., a CAR, SIR, TFP, Ab-TCR or TCR), and ectopically express or over-express one of more genes from the group of CD27, CD28, 41BB, OX40, DcR2, DcR1, BCMA, and GITR. In some embodiments, the immune cells, e.g., immune effector cells, also co-express an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5.

In one aspect, the disclosure provides a method of altering the phenotype, differentiation state and/or therapeutic efficacy of an immune cell, e.g., immune receptor-expressing cell, e.g., a cell of any of the previous claims, e.g., a CAR19-expressing cell, comprising a step of increasing the expression and/or activity of JAK1, JAK3, STAT5b, STAT3, IL2RG, CARD11, BRAF, CD27, CD28, 41BB, OX40, DcR2, DcR1, BCMA, and GITR in said cell. In some embodiments, said step comprises expressing in said cells a wild-type or constitutive active mutant of JAK1, JAK3, STAT5b, STAT3, IL2RG, CARD11, and BRAF. In some embodiments, said step comprises expressing in said cells a wild-type or mutant of CD28, 41BB, OX40, DcR2, DcR1, BCMA, and GITR.

In some embodiments, the wild-type and/or constitutive active mutants of JAK1, JAK3, STAT5b, STAT3, IL2RG, CARD11, BRAF, CD28, 41BB, OX40, DcR2, DcR1, BCMA, and GITR of the present disclosure are expressed in the cells 1) ectopically by introduction of nucleic acid sequences (DNA or mRNA) encoding their wild-type or constitutive active mutants; 2) by altering the endogenous allele or alleles using homologous recombination at the genomic locus. In some embodiments, the immune cells, e.g., immune effector cells, also co-express an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5.

In one aspect, the disclosure provides a method of increasing the diversity of immune receptor-expressing cell, e.g., cells of any of the previous claims, e.g., CAR19-expressing cells, comprising a step of altering the expression and/or activity of JAK1, JAK3, STAT5b, STAT3, IL2RG, CARD11, BRAF, CD27, CD28, 41BB, OX40, DcR2, DcR1, BCMA, and GITR in said cell. In some embodiments, said step comprises expressing in said cells a wild-type or constitutive active mutant of JAK1, JAK3, STAT5b, STAT3, IL2RG, CARD11, and BRAF. In some embodiments, said step comprises expressing in said cells a wild-type or mutant of CD28, 41BB, OX40, DcR2, DcR1, BCMA, and GITR.

In some embodiments, the wild-type and/or constitutive active mutants of JAK1, JAK3, STAT5b, STAT3, IL2RG, CARD11, BRAF, CD28, 41BB, OX40, DcR2, DcR1, BCMA, and GITR of the present disclosure are expressed in the cells 1) ectopically by introduction of nucletic acid sequences (DNA or mRNA) encoding their wild-type or constitutive active mutants; 2) by altering the endogenous allele or alleles using homologous recombination at the genomic locus. In some embodiments, the immune effector cells also co-express an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5.

In some embodiments, the genetic and/or chemical inhibitors of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, and/or signaling molecule JAK1, JAK3, STAT5b, STAT3, IL2RG, CARD11, and/or BRAF and/or a wild-type and/or mutant of CD28, 41BB, OX40, DcR2, DcR1, BCMA, and GITR may elicit one or more of the following effects in an immune cell:
1. enhances the proliferation of such immune effector cell
2. Alter cytokine secretion by immune effector cell
3. Decrease the dependence on exogenous cytokines for survival and proliferation
4. enhance cytotoxicity of immune effector cell
5. enhance the survival of immune effector cell
6. block apoptosis of immune cells
7. Delay senescence of immune cells
8. Delay exhaustion of immune cells
9. Enhance the persistence of immune cells in vivo when administered to patients
10. Enhance the efficacy of immune cells in vivo when administered to patients
11. Enhance the penetration of immune cells into diseased organs or tissues (e.g., tumor)
12. Prevent or ameliorate the toxicity, e.g., CRS and neurotoxicity, caused by administration of immune cells

In one aspect, the disclosure provides a method of treating a subject in need thereof, comprising administering to said subject an effective amount of the cells as described herein, e.g., immune cells, e.g., immune effector cells (e.g., T cell or NK cell) comprising an immune receptor, and co-expressing constitutive active mutants of JAK1, JAK3, STAT5b, STAT3, IL2RG, CARD11, and/or BRAF and/or a wild-type and/or mutant of CD28, 41BB, OX40, DcR2, DcR1, BCMA, and GITR.

In one aspects, the disclosure provides a method of treating a subject in need thereof, comprising administering to said subject an effective amount of the cells as described herein, e.g., immune effector cells (e.g., T cell or NK cell) comprising an immune receptor, and co-expressing constitutive active mutants of JAK1, JAK3, STAT5b, STAT3, IL2RG, CARD11, and/or BRAF and/or a wild-type and/or mutant of CD28, 41BB, OX40, DcR2, DcR1, BCMA, and GITR, and optionally, administering to said subject an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5.

In some embodiment, the subject receives a pre-treatment with the inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 prior to the initiation of the immune- and/or cell therapy comprising constitutive active mutants of JAK1, JAK3, STAT5b, STAT3, IL2RG, CARD11, and/or BRAF and/or a wild-type and/or mutant of CD28, 41BB, OX40, DcR2, DcR1, BCMA, and GITR. In some embodiments, the subject receives concurrent treatment with an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 and the immune- and/or cell-therapy. In some embodiments, the subject receives treatment with an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 following the administration of immune- and/or cell-therapy.

Provided herein are compositions of matter and methods of use for the treatment of a disease such as cancer and immune disorders using cell therapy products (e.g., T cells, NK cells) engineered with a chimeric antigen receptor (CAR) or a native or natural TCR. In some embodiments, the cell is engineered to express a CAR. The disclosure is not limited by the type CAR. The CARs as described herein covers both the 2^{nd} generation CARs and next generation CARs (e.g., SIRs, zSIRs, Ab-TCRs and TFP) as described in PCT/US2017/024843, WO 2014/160030 A2, WO 2016/187349 A1, PCT/US2016/058305 and PCT/US17/64379, which are incorporated herein by reference in their entirety. It is to be noted that similar composition of matters and methods of use can be used for the treatment of diseases such as cancer and immune disorder using other immune effector cells (e.g., T cells), such as Tumor Infilterating Lymphocytes (TILs) or T cells engineered with recombinant TCRs.

Sequences of some examples of various components of CARs (conventional and next generation CARs) of the instant disclosure is listed in Table 7a. The Table 7a also lists the DNA and Protein SEQ ID Nos of several exemplary conventional CARs and next generation CARs. Finally, the Table 7a lists the DNA and Protein SEQ ID NOs of several exemplary conventional CARs, next generation CARs and TCRs that also encode an accessory module comprising a signaling protein. For exemple, the construct CD8SP-FMC63-(vL-vH)-Myc-BBz-T2A-JAK3-M511I represented by SEQ ID NO (DNA): 484 and SEQ ID NO (PRT): 1911, respectively, is a second generation CAR that comprises a CD8 signal peptide, a scFv fragment derived from FMC63 monoclonal antibody targeting CD19, a Myc epitope tag, a 41BB costimulatory domain, a CD3z signaling domain, a T2A cleavage site and a JAK3-M511I mutant. The constructs represented by DNA SEQ ID NOs: 485-490 and PRT SEQ ID NO: 1912-1917, respectively, are similar in design with the exception that the JAK3-M511I module is replaced by JAK3-A573V, STAT5b-T658F, JAK1-V658F, CARD11-S615F, STAT3-Y640F and BRAF-V600E, respectively. The construct CD8SP-FMC63-vL-V5-[hTCRb-KACIAH]-F-P2A-SP-FMC63-vH-Myc-[hTCRa-CSDVP]-F-F2A-JAK3-M511I (SEQ ID NO: 491 and 1918) is a double chain SIR (Synthetic Immune Receptor) targeting CD19 and co-expressing JAK-M511I. The construct CD8SP-FMC63-(vL-vH)-Myc-z-P2A-JAK3-M511I (SEQ ID NO: 492 and 1919) is a first generation CAR coexpressing JAK3-M511I. The constructs represented by SEQ ID NO: 493-496 are TFPs targeting CD19 and co-expressing JAK3-M511I. The constructs CD8SP-FMC63-vL-[IgCL-TCRb-IAH-6MD]-F-P2A-SP-FMC63-vH-[IgG1-CH1-TCRa-SDVP-6MD]-F-F2A-JAK3-M511I (SEQ ID NO: 497 and 1924) and CD8SP-FMC63-vL-[IgCL-TCRg-6MD]-F-P2A-SP-FMC63-vH-[IgGI-CH1-TCRd-6MD]-F-F2A-JAK3-M511I (SEQ ID NO: 498 and 1925) are two Antibody-TCR (Ab-TCR) targeting CD19 and co-expressing JAK3-M511I. The construct NY-ESO-TCRa—F-P2A-NYESO-[hTCRb]-F-F2A-JAK3-M511I (SEQ ID NO: 499 and 1926) is a recombinant TCR targeting NY-ESO/HLA-A2 complex and co-expressing M511I. Table 7a also lists the SEQ ID Nos of several exemplary CARs, next generation CARs and TCRs expressing different TRAIL antagonists, e.g., DR5-Fc and DR5-fusion proteins. The above constructs represent exemplary constructs. The accessory module M511I can be replaced by other accessory modules of the disclosure listed in Table 1 by methods known in the art. Similarly, the CD19 antigen binding domains (e.g., FMC63 scFv) can be replaced by antigen binding domains targeting other antigens by methods known in the art. The patent applications PCT/US2017/024843, WO 2014/160030 A2, WO 2016/187349 A1, PCT/US2016/058305 and PCI/US17/64379 describe conventional and next generation CARs targeting several antigens.

TABLE 7a
Nucleic acid and Protein SEQ ID of conventional and next
generation CAR and CAR components

| | SEQ ID | SEQ ID |
|---|---|---|
| | (DNA) | (PRT) |
| CD8_Signal_Peptide | 407 | 1834 |
| IgH_Signal_Peptide | 408 | 1835 |
| FMC63-vL | 409 | 1836 |
| CD20-2F2-vL | 410 | 1837 |
| (GGGGS) x3_LINKER | 411 | 1838 |
| FMC63-vH | 412 | 1839 |
| CD20-2F2-vH | 413 | 1840 |
| FMC63-(vL-vH) | 414 | 1841 |
| CD20-2F2-(vL-vH) | 415 | 1842 |
| Myc-TAG | 416 | 1843 |
| V5-TAG | 417 | 1844 |
| F2A | 418 | 1845 |
| T2A | 419 | 1846 |
| T2A | 420 | 1847 |
| P2A | 421 | 1848 |
| P2a-variant | 422 | 1849 |
| E2A | 423 | 1850 |
| SGSG | 424 | 1851 |
| SGSG | 425 | 1852 |
| FURINE CLEAVAGE SITE | 426 | 1853 |
| hCD8-Hinge-TM | 427 | 1854 |
| hCD8-Hinge-TM-BBz | 428 | 1855 |
| hCD8TM-Hinge-BB | 429 | 1856 |
| 4-1BB-cytosolic-domain | 430 | 1857 |
| CD3z-cytosolic-domain | 431 | 1858 |
| CD3z-cytosolic-domain | 432 | 1859 |
| CD28-Hinge-TM-cytosolic-domain | 433 | 1860 |
| LAILR1-TM-CP | 434 | 1861 |
| CD3d-ECDTMCP-opt2 | 435 | 1862 |
| CD3d-ECDTMCP-opt | 436 | 1863 |
| CD3eECDTM-opt2 | 437 | 1864 |
| CD3e-CP-opt2 | 438 | 1865 |
| CD3eECDTMCP-opt2 | 439 | 1866 |
| CD3g-ECDTMCP-opt2 | 440 | 1867 |
| CD3g-ECDTMCP-opt | 441 | 1868 |
| CD3e-ECDTM-opt | 442 | 1869 |
| CD3e-CP-opt | 443 | 1870 |
| CD3e-ECDTMCP-opt | 444 | 1871 |
| CD3zECDTM-opt | 445 | 1872 |
| CD3z-CP-opt | 446 | 1873 |
| CD3zECDTM-opt2 | 447 | 1874 |
| CD3z-CP-opt2 | 448 | 1875 |
| CD3zECDTMCP-opt2 | 449 | 1876 |
| IgCL-TCRg-6MD | 450 | 1877 |
| IgCL-TCRb-IAH-6MD | 451 | 1878 |
| IgCL-TCRb-wt2-opt-6MD | 452 | 1879 |
| IgG1-CH1-TCRd-6MD | 453 | 1880 |
| IgG1-CH1-TCRa-SDVP-6MD | 454 | 1881 |
| IgG1-CH1-TCRa-wt2-opt-6MD | 455 | 1882 |
| hTCR-alpha-constant_X02883.1 | 456 | 1883 |
| hTCRa-WT | 457 | 1884 |
| hTCRa-CSDVP | 458 | 1885 |
| hTCRa-T48C-opt | 459 | 1886 |
| hTCRaECD-CD3zECDTMCP-opt2 | 460 | 1887 |
| hTCR-b1-constant-region_X00437.1 | 461 | 1888 |
| hTCR-b2-constant region_L34740 | 462 | 1889 |
| hTCRb-WT | 463 | 1890 |
| hTCRb-S57C-opt1 | 464 | 1891 |
| hTCRb-KACIAH | 465 | 1892 |
| hTCRbECD-CD3zECDTMCP-opt | 466 | 1893 |
| preTCRa_gb_U38996.1 | 467 | 1894 |
| preTCRa | 468 | 1895 |
| preTCRa-de148 | 469 | 1896 |
| hTCR-gamma_M27331.1 | 470 | 1897 |
| hTCR-Gamma-Opt | 471 | 1898 |
| hTCR-Delta | 472 | 1899 |
| hTCR-Delta-Opt | 473 | 1900 |
| PuroR_Variant (PAC) | 474 | 1901 |
| BlastR | 475 | 1902 |
| CD8SP-FMC63-(vL-vH)-Myc-CD28z-T2A-PAC | 476 | 1903 |
| CD8SP-FMC63-(vL-vH)-Myc-BBz-T2A-PAC | 477 | 1904 |
| CD8SP-CD20-2F2-(vL-vH)-Myc-BBz-T2A- | 478 | 1905 |
| PAC | | |
| CD8SP-FMC63-vL-V5-[hTCRb-KACIAH]-F- | 479 | 1906 |
| P2A-SP-FMC63-vH-Myc-[hTCRa-CSDVP]-F- | | |
| F2A-PAC | | |
| CD8SP-CD20-2F2-vL-V5-[hTCRb-KACIAH]- | 480 | 1907 |
| F-P2A-SP-CD20-2F2-vH-Myc-[hTCRa- | | |
| CSDVP]-F-F2A-PAC | | |
| CD8SP-FMC63-(vL-vH)-Myc-z-P2A-K13- | 481 | 1908 |
| Flag-T2A-PAC | | |
| CD8SP-CD20-2F2-(vL-vH)-Myc-z-P2A-K13- | 482 | 1909 |
| Flag-T2A-PAC | | |
| Streptag | 483 | 1910 |
| CD8SP-FMC63-(vL-vH)-Myc-BBz-T2A-JAK3- | 484 | 1911 |
| M511I | | |
| CD8SP-FMC63-(vL-vH)-Myc-BBz-T2A-JAK3- | 485 | 1912 |
| A573V | | |
| CD8SP-FMC63-(vL-vH)-Myc-BBz-T2A- | 486 | 1913 |
| Stat5b-T628S | | |
| CD8SP-FMC63-(vL-vH)-Myc-BBz-T2A-JAK1- | 487 | 1914 |
| V658F | | |
| CD8SP-FMC63-(vL-vH)-Myc-BBz-T2A- | 488 | 1915 |
| CARD11-S615F | | |
| CD8SP-FMC63-(vL-vH)-Myc-BBz-T2A- | 489 | 1916 |
| STAT3-Y640F | | |
| CD8SP-FMC63-(vL-vH)-Myc-BBz-T2A-BRAF- | 490 | 1917 |
| V600E | | |
| CD8SP-FMC63-vL-V5-[hTCRb-KACIAH]-F- | 491 | 1918 |
| P2A-SP-FMC63-vH-Myc-[hTCRa-CSDVP]-F- | | |
| F2A-JAK3-M511I | | |
| CD8SP-FMC63-(vL-vH)-Myc-z-P2A-JAK3- | 492 | 1919 |
| M511I | | |
| CD8SP-FMC63-(vL-vH)-CD3e-ECDTMCP- | 493 | 1920 |
| opt2-P2A-JAK3-M511I | | |
| CD8SP-FMC63-(vL-vH)-CD3d-ECDTMCP- | 494 | 1921 |
| opt2-P2A-JAK3-M511I | | |
| CD8SP-FMC63-(vL-vH)-CD3g-ECDTMCP- | 495 | 1922 |
| opt2-P2A-JAK3-M511I | | |
| CD8SP-FMC63-(vL-vH)-CD3z-ECDTMCP- | 496 | 1923 |
| opt2-P2A-JAK3-M511I | | |
| CD8SP-FMC63-vL-[IgCL-TCRb-IAH-6MD]-F- | 497 | 1924 |
| P2A-SP-FMC63-vH-[IgG1-CH1-TCRa-SDVP- | | |
| 6MD]-F-F2A-JAK3-M511I | | |
| CD8SP-FMC63-vL-[IgCL-TCRg-6MD]-F-P2A- | 498 | 1925 |
| SP-FMC63-vH-[IgG1-CH1-TCRd-6MD]-F- | | |
| F2A-JAK3-M511I | | |
| NY-ESO-TCRa-F-P2A-NYESO-[hTCRb]-F- | 499 | 1926 |
| F2A-JAK3-M511I | | |
| CD8SP-FMC63-(vL-vH)-Myc-BBz-T2A-DR5- | 2853 | 2945 |
| Fc | | |
| CD8SP-FMC63-vL-V5-[hTCRb-KACIAH]-F- | 2854 | 2946 |
| P2A-SP-FMC63-vH-Myc-[hTCRa-CSDVP]-F- | | |
| F2A-DR5-Fc | | |
| CD8SP-FMC63-(vL-vH)-Myc-z-P2A-DR5-Fc | 2855 | 2947 |
| CD8SP-FMC63-(vL-vH)-CD3e-ECDTMCP- | 2856 | 2948 |
| opt2-P2A-DR5-Fc | | |
| CD8SP-FMC63-(vL-vH)-CD3d-ECDTMCP- | 2857 | 2949 |
| opt2-P2A-DR5-Fc | | |
| CD8SP-FMC63-(vL-vH)-CD3g-ECDTMCP- | 2858 | 2950 |
| opt2-P2A-DR5-Fc | | |
| CD8SP-FMC63-(vL-vH)-CD3z-ECDTMCP- | 2859 | 2951 |
| opt2-P2A-DR5-Fc | | |
| CD8SP-FMC63-vL-[IgCL-TCRb-IAH-6MD]-F- | 2860 | 2952 |
| P2A-SP-FMC63-vH-[IgG1-CH1-TCRa-SDVP- | | |
| 6MD]-F-F2A-DR5-Fc | | |
| CD8SP-FMC63-vL-[IgCL-TCRg-6MD]-F-P2A- | 2861 | 2953 |
| SP-FMC63-vH-[IgG1-CH1-TCRd-6MD]-F- | | |
| F2A-DR5-Fc | | |
| CD8SP-NY-ESO-[hTCRb-KACIAH]-F-P2A-SP- | 2862 | 2954 |
| NYESO-[hTCRa-CSDVP]-F-F2A-DR5-Fc | | |
| CD8SP-NY-ESO-TCRa-F-P2A-SP-NYESO- | 2863 | 2955 |
| [hTCRb]-F-F2A-DR5-Fc | | |
| 2864 | 2956 | |
| CD8SP-FMC63-(vL-vH)-Myc-BBz-T2A-DR5- | 2865 | 2957 |
| SP-ECD-TM-CD27-CP | | |
| CD8SP-FMC63-vL-V5-[hTCRb-KACIAH]-F- | 2866 | 2958 |
| P2A-SP-FMC63-vH-Myc-[hTCRa-CSDVP]-F- | | |
| F2A-DR5-SP-ECD-TM-CD27-CP | | |
| CD8SP-FMC63-(vL-vH)-Myc-z-P2A-DR5-SP- | 2867 | 2959 |
| ECD-TM-CD27-CP | | |
| CD8SP-FMC63-(vL-vH)-CD3e-ECDTMCP- | 2868 | 2960 |
| opt2-P2A-DR5-SP-ECD-TM-CD27-CP | | |
| CD8SP-FMC63-(vL-vH)-CD3d-ECDTMCP- | 2869 | 2961 |
| opt2-P2A-DR5-SP-ECD-TM-CD27-CP | | |
| CD8SP-FMC63-(vL-vH)-CD3g-ECDTMCP- | 2870 | 2962 |
| opt2-P2A-DR5-SP-ECD-TM-CD27-CP | | |
| CD8SP-FMC63-(vL-vH)-CD3z-ECDTMCP- | 2871 | 2963 |
| opt2-P2A-DR5-SP-ECD-TM-CD27-CP | | |
| CD8SP-FMC63-vL-[IgCL-TCRb-IAH-6MD]-F- | 2872 | 2964 |
| P2A-SP-FMC63-vH-[IgG1-CH1-TCRa-SDVP- | | |
| 6MD]-F-F2A-DR5-SP-ECD-TM-CD27-CP | | |
| CD8SP-FMC63-vL-[IgCL-TCRg-6MD]-F-P2A- | 2873 | 2965 |
| SP-FMC63-vH-[IgG1-CH1-TCRd-6MD]-F- | | |
| F2A-DR5-SP-ECD-TM-CD27-CP | | |
| CD8SP-NY-ESO-[hTCRb-KACIAH]-F-P2A-SP- | 2874 | 2966 |
| NYESO-[hTCRa-CSDVP]-F-F2A-DR5-SP-ECD- | | |
| TM-CD27-CP | | |
| CD8SP-NY-ESO-TCRa-F-P2A-SP-NYESO- | 2875 | 2967 |
| [hTCRb]-F-F2A-DR5-SP-ECD-TM-CD27-CP | 2876 | 2968 |
| CD8SP-FMC63-(vL-vH)-Myc-BBz-T2A-DR5- | 2877 | 2969 |
| SP-ECD-TM-CD2 8-CP | | |
| CD8SP-FMC63-vL-V5-[hTCRb-KACIAH]-F- | 2878 | 2970 |
| P2A-SP-FMC63-vH-Myc-[hTCRa-CSDVP]-F- | | |
| F2A-DR5-SP-ECD-TM-CD28-CP | | |
| CD8SP-FMC63-(vL-vH)-Myc-z-P2A-DR5-SP- | 2879 | 2971 |
| ECD-TM-CD28-CP | | |
| CD8SP-FMC63-(vL-vH)-CD3e-ECDTMCP- | 2880 | 2972 |
| opt2-P2A-DR5-SP-ECD-TM-CD28-CP | | |
| CD8SP-FMC63-(vL-vH)-CD3d-ECDTMCP- | 2881 | 2973 |
| opt2-P2A-DR5-SP-ECD-TM-CD28-CP | | |
| CD8SP-FMC63-(vL-vH)-CD3g-ECDTMCP- | 2882 | 2974 |
| opt2-P2A-DR5-SP-ECD-TM-CD28-CP | | |
| CD8SP-FMC63-(vL-vH)-CD3z-ECDTMCP- | 2883 | 2975 |
| opt2-P2A-DR5-SP-ECD-TM-CD28-CP | | |
| CD8SP-FMC63-vL-[IgCL-TCRb-IAH-6MD]-F- | 2884 | 2976 |
| P2A-SP-FMC63-vH-[IgG1-CH1-TCRa-SDVP- | | |
| 6MD]-F-F2A-DR5-SP-ECD-TM-CD28-CP | | |
| CD8SP-FMC63-vL-[IgCL-TCRg-6MD]-F-P2A- | 2885 | 2977 |
| SP-FMC63-vH-[IgG1-CH1-TCRd-6MD]-F- | | |
| F2A-DR5-SP-ECD-TM-CD28-CP | | |
| CD8SP-NY-ESO-[hTCRb-KACIAH]-F-P2A-SP- | 2886 | 2978 |
| NYESO-[hTCRa-CSDVP]-F-F2A-DR5-SP-ECD- | | |
| TM-CD28-CP | | |
| CD8SP-NY-ESO-TCRa-F-P2A-SP-NYESO- | 2887 | 2979 |
| [hTCRb]-F-F2A-DR5-SP-ECD-TM-CD28-CP | 2888 | 2980 |
| CD8SP-FMC63-(vL-vH)-Myc-BBz-T2A-DR5- | 2889 | 2981 |
| SP-ECD-TM-41BB-CP | | |
| CD8SP-FMC63-vL-V5-[hTCRb-KACIAH]-F- | 2890 | 2982 |
| P2A-SP-FMC63-vH-Myc-[hTCRa-CSDVP]-F- | | |
| F2A-DR5-SP-ECD-TM-41BB-CP | | |
| CD8SP-FMC63-(vL-vH)-Myc-z-P2A-DR5-SP- | 2891 | 2983 |
| ECD-TM-41BB-CP | | |
| CD8SP-FMC63-(vL-vH)-CD3e-ECDTMCP- | 2892 | 2984 |
| opt2-P2A-DR5-SP-ECD-TM-41BB-CP | | |
| CD8SP-FMC63-(vL-vH)-CD3d-ECDTMCP- | 2893 | 2985 |
| opt2-P2A-DR5-SP-ECD-TM-41BB-CP | | |
| CD8SP-FMC63-(vL-vH)-CD3g-ECDTMCP- | 2894 | 2986 |
| opt2-P2A-DR5-SP-ECD-TM-41BB-CP | | |
| CD8SP-FMC63-(vL-vH)-CD3z-ECDTMCP- | 2895 | 2987 |
| opt2-P2A-DR5-SP-ECD-TM-41BB-CP | | |
| CD8SP-FMC63-vL-[IgCL-TCRb-IAH-6MD]-F- | 2896 | 2988 |
| P2A-SP-FMC63-vH-[IgG1-CH1-TCRa-SDVP- | | |
| 6MD]-F-F2A-DR5-SP-ECD-TM-41BB-CP | | |
| CD8SP-FMC63-vL-[IgCL-TCRg-6MD]-F-P2A- | 2897 | 2989 |
| SP-FMC63-vH-[IgG1-CH1-TCRd-6MD]-F- | | |
| F2A-DR5-SP-ECD-TM-41BB-CP | | |
| CD8SP-NY-ESO-[hTCRb-KACIAH]-F-P2A-SP- | 2898 | 2990 |
| NYESO-[hTCRa-CSDVP]-F-F2A-DR5-SP-ECD- | | |
| TM-41BB-CP | | |
| CD8SP-NY-ESO-TCRa-F-P2A-SP-NYESO- | 2899 | 2991 |
| [hTCRb]-F-F2A-DR5-SP-ECD-TM-41BB-CP | 2900 | 2992 |
| CD8SP-FMC63-(vL-vH)-Myc-BBz-T2A-DR5- | 2901 | 2993 |
| SP-ECD-TM-OX40-CP | | |
| CD8SP-FMC63-vL-V5-[hTCRb-KACIAH]-F- | 2902 | 2994 |
| P2A-SP-FMC63-vH-Myc-[hTCRa-CSDVP]-F- | | |
| F2A-DR5-SP-ECD-TM-OX40-CP | | |
| CD8SP-FMC63-(vL-vH)-Myc-z-P2A-DR5-SP- | 2903 | 2995 |
| ECD-TM-OX40-CP | | |
| CD8SP-FMC63-(vL-vH)-CD3e-ECDTMCP- | 2904 | 2996 |
| opt2-P2A-DR5-SP-ECD-TM-OX40-CP | | |
| CD8SP-FMC63-(vL-vH)-CD3d-ECDTMCP- | 2905 | 2997 |
| opt2-P2A-DR5-SP-ECD-TM-OX40-CP | | |
| CD8SP-FMC63-(vL-vH)-CD3g-ECDTMCP- | 2906 | 2998 |
| opt2-P2A-DR5-SP-ECD-TM-OX40-CP | | |
| CD8SP-FMC63-(vL-vH)-CD3z-ECDTMCP- | 2907 | 2999 |
| opt2-P2A-DR5-SP-ECD-TM-OX40-CP | | |
| CD8SP-FMC63-vL-[IgCL-TCRb-IAH-6MD]-F- | 2908 | 3000 |
| P2A-SP-FMC63-vH-[IgG1-CH1-TCRa-SDVP- | | |
| 6MD]-F-F2A-DR5-SP-ECD-TM-OX40-CP | | |
| CD8SP-FMC63-vL-[IgCL-TCRg-6MD]-F-P2A- | 2909 | 3001 |
| SP-FMC63-vH-[IgG1-CH1-TCRd-6MD]-F- | | |
| F2A-DR5-SP-ECD-TM-OX40-CP | | |
| CD8SP-NY-ESO-[hTCRb-KACIAH]-F-P2A-SP- | 2910 | 3002 |
| NYESO-[hTCRa-CSDVP]-F-F2A-DR5-SP-ECD- | | |
| TM-OX40-CP | | |
| CD8SP-NY-ESO-TCRa-F-P2A-SP-NYESO- | 2911 | 3003 |
| [hTCRb]-F-F2A-DR5-SP-ECD-TM-OX40-CP | 2912 | 3004 |
| CD8SP-FMC63-(vL-vH)-Myc-BBz-T2A-DR5- | 2913 | 3005 |
| SP-ECD-TM-GITR-CP | | |
| CD8SP-FMC63-vL-V5-[hTCRb-KACIAH]-F- | 2914 | 3006 |
| P2A-SP-FMC63-vH-Myc-[hTCRa-CSDVP]-F- | | |
| F2A-DR5-SP-ECD-TM-GITR-CP | | |
| CD8SP-FMC63-(vL-vH)-Myc-z-P2A-DR5-SP- | 2915 | 3007 |
| ECD-TM-GITR-CP | | |
| CD8SP-FMC63-(vL-vH)-CD3e-ECDTMCP- | 2916 | 3008 |
| opt2-P2A-DR5-SP-ECD-TM-GITR-CP | | |
| CD8SP-FMC63-(vL-vH)-CD3d-ECDTMCP- | 2917 | 3009 |
| opt2-P2A-DR5-SP-ECD-TM-GITR-CP | | |
| CD8SP-FMC63-(vL-vH)-CD3g-ECDTMCP- | 2918 | 3010 |
| opt2-P2A-DR5-SP-ECD-TM-GITR-CP | | |
| CD8SP-FMC63-(vL-vH)-CD3z-ECDTMCP- | 2919 | 3011 |
| opt2-P2A-DR5-SP-ECD-TM-GITR-CP | | |
| CD8SP-FMC63-vL-[IgCL-TCRb-IAH-6MD]-F- | 2920 | 3012 |
| P2A-SP-FMC63-vH-[IgG1-CH1-TCRa-SDVP- | | |
| 6MD]-F-F2A-DR5-SP-ECD-TM-GITR-CP | | |
| CD8SP-FMC63-vL-[IgCL-TCRg-6MD]-F-P2A- | 2921 | 3013 |
| SP-FMC63-vH-[IgG1-CH1-TCRd-6MD]-F- | | |
| F2A-DR5-SP-ECD-TM-GITR-CP | | |
| CD8SP-NY-ESO-[hTCRb-KACIAH]-F-P2A-SP- | 2922 | 3014 |
| NYESO-[hTCRa-CSDVP]-F-F2A-DR5-SP-ECD- | | |
| TM-GITR-CP | | |
| CD8SP-NY-ESO-TCRa-F-P2A-SP-NYESO- | 2923 | 3015 |
| [hTCRb]-F-F2A-DR5-SP-ECD-TM-GITR-CP | 2924 | 3016 |
| CD8SP-FMC63-(vL-vH)-Myc-BBz-T2A-DR5- | 2925 | 3017 |
| SP-ECD-TM-BCMA-CP | | |
| CD8SP-FMC63-vL-V5-[hTCRb-KACIAH]-F- | 2926 | 3018 |
| P2A-SP-FMC63-vH-Myc-[hTCRa-CSDVP]-F- | | |
| F2A-DR5-SP-ECD-TM-BCMA-CP | | |
| CD8SP-FMC63-(vL-vH)-Myc-z-P2A-DR5-SP- | 2927 | 3019 |
| ECD-TM-BCMA-CP | | |
| CD8SP-FMC63-(vL-vH)-CD3e-ECDTMCP- | 2928 | 3020 |
| opt2-P2A-DR5-SP-ECD-TM-BCMA-CP | | |
| CD8SP-FMC63-(vL-vH)-CD3d-ECDTMCP- | 2929 | 3021 |
| opt2-P2A-DR5-SP-ECD-TM-BCMA-CP | | |
| CD8SP-FMC63-(vL-vH)-CD3g-ECDTMCP- | 2930 | 3022 |
| opt2-P2A-DR5-SP-ECD-TM-BCMA-CP | | |
| CD8SP-FMC63-(vL-vH)-CD3z-ECDTMCP- | 2931 | 3023 |
| opt2-P2A-DR5-SP-ECD-TM-BCMA-CP | | |
| CD8SP-FMC63-vL-[IgCL-TCRb-IAH-6MD]-F- | 2932 | 3024 |
| P2A-SP-FMC63-vH-[IgG1-CH1-TCRa-SDVP- | | |
| 6MD]-F-F2A-DR5-SP-ECD-TM-BCMA-CP | | |
| CD8SP-FMC63-vL-[IgCL-TCRg-6MD]-F-P2A- | 2933 | 3025 |
| SP-FMC63-vH-[IgG1-CH1-TCRd-6MD]-F- | | |
| F2A-DR5-SP-ECD-TM-BCMA-CP | | |
| CD8SP-NY-ESO-[hTCRb-KACIAH]-F-P2A-SP- | 2934 | 3026 |
| NYESO-[hTCRa-CSDVP]-F-F2A-DR5-SP-ECD- | | |
| TM-BCMA-CP | | |
| CD8SP-NY-ESO-TCRa-F-P2A-SP-NYESO- | 2935 | 3027 |
| [hTCRb]-F-F2A-DR5-SP-ECD-TM-BCMA-CP | | |

TABLE 7B
Nucleic acid vectors and components

| SEQ ID | NAME |
|---|---|
| 337 | pLenti-EF1a |
| 338 | pLenti-EF1a-DWPRE |
| 339 | EFlalpha_(EF1a)_Promoter_Variant |
| 340 | SV40-polyA |
| 341 | PolyA |
| 342 | PolyA |
| 343 | PolyA |
| 344 | DNA barcode 1 |
| 345 | DNA barcode 2 |
| 346 | DNA barcode 3 |
| 347 | DNA barcode 4 |
| 348 | DNA barcode 5 |
| 349 | DNA barcode 6 |
| 350 | Plenti-EF1a-FMC63(yL-yH)-GGSG- |
| | NLuc-AcV5-U09 |
| 351 | MSCVhygro-GLuc-HA-G02 |
| 352 | pLENTI-NLuc-AcV5-Blasticidin-Pa08 |
| 353 | H1-promoter |
| 354 | H1-shRNA-BRD9 |
| 355 | pLenti-EF1a-shRNA-BRD9-DWPRE |
| 356 | pLenti-EF1a-CD8SP-FMC63-a-V5- |
| | [hTCRb-KACIAH]-F-P2A-SP- |
| | FMC63-yH-Myc-[hTCRa-CSDVP]- |
| | F-F2A-PAC-shRNA-BRD9-DWPRE |
| 357 | pLKO.1 |
| 358 | pX601-AAV-CMV-NLS-SaCas9-NLS- |
| | 3xHA-bGHpA-U6-BsaI-sgRNA |
| 359 | pLenti-CRISPR-y2 |

TABLE 7C
Exemplary CAR/TCR constructs

| | | SEQ |
|---|---|---|
| TARGET | | ID |
| ANTIGEN | NAME OF CAR/TCR CONSTRUCT | NO |
| CD19 | CD8SP-FMC63-(vL-vH)-Myc-BBz | 2822 |
| BCMA | CD8SP-BCMA-J6M0-(vL-vH)-Myc-BBz | 2823 |
| CD20 | CD8SP-CD20-2F2-(vL-vH)-Myc-BBz | 2824 |
| CD123 | CD8SP-CD123-1172-(vL-vH)-Myc-BBz | 2825 |
| CS1 | CD8SP-CS1-HuLuc64-(vL-vH)-Myc- | 2826 |
| | BBz | |
| Folate | CD8SP-FR1-huMov19-(vL-vH)-Myc- | 2827 |
| Receptor 1 | BBz | |
| GM1 | CD8SP-GM1-5B2-(vL-vH)-Myc-BBz | 2828 |
| IL13Ra2 | CD8SP-IL13Ra2-hu107-(vL-vH)-Myc- | 2829 |
| | BBz | |
| IL13Ra2 | CD8SP-IL13Ra2-Hu108-(vL-vH)-Myc- | 2830 |
| | BBz | |
| Her2 | CD8SP-Her2-Hu4D5-(vL-vH)-Myc-BBz | 2831 |
| Mesothelin | CD8SP-Mesothelin-m912-(vH-vL)- | 2832 |
| | Myc-BBz | |
| MPL | CD8SP-MPL-161-(vL-vH)-Myc-BBz | 2833 |
| WT1/MHC I | CD8SP-WT1-Ab1-(vL-vH)-Myc-BBz | 2834 |
| AFP/MHC | CD8SP-AFP-76-(vL-vH)-Myc-BBz | 2835 |
| complex | | |
| NY-ESO/MHC | NY-ESO-[TCRa]-F-P2A-NYESO- | 2836 |
| complex | [hTCRb] | |

TABLE 7D
SEQ ID NO of Extracellular Domain of Proteins that
can be expressed on APC (Antigen Presenting Cells)
or conjugated to Antigen Presenting Substrates (APS)

| | Extracellular Domain of Receptors that |
|---|---|
| SEQ | can be expressed on APC or conjugated |
| ID NO | to Antigen Presenting Substrates |
| 3038 | CD19-ECD |
| 3039 | MPL-ECD |
| 3040 | PD1-ECD |
| 3041 | CTLA4-ECD |
| 3042 | CD138-SDC1-ECD |
| 3043 | CD123-ECD |
| 3044 | CDH1-ECD |
| 3045 | CD200R1L-ECD |
| 3046 | GPNMB-ECD |
| 3047 | PTK7-ECD |
| 3048 | CD33-ECD |
| 3049 | CD34-ECD |
| 3050 | EpCAM-ECD |
| 3051 | CLEC12A-ECD |
| 3052 | CD20-ECx2-ECD |
| 3053 | CD20-ECX1-ECD |
| 3054 | CD22-v5-ECD |
| 3055 | Thyroid Stimulating Hormone Receptor |
| | (TSHR)-ECD |
| 3056 | EGFRviii-ECD |
| 3057 | BCMA-ECD |
| 3058 | SLAMF7-CS1-ECD |
| 3059 | NKG2D-ECD |

The disclosure provides cell therapy products (e.g., immune effector cells e.g., T cells, NK cells, CAR-T cells, TCR-T cells) that are engineered to contain one or more CARs (or TCRs) that target disease causing or disease associated cells, such as cancer cells. This is achieved through an antigen binding domain on the CAR or TCR that is specific for a cancer associated antigen. There are two classes of cancer associated antigens (tumor antigens) that can be targeted by the CARs and/or TCRs: (1) cancer associated antigens that are expressed on the surface of cancer cells; and (2) cancer associated antigens that itself is intracelluar, however, a fragment of such antigen (peptide) is presented on the surface of the cancer cells by MHC (major histocompatibility complex).

Accordingly, the cells therapy products (e.g., immune cells) expressing the CARs of the disclosure may target one or more of the following cancer associated antigens (tumor antigens): CD5, CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); FmsLike Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20/MS4A1; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CA1X); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanomaassociated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B 1); CCCTC-Binding Factor (Zinc Finger Protein)-Like (BORIS or Brother of the Regulator oflmprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation End products (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRI); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), MPL, Biotin, c-MYC epitope Tag, CD34, LAMP1 TROP2, GFRalpha4, CDH17, CDH6, NYBRI, CDH19, CD200R, Slea (CA19.9; Sialyl Lewis Antigen) Fucosyl-GM1, PTK7, gpNMB, CDH1-CD324, DLL3, CD276/B7H3, IL11Ra, IL13Ra2, CD179b-IGL11, ALK TCRgamma-delta, NKG2D, CD32 (FCGR2A), CSPG4-HMW-MAA, Tim1-/HVCR1, CSF2RA (GM-CSFR-alpha), TGFbetaR2, VEGFR2/KDR, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Chorionic Gonadotropin Hormone receptor (CGHR), CCR4, SLAMF6, SLAMF4, HIV1 envelope glycoprotein, HTLV1-Tax, CMV pp65, EBV-EBNA3c, influenza A hemagglutinin (HA), GAD, PDL1, Guanylyl cyclase C (GCC), KSHV-K8.1 protein, KSHV-gH protein, auto-antibody to desmoglein 3 (Dsg3), autoantibody to desmoglein 1 (Dsg1), HLA, HLA-A, HLA-A2, HLA-B, HLA-C, HLA-DP, HLA-DM, HLA-DOA, HLA-DOB, HLA-DQ, HLA-DR, HLA-G, IGE, CD99, RAS G12V, Tissue Factor 1 (TF1), AFP, GPRC5D, claudin18.2 (CLD18A2 OR CLDN18A.2)), P-glycoprotein, STEAPI, LIV1, NECTIN-4, CRIPTO, MPL, GPA33, BST1/CD157, low conductance chloride channel, Integrin B7, Muc17, C16ORF54, VISTA, Muc5Ac, FCRH5, CLDN6, MMP16, UPK1B, BMPR1B, Ly6E, WISP1 and SLC34A2.

The SEQ ID Nos of several exemplary CARs/TCRs are listed in Table 7C.

A CAR can comprise an antigen binding domain (e.g., antibody or antibody fragment, TCR or TCR fragment) that binds to a tumor-supporting antigen (e.g., a tumor-supporting antigen as described herein). In some embodiments, the tumor-supporting antigen is an antigen present on a stromal cell or a myeloid-derived suppressor cell (MDSC).

The disclosure encompasses a recombinant DNA construct comprising sequences encoding a natural (e.g., a TCR) or a Synthetic Immune Receptor (e.g., a 2nd generation CAR or a SIR).

In specific aspects, a CAR may comprise a scFv domain, wherein the scFv may be preceded by an optional leader sequence such as provided in SEQ ID NO (PRT):1834 and 1835, and followed by an optional hinge sequence and transmembrane region such as provided in SEQ ID NO: 1854, an intracellular signalling domain that includes SEQ ID NO: 1857 and a CD3 zeta sequence that includes SEQ ID NO: 1857 or SEQ ID NO:1858, e.g., wherein the domains are contiguous with and in the same reading frame to form a single fusion protein.

An exemplary leader sequence is provided as SEQ ID NO:1834.

An exemplary hinge and transmembrane domain sequence is provided as SEQ ID NO: 1854. An exemplary sequence of the intracellular signaling domain of the 4-1BB protein is provided as SEQ ID NO: 1857. An exemplary CD3zeta domain sequence is provided as SEQ ID NO: 1858 or SEQ ID NO:1859.

Exemplary second generation CARs targeting CD19 are presented in SEQ ID NO: 1903 and 1904. These CAR constructs also encode for a puromycin resistance gene (PAC) that is separted from the CAR polypeptide by a T2A cleavage sequence. The names and SEQ IDs of exemplary second generation CARs targeting CD19 and co-expressing constitutive active mutants of JAK3 (SEQ ID NO: 1911 and 1912), STAT5b (SEQ ID NO: 1913), JAK1(SEQ ID NO: 1914), CARD11 (SEQ ID NO: 1915), STAT3 (SEQ ID NO: 1916), and BRAF (SEQ ID NO: 1917) are shown in Table 7a.

The disclosure also covers immune effector cells expressing next generation CAR constructs, including K13 (vFLIP)-CAR, SIR (Synthetic Immune Receptor), Ab-TCR and TFP. Exemplary K13 coexpressing CAR constructs are represented by SEQ ID NO: 1908 and 1909. These CAR constructs also co-express a puromycin resistence gene (PAC), which is optional and can be deleted. Exemplary SIR constructs coexpressing an optional PAC gene are represented by SEQ ID NO:1906 and 1907.

The names and SEQ IDs of exemplary SIR (SEQ ID NO: 1918), K13-CAR (SEQ ID NO: 1919), Ab-TCR (SEQ ID NO: 1924-1925) and TFP (SEQ ID NO: 1920-1922) targeting CD19 and co-expressing constitutive active mutants of JAK3-M511I mutant are shown in Table 7a. Constructs co-expressing other constitutive active mutants can be constructed by replacing the cDNA encoding JAK3-M511I with the cDNAs corresponding to the other constitutive active mutants. The disclosure includes retroviral and lentiviral vector constructs (SEQ ID NO: 337 and 338) that can be used the various embodiments of the disclosure.

The disclosure also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”) (e.g., a 3′ and/or 5′ UTR described herein), a 5′ cap (e.g., a 5′ cap described herein) and/or Internal Ribosome Entry Site (IRES) (e.g., an IRES described herein), the nucleic acid to be expressed, and a polyA tail, typically 50-2000 bases in length (SEQ ID NO:341). RNA so produced can efficiently transfect different kinds of cells. In one embodiment, the template includes sequences for the CAR. In an embodiment, an RNA CAR vector is transduced into a cell, e.g., a T cell or a NKcell, by electroporation.

The CAR/TCR comprise a target-specific binding element otherwise referred to as an antigen binding domain. Conventional and next generation CARs (e.g., SIR, Ab-TCR, TFP etc) against a number of antigens are described in the art, including in patent applications PCT/US2017/024843 and PCT/US17/64379, which are incorporated herein in their entirety by reference. The CARs described in these applications can be used in combination with the methods of the disclosure to target different antigens for the prevention and treatment of various disease conditions in which the disease associated or disease-causing cells express the specific antigen targeted by the CAR. Similarly, TCR targeting different antigens and neo-antigens are known in the art and can be used in combination with the methods of the disclosure. Finally, the efficacy and safety of tumor infliterating lymphocytes and T cells generated following vaccination with neo-antigen peptides can be improved by use of the methods of the disclosure.

In one embodiment, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDRI, LC CDR2 and LC CDR3, from an antibody or single chain variable fragment described in PCT/US2017/024843 and PCT/US17/64379. In another aspect, the antigen binding domain is a cytokine, a receptor, a centryn, a non-immunoglobulin antigen binding domain.

In another aspect, the cell therapy products (e.g., CAR-T cell) described herein can further express another agent, e.g., an agent which enhances the activity of the cell therapy products (e.g. CAR-expressing cell). For example, in one embodiment, the agent can be a constitutive active mutant of JAK3 (SEQ ID NO: 1911 and 1912), STAT5b (SEQ ID NO: 1913), JAK1(SEQ ID NO: 1914), CARD11 (SEQ ID NO: 1915), STAT3 (SEQ ID NO: 1916), and BRAF (SEQ ID NO: 1917). In another embodiment, the agent can be an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5.

In another aspect, the disclosure provides a population of cell therapy products, e.g., CAR-T and/or TCR-T cells. In some embodiments, the cell therapy products comprise a mixture of cells expressing different CARs.

In another aspect, the disclosure provides a population of cells wherein at least one cell in the population expresses a CAR having an antigen binding domain to a cancer associated antigen described herein, and a second cell expressing another agent, e.g., an agent which enhances the activity of a CAR-expressing cell. In one aspect, the disclosure provides methods comprising administering a population of CAR-expressing cells, e.g., CART cells, e.g., a mixture of cells expressing different CARs, in combination with another agent, e.g., a kinase inhibitor, such as a kinase inhibitor described herein. In another aspect, the disclosure provides methods comprising administering a population of cells wherein at least one cell in the population expresses a CAR having an antigen binding domain of a cancer associated antigen described herein, and a second cell expressing another agent, e.g., an agent which enhances the activity of a CAR-expressing cell, in combination with another agent, e.g., a kinase inhibitor, such as a kinase inhibitor described herein.

The nucleic acids encoding the different constructs (e.g., CARs, SIRs, TCRs, constitutive active and dominant negative mutants of JAK3, STAT5 etc.) of the disclosure can be delivered to cells using methods known in the art. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length (SEQ ID NO:32). RNA so produced can efficiently transfect different kinds of cells. In one aspect, the template includes sequences for the CAR.

In some aspects, non-viral methods can be used to deliver a nucleic acid encoding the different constructs described herein into a cell or tissue or a subject. In some embodiments, the non-viral method includes the use of a transposon (also called a transposable element). Exemplary methods of nucleic acid delivery using a transposon include a Sleeping Beauty transposon system (SETS) and a piggyBac (PB) transposon system.

Methods of constructing and delivering vectors with different configurations of CAR, including next generation CARs, are known in the art and are described in patent applications PCT/US2017/024843, WO 2014/160030 A2, WO 2016/187349 A1, PCT/US2016/058305 and PCT/US17/64379, which are incorporated herein by reference in their entirety.

The disclosure also provides vectors in which a DNA of the disclosure is inserted. Vectors derived from retroviruses such as the lentivirus are suitable tools to achieve long-term gene transfer since they allow long-term, stable integration of a transgene and its propagation in daughter cells.

During the manufacturing of cell therapy products, immune effector cells (e.g., T cells) may be activated and expanded generally using methods known in the art. Generally, a population of immune effector cells e.g., Plerixafor mobilized cells, may be expanded by contact with a surface having attached thereto an agent that stimulates a CD3/TCR complex associated signal and a ligand that stimulates a costimulatory molecule on the surface of the T cells.

In certain aspects, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution.

The present disclosure provides novel antigen presenting cells for activation and expansion of immune cells during the manufacturing of cell therapy products. In one embodiment, cells transduced with a nucleic acid encoding a CAR (e.g., a CAR, e.g., a SIR) are expanded by co-culturing them with an antigen presenting cell. In one embodiment, T cells transduced with a nucleic acid encoding a CAR, e.g., a CAR described herein, are expanded by co-culturing them with cells expressing the antigen targeted by the CAR. In one embodiment, the T cells are transduced with a nucleic acid encoding a CAR and are expanded by co-culturing them with cells and/or cell lines derived from Mantle Cell Lymphoma. In one embodiment, the T cells are transduced with a nucleic acid encoding a CD19 CAR, e.g., a CD19 CAR described herein, e.g., a CAR represented by SEQ ID NO: 2822, 479, 484-498, and are expanded by co-culturing them with cells and cell lines derived from Mantle Cell Lymphoma. In one embodiment, the T cells are transduced with a nucleic acid encoding a CD20 CAR, e.g., a CD20 CAR described herein, e.g., a CAR represented by SEQ ID NO: 2824, 480 or 482, and are expanded by co-culturing them with cells and cell lines derived from Mantle Cell Lymphoma. In one embodiment, the T cells are transduced with a nucleic acid encoding a CD22 CAR, e.g., a CD22 CAR described herein, and are expanded by co-culturing them with cells and cell lines derived from Mantle Cell Lymphoma. In one embodiment, the T cells are transduced with a nucleic acid encoding a BCMA CAR, e.g., a BCMA CAR described herein, e.g., a CAR represented by SEQ ID NO: 2823, and are expanded by co-culturing them with cells and cell lines derived from Mantle Cell Lymphoma. Exemplary cell lines derived from Mantle cell lymphoma that can be used to expand and/or activate CAR-T cells targeting CD19, CD20, CD22 and BCMA incude REC-1, GRANTA-519, MINO and JEKO. In a preferred embodiment, the Mantle Cell Lymphoma cell line is REC-1.

In one embodiment, the T cells are transduced with a nucleic acid encoding a CAR and are expanded by co-culturing them with cells and cell lines that have been treated with a drug (e.g, mitomycin) or irradiation to render them replication incompetent. Methods to render cells and cell lines replication incompetent are known in the art and/or can be determined using methods known in the art.

In one embodiment, the T cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. In one embodiment, the cells are expanded for a period of 8 days or less, e.g., 7, 6 or 5 days.

In one embodiment, the cells, e.g., a CD19 CAR-T cell described herein, are expanded in culture for 5 days, and the resulting cells are more potent than the same cells expanded in culture for 9 days under the same culture conditions. In one embodiment, the CAR-T cells, e.g., CD19 CAR-T cells described herein, are expanded in culture for 14 days with Mantle Cell Lymphoma cells, e.g, REC-1 cells, and the resulting cells are more potent than the same CAR-T cells, e.g., CD19 CAR-T cells described herein, expanded in culture under the same culture conditions but without the Mantle Cell Lymphoma cells, e.g., REC-1 cells. In one embodiment, the CAR-T cells, e.g., CD20 CAR-T cells described herein, are expanded in culture for 14 days with Mantle Cell Lymphoma cells, e.g, REC-1 cells, and the resulting cells are more potent than the same CAR-T cells, e.g., CD20 CAR-T cells described herein, expanded in culture under the same culture conditions but without the Mantle Cell Lymphoma cells, e.g., REC-1 cells. In one embodiment, the CAR-T cells, e.g., CD22 CAR-T cells described herein, are expanded in culture for 14 days with Mantle Cell Lymphoma cells, e.g, REC-1 cells, and the resulting cells are more potent than the same CAR-T cells, e.g., CD22 CAR-T cells described herein, expanded in culture under the same culture conditions but without the Mantle Cell Lymphoma cells, e.g., REC-1 cells. In one embodiment, the CAR-T cells, e.g., BCMA CAR-T cells described herein, are expanded in culture for 14 days with Mantle Cell Lymphoma cells, e.g, REC-1 cells, and the resulting cells are more potent than the same CAR-T cells, e.g., BCMA CAR-T cells described herein, expanded in culture under the same culture conditions but without the Mantle Cell Lymphoma cells, e.g., REC-1 cells.

Potency can be defined, e.g., by various T cell functions, e.g. proliferation, target cell killing, cytokine production, activation, migration, or combinations thereof. In one embodiment, the cells, e.g., a CD19 CAR-T cell described herein, expanded for 5 days show at least a one, two, three or four-fold increase in cells doublings upon antigen stimulation as compared to the same cells expanded in culture for 9 days under the same culture conditions. In one embodiment, the cells, e.g., the cells expressing a CD19 CAR described herein, are expanded in culture for 5 days, and the resulting cells exhibit higher proinflammatory cytokine production, e.g., IFN-γ and/or GM-CSF levels, as compared to the same cells expanded in culture for 9 days under the same culture conditions. In one embodiment, the cells, e.g., a CD19 CAR-T cell described herein, expanded for 5 days show at least a one, two, three, four, five, ten fold or more increase in pg/ml of proinflammatory cytokine production, e.g., IFN-γ and/or GM-CSF levels, as compared to the same cells expanded in culture for 9 days under the same culture conditions.

Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGF, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In one embodiment, the cells are expanded in an appropriate media (e.g., media described herein) that includes one or more interleukin that result in at least a 200-fold (e.g., 200-fold, 250-fold, 300-fold, 350-fold) increase in cells over a 14 days expansion period, e.g., as measured by a method described herein such as flow cytometry. In one embodiment, the cells are expanded in the presence of IL-15 and/or IL-7 (e.g., IL-15 and IL-7).

In one embodiment of the disclosure, the cells (e.g., immune cells, e.g., T cells or NK cells), e.g., immune effector cells, e.g., T cells, e.g., CART-T cells, TCR-T cells or TILs, are expanded in an appropriate media (e.g., media described herein) that includes one or more inhibitors of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL (TNFSF10) and/or Death Receptor 5 (DR5 or TNFRSF10B) that result in at least a 1.5-fold (e.g., 2-fold, 5-fold, 10-fold, 50-fold) increase in T stem cells or T stem memory cells over a 14 day expansion period, e.g., as measured by a method described herein such as flow cytometry. In one embodiment, the cells are expanded in the presence of IL-15 and/or IL-7 (e.g., IL-15 and IL-7). In one embodiment, the cells are expanded in the presence of an inhibitor of TRAIL and/or DR5. In an embodiment, the TRAIL inhibitor is a TRAIL antibody that blocks or neutralizes the action binding or activity of TRAIL on its receptors DR5 and/or DR4. In an exemplary embodiment, the cells are expanded in the presence of a neutralizing antibody against TRAIL. An exemplary neutralizing antibody against TRAIL is represented by the TRAIL antibody MAB375-SP which is available from R&D Systems. In an embodiment, the cells are expanded in the presence of at least 1 ng/ml (e.g., 2 ng/ml, 5 ng/ml, 10 ng/ml, 20 ng/ml, 50 ng/ml) of a TRAIL antibody, e.g., MAB375-SP. In an embodiment, the cells are expanded in the presence of a TRAIL antibody, e.g., MAB375-SP for at least 1 day (e.g., 2 days, 5 days, 10 days, 15 days, 20 days). In an embodiment, the TRAIL inhibitor is a soluble form of TRAIL receptor. Exemplary soluble TRAIL receptor include DR5-Fc fusion protein (Sigma-Aldrich; D9563), DR5-SP-ECD-hIgFc (SEQ ID NO: 2428), Recombinant Human TRAIL R1/TNFRSF10A Fc Chimera Protein, CF (R&D Systems), DR4-Fc fusion protein (Sigma-Aldrich; D9438), DR4-SP-ECD-hIgFc (SEQ ID NO: 2441), Recombinant Human TRAIL R3/TNFRSF10C Fc Chimera Protein (R&D Systems); DcR1-SP-ECD-hIgFc (SEQ ID NO: 2448); Recombinant Human TRAIL R3/TNFRSF10C Fc Chimera Protein (R&D Systems) and DcR2-ECD-hIgFc (SEQ ID NO: 2455). In an embodiment, the cells are expanded in the presence of at least 1 ng/ml (e.g., 2 ng/ml, 5 ng/ml, 10 ng/ml, 20 ng/ml, 50 ng/ml, 100 ng/ml) of purified soluble TRAIL receptor, e.g., DR5-Fc fusion protein (Sigma-Aldrich; D9563) or DR4-Fc fusion protein (Sigma-Aldrich; D9438). In an embodiment, the cells are expanded in the presence of a soluble TRAIL receptor (e.g., DR5-Fc, DR4-Fc, DcR1-Fc, or DcR2-Fc) for at least 1 day (e.g., 2 days, 5 days, 10 days, 15 days, 20 days). In some embodiments, the cells are expanded in the presence of both a neutralizing TRAIL antibody (e.g., MAB375-SP) and a soluble TRAIL receptor (e.g., DR5-Fc, DR4-Fc, DcR1-Fc, or DcR2-Fc). In an embodiment, the cells are expanded in the presence of a nucleic acid inhibitor (e.g., shRNA, siRNA or gRNA) of TRAIL and/or DR5. In an embodiment, the cells are expanded in the presence of a TRAIL inhibitor and an inhibitor of one or more of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2 and AKT. In an embodiment, the cells are expanded in the presence of a TRAIL inhibitor and one or more of an CD3 antibody, CD28 antibody, 41BB antibody (Utomilumab), bispecific/multispecific T cell engager, e.g, a bispecific or multispecific engager described herein, e.g., Blinatumomab. In an embodiment, the cells are expanded in the presence of a TRAIL inhibitor and a bispecific/multispecific T cell engager, e.g, a bispecific or multispecific engager described herein, in the presence of Antigen Presenting Cells (APC) or Antigen Presenting Substrate (APS), e.g., APC or APS described herein, e.g., REC-1 cells or CD19-Ectodomain (amino acid residues 61-867)-coated Beads.

The present disclosure also pertains, at least in part, to methods for improving the expansion and/or activation (e.g., in vitro and in vivo expansion and/or activation) of cells (e.g., immune cells, e.g., T cells or NK cells), e.g., immune effector cells, e.g., immune effector T cells, e.g., CAR-T cells or TCR-T cells or TILs, for the purpose of adoptive cellular therapy. Non-limiting examples of cells (e.g., immune cells, e.g., T cells or NK cells) that can be activated and/or expanded by the method of the disclosure include T cells, CD8+ T cells, CD4+ T cells, NKT cells, NK cells, monocytes and macrophages etc. In an embodiment, the cells are mobilized (e.g., Plerixafor-mobilized) cells. In an embodiment, the cells express an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2 and AKT.

In some embodiments, the method includes contacting an immune cell e.g., an immune effector cell, e.g., an immune effector cell described herein, e.g., a T cell, e.g., a CAR-T cell or a TCR-T cell, with a bispecific or multi-specific engager with two or more antigen binding modules where at least one of the antigen binding modules binds to or engages an immune cell and where at least one of the other modules binds to or engages an antigen presenting cell (APC) or an antigen presenting substrate (APS) under conditions that result in the activation and/or expansion of the immune cells. In some embodiments, the one or all of the antigen binding modules of the bispecific or multi-specific engager comprise of or consist of (1) an antibody; (2) an antibody fragment (e.g. a Fv, a Fab, a (Fab′)2); (3) a heavy chain variable region of an antibody (vH domain) or a fragment thereof, (4) a light chain variable region of an antibody (vL domain) or a fragment thereof, (5) a single chain variable fragment (scFv) or a fragment thereof, (6) a single domain antibody (SDAB) or a fragment thereof, (7) a camelid VHH domain or a fragment thereof, (8) a monomeric variable region of an antibody; (9) a non-immunoglobulin antigen binding scaffold such as a DARPIN, an affibody, an affilin, an adnectin, an affitin, an obodies, a repebody, a fynomer, an alphabody, an avimer, an atrimer, a centyrin, a pronectin, an anticalin, a kunitz domain, an Armadillo repeat protein or a fragment thereof; (10) a receptor or a fragment thereof; (11) a ligand or a fragment thereof. In some embodiments, the two or more of the antigen binding modules of the bispecific or multispecific engager are of the same type, e.g., scFVs or vHH. In some embodiments, the two or more of the antigen binding modules of the bispecific or multispecific engager are of the different types, e.g., a scFVs and a vHH domain; a scFv and an centyrin; a vHH domain and an affibody; a scFv, a vHH domain and a centyrin.

In an embodiment a bispecific or multispecific engager is a bispecific antibody molecule, e.g., Blinatumomab. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and ahalf antibody, or fragment thereof, having binding specificity for a second epitope.

In an embodiment a bispecific antibody molecule comprises a scFv, or fragment thereof, have binding specificity for a first epitope and a scFv, or fragment thereof, have binding specificity for a second epitope.

In certain embodiments, the engager is an antibody molecule that is a multi-specific (e.g., a bispecific or a trispecific) molecule. Protocols for generating bispecific or heterodimeric antibody molecules are known in the art.

Within each antibody or antibody fragment (e.g., scFv) of abispecific antibody molecule, the VH can be upstream or downstream of the VL. In some embodiments, the upstream antibody or antibody fragment (e.g., scFv) is arranged with its VH (VH1) upstream of its VL (VL1) and the downstream antibody or antibody fragment (e.g., scFv) is arranged with its VL (VL2) upstream of its VH (VH2), such that the overall bispecific antibody molecule has the arrangement VH1-VL1-VL2-VH2. In other embodiments, the upstream antibody or antibody fragment (e.g., scFv) is arranged with its VL (VL1) upstream of its VH (VH1) and the downstream antibody or antibody fragment (e.g., scFv) is arranged with its VH (VH2) upstream of its VL (VL2), such that the overall bispecific antibody molecule has the arrangement VL1-VH1-VH2-VL2.

In some embodiments, the bispecific or multispecific engager targets at least one antigen (e.g., CD3, CD28, CD27, 41BB etc) expressed by an immune cell to be activated and/or expanded and at least one other antigen (e.g., CD19, Mesothelin, Her2, Her3, EGFR viii etc.) expressed by a cell other than the immune cell that is to be activated and/or expanded. In some embodiments, the other antigen is expressed or presented by an antigen presenting cell (APC) or an antigen presenting substrate (APS), e.g., CD19 ectodomain coated beads. In some embodiments, the at least one other antigen expressed by the APC or APS (e.g., CD19, CD20/MS4A1, CD22, CD23, CD123, MPL, BCMA, CS1, CD138, CD38 etc.) is expressed on hematopoietic cells, e.g., B lineage cell, a myeloid lineage cell or a plasma cell or cell lines, e.g., REC-1, NALM6, HL60, K562, BC-1, U266 etc. In some embodiments, the at least one other antigen expressed by the APC or APS (e.g., Her2, Her3, EGFR, Mesothelin, CDH19, CDH6 etc.) is expressed on non-hematopoietic cells, e.g., breast cells, lung cells, colon cells, skin cells etc, or cell lines, e.g., breast cancer cell line, e.g., MCF7, lung cancer cell line, e.g., H460, or colon cancer cell line, e.g., SW480 etc.

Non-limiting examples of the antigens expressed by the APC or APS that can be recognized by the bispecific or multispecific engager of the disclosure to activate and/or expand the immune cells include one or more of the following: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); FmsLike Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20/MS4A1; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CA1X); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanomaassociated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B 1); CCCTC-Binding Factor (Zinc Finger Protein)-Like (BORIS or Brother of the Regulator oflmprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation End products (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRI); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), MPL, Biotin, c-MYC epitope Tag, CD34, LAMP1 TROP2, GFRalpha4, CDH17, CDH6, NYBRI, CDH19, CD200R, Slea (CA19.9; Sialyl Lewis Antigen) Fucosyl-GM1, PTK7, gpNMB, CDH1-CD324, DLL3, CD276/B7H3, IL11Ra, IL13Ra2, CD179b-IGL11, ALK TCRgamma-delta, NKG2D, CD32 (FCGR2A), CSPG4-HMW-MAA, Tim1-/HVCR1, CSF2RA (GM-CSFR-alpha), TGFbetaR2, VEGFR2/KDR, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Chorionic Gonadotropin Hormone receptor (CGHR), CCR4, SLAMF6, SLAMF4, HIV1 envelope glycoprotein, HTLV1-Tax, CMV pp65, EBV-EBNA3c, influenza A hemagglutinin (HA), GAD, PDL1, Guanylyl cyclase C (GCC), KSHV-K8.1 protein, KSHV-gH protein, auto-antibody to desmoglein 3 (Dsg3), autoantibody to desmoglein 1 (Dsg1), HLA, HLA-A, HLA-A2, HLA-B, HLA-C, HLA-DP, HLA-DM, HLA-DOA, HLA-DOB, HLA-DQ, HLA-DR, HLA-G, IGE, CD99, RAS G12V, Tissue Factor 1 (TF1), AFP, GPRC5D, claudin18.2 (CLD18A2 OR CLDN18A.2)), P-glycoprotein, STEAPI, LIV1, NECTIN-4, CRIPTO, MPL, GPA33, BST1/CD157, low conductance chloride channel, Integrin B7, Muc17, C16ORF54, VISTA, Muc5Ac, FCRH5, CLDN6, MMP16, UPK1B, BMPR1B, Ly6E, WISP1 and SLC34A2.

Some embodiments described herein provide for expansion and/or activation of immune T cells by exposing them to a bispecific or multi-specific engager that contains one (or first) antigen binding domain capable of engaging the T cells and the other (or second) antigen binding domain capable of engaging an antigen presenting cell (APC) or antigen presenting substrate (APS). It is to be noted that the order of the antigen binding domains of a bispecific engager can be reversed without affecting its activity. Thus, a CD19 directed bispecific engager may have the configuration CD19×CD3 or CD3×CD19. In some embodiment, the APC is a hematopoietic cell. In some embodiments, the method further involves exposing the immune T cells to an agonist, such as an antibody (e.g., Utomilumab) or a ligand (41BBL), capable of activating a costimulatory receptor (e.g., CD28, 41BB, CD27 etc.) on T cells.

In some embodiment, the bispecific/multispecific engager comprises at least one (or first) binding domain capable of binding to and activating the T cell receptor (TCR) complex of T cells. In some embodiment, the bispecific/multispecific comprises at least one (or first) binding domain capable of binding to and activating the CD3 subunit of the TCR complex. In some embodiments, the bispecific/multispecific comprises at least one (or one) binding domain capable of binding to and activating the CD3-epsilon subunit of the TCR complex.

In some embodiments, the bispecific/multispecific engager comprises at least one (or first) binding domain capable of binding to and activating a receptor on the T cells that provides co-stimulation; i.e., a co-stimulatory receptor. Exemplary co-stimulatory receptor bound by the bispecific engager include CD28, CD137 (4-1BB), CD134 (OX40), Dap10, CD27, CD2, CD5, ICAM-1, LFA-1, TNFR-I, TNFR-II, Fas, CD30 and CD40.

In some embodiment, the bispecific/multispecific engager in the presence of APC or APS activates the signaling through the TCR complex. In some embodiment, the bispecific/multispecific engager in the presence of APC or APS activates T cells via signaling through a co-stimulatory receptor.

In some embodiment, the bispecific/multispecific engager comprises at least one (or second) binding domain capable of binding to the hematopoietic cells. In some embodiment, the bispecific/multispecific engager comprises at least one (or second) binding domain capable of binding to the lymphoid-lineage hematopoietic cells. In some embodiment, the bispecific/multispecific engager comprises at least one (or second) binding domain capable of binding to the B-lymphoid-lineage hematopoietic cells. Exemplary B-lineage lymphoid cells bound by the bispecific/multispecific engager, e.g., bispecific antibody, include immature B cells, mature B cells and plasma cells and combination thereof. In some embodiment, the bispecific/multispecific engager, e.g., bispecific antibody, comprises at least one (or second) binding domain capable of binding to an antigen expressed on B-lymphoid-lineage hematopoietic cells. Non-limiting examples of antigens bound by the second binding domain of the bispecific/multispecific engager, e.g., bispecific antibody, include CD19, CD20/MS4A1, CD22, CD23, BCMA, CS1/SLAMF7, CD30, CD32b, CD70, CD79b, CD123, CD33, CD138, CD179b, GPRC5D, Lyml, Lym2, and FCRH5.

In some embodiments, at least one (first) antigen binding domain of the bispecific/multispecific engager, e.g., bispecific antibody, binds to CD3e (or CD3ε) and the at least one other (or the second) antigen binding domain binds to CD19. In some embodiments, the bispecific/multispecific engager, e.g., bispecific antibody, is Blinatumomab. In some embodiment, the bispecific/multispecific engager, e.g., bispecific antibody, is a CD19×CD3 DART as described in Moore P A et al, Blood, 2011; 117(17):4542-4551.

In some embodiments, at least one (first) antigen binding domain of the bispecific/multispecific engager, e.g., bispecific antibody, binds to CD3e (or CD3ε) and the at least one other (or the second) antigen binding domain binds to CD22. In some embodiments, at least one (first) antigen binding domain of the bispecific/multispecific engager, e.g., bispecific antibody, binds to CD3e (or CD3ε) and the at least one other (or the second) antigen binding domain binds to CD20/MS4A1.

In some embodiments, at least one (first) antigen binding domain of the bispecific/multispecific engager, e.g., bispecific antibody, binds to CD3e (or CD3ε) and the at least one other (or the second) antigen binding domain binds to CD23.

In some embodiments, at least one (first) antigen binding domain of the bispecific/multispecific engager, e.g., bispecific antibody, binds to CD3e (or CD3ε) and the at least one other (or the second) antigen binding domain binds to BCMA. In some embodiments, the bispecific antibody is BI 836909 (AMG 420).

In some embodiments, at least one (first) antigen binding domain of the bispecific/multispecific engager, e.g., bispecific antibody, binds to CD3e (or CD3ε) and the at least one other (or the second) antigen binding domain binds to CS1/SLAMF7.

In some embodiments, at least one (first) antigen binding domain of the bispecific/multispecific engager, e.g., bispecific antibody, binds to CD3e (or CD3ε) and the at least one other (or the second) antigen binding domain binds to CD138.

In some embodiments, at least one (first) antigen binding domain of the bispecific/multispecific engager, e.g., bispecific antibody, binds to CD3e (or CD3ε) and the at least one other (or the second) antigen binding domain binds to CD123.

In some embodiments, at least one (first) antigen binding domain of the bispecific/multispecific engager, e.g., bispecific antibody, binds to CD3e (or CD3ε) and the at least one other (or the second) antigen binding domain binds to MPL. Table 7E provides the SEQ ID NOs of several bispecific antibodies whose first (or one of the) antigen binding domain binds to CD3e, CD28 or 41BB and whose second (or the other) antigen binding domain binds to different antigens, such as CD19, CD20/MS4A1, CD22, BCMA, CD33, CD123, MPL, Folate Receptor 1 etc.

In some embodiments, the activation and expansion of T cells involves exposing them to the bispecific/multispecific engager in the presence of a cell expressing a cognate ligand (e.g. an antigen) bound by at least one (second) antigen binding domain of the engager.

In some embodiments, the activation and expansion of T cells involves exposing them to the bispecific/multispecific engager in the presence of a solid substrate expressing a cognate ligand (e.g. an antigen or anti-idiotype antibody) bound by at least one (second) antigen binding domain of the engager.

In some embodiments, the method involves activation/expansion of immune T cells by exposing them to two different bispecific/multispecific engagers, e.g., bispecific antibodies, where at least one of the antigen binding domains of the first bispecific/multispecific engager, e.g., bispecific antibody, binds to and activates the T cell receptor (e.g., by binding to CD3ε) and at least one of the antigen binding domains of the second bispecific/multispecific engager, e.g., bispecific antibody, binds to and activates a costimulatory receptor (e.g., 41BB or CD28) and the at least one of antigen binding domains of the two bispecific/multispecific engagers, e.g., bispecific antibodies, binds to an antigen expressed on hematopoietic cells (e.g., CD19, CD22, CD20/MS4A1 and/or BCMA, etc.). In an exemplary embodiment, the method involves activation/expansion of immune T cells by exposing them to a CD19×CD3 and CD19×41BB bispecific antibodies in the presence of REC-1 cells.

In an alternate embodiment, the method involves activation/expansion of immune T cells by exposing them to a CD19×CD3 and CD22×CD28 bispecific antibodies in the presence of REC-1 cells. In another alternate embodiment, the method involves activation/expansion of immune T cells by exposing them to a CD20×CD3 and CD22×CD28 bispecific antibodies in the presence of REC-1 cells. Exemplary combinations of bispecific engagers, activating antibodies (e.g., CD3), APC, APS, and cytokines that can be used to activate immune cells, e.g., T cells, in various embodiments of the methods of the disclosure are presented in Table 17.

The aforesaid methods can be carried out in vitro, ex vivo or in vivo.

In some embodiments, the population of immune cells used in the methods described herein is acquired, e.g., obtained, from a blood sample from a subject (e.g., a cancer patient). In one embodiment, the population of immune cells is obtained by apheresis. In one embodiment, the population of immune cells is obtained by apheresis from a subject who has exercised or has been administered a CXCR4 antagonist (e.g, Plerixafor or Mozibil), a cytokine (e.g., G-CSF or GM-CSF), a Beta2 adregnergic agonist (e.g., epinephrine), a Tyrosine Kinase inhibitor (e.g., Dasatinib), a chemotherapy drug (e.g., Cyclophosphamide, doxorubicine), or a combination of the above.

In some embodiments, the immune cell population includes immune effector cells, e.g., as described herein. Exemplary immune effector cells include T cells, e.g., alpha/beta T cells and gamma/delta T cells, natural killer T (NKT) cells, bone marrow resident mono-nuclear cells, tissue resident mononuclear cells or a combination thereof.

In some embodiments, the immune cell population includes peripheral blood mononucleated cells (PBMCs), or cord blood cells, or a combination thereof.

In certain embodiments, the immune cell population includes primary T cells or subsets of lymphocytes, including, for example, anergized T cells, naive T cells, T-regulatory cells, Th-17 cells, stem T cells, tissue-resident T cells, tumor infilterating T cells or a combination thereof.

In certain embodiments, the immune cell population includes T cells that have been engineered to express a natural or a synthetic receptor targeting a specific antigen. An exemplary natural receptor includes a T cell receptor (TCR) targeting NY-ESO1 or WTI. Exemplary synthetic receptors include a CAR or a next generation CARS (e.g., K13-CAR, SIR, zSIR, Ab-TCR, TFP etc.) or a recombinant TCR (rTCR). Non-limiting exemplary target antigens that can be targeted by the T cells which are activated/expanded by the methods of the disclosure include: Mesothelin, epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); Tn antigen ((Tn Ag) or (GalNAcα-Ser/Thr)); prostate-specific membrane antigen (PSMA); Receptor tyrosine kinase-like orphan receptor 1 (ROR1); Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Interleukin 11 receptor alpha (IL-11Ra); prostate stem cell antigen (PSCA); vascular endothelial growth factor receptor 2 (VEGFR2); Lewis(Y) antigen; Stage-specific embryonic antigen-4 (SSEA-4); Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CA1X); tyrosinase; ephrin type-A receptor 2 (EphA2); sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR5IE2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); CD19; CD20/MS4A1; CD123; CD22; CD23, CD30; CD33; CD171; CS-1 (also referred to as CD2 subset 1, CRACC, SLAMF7, TNF receptor family member B cell maturation (BCMA); CD319, and 19A24); C-type lectin-like molecule-1 (CLL-1 or CLECL1); Fins Like Tyrosine Kinase 3 (FLT3); Tumor-associated glycoprotein 72 (TAG72); CD38; CD44v6; a glycosylated CD43 epitope expersed on acute leukemia or lymphoma but not on hematopoietic progenitors, a glycosylated CD43 epitope expressed on non-hematopoietic cancers, Protease Serine 21 (Testisin or PRSS21); CD24; Platelet-derived growth factor receptor beta (PDGFR-beta); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B 1); CCCTC-Binding Factor (Zinc Finger Protein)-Like (BORIS or Brother of the Regulator ofimprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen); Fucosyl-GM1, PTK7, gpNMB, CDH1-CD324, DLL3, CD276/B7H3, IL11Ra, IL13Ra2, CD179b-IGL11, TCRgamma-delta, NKG2D, CD32 (FCGR2A), Tn ag, Tim1-/HVCR1, CSF2RA (GM-CSFR-alpha), TGFbetaR2, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, HIV1 envelope glycoprotein, HTLV1-Tax, CMV pp65, EBV-EBNA3c, KSHV K8.1, KSHV-gH, influenza A hemagglutinin (HA), GAD, PDL1, GUANYLYL CYCLASE C (GCC), autoantibody to desmoglein 3 (Dsg3), MPL, autoantibody to desmoglein 1 (Dsg1), HLA, HLA-A, HLA-A2, HLA-B, HLA-C, HLA-DP, HLA-DM, HLA-DOA, HLA-DOB, HLA-DQ, HLA-DR, HLA-G, IGE, CD99, RAS G12V, TISSUE FAACTOR 1 (TF1), AFP, GPRC5D, CLAUDIN18.2 (CLD18A2 OR CLDN18A.2)), P-GLYCOPROTEIN, STEAPI, LIV1, NECTIN-4, CRIPTO, GPA33, BST1/CD157, LOW CONDUCTANCE CHLORIDE CHANNEL, VISTA, CD16ORF54, Muc5Ac, EMR2, Robo4, RNF43, CLDN6, MMP16, UPK1B, BMPR1B, Ly6E, STEAPI, WISP1, SLC34A2 and antigen recognized by TNT antibody.

In the preferred embodiment, the T cells are targeted against an antigen expressed in solid tumors (e.g., Mesothelin, EGFR viii, CHD6, CDH17, CDH19, DLL3, CLD18A2, ALK, CD276, CD324, B7H4, EGFR, EBNA3c, EpCaml, LlCAM, Folate Receptor 1, GFRa4, STEAPI, Livl, Nectin4, Cripto, gpA33, ILIRAP, GD2, GD3, gp100, ROR1, SLea, PTK7, Prolactin Receptor, LHR, TSHR, Lewis Y, Her2, GCC, SSEA4, IL-13Ra2, PSMA, PSCA, NY-ESO1, WT1, MART1, MAGE1, AFP, TIM1, TROP2, hTERT, MMP16, UPK1B, BMPR1B, Ly6E, STEAPI, WISP1, SLC34A2, VEGFR3, Tn-Muc1, and Tyrosinase etc.) and the bispecific/multispecific engager, e.g., bispecific antibody, targets an antigen expressed on B cells (e.g., CD19, CD22, CD20/MS4A1 etc.), plasma cells (e.g., BCMA, CD138, SLAMF7 etc.) and myeloid cells (e.g., CD123, MPL, CD33).

In some embodiments, the target antigen of the bispecific/multispecific engager, e.g., bispecific antibody, is expressed in a cell e.g., a cell expressing the cognate antigen on its surface. In one embodiment, the cognate antigen is heterologous to the cell, e.g., is a recombinant antigen expressed on the cell surface. In another embodiment, the cognate antigen is endogenously expressed on a cell, e.g., a tumor cell. In the aforesaid embodiments, the immune effector cell population can be expanded in vitro, ex vivo or in vivo. In one embodiment, T cells are expanded in vivo, e.g., by administration of the bispecific/multispecific engager, e.g., bispecific antibody, subcutaneously, intravenously or intratumorally.

In another embodiment, the target antigen of the bispecific/multispecific engager, e.g., bispecific antibody, is present on a non-cellular substrate. The non-cellular substrate can be a solid support chosen from, e.g., a plate (e.g., a microtiter plate), a membrane (e.g., a nitrocellulose membrane), a matrix, a chip or a bead. In embodiments, the target antigen of the bispecific/multispecific engager, e.g., bispecific antibody, molecule is present in the substrate (e.g., on the substrate surface). The target antigen can be immobilized, attached, or associated covalently or non-covalently (e.g., cross-linked) to the substrate. In one embodiment, the target antigen is attached (e.g., covalently attached) to a bead. The SEQ ID NO of several exemplary target antigens that can be attached to substrates are provided in Table 7D.

In one aspect, more than one target antigens are immobilized on beads, either on the same bead, i.e., “cis,” or to separate beads, i.e., “trans.” By way of example, the agent providing the primary activation signal in the presence of a CD19×CD3 bispecific antibody is CD19-extracellular domain (CD19-ECD) or fragment thereof and the agent providing the costimulatory signal is an anti-CD28 antibody; and both agents are co-immobilized to the same bead in equivalent molecular amounts. In one aspect, a 1:1 ratio of each agent bound to the beads for CD4+ T cell expansion and T cell growth is used. In certain aspects of the disclosure, a ratio of the two agents bound to the beads is used such that an increase in T cell expansion is observed in the presence of CD19×CD3 bispecific antibody as compared to the expansion observed using a ratio of 1:1. In one particular aspect an increase of from about 1 to about 3 fold is observed as compared to the expansion observed using a ratio of 1:1. In one aspect, the ratio of the two agents bound to the beads ranges from 100:1 to 1:100 and all integer values there between. In one aspect, more anti-CD28 antibody is bound to the particles than CD19-ECD, i.e., the ratio of CD19-ECD:CD28 antibody is less than one. In certain aspects, the ratio of anti CD28 antibody to CD19-ECD bound to the beads is greater than 2:1. In one particular aspect, a 1:100 CD19-ECD:CD28 antibody ratio of agents bound to beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell.

For example, small sized beads could only bind a few cells, while larger beads could bind many. In certain aspects the ratio of cells to particles ranges from 1:100 to 100:1 and any integer values in-between and in further aspects the ratio comprises 1:9 to 9:1 and any integer values in between, can also be used to stimulate T cells. The ratio of CD19-ECD- and anti-CD28-coupled particles to T cells that result in T cell stimulation in the presence of an exemplary CD19×CD3 bispecific antibody can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1,4:1, 5:1, 6:1,7:1, 8:1, 9:1, 10:1, and 15:1 with one preferred ratio being at least 1:1 particle per T cell. In one aspect, a ratio of particles to cells of 1:1 or less is used. In one particular aspect, a preferred particle: cell ratio is 1:5. In further aspects, the ratio of particles to cells can be varied depending on the day of stimulation. For example, in one aspect, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular aspect, the ratio of particles to cells is 1:1 on the first day of stimulation and adjusted to 1:5 on the third and fifth days of stimulation. In one aspect, particles are added on a daily or every other day basis to a final ratio of 1:1 on the first day, and 1:5 on the third and fifth days of stimulation. In one aspect, the ratio of particles to cells is 2:1 on the first day of stimulation and adjusted to 1:10 on the third and fifth days of stimulation. In one aspect, particles are added on a daily or every other day basis to a final ratio of 1:1 on the first day, and 1:10 on the third and fifth days of stimulation. One of skill in the art will appreciate that a variety of other ratios may be suitable for use in the disclosure. In particular, ratios will vary depending on particle size and on cell size andtype. In one aspect, the most typical ratios for use are in the neighborhood of 1:1, 2:1 and 3:1 on the first day.

In further aspects, the cells, such as T cells, are combined with agent-coated beads, the beads and the cells are subsequently separated, and then the cells are cultured. In an alternative aspect, prior to culture, the agent-coated beads and cells are not separated but are cultured together. In a further aspect, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which CD19-ECD and anti-CD28 are attached (CD19-ECD×CD28 beads) to contact the T cells. In one aspect the cells (for example, 10⁴ to 10⁹ T cells) and beads (at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the disclosure. In certain aspects, it may be desirable to significantly decrease the volume in which particles and cells are mixed together (i.e., increase the concentration of cells), to ensure maximum contact of cells and particles. For example, in one aspect, a concentration of about 10 billion cells/ml, 9 billion/ml, 8 billion/ml, 7 billion/ml, 6 billion/ml, 5 billion/ml, or 2 billion cells/ml is used. In one aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain aspects. For example, using high concentration of cells allows more efficient selection of CD8+ T cells that normally have weaker CD28 expression.

In one embodiment, cells transduced with a nucleic acid encoding a CAR, e.g., a CAR described herein, are expanded, e.g., by a method described herein. In one embodiment, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. In one embodiment, the cells are expanded for a period of 8 days or less, e.g., 7, 6 or 5 days. In one embodiment, the cells, e.g., a CD19 CAR cell described herein, are expanded in culture for 5 days, and the resulting cells are more potent than the same cells expanded in culture for 9 days under the same culture conditions. Potency can be defined, e.g., by various T cell functions, e.g. proliferation, target cell killing, cytokine production, activation, migration, or combinations thereof. In one embodiment, the cells, e.g., a CD19 CAR cell described herein, expanded for 5 days show at least a one, two, three or four-fold increase in cells doublings upon antigen stimulation as compared to the same cells expanded in culture for 9 days under the same culture conditions. In one embodiment, the cells, e.g., the cells expressing a CD19 CAR described herein, are expanded in culture for 5 days, and the resulting cells exhibit higher proinflammatory cytokine production, e.g., IFN-γ and/or GM-CSF levels, as compared to the same cells expanded in culture for 9 days under the same culture conditions. In one embodiment, the cells, e.g., a CD19 CAR cell described herein, expanded for 5 days show at least a one, two, three, four, five, ten fold or more increase in pg/ml of proinflammatory cytokine production, e.g., IFN-γ and/or GM-CSF levels, as compared to the same cells expanded in culture for 9 days under the same culture conditions.

In the aforesaid embodiments, the immune cell population can be expanded in vitro or ex vivo.

In other embodiments, the strength of the immune cell stimulation in vitro is customized to a desired level, e.g., by adjusting one or more of: dose of the bispecific/multispecific engager, e.g., bispecific antibody, dose of the substrate expressing the target antigen (e.g., number of beads or cells expressing the CD19 antigen; density of the target antigen on the substrate, duration of exposure of the T cells to the bispecific/multispecific engager, affinity of the bispecific/multispecific engager, e.g., bispecific antibody for the target antigen.

In one embodiment, the immune cells are cultured ex vivo in the presence of the bispecific/multispecific engager, e.g., bispecific antibody and the substrate (e.g., cells or beads) expressing the cognate ligand (e.g., an antigen or an anti-idiotype antibody) of the bispecific antibody for a predetermined period (e.g., about 1, 2, 3, 4, 5, 6, 7, 8, 9, 10, 15, 18, 21, 22, 23 or 24 hours) or (e.g., 1, 5, 10, 15, 20, 25, 30, 35, 40, 45 or 50 days). In one embodiment, the immune cells are cultured for a period of 4 to 9 days. In one embodiment, the immune-expressing cells are cultured for a period of 8 days or less, e.g., 7, 6 or 5 days.

In one embodiment, the immune T cells activated/expanded ex vivo in the presence of at least 0.1 μg/ml, 0.5 μg/ml, 1 μg/ml, 10 μg/ml, 100 μg/ml, 1 ng/ml, 10 ng/ml, 50 ng/ml, 100 ng/ml, 500 ng/ml, 1000 ng/ml or 5000 ng/ml of the bispecific/multispecific engager, e.g., bispecific antibody.

In one embodiment, the immune effector T cells are activated/expanded ex vivo in the presence of the bispecific/multispecific engager, e.g., bispecific antibody, and cognate antigen expressing target cells at an Effector:Target ratio of about 0.1:1, 0.5:1, 1:1, 5:1, 10:1, 20:1 or 50:1.

In one embodiment, the immune effector T cells are activated/expanded ex vivo in the presence of the bispecific/multispecific engager, e.g., bispecific antibody, and cognate antigen expressing beads at an Effector:Bead ratio of about 0.1:1, 0.2:1, 0.5:1, 1:1, 2:1, 5:1, 10:1, 20:1 or 50:1.

In some embodiment, the immune cell population shows at least 2, 3, 4, 5, 6, 7, 8, 9, 10, 11 or 12 or higher population doublings when exposed to bispecific/multispecific engager, e.g, a bispecific antibody, and its target antigen-expressing substrate (e.g., cells or beads). In one embodiment, the immune cell population shows a total of 8-10, or about 9 population doublings.

In one embodiment, the immune T cell population expands to a total of 10-, 50-, 100-, 200-, 300-, 400-, 450-, 500-, 550-, 600-, 750-fold or higher expansion per cell. In one embodiment, the immune T cell population are expanded about 500-fold. In one embodiment, an average cell multiplies to over 400-600, or about 500 cells. In some embodiments, the cell expansion is measured by a method described herein, such as flow cytometry. In one embodiment, the cell expansion is measured at about 5, 10, 15, 20, 25, 30, 35, 40, 45, or 50 days after stimulation with the bispecific antibody in the presence of the target antigen of the bispecific antibody. In one embodiment, the cell expansion is measured between 10 and 25 days after stimulation with the bispecific antibody and the target antigen of the bispecific antibody. In one embodiment, the expansion is measured 8, 9, 10, 11, 12, 13, 14, 15, 16, 17, 18, 19, 20, 21, 22, 23, 24, or 25 days after stimulation with the bispecific antibody and the target antigen of the bispecific antibody.

In one embodiment, the immune cells are activated/expanded in vivo by the administration of the bispecific/multispecific engager, e.g., bispecific antibody, for a predetermined period (e.g., about 1, 2, 3, 4, 5, 6, 7, 8, 9, 10, 15, 18, 21, 22, 23 or 24 hours) or (e.g., 1, 5, 10, 15, 20, 25, 30, 35, 40, 45, 50, 60, 70, 90, 100, 120 days). In one embodiment, the immune T cells are expanded in vivo for a period of 2 to 20 days. In one embodiment, the immune T cells are expanded for a period of 12 days or less, e.g., 10, 7, 6 or 5 days. In one embodiment, bispecific antibody is administered in multiple cycles of 1-28 days followed by a rest period of 1-14 days.

In other embodiments, the bispecific/multispecific antibody can be used for activation and/or expansion of immune cells (e.g., T cell, NK cells, TILs etc.) in vivo. The strength of the immune cell stimulation in vivo is customized to a desired level, e.g., by adjusting one or more of: dose of the bispecific antibody, duration of exposure (e.g, duration of infusion) of the bispecific antibody, frequency of administration of the bispecifc antibody, half-life of the bispecific antibody, affinity of the bispecific antibody for the target antigen. For example, increasing the dose of the bispecific antibody may increase the stimulation and expansion of T cells, including engineered T cells. Alternatively, interrupting the administration of the bispecific antibody would be expected to decrease the stimulation and expansion of T cells, including engineered T cells.

In one embodiment, the bispecific/multispecific engager, e.g., bispecific antibody, is administered by continuous infusion. In one embodiment, the bispecific/multispecific engager, e.g., bispecific antibody, is administered by injection intravenously, subcutaneously, transdermally, and/or intramuscularly.

In one embodiment, the immune effector cells and the bispecific/multispecific engager, e.g., bispecific antibody, are administered to the subject after administration of lymphodepleting chemotherapy. Several lymphodepleting chemotherapy regimens are known in the art. An exemplary lymphodepleting chemotherapy regimen includes 30 mg/m2/day fludarabine intravenously plus 500 mg/m2/day cyclophosphamide intravenously x 3 days. In an exemplary embodiment, the immune effector cells are administered to the patient 1 day after finishing the administration of the lymphodepleting chemotherapy. In an exemplary embodiment, the immune effector cells are administered to the patient 2 day after finishing the administration of the lymphodepleting chemotherapy. The bispecific/multispecific engager, e.g., bispecific antibody, can be administered to the subject concurrently with the administration of the immune effector cells, prior to the administration of the immune effector cells or after the administration of the immune effector cells. In the preferred embodiment, the bispecific/multispecific engager, e.g., bispecific antibody, is administered to the subject after the administration of the immune effector cells.

In an exemplary embodiment, the bispecific/multispecific engager, e.g., bispecific antibody, is Blinatumomb (BLINCYTO) and Blinatumomab is administered to the subject as a continuous intravenous infusion at a constant flow rate using an infusion pump which is programmable, lockable, non-elastomeric, and has an alarm as described in its prescribing information. In an exemplary embodiment, Blinatumomab is administered at a dose of 1 mcg/day, 2 mcg/day, 5 mcg/day, 10 mcg/day, 20 mcg/day, 25 mcg/day, 28 mcg/day, 30 mcg/day or 50 mcg/day by continuous infusion. In the preferred embodiment, Blinatumomab is administered after premedication with prednisone 100 mg intravenously or equivalent (e.g., dexamethasone 16 mg) 1 hour prior to the first dose of BLINCYTO in each cycle.

The in vivo administration of the suitable composition of the bispecific antibody may be effected by different ways, e.g., by intravenous, intraperitoneal, subcutaneous, intramuscular, topical or intradermal administration. In particular, the disclosure provides for an uninterrupted administration of the suitable composition. As a non-limiting example, uninterrupted, i.e. continuous administration may be realized by a small pump system worn by the patient for metering the influx of therapeutic agent into the body of the patient. The pharmaceutical composition comprising the bispecific/multispecific engager, e.g., bispecific antibody, can be administered by using said pump systems. Such pump systems are generally known in the art, and commonly rely on periodic exchange of cartridges containing the therapeutic agent to be infused. When exchanging the cartridge in such a pump system, a temporary interruption of the otherwise uninterrupted flow of therapeutic agent into the body of the patient may ensue. In such a case, the phase of administration prior to cartridge replacement and the phase of administration following cartridge replacement would still be considered within the meaning of the pharmaceutical means and methods of the disclosure together make up one “uninterrupted administration” of such therapeutic agent.

The continuous or uninterrupted administration of the bispecific/multispecific engager, e.g., bispecific antibody, described herein may be intravenous or subcutaneous by way of a fluid delivery device or small pump system including a fluid driving mechanism for driving fluid out of a reservoir and an actuating mechanism for actuating the driving mechanism. Pump systems for subcutaneous administration may include a needle or a cannula for penetrating the skin of a patient and delivering the suitable composition into the patient's body. Said pump systems may be directly fixed or attached to the skin of the patient independently of a vein, artery or blood vessel, thereby allowing a direct contact between the pump system and the skin of the patient. The pump system can be attached to the skin of the patient for 24 hours up to several days. The pump system may be of small size with a reservoir for small volumes. As a non-limiting example, the volume of the reservoir for the suitable pharmaceutical composition to be administered can be between 0.1 and 50 ml.

The continuous administration may be transdermal by way of a patch worn on the skin and replaced at intervals. One of skill in the art is aware of patch systems for drug delivery suitable for this purpose. It is of note that transdermal administration is especially amenable to uninterrupted administration, as exchange of a first exhausted patch can advantageously be accomplished simultaneously with the placement of a new, second patch, for example on the surface of the skin immediately adjacent to the first exhausted patch and immediately prior to removal of the first exhausted patch. Issues of flow interruption or power cell failure do not arise.

The inventive compositions may further comprise a pharmaceutically acceptable carrier. Examples of suitable pharmaceutical carriers are well known in the art and include solutions, e.g. phosphate buffered saline solutions, water, emulsions, such as oil/water emulsions, various types of wetting agents, sterile solutions, liposomes, etc. Compositions comprising such carriers can be formulated by well known conventional methods. Formulations can comprise carbohydrates, buffer solutions, amino acids and/or surfactants. Carbohydrates may be non-reducing sugars, preferably trehalose, sucrose, octasulfate, sorbitol or xylitol. In general, as used herein, “pharmaceutically acceptable carrier” means any and all solvents, dispersion media, coatings, antibacterial and antifungal agents, isotonic and absorption delaying agents, compatible with pharmaceutical administration. The use of such media and agents for pharmaceutically active substances is well known in the art.

Acceptable carriers, excipients, or stabilizers are nontoxic to recipients at the dosages and concentrations employed and include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counter-ions, such as sodium, polyhydric sugar alcohols; amino acids, such as alanine, glycine, asparagine, 2-phenylalanine, and threonine; sugars or sugar alcohols, such as trehalose, sucrose, octasulfate, sorbitol or xylitol stachyose, mannose, sorbose, xylose, ribose, myoinisitose, galactose, lactitol, ribitol, myoinisitol, galactitol, glycerol, cyclitols (e.g., inositol), polyethylene glycol; sulfur containing reducing agents, such as glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone.

Such formulations may be used for continuous administrations which may be intravenuous or subcutaneous with and/or without pump systems. Amino acids may be charged amino acids, preferably lysine, lysine acetate, arginine, glutamate and/or histidine. Surfactants may be detergents, preferably with a molecular weight of >1.2 KD and/or a polyether, preferably with a molecular weight of >3 KD. Non-limiting examples for preferred detergents are Tween 20, Tween 40, Tween 60, Tween 80 or Tween 85. Non-limiting examples for preferred polyethers are PEG 3000, PEG 3350, PEG 4000 or PEG 5000. Buffer systems used in the disclosure can have a preferred pH of 5-9 and may comprise citrate, succinate, phosphate, histidine and acetate.

The compositions of the disclosure comprising the bispecific/multispecific engager, e.g., bispecific antibody, in a single or separate formulations can be administered to the subject at a suitable dose which can be determined e.g. by dose escalating studies by administration of increasing doses of the polypeptide described herein to non-chimpanzee primates, for instance macaques. The composition or these compositions can also be administered in combination with additional other proteinaceous and non-proteinaceous drugs and cellular therapy products. These drugs may be administered simultaneously with the composition comprising the bispecific/multispecific engagers described herein as defined herein or separately before or after administration of said bispecific/multispecific engagers in timely defined intervals and doses. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, inert gases and the like. In addition, the composition of the disclosure, e.g., bispecific/multispecific engagers might comprise proteinaceous carriers, like, e.g., serum albumin or immunoglobulin, preferably of human origin. It is envisaged that the composition of the disclosure might comprise, in addition to the polypeptide described herein, further biologically active agents, depending on the intended use of the composition. Such agents might be drugs acting on the gastro-intestinal system, drugs acting as cytostatica, drugs preventing hyperuricemia, drugs inhibiting immunoreactions (e.g. corticosteroids), drugs modulating the inflammatory response, drugs acting on the circulatory system and/or agents such as cytokines known in the art. It is also envisaged that the composition of the disclosure comprising the bispecific/multispecific engager in a single or separate formulations is applied in an additional co-therapy, i.e., in combination with another anti-cancer medicament.

In certain embodiments, the methods disclosed herein further include contacting the immune cell population with a nucleic acid encoding a CAR (including next generation a CAR, such as K13-CAR, SIR, Ab-TCR, TFP etc.) or a TCR molecule, e.g., a vector comprising a nucleic acid encoding a CAR or a TCR, thereby producing a CAR or a TCR-expressing cell population. In certain embodiments, the immune cell population is activated/expanded by the bispecific/multispecific engager, e.g., a bispecific antibody, in the presence of its target antigen (e.g., Blinatumomab in the presence of CD19+ B cells) first and is then followed by contact with nucleic acid encoding a CAR (including next generation a CAR, such as K13-CAR, SIR, Ab-TCR, TFP etc.) or a TCR molecule.

In certain embodiments, the immune cell population is activated/expanded by the bispecific/multispecific engager, e.g., a bispecific antibody, in the presence of its target antigen (e.g., Blinatumomab in the presence of CD19+ B cells) after contact with nucleic acid encoding a CAR (including next generation a CAR, such as K13-CAR, SIR, Ab-TCR, TFP etc.) or a TCR molecule. In certain embodiments, the immune cell population is activated/expanded by the bispecific/multispecific engager, e.g., a bispecific antibody, in the presence of its target antigen (e.g., Blinatumomab in the presence of CD19+ B cells) concurrently with contact with nucleic acid encoding a CAR (including next generation a CAR, such as K13-CAR, SIR, Ab-TCR, TFP etc.) or a TCR molecule.

The steps of activation/expansion by the bispecific/multispecific engager, e.g., a bispecific antibody, plus its cognate antigen and contact with nucleic acid encoding a CAR (including next generation a CAR, such as K13-CAR, SIR, Ab-TCR, TFP etc.) or a TCR may occur in vitro, ex vivo, in vivo or in various combination. In certain embodiments, immune T cells are activated/expanded by the bispecific/multispecific engager, e.g., a bispecific antibody, in the presence of its target antigen in vitro and contacted with nucleic acid encoding a CAR (including next generation a CAR, such as K13-CAR, SIR, Ab-TCR, TFP etc.) or a TCR molecule in vitro. In certain embodiments, immune T cells are activated/expanded by the bispecific/multispecific engager, e.g., a bispecific antibody, in the presence of its target antigen both in vitro and in vivo and contacted with nucleic acid encoding a CAR (including next generation a CAR, such as K13-CAR, SIR, Ab-TCR, TFP etc.) or a TCR molecule in vitro. In certain embodiments, immune T cells are activated/expanded by the bispecific/multispecific engager, e.g., a bispecific antibody, in the presence of its target antigen both in vitro and in vivo and contacted with nucleic acid encoding a CAR or a TCR molecule in vivo. In one embodiment, immune T cells are activated/expanded by the bispecific/multispecific engager, e.g., a bispecific antibody, in the presence of its target antigen (cognate ligand) and a ligand or an agonist antibody directed against a costimulatory molecule. Exemplary costimulatory molecules include 41BB, CD28 and CD27. In one embodiment, the agonist 41BB antibody is Utomilumab. In one embodiment, immune T cells are activated/expanded by the exposing them to Blinatumomb and Utomilumab in the presence of CD19 expressing B cells. In one embodiment, immune T cells are activated/expanded by administering Blinatumomb and Utomilumab to the subject.

In one embodiment, the nucleic acid encoding the CAR/TCR molecule is selected from the group consisting of a DNA, an RNA, a plasmid, a lentivirus vector, adenoviral vector, or a retrovirus vector. In one embodiment, the nucleic acid encoding the CAR (including next generation a CAR, such as K13-CAR, SIR, Ab-TCR, TFP etc.) or a TCR molecule vector is a lentivirus.

In other embodiments, the nucleic acid encoding a CAR (including next generation a CAR, such as K13-CAR, SIR, Ab-TCR, TFP etc.) or a TCR molecule is an IVT RNA.

In some embodiment, the bispecific/multispecific engager, e.g., a bispecific antibody, and the CAR molecules are directed to the same antigen, e.g., the same tumor cell antigen. In such embodiments, the immune cell population is expanded and/or activated in vitro or ex vivo, e.g., by contacting said immune cell population with the antigen targeted by the second antigen binding domain of the bispecific antibody or an anti-idiotypic antibody (e.g., a CD19-antigen or anti-CD19 idiotypic antibody immobilized onto a non-cellular or cellular substrate as described herein).

Alternatively, or in combination, the immune cell population is expanded and/or activated in vivo, e.g. by contacting an endogenous cell antigen (e.g., CD19). Alternatively, or in combination, the immune cell population is exposed to the bispecific/multispecific engager, e.g., a bispecific antibody, ex vivo and then activated by infusion in vivo, e.g. by contacting an endogenous cell antigen (e.g., CD19). In one embodiment, the immune cell is administered to a subject, e.g., as part of a therapeutic protocol.

In other embodiments, the bispecific/multispecific engager, e.g., a bispecific antibody, and the CAR/TCR molecules are directed to different antigens, e.g., different tissue and/or tumor cell antigens. In such embodiments, the immune cell population is expanded and/or activated in vitro or ex vivo, e.g., by contacting said immune cell population with the antigen targeted by the second antigen binding domain of the bispecific antibody or an anti-idiotypic antibody (e.g., a CD19-antigen or anti-CD19 idiotypic antibody immobilized onto a non-cellular or cellular substrate as described herein). Alternatively, or in combination, the immune cell population is expanded and/or activated in vivo, e.g. by contacting an endogenous cell antigen (e.g., CD19). Alternatively, or in combination, the immune cell population is exposed to the bispecific/multispecific engager, e.g., a bispecific antibody, ex vivo and then activated by infusion in vivo, e.g. by contacting an endogenous cell antigen (e.g., CD19). In one embodiment, the immune cell is administered to a subject, e.g., as part of a therapeutic protocol.

In one embodiment, the antigen bound by at least one of antigen binding domains of the bispecific/multispecific engager, e.g., a bispecific antibody, is chosen from CD19, CD20/MS4A1, CD22, CD23, CD123, FLT3, BCMA, CS1/SLAMF7, CD30, CD32b, CD70, CD79b, CD123, CD138, CD179b, GPRC5D, Lyml, Lym2, and FCRH5 and CAR/TCR targets an antigen chosen from Mesothelin, EGFR viii, CHD6, CDH17, CDH19, DLL3, CLD18A2, ALK, CD276, CD324, B7H4, EGFR, EBNA3c, EpCaml, LlCAM, Folate Receptor 1, GFRa4, STEAPI, Livl, Nectin4, Cripto, gpA33, ILIRAP, GD2, GD3, gp100, ROR1, SLea, PTK7, Prolactin Receptor, LHR, TSHR, Lewis Y, Her2, GCC, SSEA4, IL-13Ra2, PSMA, PSCA, NY-ESO1, WT1, MART1, MAGE1, AFP, TIM1, TROP2, hTERT, MMP16, UPK1B, BMPR1B, Ly6E, STEAPI, WISP1, SLC34A2, VEGFR3, Tn-Mucd, and Tyrosinase.

In other embodiments, the immune cells express more than CAR or TCRs. In other embodiments, the immune cells may express accessory module that modulate the activity of CAR or TCR. Exemplary accessory modules include vFLIP K13, vFLIP MC159, NEMO-K277A, and constitutive active mutants of JAK1, JAK3, STAT5b and BRAF. In other embodiments, the immune cells may express an inhibitor of one or more genes from the group of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL (TNFSF10) and Death Receptor 5 (DR5 or TNFRSF10B).

In certain embodiments, the methods further comprise storing the expanded and/or activated immune cell population after the appropriate expansion period and/or contact with nucleic acid encoding a CAR and/or TCR. In one embodiment, the expanded and/or activated and/or CAR/TCR expressing immune cell population is cryopreserved according to a method described herein. In one embodiment, the expanded and/or activated and/or CAR/TCR expressing immune cell population is cryopreserved in an appropriate media, e.g., an infusible media, e.g., as described herein.

In another aspect, the disclosure features a method of treating a disorder or condition (e.g., a disorder or condition as described herein), in a subject. The method includes administering to the subject an expanded and/or activated immune cell population made according to one or more of the methods described herein. In embodiments, the method includes acquiring (e.g., obtaining) the expanded and/or activated immune cell population. The expanded and/or activated immune cell population can be obtained from a suitable storage condition, e.g., cryopreservation.

In some embodiments, the immune cell population includes immune effector cells, e.g., a described herein. Exemplary immune effector cells include T cells, e.g., alpha/beta T cells and gamma/delta T cells, and natural killer T (NKT) cells.

In yet another aspect the disclosure features a method of treating, or providing anti-tumor immunity to, a subject having a cancer. The method includes administering to the subject an effective amount of an immune effector T cell population (e.g., an expanded and/or activated immune cell population as described herein) that optionally expresses a CAR and/or exogenous TCR molecule (e.g., a first and/or second CAR/TCR molecule as described herein), alone or in combination with an additional therapy, e.g., a second therapy as described herein.

In some embodiments, the treatment method includes acquiring (e.g., obtaining) the expanded and/or activated immune cell population using one or more of the methods described herein. For example, the expanded and/or activated immune cell population may have been previously obtained by using Plerixafor-mobilized T cells, introducing a CAR molecule (e.g., a nucleic acid molecule encoding the CAR molecule as described herein, e.g., an IVT RNA encoding the first CAR) under conditions suitable for expression of the CAR molecule; and contacting said CAR-expressing cell population with a bispecific/multispecific engager, e.g., a bispecific antibody, described herein in the presence of the target antigen expressing cell/substrate of the bispecific antibody, e.g., a cell/substrate that expresses the target antigen bound by at least one antigen binding domains of the bispecific/multispecific engager, e.g., a bispecific antibody, (e.g., a cognate antigen molecule (e.g., a recombinant antigen) or an anti-idiotypic antibody molecule), under conditions such that immune cell expansion and/or activation occurs. In embodiments, the target antigen of the bispecific/multispecific engager, e.g., a bispecific antibody, is present in/on (e.g., immobilized or attached to) a substrate, e.g., a non-naturally occurring substrate, as described herein. The expanded and/or activated immune cell population can be stored under suitable conditions, e.g., cryopreservation, as described herein.

In one exemplary embodiment, the CAR is directed to mesothelin (e.g., a CAR represented by SEQ ID NO: 2832) and the mesothelin CAR-expressing cell is contacted with Blinatumomab (a CD19×CD3 bispecific antibody) in the presence of CD19 expressing B cells. In one exemplary embodiment, the CAR is directed to mesothelin and the mesothelin CAR-expressing cell is contacted with CD19×CD3 DART in the presence of CD19 expressing B cells. In an ememplary embodiment, the CD19-expressing cell is anormal peripheral blood B lymphocyte or a CD19-expressing cell line. Exemplary CD19+ cell lines that can be used in vitro for expansion of T cells in the presence of a CD19×CD3 bispecific engager include REC-1, JEKO-1, GRANTA-519, MINO, Nalm6 and RAJI cell lines. A preferred CD19+ cell line that can be used in vitro for expansion of T cells in the presence of a CD19×CD3 bispecific engager is REC-1. In one exemplary embodiment, the CAR is directed to mesothelin and the mesothelin CAR-expressing cell is contacted with BCMA x CD3 DART in the presence of BCMA expressing cells. A preferred BCMA+ cell line that can be used in vitro for expansion of T cells in the presence of a BCMA x CD3 bispecific engager, e.g., a BCMA x CD3 DART is REC-1. In one exemplary embodiment, the CAR is directed to mesothelin and the mesothelin CAR-expressing cell is contacted with CD123×CD3 DART in the presence of CD123 expressing blood cells. In one exemplary embodiment, the CAR is directed to Her2 (e.g. a CAR represented by SEQ ID NO: 2831) and the Her2 CAR-expressing cell is contacted with Blinatumomab (a CD19×CD3 bispecific antibody) in the presence of CD19 expressing B cells or B cell line, e.g., REC-1. In one exemplary embodiment, the CAR is directed to Prolactin Receptor and the Prolactin Receptor CAR-expressing cell is contacted with Blinatumomab (a CD19×CD3 bispecific antibody) in the presence of CD19-expressing B cells. In one exemplary embodiment, the CAR is directed to ROR1 and the ROR1CAR-expressing cell is contacted with Blinatumomab (a CD19×CD3 bispecific antibody) in the presence of CD19 expressing B cells. In another exemplary embodiment, the CAR is directed to AFP/MHC I complex and the AFP-CAR-expressing T cell is contacted with Blinatumomab (a CD19×CD3 bispecific antibody) in the presence of CD19 expressing B cells. In another exemplary embodiment, the TCR is directed to NY-ESO/MHC complex (e.g., SEQ ID NO: 2836) and the NY-ESO-TCR-expressing T cell is contacted with Blinatumomab (a CD19×CD3 bispecific antibody) in the presence of CD19 expressing B cells. In another exemplary embodiment, the TCR is directed to WT1 and the WT1-TCR-expressing T cell is contacted with Blinatumomab (a CD19×CD3 bispecific antibody) in the presence of CD19-expressing B cells. In another exemplary embodiment, the TCR is directed to MART1 and the MART1-TCR-expressing T cell is contacted with Blinatumomab (a CD19×CD3 bispecific antibody) in the presence of CD19-expressing B cells.

The Table 7E provides the SEQ ID (DNA) and SEQ ID (PRT) of several exemplary bispecific antibodies that bind to different antigens (e.g., CD19, CD20/MS4A1, CD22, BCMA etc.) through one of their antigen binding domains and bind to CD3, CD28 or 41BB through their other antigen binding domain. As an example, a bispecific antibody FMC63×CD3 is represented by SEQ ID NO (DNA): 2470 and SEQ ID NO (PRT): 2646. This bispecific antibody is designed to provide activation signal to T cells, including T cells expressing a CAR, e.g., a mesothelin-CAR, or to T cells expressing a native or a recombinant TCR, e.g., a NYESO-TCR or WT1-TCR, when the T cells are exposed to a CD19-expressing target cell (e.g., REC-1 or NALM6 cells) or to CD19-beads. It is to be noted that this bispecific antibody is designed to provide the activation signal even if CAR or TCR is not directed to CD19 and even if the T cells do not express any recombinant CAR or recombinant TCR.

The bispecific antibody FMC63×CD28 is represented by SEQ ID NO (DNA): 2526 and SEQ ID NO (PRT): 2702. This bispecific antibody is designed to provide co-stimulation to T cells, including T cells expressing a CAR, e.g., a mesothelin-CAR, or T cells expressing a native or a recombinant TCR, e.g., a NY-ESO1-TCR or WT1-TCR when the T cells are exposed to a CD19-expressing target cell (e.g., REC-1 or NALM6 cells) or to CD19-bead. It is to be noted that this bispecific antibody is designed to provide the co-stimulatory signal even if CAR or TCR is not directed to CD19 and even if the T cells do not express any recombinant CAR or recombinant TCR.

The bispecific antibody FMC63×41BB is represented by SEQ ID NO (DNA): 2582 and SEQ ID NO (PRT): 2758. This bispecific antibody is designed to provide co-stimulation to T cells, including T cells expressing a CAR, e.g., a mesothelin-CAR, or T cells expressing a native or a recombinant TCR, e.g., a NY-ESOI-TCR or WT1-TCR when the T cells are exposed to a CD19-expressing target cell (e.g., REC-1 or NALM6 cells) or to CD19-bead. It is to be noted that this bispecific antibody is designed to provide the co-stimulatory signal even ifCAR or TCR is not directed to CD19 and even ifthe T cells do not express any recombinant CAR or recombinant TCR.

| | | Ag × CD3 | | Ag × CD28 | | Ag × 41BB | |
|---|---|---|---|---|---|---|---|
| | | SEQ | SEQ | SEQ | SEQ | SEQ | SEQ |
| Antigen | Antigen Binding | ID NO | ID NO | ID NO | ID NO | ID NO | ID NO |
| (Ag) | Domain | (DNA) | (PRT) | (DNA) | (PRT) | (DNA) | (PRT) |
| CD19 | FMC63 | 2470 | 2646 | 2526 | 2702 | 2582 | 2758 |
| CD19 | huFMC63-11 | 2471 | 2647 | 2527 | 2703 | 2583 | 2759 |
| CD19 | huFMC63-11-N203Q | 2472 | 2648 | 2528 | 2704 | 2584 | 2760 |
| CD19 | CD19Bu12 | 2473 | 2649 | 2529 | 2705 | 2585 | 2761 |
| CD19 | CD19MM | 2474 | 2650 | 2530 | 2706 | 2586 | 2762 |
| CD19 | Ritx-CD19-MOR0028 | 2475 | 2651 | 2531 | 2707 | 2587 | 2763 |
| CD19 | CD19-hu-mROO5-1 | 2476 | 2652 | 2532 | 2708 | 2588 | 2764 |
| BCMA | BCMA-J6M0 | 2477 | 2653 | 2533 | 2709 | 2589 | 2765 |
| BCMA | BCMA-huC12A3-L3H3 | 2478 | 2654 | 2534 | 2710 | 2590 | 2766 |
| BCMA | BCMA-huC11.D5.3 | 2479 | 2655 | 2535 | 2711 | 2591 | 2767 |
| | L1H3 | | | | | | |
| BCMA | BCMA-huC13-F12 | 2480 | 2656 | 2536 | 2712 | 2592 | 2768 |
| CD20 | CD20-2F2 | 2481 | 2657 | 2537 | 2713 | 2593 | 2769 |
| CD20 | CD20-GA101 | 2482 | 2658 | 2538 | 2714 | 2594 | 2770 |
| CD20 | CD20-2H7 | 2483 | 2659 | 2539 | 2715 | 2595 | 2771 |
| CD20 | CD2O-Ubli-v4 | 2484 | 2660 | 2540 | 2716 | 2596 | 2772 |
| CD20 | CD20-2H7 | 2485 | 2661 | 2541 | 2717 | 2597 | 2773 |
| CD20 | CD20-7D8 | 2486 | 2662 | 2542 | 2718 | 2598 | 2774 |
| CD22 | CD22-h10F4v2 | 2487 | 2663 | 2543 | 2719 | 2599 | 2775 |
| CD22 | CD22-H22Rhov2A | 2488 | 2664 | 2544 | 2720 | 2600 | 2776 |
| CD22 | CD22-m971-HL | 2489 | 2665 | 2545 | 2721 | 2601 | 2777 |
| CD22 | CD22-5-HL | 2490 | 2666 | 2546 | 2722 | 2602 | 2778 |
| CD22 | CD22-10-HL | 2491 | 2667 | 2547 | 2723 | 2603 | 2779 |
| CD22 | CD22-HA22 | 2492 | 2668 | 2548 | 2724 | 2604 | 2780 |
| CD30 | CD30-5F11 | 2493 | 2669 | 2549 | 2725 | 2605 | 2781 |
| CD30 | CD30-Ac10 | 2494 | 2670 | 2550 | 2726 | 2606 | 2782 |
| CD32 | CD32-Med9 | 2495 | 2671 | 2551 | 2727 | 2607 | 2783 |
| CD33 | CD33-AFS | 2496 | 2672 | 2552 | 2728 | 2608 | 2784 |
| CD33 | CD33-huMyc9 | 2497 | 2673 | 2553 | 2729 | 2609 | 2785 |
| CD33 | CD33-Him3-4 | 2498 | 2674 | 2554 | 2730 | 2610 | 2786 |
| CD33 | CD33-SGNh2H12 | 2499 | 2675 | 2555 | 2731 | 2611 | 2787 |
| CD33 | CD33-15G15-33 | 2500 | 2676 | 2556 | 2732 | 2612 | 2788 |
| CD33 | CD33-33H4 | 2501 | 2677 | 2557 | 2733 | 2613 | 2789 |
| CD123 | CD123-CSL362 | 2502 | 2678 | 2558 | 2734 | 2614 | 2790 |
| CD123 | CD123-1172 | 2503 | 2679 | 2559 | 2735 | 2615 | 2791 |
| CD123 | CD123-DART-1 | 2504 | 2680 | 2560 | 2736 | 2616 | 2792 |
| CD123 | CD123-DART-2 | 2505 | 2681 | 2561 | 2737 | 2617 | 2793 |
| CD123 | CD123-9D7 | 2506 | 2682 | 2562 | 2738 | 2618 | 2794 |
| CD123 | CD123-3B10 | 2507 | 2683 | 2563 | 2739 | 2619 | 2795 |
| CD138 | CD138 | 2508 | 2684 | 2564 | 2740 | 2620 | 2796 |
| CS1 | CS1-HuLuc64 | 2509 | 2685 | 2565 | 2741 | 2621 | 2797 |
| CS1 | CS1-huLuc90 | 2510 | 2686 | 2566 | 2742 | 2622 | 2798 |
| FLT3 | FLT3-NC7 | 2511 | 2687 | 2567 | 2743 | 2623 | 2799 |
| MPL | MPL-175 | 2512 | 2688 | 2568 | 2744 | 2624 | 2800 |
| MPL | MPL-161 | 2513 | 2689 | 2569 | 2745 | 2625 | 2801 |
| MPL | MPL-111 | 2514 | 2690 | 2570 | 2746 | 2626 | 2802 |
| MPL | Hu-161-2 | 2515 | 2691 | 2571 | 2747 | 2627 | 2803 |
| MPL | MPL-hu-175-2 | 2516 | 2692 | 2572 | 2748 | 2628 | 2804 |
| MPL | MPL-hu-111-2 | 2517 | 2693 | 2573 | 2749 | 2629 | 2805 |
| Lym1 | Lym1 | 2518 | 2694 | 2574 | 2750 | 2630 | 2806 |
| Lym2 | Lym2 | 2519 | 2695 | 2575 | 2751 | 2631 | 2807 |
| CD70 | CD70-h1F6 | 2520 | 2696 | 2576 | 2752 | 2632 | 2808 |
| CD79b | CD79b-2F2 | 2521 | 2697 | 2577 | 2753 | 2633 | 2809 |
| CD179b | CD179b | 2522 | 2698 | 2578 | 2754 | 2634 | 2810 |
| GPRC5D | GPRC5D-ET150-5 | 2523 | 2699 | 2579 | 2755 | 2635 | 2811 |
| GPRC5D | GPRC5D-ET150-18 | 2524 | 2700 | 2580 | 2756 | 2636 | 2812 |
| FR1 | FR1-huMov19 | 2525 | 2701 | 2581 | 2757 | 2637 | 2813 |
| EGFR | Cetuxi | 2638 | 2814 | 2639 | 2815 | 2640 | 2816 |
| FITC | FITC-4M-53 | 2641 | 2817 | 2642 | 2818 | 2643 | 2819 |
| Streptag | Streptag | 2644 | 2820 | 2645 | 2821 | 2851 | 2852 |

The activation/expansion of the T cells using bispecific/multispecific engagers can be further improved by altering the binding affinity of the bispecific/multispecific engagers to their cognate antigens using methods known in the art.

In one embodiment, the population of cells (e.g., immune effector cells, e.g., T cells) are autologous to the subject to whom the cells will be administered for treatment. In one embodiment, the population of immune effector cells are allogeneic to the subject to whom the cells will be administered to for treatment.

In one embodiment, the population of immune effector cells are T cells isolated from peripheral blood lymphocytes. In one embodiment, the population of immune effector cells are obtained from a subject who has received an agent to mobilize the immune cells from tissues. In one embodiment, the population of immune effector cells are obtained from a subject who has been administered a CXCR4 antagonist, a cytokine (e.g., G-CSF, GM-CSF, etc.) a beta2 adrenergic agonist, dasatinib or has been made to exercise. In an embodiment, the population of T cells are obtained by lysing the red blood cells and/or by depleting the monocytes. In an embodiment, the population of T cells is isolated from peripheral lymphocytes using, e.g., a method described herein. In one embodiment, the T cells comprise CD4+ T cells. In another embodiment, the T cells comprise CD8+ T cells. In another embodiment, the T cells comprise Pgp+ T cells. In a further embodiment, the T cells comprise T stem memory cells. In another embodiment, the T cells comprise tissue-resident or tumor-infilterating T cells. In a further embodiment, the T cells comprise naive T-cells. In one embodiment, the immune cells, e.g, immune effector cells, comprise hematopoietic stem cells (e.g., cord blood cells) that can give rise to immune cells. In one embodiment, the immune cells, e.g., immune effector cells, are derived from induced pluripotent stem cells (e.g., iPSC) that can give rise to immune cells. In another embodiment, the immune effector cells comprise stem T cells. In another embodiment, the immune effector cells comprise NKT cells. In one embodiment, the population of immune cells, e.g, immune effector cells, can be obtained from a blood sample from a subject, e.g., obtained by apheresis.

In one embodiment, the method comprises obtaining a population of immune effector T cells from the tumor tissue of a subject (i.e. Tumor infiltrating lymphocytes).

In one embodiment, the method further comprises generating a population of engineered immune cells expressing exogenous RNA or DNA from the population of immune cells.

In one embodiment, the immune cells are expanded and/or activated by culturing the immune cells in the presence of bispecific/multispecific engager and an immobilized ligand, e.g., a cognate antigen molecule or an anti-idiotype antibody. In one embodiment, the immune effector cells are contacted with the T cell bispecific antibody and cognate ligand (e.g., antigen molecule or anti-idiotype antibody) at least, 2, 3, 4, 5, 6, 7, 8, 9, 10, 11, 12, 13, 14, 15, 16, 17, 18, 19, 20, 21, 22, 23, 24, 28, 32, 36, 36, or 48 hours after the nucleic acid (RNA or DNA) encoding the CAR or the TCR is introduced into the immune cells. In one embodiment, the immune cells are contacted with the bispecific/multispecific engager and its immobilized cognate ligand (e.g. antigen molecule or an i anti-idiotype antibody) less than 24, 15, 12, 10, or 8 hours after nucleic acid (RNA or DNA) encoding the CAR or the TCR is introduced into the immune cells, e.g, immune effector cells.

In one embodiment, the immune cells are expanded and/or activated by culturing the immune cells (e.g., CAR-T cells) in the presence of bispecific/multispecific engager, e.g., a bispecific antibody, and an immobilized ligand, (e.g., a cognate antigen molecule or an anti-idiotype antibody) and an antibody against a co-stimulatory receptor expressed on the immune T cells. Exemplary co-stimulatory receptors include CD28, 41BB and CD27. In one embodiment, the 41BB antibody is Utomilumab.

In one embodiment, the ligand is a molecule that activates the T cell upon binding to the bispecific/multispecifc engager, e.g, a bispecific antibody. In one embodiment, the bispecific antibody is Blinatumomab and the immobilized cognate ligand is a CD19-expressing B cell. In one embodiment, the bispecific antibody is Blinatumomab and the immobilized cognate ligand is a bead coated with CD19 extracellular domain or a fragment thereof. In one embodiment, the cognate ligand is a recombinant antigen recognized by the second antigen binding portion of the bispecific antibody. In one embodiment, the ligand is an anti-idiotype antibody or antibody fragment or non-immunoglobulin antigen binding scaffold (e.g., it is an antibody molecule that binds to the second antigen binding domain of the bispecific antibody) e.g., an anti-CD19 idiotype antibody.

In one embodiment, the ligand is attached to a substrate. In one embodiment, the substrate is a solid support. In one embodiment, the substrate is selected from microtiter plates (e.g., ELISA plates); membranes (e.g., nitrocellulose membranes, PVDF membranes, nylon membranes, acetate derivatives, and combinations thereof); fiber matrix, Sepharose matrix, sugar matrix; plastic chips; glass chips; or any type of bead (e.g., Luminex beads, Dynabeads, magnetic beads, flow-cytometry beads, and combinations thereof). In one embodiment, the substrate is an ELISA plate. In another embodiment, the substrate is a bead, e.g., Dynabeads.

In one embodiment, the immune effector cells (e.g., CAR-T or TCR-T cells) are contacted with the ligand, e.g., antigen-coated beads at a ratio of 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, or 15:1 beads per immune effector cell. In one embodiment, the immune effector cells are contacted with antigen coated beads at a ratio of 3:1 beads per immune effector cell.

In one embodiment, the immune effector cells (e.g., CAR-T or TCR-T) are further expanded in an appropriate media (e.g., media described herein) that may, optionally, contain one or more factors for proliferation and/or viability, including serum (e.g., fetal bovine or human serum), interleukin-2 (IL-2), insulin, IFN-γ, IL-4, IL-7, GM-CSF, IL-10, IL-12, IL-15, IL-21, TGF, and TNF-α or any other additives for the growth of cells. In one embodiment, the cells are expanded in the presence of IL-15 and/or IL-7 (e.g., IL-15 and IL-7). In one embodiment, the cells are expanded in the presence of one or more inhibitors of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL (TNFSF10) and/or Death Receptor 5 (DR5 or TNFRSF10B). In one embodiment, the cells are expanded in the presence of AKT inhibitor. In one embodiment, the immune cells are expanded in the presence of IL-2. In one embodiment, the immune cells are expanded in the presence of CD28 antibody coated beads. In one embodiment, the immune cells are expanded in the presence of 41BB antibody coated beads. In one embodiment, the immune cells are expanded in the presence of CD28 antibody. In one embodiment, the immune cells are expanded in the presence of 41BB antibody. In one embodiment, the 41BB antibody is Utomilumab.

In one embodiment, immune effector cells transduced with a nucleic acid encoding a CAR or TCR, e.g., a CAR or TCR described herein, e.g., a CD19 CAR or a NY-ESO-1 TCR described herein, are expanded in culture in the presence of bispecific/multispecific engager, e.g., a bispecific antibody, and its cognate ligand for a period of several hours (e.g., about 2, 3, 4, 5, 6, 7, 8, 9, 10, 15, 18, 21 hours) to about 40 days (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 days). In one embodiment, the cells are expanded for a period of 4 to 9 days. In one embodiment, the cells are expanded for a period of 8 days or less, e.g., 7, 6,5, 4, or 3 days.

Potency of the immune cells, e.g. immune effector cells, e.g., CAR-T or TCR-T cells, can be defined, e.g., by various T cell functions, e.g. proliferation, target cell killing, cytokine production, activation, migration, or combinations thereof. In one embodiment, the immune cells, e.g., a CD19 CAR cell described herein, expanded in the presence of the bispecific/multispecific engager, e.g., T cell bispecific antibody, and cognate ligand for 5 days show at least one, two, three or four-fold increase in cells doublings upon antigen stimulation as compared to the same cells expanded in culture without the bispecific/multispecific engager, e.g., T cell bispecific antibody, and cognate ligand. In one embodiment, the immune effector cells, e.g., the cells expressing a CD19 CAR described herein, are expanded in culture for 5 days in the presence of the bispecific/multispecific engager, e.g., T cell bispecific antibody, and cognate ligand, and the resulting cells exhibit higher proinflammatory cytokine production, e.g., IFN-γ and/or GM-CSF levels, as compared to the same cells expanded in culture without bispecific/multispecific engager, e.g., T cell bispecific antibody, and cognate ligand under the same culture conditions. In one embodiment, the immune effector cells, e.g., a CD19 CAR cell described herein, expanded for 5 days in the presence of the bispecific/multispecific engager, e.g., T cell bispecific antibody, and cognate ligand show at least a one, two, three, four, five, ten fold or more increase in pg/ml of proinflammatory cytokine production, e.g., IFN-γ and/or GM-CSF levels, as compared to the same cells expanded in culture without the bispecific/multispecific engager and cognate ligand under the same culture conditions.

In one embodiment, the immune cells, e.g., immune effector cells expressing a CAR, are expanded at least a 200-fold (e.g., 200-fold, 250-fold, 300-fold, 350-fold, 400-fold, 450-fold, 500-fold, 550-fold, or 650-fold) as measured by a method described herein such as flow cytometry. In one embodiment, the cells are expanded about 500 fold.

In one embodiment, the cell expansion is measured at about 5, 10, 15, 20, 25, 30, 35, 40, 45, or 50 days after stimulation with the bispecific/multispecific engager, e.g., bispecific antibody, and its cognate ligand, e.g., the cognate antigen molecule. In one embodiment, the cell expansion is measured between 10 and 25 days after stimulation with the ligand, e.g., the cognate antigen molecule. In one embodiment, the expansion is measured 8, 9, 10, 11, 12, 13, 14, 15, 16, 17, 18, 19, 20, 21, 22, 23, 24, or 25 days after stimulation with the ligand, e.g., the cognate antigen molecule.

In one embodiment, the immune cells, e.g., immune effector cells, are cryopreserved after the appropriate expansion period. In one embodiment, the cells are cryopreserved according to a method described herein. In one embodiment, the expanded cells are cryopreserved in an appropriate media, e.g., an infusible media, e.g., as described herein.

In another aspect, the disclosure features a reaction mixture comprising a population of immune effector cells wherein a plurality of the cells of the population in the reaction mixture comprise a nucleic acid molecule that comprises a CAR/TCR encoding sequence, e.g., a CD19 CAR encoding sequence or aNYESO-1 TCR encoding sequence, e.g., as described herein.

In one embodiment, at least at least 20%, 50%, 80%, 85%, 86%, 87%, 88%, 89%, 90%, 91%, 92%, 93%, 94%, 95%, 96%, 97%, 98%, 99% or 100% of the immune effector cells express the CAR/TCR mRNA.

In another embodiment, at least at least at least 80%, 85%, 86%, 87%, 88%, 89%, 90%, 91%, 92%, 93%, 94%, 95%, 96%, 97%, 98%, 99% or 100% of the immune cells, e.g., immune effector cells, express the CAR/TCR on their cell surface.

In one embodiment, the reaction mixture can further comprise a bispecific/multispecific engager, e.g., bispecific antibody, and its cognate ligand as described herein (e.g., a cognate antigen molecule or an anti-idiotype antibody). In one embodiment, the ligand is an antigen (e.g., CD19) that is expressed on the surface of a cell and is bound by one of the antigen binding domains of the T cell bispecific antibody (e.g., Blinatumomab). In another embodiment the ligand is an anti-idiotype antibody, e.g., an anti-CD19 idiotype antibody.

In one embodiment, the immune effector cells, e.g., CAR/TCR-expressing T cells, and the ligand (e.g., antigen) coated beads are present in a ratio of 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, or 15:1 beads per immune effector cell. In one embodiment, the immune effector cells and the ligand (e.g., antigen) coated beads are present in a ratio of 3:1 beads per immune effector cell. The reaction mixture further comprises the T cell bispecific antibody. In one embodiment, the reaction mixture further comprises a co-stimulatory molecule, such as a 41BB agonist (e.g., 41BBL or Utomilumab).

In one embodiment, the reaction mixture further comprises a cryoprotectant or stabilizer such as, e.g., a saccharide, an oligosaccharide, a polysaccharide and a polyol (e.g., trehalose, mannitol, sorbitol, lactose, sucrose, glucose and dextran), salts and crown ethers. In one embodiment, the cryoprotectant is dextran.

In accordance with the methods, preparations, and reaction mixtures described herein, an immune effector cell, e.g., obtained by a method described herein and activated/expanded by bispecific antibodies described herein, can be engineered to contain a CAR, a next generation CAR (e.g., SIR, zSIR, TFP, Ab-TCR) and/or a recombinant TCR molecule that targets one or more cancer associated antigens. In some embodiments, the tumor antigen is a tumor antigen described in International Applications WO2015/142675, filed Mar. 13, 2015, which is herein incorporated by reference in its entirety. In some embodiments, CAR, a recombinant TCR, a TCR receptor fusion proteins or TFP, Antibody TCR or AbTCRs, and synthetic immune receptors or SIR are as described in PCT/US2017/024843, WO 2014/160030 A2, WO 2016/187349 A1, PCT/US2016/058305 and PCT/US17/64379, which are herein incorporated by reference in their entirety.

In another aspect the disclosure features a method of treating, or providing anti-tumor immunity to, a subject having a cancer. The method includes administering to the subject an effective amount of an immune effector cell population, wherein the immune effector cell population is, or was previously, expanded by contacting the immune effector cell population, with a bispecific/multispecific engager, e.g., bispecific antibody, wherein the bispecific antibody targets a cognate antigen molecule expressed preferentially on hematopoietic cells; and expanding/activating the population of immune effector cells in the presence of a ligand, e.g., the cognate antigen molecule or an anti-idiotypic antibody molecule. In one embodiment, the nucleic acid is RNA, e.g., in vitro transcribed RNA. In another embodiment, the cognate antigen molecule is an antigen expressed on hematopoietic cells. In one embodiment, the cognate antigen molecule or the anti-idiotypic antibody molecule is attached to a substrate, e.g., a bead. In some embodiments, the method further includes administering to the subject an immune cell population comprising a CAR or a recombinant TCR (e.g., a vector comprising a nucleic acid encoding a CAR or a recombinant TCR or rTCR), wherein the immune effector cell population is, or was previously, expanded as described herein. In one embodiment, the vector is selected from the group consisting of a DNA, a RNA, a plasmid, a lentivirus vector, adenoviral vector, or a retrovirus vector.

In one embodiment, the population of immune effector cells is transduced with a vector once, e.g., within one day after population of immune effector cells are obtained from a blood sample from a subject, e.g., obtained by apheresis.

In one embodiment, the bispecific antibody targets a cognate antigen molecule (e.g., CD19) and the CAR/recombinant TCR targets a different antigen molecule (e.g., Mesothelin or NY-ESO-1). In one embodiment, the bispecific antibody targets a cognate antigen molecule (e.g., CD19) and the CAR/rTCR targets the same cognate antigen molecule.

In one embodiment, the bispecific antibody targets an antigen expressed on hematopoietic cells described herein, e.g., CD19, and the CAR/rTCR targets a cancer associated antigen described herein, e.g., Mesothelin or NY-ESO-1.

In one embodiment, the bispecific antibody targets an antigen expressed on B cells (e.g., CD19, CD20/MS4A1 or CD22) and CAR/rTCR targets an antigen (e.g., Mesothelin, Her2, Her3, TSHR, LHR, EGFRviii, EGFR, Folate Receptor alpha, ROR1, NY-ESO-1, AFP etc.) expressed on solid tumors (e.g., Breast, prostate, brain, lung, gastrointestinal, kidney, thyroid, pancreatic, hepatic, skin, ovarian, cervical, endometrial, endocrine and soft tissue cancers).

In one embodiment, the bispecific antibody targets an antigen (e.g., BCMA, CD138, SLAMF7 etc.) expressed on plasma cells and CAR/rTCR targets an antigen (e.g., Mesothelin, Her2, Her3, TSHR, LHR, EGFRviii, EGFR, Folate Receptor alpha, ROR1 etc.) expressed on solid tumors (e.g., Breast, prostate, brain, lung, gastrointestinal, kidney, thyroid, pancreatic, hepatic, skin, ovarian, cervical, endometrial, endocrine and soft tissue cancers).

In one embodiment, the bispecific antibody targets an antigen (e.g., CD33, CD123, MPL etc.) expressed on myeloid cells and CAR/rTCR targets an antigen (e.g., Mesothelin, Her2, Her3, TSHR, LHR, EGFRviii, EGFR, Folate Receptor alpha, ROR1, NY-ESO-1, AFP etc.) expressed on solid tumors (e.g., Breast, prostate, brain, lung, gastrointestinal, kidney, thyroid, pancreatic, hepatic, skin, ovarian, cervical, endometrial, endocrine and soft tissue cancers).

In one embodiment, the bispecific antibody targets CD19, CD20/MS4A1, CD22, CD23, BCMA, CS1/SLAMF7, CD30, CD32b, CD70, CD79b, CD123, CD33, CD138, CD179b, GPRC5D, Lyml, Lym2, and/or FCRH5 and CAR/rTCR targets Mesothelin, epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); Tn antigen ((Tn Ag) or (GalNAcα-Ser/Thr)); prostate-specific membrane antigen (PSMA); Receptor tyrosine kinase-like orphan receptor 1 (ROR1); Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Interleukin 11 receptor alpha (IL-11Ra); prostate stem cell antigen (PSCA); vascular endothelial growth factor receptor 2 (VEGFR2); Lewis(Y) antigen; Stage-specific embryonic antigen-4 (SSEA-4); Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CA1X); tyrosinase; ephrin type-A receptor 2 (EphA2); sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR5IE2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); CD19; CD20/MS4A1; CD123; CD22; CD23, CD30; CD33; CD171; CS-1 (also referred to as CD2 subset 1, CRACC, SLAMF7, TNF receptor family member B cell maturation (BCMA); CD319, and 19A24); C-type lectin-like molecule-1 (CLL-1 or CLECL1); Fins Like Tyrosine Kinase 3 (FLT3); Tumor-associated glycoprotein 72 (TAG72); CD38; CD44v6; a glycosylated CD43 epitope expersed on acute leukemia or lymphoma but not on hematopoietic progenitors, a glycosylated CD43 epitope expressed on non-hematopoietic cancers, Protease Serine 21 (Testisin or PRSS21); CD24; Platelet-derived growth factor receptor beta (PDGFR-beta); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYPlB 1); CCCTC-Binding Factor (Zinc Finger Protein)-Like (BORIS or Brother of the Regulator ofimprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), MPL, Biotin, c-MYC epitope Tag, CD34, LAMP1 TROP2, GFRalpha4, CDH17, CDH6, NYBRI, CDH19, CD200R, Slea (CA19.9; Sialyl Lewis Antigen); Fucosyl-GM1, PTK7, gpNMB, CDH1-CD324, DLL3, CD276/B7H3, IL11Ra, IL13Ra2, CD179b-IGL11, TCRgamma-delta, NKG2D, CD32 (FCGR2A), Tn ag, Tim1-/HVCR1, CSF2RA (GM-CSFR-alpha), TGFbetaR2, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, HIV1 envelope glycoprotein, HTLV1-Tax, CMV pp65, EBV-EBNA3c, KSHV K8.1, KSHV-gH, influenza A hemagglutinin (HA), GAD, PDL1, GUANYLYL CYCLASE C (GCC), autoantibody to desmoglein 3 (Dsg3), MPL, autoantibody to desmoglein 1 (Dsg1), HLA, HLA-A, HLA-A2, HLA-B, HLA-C, HLA-DP, HLA-DM, HLA-DOA, HLA-DOB, HLA-DQ, HLA-DR, HLA-G, IGE, CD99, RAS G12V, TISSUE FAACTOR 1 (TF1), AFP, GPRC5D, CLAUDIN18.2 (CLD18A2 OR CLDN18A.2)), P-GLYCOPROTEIN, STEAP1, LIV1, NECTIN-4, CRIPTO, GPA33, BST1/CD157, LOW CONDUCTANCE CHLORIDE CHANNEL, VISTA, CD16ORF54, Muc5Ac, EMR2, Robo4, RNF43, CLDN6, MMP16, UPK1B, BMPR1B, Ly6E, STEAPI, WISP1, SLC34A2 and antigen recognized by TNT antibody.

In accordance with methods of treating a disorder as described herein (e.g., a cancer) and providing anti-tumor immunity described herein, in some embodiments, the method comprises administering to a subject a population of immune cells (e.g., CAR-T cells) made by a method described herein along with a T cell bispecific antibody. In some embodiment the population of immune cells is engineered to express a CAR or a rTCR molecule, e.g. a CAR or a rTCR described herein, e.g., a CD19 CAR or a NY-ESO-1 rTCR described herein.

Also provided herein is a composition comprising an immune cell (e.g., CAR-T cells) and a bispecific/multispecific engager, e.g., a bispecific antibody, as described herein for use in the treatment of a subject having a disease associated with expression of a tumor antigen, e.g., a disorder as described herein.

In one embodiment, the cancer is a hematological cancer such as, e.g., ALL or CLL. In embodiments, the disease associated with expression of the tumor antigen is selected from the group consisting of a proliferative disease, a precancerous condition, a cancer, and a non-cancer related indication associated with expression of the tumor antigen.

In a preferred embodiment, the disease associated with a tumor antigen described herein is a solid tumor.

In preferred embodiments of any of the aforesaid methods or uses, the tumor antigen associated with the disease is chosen from one or more of: Mesothelin, EGFR viii, CHD6, CDH17, CDH19, DLL3, CLD18A2, ALK, CD276, CD324, B7H4, EGFR, EBNA3c, EpCaml, LICAM, Folate Receptor 1, GFRa4, STEAPI, Livl, Nectin4, Cripto, gpA33, ILIRAP, GD2, GD3, gp100, ROR1, SLea, PTK7, Prolactin Receptor, LHR, TSHR, Lewis Y, Her2, GCC, SSEA4, IL-13Ra2, PSMA, PSCA, NY-ESO1, WT1, MART1, MAGE1, AFP, TIM1, TROP2, hTERT, MMP16, UPK1B, BMPR1B, Ly6E, STEAPI, WISP1, SLC34A2, VEGFR3, Tn-Mucd, and Tyrosinase.

In one embodiment, the population of cells are autologous to the subject administered the population. In one embodiment, the population of cells is allogeneic to the subject administered the population. In one embodiment, the subject is a human.

In one embodiment, the subject is administered 10⁴ to 10⁶ immune cells, e.g., immune effector cells, per kg body weight of the subject. In one embodiment, the subject receives an initial administration of a population of immune effector cells (e.g., an initial administration of 10⁴ to 10⁶ immune effector cells per kg body weight of the subject, e.g., 10⁴ or 10⁵ immune effector cells per kg body weight of the subject), and one or more subsequent administrations of a population of immune effector cells (e.g., one or more subsequent administration of 10⁴ to 10⁶ immune effector cells per kg body weight of the subject, e.g., 10⁴ to 10⁶ immune effector cells per kg body weight of the subject). In one embodiment, the one or more subsequent administrations are administered less than 15 days, e.g., 14, 13, 12, 11, 10, 9, 8, 7, 6, 5, 4, 3, or 2 days after the previous administration, e.g., less than 4, 3, 2 days after the previous administration. In one embodiment, the subject receives a total of about 10⁶ immune effector cells per kg body weight of the subject over the course of at least three administrations of a population of immune effector cells, e.g., the subject receives an initial dose of 1×10⁵ immune effector cells, a second administration of 3×10⁵ immune effector cells, and a third administration of 6×10⁵ immune effector cells, and, e.g., each administration is administered less than 4, 3, 2 days after the previous administration.

In some embodiments, the subject is administered immune effector cells that have been engineered to express a recombinant receptor, such as a chimeric antigen receptor (CAR), a next generation CAR (e.g., a SIR, a zSIR, a TFP, an Ab-TCR, a cTCR) or a recombinant TCR. In some embodiments, the subject is administered immune effector cells where more than 10%, 20%, 50%, 75%, 80%, 90%, 95%, 99% of cells express a recombinant receptor.

In some embodiments, the subject is administered immune effector cells that have been isolated from a tumor (e.g. tumor infiltrating lymphocytes).

In some embodiments, the subject is administered immune effector T cells that have been activated by cytokines or chemokines to attack a tumor (e.g., cytokine activated killer cells).

In some embodiment, the subject is administrated a cell therapy product (e.g., CAR-T) and a bispecific/multispecific engager, e.g., a bispecific antibody, e.g., a bispecific antibody described herein, for a predetermined period (e.g., about 1, 2, 3, 4, 5, 6, 7, 8, 9, 10, 15, 18, 21, 22, 23 or 24 hours) or (e.g., 1, 5, 10, 15, 20, 25, 30, 35, 40, 45, 50, 60, 70, 90, 100, 120 days). In one embodiment, the subject receives a bispecific/multispecific engager, e.g., a bispecific antibody (e.g., a bispecific antibody described herein) for period of 2 to 20 days. In one embodiment, the subject receives a bispecific/multispecific engager, e.g., a bispecific antibody (e.g., a bispecific antibody described herein) for a period of 28 days or less, e.g., 20, 10, 7, 6 or 5 days. In one embodiment, bispecific/multispecific engager, e.g., a bispecific antibody, is administered in multiple cycles of 1-28 days followed by a rest period of 1-14 days.

In an exemplary embodiment, the subject is administered the bispecific antibody Blinatumomb (BLINCYTO) after the administration of immune effector T cells (e.g., CAR-T cells) and Blinatumomab is administered to the subject as a continuous intravenous infusion at a constant flow rate using an infusion pump which is programmable, lockable, non-elastomeric, and has an alarm as described in its prescribing information. In an exemplary embodiment, Blinatumomab is administered at a dose of 1 mcg/day, 2 mcg/day, 5 mcg/day, 10 mcg/day, 20 mcg/day, 25 mcg/day, 28 mcg/day, 30 mcg/day or 50 mcg/day by continuous infusion. In the preferred embodiment, Blinatumomab is administered after premedication with prednisone 100 mg intravenously or equivalent (e.g., dexamethasone 16 mg) 1 hour prior to the first dose of Blinatumomab in each cycle.

In an embodiment, the subject is administered cell therapy product (e.g., CAR-T) and a bispecific/multispecific engager, e.g., a bispecific antibody, as described herein (e.g., Blinatumomab) along with a costimulatory molecule as described herein, e.g., a 41BB agonist as described herein, e.g., Utomilumab. In an embodiment, Utomilumab is administered intravenously at doses ranging from 0.05 mg/kg to 5.0 mg/kg.

In one embodiment, subject is administered the cell therapy product (e.g., CAR-T) and the bispecific/multispecific engager, e.g., a bispecific antibody, after administration of lymphodepleting chemotherapy. Several lymphodepleting chemotherapy regimens are known in the art. An exemplary lymphodepleting chemotherapy regimen includes 30 mg/m²/day fludarabine intravenously plus 500 mg/m²/day cyclophosphamide intravenously x 3 days. In an exemplary embodiment, the subject is administered cell therapy product (e.g., CAR-T) 1 day after finishing the administration of the lymphodepleting chemotherapy. The bispecific/multispecific engager, e.g., a bispecific antibody, can be administered to the subject concurrently with the administration of the cell therapy product (e.g., CAR-T), prior to the administration of the cell therapy product (e.g., CAR-T) or after the administration of the cell therapy product (e.g., CAR-T). In the preferred embodiment, the subject is administered the bispecific/multispecific engager, e.g., a bispecific antibody, after the administration of the cell therapy product (e.g., CAR-T).

In certain embodiments, the methods or uses are carried out in combination with an agent that increases the efficacy of the cell therapy product (e.g., CAR-T), e.g., an agent as described herein. For example, in one embodiment, the agent can be an agent, which inhibits an inhibitory molecule. Examples of inhibitory molecules include PDi, PD-L1, CTLA4, TIM3, CEACAM (e.g., CEACAM-1, CEACAM-3 and/or CEACAM-5), LAG3, VISTA, BTLA, TIGIT, LAIR1, CD160, 2B4 and TGF beta. In one embodiment, the agent is a PD-1 antibody, e.g., Pembrolizumab.

In one embodiment, the cell therapy product (e.g., CAR-T) and the bispecific antibody described herein, are administered in combination with an agent that ameliorates one or more side effect associated with administration of a cell expressing immune effector cells and/or bispecific antibody, e.g., an agent described herein. For example, an immune effector T cell and the bispecific antibody is administered in combination steroids, an IL6R antagonist (e.g. Tocilizumab), an IL1R antagonist (e.g., Anakinra), a TRAIL antagonist (e.g. a DR5-Fc or a neutralizing antibody against TRAIL, e.g., MAB375-SP).

In one embodiment, the reaction mixture further comprises a cryoprotectant or stabilizer such as, e.g., a saccharide, an oligosaccharide, a polysaccharide and a polyol (e.g., trehalose, mannitol, sorbitol, lactose, sucrose, glucose and dextran), salts and crown ethers. In one embodiment, the cryoprotectant is dextran.

In one embodiment, the subject, e.g., the subject from which immune cells are acquired and/or the subject treated, is a human, e.g., a cancer patient. In some embodiments, the subject from whom the immune cells are acquired is a healthy donor. In some embodiments, the subject from whom the immune cells are acquired has received a mobilizing agent, e.g., a CXCR4 antagonist.

In certain embodiments, the subject has a disease associated with expression of a tumor- or cancer associated-antigen, e.g., a disease as described herein. In one embodiment, the subject has a cancer, e.g., a cancer as described herein.

In one embodiment, the subject has a cancer that is chosen from a hematological cancer, a solid tumor, or a metastatic lesion thereof. In preferred embodiment, the cancer is a solid tumor.

In embodiments, the subject does not have a relapsed cancer. In other embodiments, the subject has a relapsed cancer.

In one embodiment, the immune cell (e.g., the population of immune effector cells) is acquired, e.g., obtained, from a subject having a solid tumor, e.g., breast, lung, prostate, skin, gastrointestinal, brain, endocrine, endometrial, ovarian, cervical and hepatic cancer.

In one embodiment, the immune cell (e.g., the population of immune effector cells) is acquired, e.g., obtained, from a subject who is an allogeneic donor, e.g., a healthy donor, e.g., a person who does not have cancer.

The following assays can be used to assay the phenotype and funcational activity of immune cells, e.g., T cells that have been expanded/activated by use of bispecific/multispecific engagers, e.g., bispecific antibodies, as described herein. These assay can be also used to assay T cells that express a CAR or a recombinant TCR and have been expanded using other means, such as using CD3×CD28 beads or co-culture with Mantle Cell Lymphoma derived cell lines, e.g., REC-1 cells. The assays can be also used to assay the phenotype and funcational activity of immune cells generated using other methods of the disclosure, e.g., using inhibitors of TRAIL or by expressing constitutive active mutant of JAK3.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once a CAR described herein is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of a cars of the disclosure are described in further detail below

Western blot analysis of CAR expression in primary T cells can be used to detect the presence of monomers and dimers. See, e.g., Milone et al., Molecular Therapy 17(8): 1453-1464 (2009).

In vitro expansion of CAR+ T cells following antigen stimulation can be measured by flow cytometry. Sustained CAR+ T cell expansion in the absence of re-stimulation can also be measured. See, e.g., Milone et al., Molecular Therapy 17(8): 1453-1464 (2009). Animal models can also be used to measure a CART activity.

Assessment of cell proliferation and cytokine production has been previously described, e.g., at Milone et al., Molecular Therapy 17(8): 1453-1464 (2009).

Imaging technologies can be used to evaluate specific trafficking and proliferation of CARs in tumor-bearing animal models. Such assays have been described, for example, in Barrett et al., Human Gene Therapy 22:1575-1586 (2011).

Other assays, including those described in the Example section herein as well as those that are known in the art can also be used to evaluate the CARs, TCRs, TILs and other immune effector cells described herein.

In one aspect, the disclosure provides methods for treating a disease associated with expression of a cancer associated antigen described herein.

In one aspect, the disclosure provides methods of treating cancer by providing to the subject in need thereof immune effector cells (e.g., T cells, NK cells) that are engineered to express an XCAR or an XTCR, wherein X represents a tumor antigen as described herein, and wherein the cancer cells express said X tumor antigen.

In one aspect, the disclosure provides methods of treating cancer by providing to the subject in need thereof immune effector cells (e.g., T cells, NK cells) that are engineered to express a XCAR or a XTCR described herein, wherein the cancer cells express X. In one embodiment, X is expressed on both normal cells and cancers cells, but is expressed at lower levels onirnormal cells. In one embodiment, the method further comprises selecting a CAR or a TCR that binds X with an affinity that allows the XCAR to bind and kill the cancer cells expressing X but less than 30%, 25%, 20%, 15%, 10%, 5% or less of the normal cells expressing X are killed, e.g., as determined by an assay described herein. Tabe 7F provides a list of different antigens and the exemplary diseases that can be prevented, inhibited or treated using immune effector cells expressing CAR/TCR targeting these antigens and using the methods of the disclosure.

TABLE 7F

| CAR/TCR “X” | EXEMPLARY DISEASE TARGETED BY CARs (i.e. conventional CARs |
|---|---|
| TARGET | and next generation CARs. E.g., SIR, Ab-TCR, TFP and zSIR) or TCRs |
| CD19 | ALL, CLL, lymphoma, lymphoid blast crisis of CML, multiple myeloma, |
| | immune disorders |
| ALK | Non Small Cell Lung Cancer (NSCLC), ALCL (anaplastic large cell |
| | lymphoma), IMT (inflammatory myofibroblastic tumor), or neuroblastoma |
| CD45 | Blood cancers |
| BCMA | Myeloma, PEL, plasma cell leukemia, Waldenstrom's macroglobinemia |
| CD5 | Blood cancer, T cell leukemia, T cell lymphoma |
| CD20/MS4A1 | Blood cancers, Leukemia, ALL, CLL, lymphoma, immune disorders |
| CD22 | Blood cancers, Leukemia, ALL, CLL, lymphoma, lymphoid blast crisis of |
| | CML, immune disorders |
| CD23 | Blood cancers, Leukemia, ALL, CLL, lymphoma, autoimmune disorders |
| CD30 | Hodgkins's lymphoma, Cutaneous T cell lymphoma |
| CD32 | Solid tumors |
| CD33 | Blood cancers, AML, MDS |
| CD34 | Blood cancers, AML, MDS |
| CD44v6 | Blood cancers, AML, MDS |
| CD70 | Blood cancers, lymphoma, myeloma, waldenstrom's macroglobulinemia |
| CD79b | Blood cancers, ALL, Lymphoma |
| CD123 | Blood cancers, AML, MDS |
| CD138 | Blood cancers, Myeloma, PEL, plasma cell leukemia, waldenstrom's |
| | macroglobulinemia |
| CD179b | Blood cancers, ALL, Lymphoma |
| CD276/B7-H3 | Ewing's sarcoma, neuroblastoma, rhabdomyosarcoma, ovarian, colorectal and |
| | lung cancers |
| CD324 | Solid tumors, esophageal, prostate, colorectal, breast, lung cancers |
| CDH6 | Solid tumors, renal, ovarian, thyroid cancers |
| CDH17 | Adenocarciniomas, gastrointestinal, lung, ovarian, endometrial cancers |
| CDH19 | Solid tumor, Melanoma |
| EGFR | Colon cancer, lung cancer |
| CLEC5A | Blood cancers, Leukemia, AML |
| GR/LHR | Prostate cancer, ovarian cancer or breast cancer |
| CLL1 | Blood cancer, Leukemia |
| CMVpp65 | CMV infection, CMV colitis, CMV pneumonitis |
| CS1 | Blood cancers, myeloma, PEL, plasma cell leukemia |
| CSF2RA | AML, CML, MDS |
| CD123 | Blood cancers, AML, MDS |
| DLL3 | Melanoma, lung cancer or ovarian cancer |
| EBNA3c/MHC I | Epstein Barr virus infection and related diseases including cancers |
| EBV-gp350 | Epstein Barr virus infection and related diseases |
| EGFR | Solid tumors, Colon cancer, lung cancer |
| EGFRvIII | Solid tumors, glioblastoma |
| EpCam1 | Gastrointestinal cancer |
| FLT3 | Blood cancers, AML, MDS, ALL |
| Folate Receptor | Ovarian cancer, NSCLC, endometrial cancer, renal cancer, or other |
| alpha(FR1 or FOLR1) | solid tumors |
| FSHR | Prostate cancer, ovarian cancer or breast cancer |
| GD2 | Neuroblastoma |
| GD3 | Melanoma |
| GFRa4 | Cancer, thyroid medullary cancer |
| Fucosyl-GM1(GM1) | Small cell lung cancer |
| GPRC5D | Myeloma, PEL, plasma cell leukemia, waldenstrom's macroglobulinemia |
| gp100 | Melanoma |
| GPC3 | Solid tumors, Lung cancer |
| gpNMB | Melanoma, brain tumors, gastric cancers |
| GRP78 | Myeloma |
| Her2 | Solid tumors, breast cancer, stomach cancer |
| Her3 | Colorectal, breast cancer |
| HMW-MAA | Melanoma |
| HTLV1-TAX/MHC I | HTLV1 infection associated diseases, Adult T cell leukemia-lymphoma |
| IL11Ra | Blood cancers, AML, ALL, CML, MDS, sarcomas |
| IL6Ra | Solid tumors, Liver cancer |
| IL13Ra2 | Glioblastomas |
| KSHV-K8.1 | Kaposi's sarcoma, PEL, Multicentric Castleman's disease |
| LAMP1 | Blood cancers, AML, ALL, MDS, CLL, CML |
| LewisY | Cancers |
| L1CAM | Solid tumors, ovarian, breast, endometrial cancers, melanoma |
| LHR | Prostate cancer, ovarian cancer or breast cancer |
| Lym1 | Blood cancer, Leukemia, Lymphoma |
| Lym2 | Blood cancer, Leukemia, Lymphoma |
| CD79b | Blood cancers, lymphoma |
| MART1/MHC I | Melanoma |
| Mesothelin | Mesothelioma, ovarian cancer, pancreatic cancer |
| Muc1/MHC I | Breast cancer, gastric cancer, colorectal cancer, lung cancer, or |
| | other solid tumors |
| Muc16 | Ovarian cancer |
| NKG2D | Leukemia, lymphoma or myeloma |
| NYBR1 | Breast cancer |
| PSCA | Prostate cancer |
| PR1/MHC I | Blood cancer, Leukemia |
| Prolactin Receptor | Breast cancer, chromophobe renal cell cancer |
| PSMA | Prostate cancer |
| PTK7 | Melanoma, lung cancer or ovarian cancer |
| ROR1 | Blood cancer, B cell malignancy, lymphoma, CLL |
| SLea | Pancreatic cancer, colon cancer |
| SSEA4 | Pancreatic cancer |
| Tyrosinase/MHC I | Melanoma |
| TCRB1 | T cell leukemias and lymphomas, autoimmune disorders |
| TCRB2 | T cell leukemias and lymphomas, autoimmune disorders |
| TCRgd | T cell leukemias and lymphomas, autoimmune disorders |
| hTERT | Solid tumors, blood cancers |
| TGFBR2 | Solid tumors, keloid |
| TIM1/HAVCR1 | Kidney cancer, liver cancer |
| TROP2 | Solid tumors, Breast cancer, prostate cancer |
| TSHR | Thyroid cancer, T cell leukemia, T cell Lymphoma |
| TSLPR | Blood cancers, Leukemias, AML, MDS |
| Tyrosinase/MHC I | Melanoma |
| VEGFR3 | Solid tumors |
| WT1/MHC I | Blood cancers, AML |
| Folate Receptorβ | AML, Myeloma |
| B7H4 | Breast cancer or ovarian cancer |
| CD23 | Blood cancers, Leukemias, CLL |
| GCC | Gastrointestinal cancer |
| CD200R | Blood cancers, AML, MDS |
| AFP/MHC I | Solid tumors, Liver cancer |
| CD99 | Liver cancer |
| GPRC5D | Myeloma, waldenstrom's macroglobinemia |
| HPV16-E7/MHC I | HPV16 associated cancers, cervical cancer, head and neck cancers |
| Tissue Factor 1 | Solid tumors |
| (TF1) | |
| Tn-Muc1 | Solid tumors and blood cancers |
| Igk-Light Chain | Myeloma, plasma cell leukemia |
| Ras G12V/MHC I | Solid tumors and blood cancers |
| CLD18A2 | Gastric, pancreatic, esophageal, ovarian, or lung cancer |
| (Claudin 18.2) | |
| CD43 | Blood cancers, AML |
| NY-ESO-1/MHC I | Myeloma |
| MPL/TPO-R | Blood cancer, AML, MDS, CML, ALL |
| P-glycoprotein | Renal cancer, liver cancer, Myeloma |
| (MDR1) | |
| CD179a | Blood cancers, Acute Leukemia, CLL, ALL, Lymphoma |
| STEAP1 | Gastric or prostate cancer, or lymphoma |
| Liv1 (SLC39A6) | Breast or prostate cancer |
| Nectin4 (PVRL4) | Bladder, renal, cervical, lung, head and neck or breast cancer |
| Cripto (TDGF1) | Colorectal or endometrial or ovarian cancer |
| gpA33 | Colorectal or endometrial or ovarian cancer |
| FLT3 | Blood cancers, AML, ALL, MDS |
| BST1/CD157 | Blood cancers, AML, MDS |
| IL1RAP | Liver, colorectal, cervical, lung or ovarian cancer |
| Chloride channel | Glioma |
| IgE | Allergy |
| HLA-A2 | Graft vs host disease, tissue rejection (SIR Expressed in regulatory T cells) |
| Amyloid | Amyloidoses, alzheimer's disease |
| HIV1-env | HIVI/AIDS and related conditions |
| HIV1-gag | HIV1/AIDS and related conditions |
| Influenza A HA | Influenza A infection |
| Integrin B7 | Plasma cell neoplasms, primary effusion lymphoma |
| Muc17 | Pancreatic cancer, colon cancer |
| CD16ORF54 | Blood cancers |
| VISTA | Blood cancers |
| Muc5Ac | Pancreatic cancer, stomach cancer, colon cancer |
| FCRH5 | Plasma cell neoplasm |
| LYPD1 | Ovarian cancer, endometrial cancer, melanoma |
| EMR2 | Acute Leukemia, Lymphoma, breast cancer, colon cancer |
| gpNMB | Melanoma, brain cancer, breast cancer, solid tumors |
| RNF43 | Colorectal cancer, breast cancer, endometrial cancer |
| CD44v6 | Epithelial cancers |
| Robo4 | Renal, colon, breast cancer, solid tumors |
| GPC3 | Liver cancer, lung cancer, breast cancer |
| FOLR1 | Ovarian cancer, lung cancer, kidney cancer, solid tumors |
| CLDN6 | Ovarian cancer, liver cancer |
| MMP16 | Melanoma, brain cancer, small lung cancer, neuroblastoma |
| BMPR1B | Prostat cancer, breast cancer, ovarian cancer |
| Ly6E | Breast, ovarian, pancreatic, lung |
| WISP1 | Glioblastoma, breast cancer |
| SLC34A2 | Lung cancer, ovarian cancer, endometrial cancer |
| CD133 | Lung cancer, brain cancer |

In one embodiment, the disclosure provides methods of treating cancer by providing to the subject in need thereof immune effector cells (e.g., T cells, NK cells) that are engineered to express CDT19 CAR, wherein the cancer cells express CDT19. In one embodiment, the cancer to be treated is ALL (acute lymphoblastic leukemia), CLL (chronic lymphocytic leukemia), DLBCL (diffuse large B-cell lymphoma), MCL (Mantle cell lymphoma, or MM (multiple myeloma).

In one embodiment, the disclosure provides methods of treating an inmm-une disorder by providing to the subject in need thereof immune effector cells (e.g., T cells, NK cells) that are engineered to express CDT9 CAR, wherein the disease causing or disease associated cells express CDT9. In one embodiment, the immune disorder is an autoimmune disorder (e.g., lupus, SLE, rheumatoid arthritis, sjogren syndrome, sarcoidosis etc.).

In one embodiment, the disclosure provides methods of treating cancer by providing to the subject in need thereof immune effector cells (e.g., T cells, NK cells) that are engineered to express CD22 CAR, wherein the cancer cells express CD22. In one embodiment, the cancer to be treated is ALL (acute lymphoblastic leukemia), CLL (chronic lymphocytic leukemia), DLBCL (diffuse large B-cell lymphoma), or MCL (Mantle cell lymphoma.

In one embodiment, the disclosure provides methods of treating cancer by providing to the subject in need thereof immune effector cells (e.g., T cells, NK cells) that are engineered to express CD20/MS4A1 CAR, wherein the cancer cells express CD20/MS4A1. In one embodiment, the cancer to be treated is ALL (acute lymphoblastic leukemia), CLL (chronic lymphocytic leukemia), DLBCL (diffuse large B-cell lymphoma), or MCL (Mantle cell lymphoma.

In one embodiment, the disclosure provides methods of treating an immune disorder by providing to the subject in need thereof immune effector cells (e.g., T cells, NK cells) that are engineered to express CD20, CD22, BCMA, CS1 or CD138 CAR, wherein the disease causing or disease associated cells express CD20, CD22, BCMA, CS1 or CD138. In one embodiment, the immune disorder is an autoimmune disorder (e.g., lupus, SLE, rheumatoid arthritis, sjogren syndrome, sarcoidosis etc.).

In one embodiment, the disclosure provides methods of treating cancer by providing to the subject in need thereof immune effector cells (e.g., T cells, NK cells) that are engineered to express BCMA CAR, wherein the cancer cells express BCMA. In one embodiment, the cancer to be treated is plasma cell disorder (e.g., myeloma) or primary effusion lymphoma.

In one aspect, the disclosure provides methods of treating cancer by providing to the subject in need thereof immune effector cells (e.g., T cells, NK cells) that are engineered to express an EGFRvIIICAR (or EGFRviiiCAR), wherein the cancer cells express EGFRvlll (or EGFRviii). In one embodiment, the cancer to be treated is glioblastoma.

In one aspect, the disclosure provides methods of treating cancer by providing to the subject in need thereof immune effector cells (e.g., T cells, NK cells) that are engineered to express a mesothelinCAR (MSLN-CAR), wherein the cancer cells express mesothelin (MSLN). In one embodiment, the cancer to be treated is mesothelioma, pancreatic cancer, gastrointestinal cancer, lung cancer, breast cancer or ovarian cancer.

In one aspect, the disclosure provides methods of treating cancer by providing to the subject in need thereof immune effector cells (e.g., T cells, NK cells) that are engineered to express a CD123CAR, wherein the cancer cells express CD123. In one embodiment, the cancer to be treated is AML.

In one aspect, the disclosure provides methods of treating cancer by providing to the subject in need thereof immune effector cells (e.g., T cells, NK cells) that are engineered to express a CS-1CAR, wherein the cancer cells express CS-1. In one embodiment, the cancer to be treated is multiple myeloma or primary effusion lymphoma.

In one aspect, the disclosure provides methods of treating cancer by providing to the subject in need thereof immune effector cells (e.g., T cells, NK cells) that are engineered to express a CLL-1CAR, wherein the cancer cells express CLL-1. In one embodiment, the cancer to be treated is AML.

In one aspect, the disclosure provides methods of treating cancer by providing to the subject in need thereof immune effector cells (e.g., T cells, NK cells) that are engineered to express a CD33CAR, wherein the cancer cells express CD33. In one embodiment, the cancer to be treated is AML.

In one aspect, the disclosure provides methods of treating cancer by providing to the subject in need thereof immune effector cells (e.g., T cells, NK cells) that are engineered to express a MPL-CAR, wherein the cancer cells express MPL (or Thrombopoietin receptor). In one embodiment, the cancer to be treated is AML or MDS.

In one aspect, the disclosure provides methods of treating cancer by providing to the subject in need thereof immune effector cells (e.g., T cells, NK cells) that are engineered to express a GD2CAR, wherein the cancer cells express GD2. In one embodiment, the cancer to be treated is neuroblastoma.

In one aspect, the disclosure provides methods of treating cancer by providing to the subject in need thereof immune effector cells (e.g., T cells, NK cells) that are engineered to express a TnCAR, wherein the cancer cells express Tn antigen. In one embodiment, the cancer to be treated is ovarian cancer.

In one aspect, the disclosure provides methods of treating cancer by providing to the subject in need thereof immune effector cells (e.g., T cells, NK cells) that are engineered to express a PSMACAR, wherein the cancer cells express PSMA. In one embodiment, the cancer to be treated is prostate cancer.

In one aspect, the disclosure provides methods of treating cancer by providing to the subject in need thereof immune effector cells (e.g., T cells, NK cells) that are engineered to express a ROR1CAR, wherein the cancer cells express ROR1. In one embodiment, the cancer to be treated is B cell malignancies.

In one aspect, the disclosure provides methods of treating cancer by providing to the subject in need thereof immune effector cells (e.g., T cells, NK cells) that are engineered to express a FLT3 CAR, wherein the cancer cells express FLT3. In one embodiment, the cancer to be treated is AML.

In one aspect, the disclosure provides methods of treating cancer by providing to the subject in need thereof immune effector cells (e.g., T cells, NK cells) that are engineered to express a CD38CAR, wherein the cancer cells express CD38. In one embodiment, the cancer to be treated is multiple myeloma.

In one aspect, the disclosure provides methods of treating cancer by providing to the subject in need thereof immune effector cells (e.g., T cells, NK cells) that are engineered to express a CD44v6CAR, wherein the cancer cells express CD44v6. In one embodiment, the cancer to be treated is cervical cancer, AML, or MM.

In one aspect, the disclosure provides methods of treating cancer by providing to the subject in need thereof immune effector cells (e.g., T cells, NK cells) that are engineered to express a CEACAR, wherein the cancer cells express CEA. In one embodiment, the cancer to be treated is pastrointestinal cancer, or pancreatic cancer.

In one aspect, the disclosure provides methods of treating cancer by providing to the subject in need thereof immune effector cells (e.g., T cells, NK cells) that are engineered to express a B7H3CAR, wherein the cancer cells express B7H3.

In one aspect, the disclosure provides methods of treating cancer by providing to the subject in need thereof immune effector cells (e.g., T cells, NK cells) that are engineered to express a KITCAR, wherein the cancer cells express KIT. In one embodiment, the cancer to be treated is gastrointestinal cancer.

In one aspect, the disclosure provides methods of treating cancer by providing to the subject in need thereof immune effector cells (e.g., T cells, NK cells) that are engineered to express an IL-13Ra2CAR, wherein the cancer cells express IL-13Ra2. In one embodiment, the cancer to be treated is glioblastoma.

In one aspect, the disclosure provides methods of treating cancer by providing to the subject in need thereof immune effector cells (e.g., T cells, NK cells) that are engineered to express a CD30CAR, wherein the cancer cells express CD30. In one embodiment, the cancer to be treated is lymphomas, or leukemias.

In one aspect, the disclosure provides methods of treating cancer by providing to the subject in need thereof immune effector cells (e.g., T cells, NK cells) that are engineered to express a GD3CAR, wherein the cancer cells express GD3. In one embodiment, the cancer to be treated is melanoma.

In one aspect, the disclosure provides methods of treating cancer by providing to the subject in need thereof immune effector cells (e.g., T cells, NK cells) that are engineered to express an IL-11RaCAR, wherein the cancer cells express IL-11Ra. In one embodiment, the cancer to be treated is osteosarcoma.

In one aspect, the disclosure provides methods of treating cancer by providing to the subject in need thereof immune effector cells (e.g., T cells, NK cells) that are engineered express a PSCACAR, wherein the cancer cells express PSCA. In one embodiment, the cancer to be treated is prostate cancer.

In one aspect, the disclosure provides methods of treating cancer by providing to the subject in need thereof immune effector cells (e.g., T cells, NK cells) that are engineered to express a VEGFR2CAR, wherein the cancer cells express VEGFR2. In one embodiment, the cancer to be treated is a solid tumor.

In one aspect, the disclosure provides methods of treating cancer by providing to the subject in need thereof immune effector cells (e.g., T cells, NK cells) that are engineered to express a LewisYCAR, wherein the cancer cells express LewisY. In one embodiment, the cancer to be treated is ovarian cancer, or AML.

In one aspect, the disclosure provides methods of treating cancer by providing to the subject in need thereof immune effector cells (e.g., T cells, NK cells) that are engineered to express a CD24CAR, wherein the cancer cells express CD24. In one embodiment, the cancer to be treated is pancreatic cancer.

In one aspect, the disclosure provides methods of treating cancer by providing to the subject in need thereof immune effector cells (e.g., T cells, NK cells) that are engineered to express a Folate receptor alphaCAR, wherein the cancer cells express folate receptor alpha. In one embodiment, the cancer to be treated is ovarian cancer, NSCLC, endometrial cancer, renal cancer, or other solid tumors.

In one aspect, the disclosure provides methods of treating cancer by providing to the subject in need thereof immune effector cells (e.g., T cells, NK cells) that are engineered to express an ERBB2CAR, wherein the cancer cells express ERBB2 (Her2/neu). In one embodiment, the cancer to be treated is breast cancer, gastric cancer, colorectal cancer, lung cancer, or other solid tumors.

In one aspect, the disclosure provides methods of treating cancer by providing to the subject in need thereof immune effector cells (e.g., T cells, NK cells) that are engineered to express a MUCICAR, wherein the cancer cells express MUCl. In one embodiment, the cancer to be treated is breast cancer, lung cancer, or other solid tumors.

In one aspect, the disclosure provides methods of treating cancer by providing to the subject in need thereof immune effector cells (e.g., T cells, NK cells) that are engineered to express an EGFRCAR, wherein the cancer cells express EGFR. In one embodiment, the cancer to be treated is glioblastoma, SCLC (small cell lung cancer), SCCHN (squamous cell carcinoma of the head and neck), NSCLC, or other solid tumors.

In one aspect, the disclosure provides methods of treating cancer by providing to the subject in need thereof immune effector cells (e.g., T cells, NK cells) that are engineered to express a CAIXCAR, wherein the cancer cells express CAIX. In one embodiment, the cancer to be treated is renal cancer, CRC, cervical cancer, or other solid tumors.

In one aspect, the disclosure provides methods of treating cancer by providing to the subject in need thereof immune effector cells (e.g., T cells, NK cells) that are engineeredto express a GD3CAR, wherein the cancer cells express GD3. In one embodiment, the cancer to be treated is melanoma.

In one aspect, the disclosure provides methods of treating cancer by providing to the subject in need thereof immune effector cells (e.g., T cells, NK cells) that are engineered to express a Fucosyl GMICAR, wherein the cancer cells express Fucosyl GM.

In another aspect, a method of treating a subject, e.g., reducing or ameliorating, a hyperproliferative condition or disorder (e.g., a cancer), e.g., solid tumor, a soft tissue tumor, or a metastatic lesion, in a subject is provided. Without wishing to be bound by any particular theory, the anti-tumor immunity response elicited by the cell therapy product (e.g., CAR-modified immune effector cells; e.g., T cells, NK cells) may be an active or a passive immune response, or alternatively may be due to a direct vs indirect immune response.

In one aspect, the cell therapy products (e.g., CAR-expressing T cells, NK cells) of the disclosure may be a type of vaccine for ex vivo immunization and/or in vivo therapy in a mammal. In one aspect, the mammal is a human.

In addition to using a cell-based vaccine in terms of ex vivo immunization, the disclosure also provides compositions and methods for in vivo immunization to elicit an immune response directed against an antigen in a patient.

Generally, the cells activated and expanded as described herein may be utilized in the treatment and prevention of diseases that arise in individuals who are immunocompromised. In particular, the cell therapy products (e.g., CAR/TCR-modified immune effector cells; e.g., T cells, NK cells) of the disclosure are used in the treatment of diseases, disorders and conditions associated with expression of a cancer associate antigen as described herein. In certain aspects, the cell therapy products of the disclosure are used in the treatment of patients at risk for developing diseases, disorders and conditions associated with expression of a cancer associate antigen as described herein. Thus, the disclosure provides methods for the treatment or prevention of diseases, disorders and conditions associated with expression of a cancer associate antigen as described herein comprising administering to a subject in need thereof, a therapeutically effective amount of the cell therapy products of the disclosure.

In one aspect the cell therapy products (e.g., CAR/TCR-modified immune effector cells; e.g., T cells, NK cells) of the disclosures may be used to treat a proliferative disease such as a cancer or malignancy or is a precancerous condition such as a myelodysplasia, a myelodysplastic syndrome or a preleukemia. Further a disease associated with a cancer associate antigen as described herein expression include, but not limited to, e.g., atypical and/or non-classical cancers, malignancies, precancerous conditions or proliferative diseases expressing a cancer associated antigen as described herein. Non-cancer related indications associated with expression of a cancer associate antigen as described herein include, but are not limited to, e.g., autoimmune disease, (e.g., lupus), inflammatory disorders (allergy and asthma) and transplantation.

The cell therapy products (e.g., CAR/TCR-modified immune effector cells; e.g., T cells, NK cells) of the disclosure may be administered either alone, or as a pharmaceutical composition in combination with diluents and/or with other components such as IL-2 or other cytokines or cell populations.

The disclosure provides methods for preventing relapse of cancer associated with a cancer associated antigen as described herein-expressing cells, the methods comprising administering to a subject in need thereof a cell therapy product (e.g., CAR/TCR-modified immune effector cells; e.g., T cells, NK cells) of the disclosure that binds to a cancer associated antigen as described herein-expressing cell. In one aspect, the methods comprise administering to the subject in need thereof an effective amount of a cell therapy product (e.g., CAR/TCR-modified immune effector cells; e.g., T cells, NK cells) described herein that binds to a cancer associated antigen as described herein-expressing cell in combination with an effective amount of another therapy.

A cell therapy product (e.g., CAR/TCR-modified immune effector cells; e.g., T cells, NK cells) described herein may be used in combination with other known agents and therapies.

A cell therapy product (e.g., CAR/TCR-modified immune effector cells; e.g., T cells, NK cells) described herein and the at least one additional therapeutic agent can be administered simultaneously, in the same or in separate compositions, or sequentially. For sequential administration, the cell therapy products (e.g., CAR/TCR-modified immune effector cells; e.g., T cells, NK cells) described herein can be administered first, and the additional agent can be administered second, or the order of administration can be reversed.

The cell therapy products (e.g., CAR/TCR-modified immune effector cells; e.g., T cells, NK cells) and/or other therapeutic agents, procedures or modalities can be administered during periods of active disorder, or during a period of remission or less active disease. The cell therapy products (e.g., CAR/TCR-modified immune effector cells; e.g., T cells, NK cells) can be administered before the other treatment, concurrently with the treatment, post-treatment, or during remission of the disorder.

When administered in combination, the cell therapy products (e.g., CAR/TCR-modified immune effector cells; e.g., T cells, NK cells) and the additional agent (e.g., second or third agent), or all, can be administered in an amount or dose that is higher, lower or the same than the amount or dosage of each agent used individually, e.g., as amonotherapy. In certain embodiments, the administered amount or dosage of the cell therapy, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the cell therapy, the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

In further aspects, a cell therapy product (e.g., CAR/TCR-modified immune effector cells; e.g., T cells, NK cells) described herein may be used in a treatment regimen in combination with intratumoral administration of an immune modulating agent. In some embodiment, the immune modulating agent is IL2. In some embodiments, the immune modulating agent is an agent that activates NF-κB signaling pathway. Exemplary immune modulating agents that can activate NF-κB signaling pathway include one or more of vFlip K13 (SEQ ID NO (DNA): 2357; SEQ ID NO (PRT): 2467), human NEMO K277A mutant (SEQ ID NO (DNA): 2358 and SEQ ID NO (PRT)): 2468) and human IKK2-S177E-S181E mutant (SEQ ID NO (DNA): 2359 and SEQ ID NO (PRT): 2469). In some embodiment, the immune modulating agent, e.g., IL2, vFLIP K13 SEQ ID NO (DNA): 2357, human NEMO K277A mutant (SEQ ID NO (DNA) and/or human IKK2-S177E-S181E mutant (SEQ ID NO (DNA): 2359 are administered to the tumor by transfection, e.g. by electroporation, of the nucleic acids encoding the corresponding gene or cDNA. In some embodiment, the immune modulating agent, e.g., IL2, vFLIP K13 SEQ ID NO (DNA): 2357, human NEMO K277A mutant (SEQ ID NO (DNA) and/or human IKK2-S177E-S181E mutant (SEQ ID NO (DNA): 2359 are administered to the tumor using viral vectors, e.g, viral vectors described herein, e.g., lentiviral vector, AAV vector or Adenoviral vector, encoding the corresponding gene or cDNA. In some embodiments, the immune modulatory agent is delivered about 2 days (e.g., 1 day, 2 days, 3 days, 7 days etc.) before the administration of immune effector cells. In some embodiments, the immune modulatory agent is delivered about 2 days (e.g., 1 day, 2 days, 3 days, 7 days etc.) after the administration of immune effector cells.

In one embodiment, a cell therapy product (e.g., CAR/TCR-modified immune effector cells; e.g., T cells, NK cells) described herein can be used in combination with a chemotherapeutic agent.

In an exemplary embodiment, a cell therapy product described herein is a CD19 CART product and administered to a subject who has a CD19+ lymphoma, e.g., a CD19+ Non-Hodgkin's Lymphoma (NHL), a CD19+FL, or a CD19+ DLBCL. In embodiments, the subject has a relapsed or refractory CD19+ lymphoma. In embodiments, a lymphodepleting chemotherapy is administered to the subject prior to, concurrently with, or after administration (e.g., infusion) of CD19 CAR-T cells. In an example, the lymphodepleting chemotherapy is administered to the subject prior to administration of CD19 CART cells. For example, the lymphodepleting chemotherapy ends 1-4 days (e.g., 1, 2, 3, or 4 days) prior to CD19 CART cell infusion. In embodiments, multiple doses of CD19 CART cells are administered, e.g., as described herein. For example, a single dose comprises about 5×10′ CD19 CART cells. In embodiments, a lymphodepleting chemotherapy is administered to the subject prior to, concurrently with, or after administration (e.g., infusion) of a CAR-expressing cell described herein, e.g., anon-CD19 CAR-expresing cell. In embodiments, a CD19 CART is administered to the subject prior to, concurrently with, or after administration (e.g., infusion) of a non-CD19 CAR-expressing cell, e.g., a non-CD19 CAR-expressing cell describedherein.

In some embodiments, a CAR-expressing cell described herein is administered to a subject in combination with an interleukin-15 (IL-15) polypeptide, an interleukin-15 receptor alpha (IL-15Ra) polypeptide, or a combination of both a IL-15 polypeptide and a IL-15Ra polypeptide e.g., hetIL-15 (Admune Therapeutics, LLC).

In one embodiment, the subject can be administered an agent which reduces or ameliorates a side effect associated with the administration of a CAR-expressing cell. Side effects associated with the administration of a CAR-expressing cell include, but are not limited to CRS, and hemophagocytic lymphohistiocytosis (HLH), also termed Macrophage Activation Syndrome (MAS) and neurological complications (e.g., seizures, aphasia, confusion, coma etc.).

Accordingly, the methods described herein can comprise administering a CAR-expressing cell described herein to a subject and further administering one or more agents to manage elevated levels of a soluble factor resulting from treatment with a CAR-expressing cell. In one embodiment, the soluble factor elevated in the subject is one or more of IFN-γ, TNFα, IL-2 and IL-6. In an embodiment, the factor elevated in the subject is one or more of IL-1, GM-CSF, IL-10, IL-8, IL-5 and fraktalkine. Therefore, an agent administered to treat this side effect can be an agent that neutralizes one or more of these soluble factors. In one embodiment, the agent that neutralizes one or more of these soluble forms is an antibody or antigen binding fragment thereof. Examples of such agents include, but are not limited to a steroid (e.g., corticosteroid), an inhibitor of TNFα, and an inhibitor of IL-6.

The present disclosure also provides inhibitors of TRAIL and/or DR5 and method of use of such inhibitors for reducing, preventing and/or treating the side effects of immune therapy and cell therapy products, e.g., CAR-T, TCR-T cells and Bispecific T cell engagers (BiTE). In an embodiment, the inhibitors of TRAIL and/or DR5 are used to reduce, prevent and/or treat cytokine release syndrome (CRS) and neurological complications associated with the use of immune therapy and cell therapy, e.g., immune effector cell therapy, product. In an embodiment, the BiTE is Blinatumomab.

In another embodiment of the disclosure, the subject is administered a TRAIL antagonist to manage elevated levels of a soluble factor (e.g., cytokines) resulting from treatment with a cell therapy, e.g., immune effector cell, e.g., CAR-expressing cell. In an embodiment, the subject is administered a TRAIL antagonist to to manage elevated levels of a soluble factor (e.g., cytokines) resulting from treatment with a bispecific antibody (e.g., Blinatumomab or BCMA x CD3 bispecific antibody) that binds to immune effector cell. In an embodiment, the subject is administered a TRAIL antagonist to manage side effects (e.g. CRS and neurotoxicity) resulting from treatment with an immune effector cell (e.g., CAR-T cells or TCR-T cells or TILs) or a bispecific antibody (e.g., Blinatumomab) that binds to immune effector cell. In an embodiment, the subject is administered a TRAIL antagonist to manage elevated levels of a soluble factor (e.g., cytokines) resulting from treatment with a bispecific antibody that binds to immune effector cell. In an embodiment, the subject is administered a TRAIL antagonist to manage elevated levels of a soluble factor resulting from treatment with a TCR-expressing cell. In an embodiment, the subject is administered a TRAIL antagonist to to manage elevated levels of a soluble factor (e.g., cytokines) resulting from treatment with any immune effector cell.

An example of a TRAIL antagonist is a neutralizaing antibody against TRAIL e.g. MAB375-SP. In an embodiment, MAB375-SP is administered to a subject for the prevention or treatment of cytokine release syndrome and other toxicities, including neurotoxicity, resulting from administration of immune effector cell therapy (e.g., CAR-T, TCR-T, TILs, Blinatumomab, BCMA x CD3 BiTE etc.) at a dose of about 5 mg/kg (e.g. 5 mg/kg, 10 mg/kg, 20 mg/kg, 50 mg/kg, 100/kg mg) by subcutaneous or intravenous injection. In some embodiments, MAB375-SP is administered by intra-thecal or intra-ventricular injection to prevent or treat neurotoxicity associated with administration of cell therapy products. In some embodiment, the dose of MAB375-SP for intrathecal or intra-ventricular injection is about 20 mg (e.g., 20 mg, 50 mg, 100 mg) every week. In an embodiment, more than one dose of MAB375-SP is administered in case of no response to first dose. In an embodiment, MAB375-SP is administered about once every week, (e.g., once every week, twice every week, three times every week, once every two weeks, once every month etc.). In an embodiment, MAB375-SP is administered prophylactically, i.e., to prevent the development of CRS. In other embodiment, MAB375-SP is administered to treat CRS. In yet other embodiment, MAB375-SP is administered at the earliest signs and symptoms of CRS and/or neurotoxicity, such as fever >38.5° C., drop in systolic or diastolic blood pressure of more than 10 mm Hg, systolic blood pressure of <100 mm Hg or diastolic blood pressure of <70 mm Hg. In an embodiment, MAB375-SP is administered as a monotherapy. In other embodiments, MAB375-SP is administered in combination with other agents, e.g., corticosteroids, DR5-Fc, tocilizumab or anakinra.

Another example of a TRAIL antagonist is an antibody or a protein that binds to DR5 and prevent its binding to TRAIL. An example of a TRAIL antagonist is an antibody or a protein that binds to TRAIL and prevent or compete with its binding to DR5. An example of a protein that binds to TRAIL and compete with its binding to DR5 is DR5-Fc. In an embodiment, DR5-Fc is administered to a subject for the prevention or treatment of cytokine release syndrome and other toxicities, including neurotoxicity, resulting from administration of immune therapy, e.g., immune effector cell therapy (e.g., CAR-T, TCR-T, TILs, Blinatumomab, BCMA x CD3 BiTE etc.) at a dose of about 10 mg (e.g. 10 mg, 20 mg, 50 mg, 100 mg) by subcutaneous or intravenous injection. In an embodiment, DR5-Fc is administered about once weekly, (e.g., once weekly, twice weekly, or three times every week). In some embodiments, DR5-Fc is administered by intra-thecal or intra-ventricular injection to prevent or treat neurotoxicity associated with administration of cell therapy products. In some embodiment, the dose of DR5-Fc for intrathecal or intra-ventricular injection is about 20 mg (e.g., 20 mg, 50 mg, 100 mg) every week. In an embodiment, more than one dose of DR5-Fc is administered in case of no response to first dose. In an embodiment, DR5-Fc is administered as a monotherapy. In other embodiments, DR5-Fc is administered in combination with other agents, e.g., MAB375-SP, corticosteroids, tocilizumab or anakinra.

Other examples of proteins that bind to TRAIL and compete with its binding to DR5 are known in the art, e.g., DR4-Fc (SEQ ID NO: 2441), DcR1-Fc (SEQ ID NO: 2448), DcR2-Fc, DR5-ECD (SEQ ID NO: 2392), DR4-ECD (SEQ ID NO: 2386), DcR1-ECD (SEQ ID NO: 2375), and DcR2-ECD (SEQ ID NO: 2380) and can be used in alternate embodiments of the disclosure.

Provided in some aspects are methods of treatment including administering to a subject an agent, e.g., a TRAIL antagonist, e.g., DR5-Fc and/or MAB375-SP, capable of treating, preventing, delaying, or attenuating the development of a toxicity. In some cases, at the time of said administration, the subject has been previously administered a therapy, such as a therapy including an immunotherapy and/or a cell therapy. In some embodiments, the administration of the agent or other treatment is at a time that is less than or no more than ten, seven, six, five, four or three days after initiation of the administration of the therapy. In some of any such embodiments, the agent, e.g., a TRAIL antagonist, e.g., DR5-Fc and/or MAB375-SP or other treatment is administered at a time at which the subject exhibits grade 1 CRS or is administered within 24 hours after the subject exhibits a first sign or symptom of grade 1 CRS. In some cases, the agent or other treatment is administered at a time at which the subject exhibits a sign or symptom of CRS and/or exhibits grade 1 CRS. In some cases, the agent or other treatment is administered within 24 hours after the subject exhibits a first sign or symptom of grade 1 CRS following the initiation of administration of the therapy.

In some embodiments, a sign or symptom of grade 1 CRS is a fever. In some cases, the agent, e.g., a TRAIL antagonist, e.g., DR5-Fc and/or MAB375-SP or other treatment is administered within 24 hours after the first sign of a fever following initiation of administration of the therapy. In some aspects, the agent, e.g., a TRAIL antagonist, e.g., DR5-Fc and/or MAB375-SP or other treatment is administered within about 16 hours, within about 12 hours, within about 8 hours, within about 2 hours or within about 1 hour after the first sign of a fever following initiation of administration of the therapy.

In some embodiments, the fever is a sustained fever. In some cases, the fever is not reduced or not reduced by more than 1° C. after treatment with an antipyretic. In some aspects, the fever is a fever that is not reduced or not reduced by more than 1° C. after treatment with an antipyretic. In some instances, the fever has not been reduced by more than 1° C., following treatment of the subject with an antipyretic.

In some embodiments, the fever includes a temperature of at least or at least about 38.0° C. In some aspects, the fever includes a temperature that is between or between about 38.0° C. and 42.0° C., 38.0° C. and 39.0° C., 39.0° C. and 40.0° C. or 40.0° C. and 42.0° C., each inclusive. In some embodiments, the fever includes a temperature that is greater than or greater than about or is or is about 38.5° C., 39.0° C., 39.5° C., 40.0° C., 41.0° C., 42.0° C.

In some embodiments, the agent, e.g., a TRAIL antagonist, e.g., DR5-Fc and/or MAB375-SP or other treatment is administered less than five days after initiation of administration of the therapy, less than four days after initiation of administration of the therapy or less than three days after initiation of administration of the therapy.

In some embodiments, the therapy is or comprises a cell therapy. In some cases, the cell therapy is or comprises an adoptive cell therapy. In some aspects, the therapy is or comprises a tumor infiltrating lymphocytic (TIL) therapy, a transgenic TCR therapy or a recombinant receptor-expressing cell therapy, which optionally is a T cell therapy. In some embodiments, the therapy is a chimeric antigen receptor (CAR)-expressing T cell therapy. In some embodiments, the therapy is a bispecific/multispecific T cell engager therapy. In an exemplary embodiment, the therapy is Blinatumomab. In some embodiments, the therapy is a CD123×CD3 Bispecific antibody. In some embodiment, the therapy is a CD33×CD3 bispecific antibody therapy. In some embodiment, the therapy is a CD123×CD3 DART or a CD19×CD3 DART.

In some cases, the agent, e.g., a TRAIL antagonist, e.g., DR5-Fc and/or MAB375-SP is administered in combination with other treatment including a steroid, or an antagonist or inhibitor of a cytokine receptor or cytokine selected from among IL-10, IL-IOR, IL-6, IL-6 receptor, IFNγ, IFNGR, IL-2, IL-2R/CD25, MCP-1, CCR2, CCR4, MIPIP, CCR5, TNFalpha, TNFR1, IL-1, and IL-1Ralpha/IL-1beta.

In some aspects, a TRAIL antagonist is administered in combination with an agent selected from among an antibody or antigen-binding fragment, a small molecule, a protein or peptide and a nucleic acid. In some cases, the agent or other treatment is or comprises an agent selected from among tocilizumab, anakinra, situximab, sarilumab, olokizumab (CDP6038), elsilimomab, ALD518/BMS-945429, sirukumab (CNTO 136), CPSI-2634, ARGX-109, FE301 and FMl01.

In some embodiments, a TRAIL antagonist is administered in combination with tocilizumab. In some such embodiments, the tocilizumab is administered in a dosage amount of from or from about 1 mg/kg to 10 mg/kg, 2 mg/kg to 8 mg/kg, 2 mg/kg to 6 mg/kg, 2 mg/kg to 4 mg/kg or 6 mg/kg to 8 mg/kg, each inclusive, or the tocilizumab is administered in a dosage amount of at least or at least about or about 2 mg/kg, 4 mg/kg, 6 mg/kg or 8 mg/kg.

In some embodiments, a TRAIL antagonist is administered in combination with anakinra. In some such embodiments, the anakinra is administered in a dosage amount of from or from about 1 mg/kg to 10 mg/kg, 2 mg/kg to 8 mg/kg, 2 mg/kg to 6 mg/kg, 2 mg/kg to 4 mg/kg or 6 mg/kg to 8 mg/kg, each inclusive, or the anakinra is administered in a dosage amount of at least or at least about or about 2 mg/kg, 4 mg/kg, 6 mg/kg or 8 mg/kg.

In some aspects, the method further includes administering a steroid to the subject in combination with a TRAIL antagonist. In some such aspects, the steroid is administered at a time that is within 7 days, 8 days or 9 days after administration of the therapy. In some cases, the steroid is administered at a time that is within 24 hours after the first sign of hypotension following administration of the therapy. In some instances, the steroid is administered at a time in which the subject exhibits grade 2 cytokine release syndrome (CRS) or within 24 hours after the subject exhibits a first sign of grade 2 CRS following administration of the therapy. In some embodiments, the steroid is administered at a time in which the subject exhibits grade 2 neurotoxicity or within 24 hours after the subject exhibits a first sign or symptom of grade 2 neurotoxicity following administration of the therapy.

In some embodiments, the agent that is administered in combination with a TRAIL antagonist is or comprises a steroid that is or comprises a corticosteroid. In some aspects, the agent is a steroid that is or comprises a glucocorticoid. In some cases, the corticosteroid is or comprises dexamethasone or prednisone. In some cases, the steroid is administered intravenously or orally.

In some instances, the steroid is administered in an equivalent dosage amount of from or from about 1.0 mg to 20 mg dexamethasone per day, 1.0 mg to 10 mg dexamethasone per day, or 2.0 mg to 6.0 mg dexamethasone per day, each inclusive.

In some aspects, the TRAIL antagonist, e.g., MAB375-SP or DR5-Fc is administered within 24 hours after or contemporaneously with the first sign of hypotension following initiation of administration of the therapy. In some cases, the TRAIL antagonist, e.g., MAB375-SP or DR5-Fc is administered simultaneously with initiation of a pressor therapy. In some instances, hypotension includes systolic blood pressure less than or about less than 90 mm Hg, 80 mm Hg, or 70 mm Hg. In some instances, hypotension includes diastolic blood pressure less than 60 mm Hg, 50 mm Hg or 40 mm Hg.

In some embodiments, the therapy is or comprises a cell therapy and the cells are administered in a single pharmaceutical composition containing the cells. In some cases, the therapy is or comprises a cell therapy and the dose of cells is a split dose, wherein the cells of the dose are administered in a plurality of compositions, collectively containing the cells of the dose, over a period of no more than three days.

In some embodiments, the disease or condition for which a TRAIL antagonist is administered is or comprises a tumor or a cancer. In some embodiments, the therapy is a cell therapy including a dose of cells expressing a recombinant receptor. In some aspects, the recombinant receptor binds to, recognizes or targets an antigen associated with the disease or condition. In some cases, the recombinant receptor is a T cell receptor or a functional non-T cell receptor. In some instances, the recombinant receptor is a chimeric antigen receptor (CAR).

In some embodiments, the therapy is or comprises a therapy containing a dose of cells containing T cells. In some cases, the T cells are CD4+ or CD8+. In some embodiments, the T cells are autologous to the subject. In some embodiments, the T cells are allogeneic to the subject.

In some embodiments, the method further includes administering a chemotherapeutic agent prior to administering the therapy. In some instances, the subject has been previously treated with a chemotherapeutic agent prior to the initiation of administration of the therapy. In some aspects, the chemotherapeutic agent includes an agent selected from the group consisting of cyclophosphamide, fludarabine, and/or a combination thereof. In some embodiments, the chemotherapeutic agent is administered between 2 and 5 days prior to the initiation of administration of the therapy. In some cases, the chemotherapeutic agent is administered at a dose of between at or about 1 g/m2 of the subject and at or about 3 g/m2 of the subject.

In some embodiments, toxicity for which a TRAIL antagonist is administered is a neurotoxicity. In some embodiments, a CNS-related outcome in the subject at day up to or up to about day 7, 8, 9, 10, 11, 12, 13, 14, 15, 16, 17, 18, 19, 20, 21, 22, 23, 24, 25, 26, 27, 28, 29 or 30 following administration of the therapy, e.g., CAR-T therapy, is not detectable or is reduced as compared to a method including an alternative treatment regimen wherein the subject is administered the agent or other treatment after severe CRS or neurotoxicity has developed or after grade 2 or higher CRS or neurotoxicity has developed. In some embodiments, the toxic outcome is a symptom associated with grade 3 or higher neurotoxicity or is a symptom associated with grade 2 or higher CRS. In some embodiments, the toxic outcome is reduced by greater than 50%, 60%, 70%, 80%, 90% or more. In some cases, the toxic outcome is a symptom associated with grade 3 or higher neurotoxicity. In some embodiments, the toxic outcome is selected from among grade 3 or higher neurotoxicity include confusion, delirium, expressive aphasia, obtundation, myoclonus, lethargy, altered mental status, convulsions, seizure-like activity and seizures.

In some embodiments, the toxic outcome is grade 2 or higher CRS comprising one or more symptom selected from among persistent fever greater than at or about 38 degrees Celsius, for at least three consecutive days; hypotension requiring high dose vasopressor or multiple vasopressors; hypoxia, which optionally comprises (e.g., plasma oxygen (p02) levels of less than at or about 90% and respiratory failure requiring mechanical ventilation. In some embodiments, the therapy is a cell therapy comprising a dosage of cells and the cells exhibit increased or prolonged expansion and/or persistence in the subject as compared to administration of the cell therapy (in the subject or in a corresponding subject in an alternative cohort or treatment group) using alternative treatment regimen, wherein said alternative treatment regimen comprises administering the cell therapy and subsequently administering the agent or other treatment after severe CRS has developed or after grade 2 or higher CRS has developed, and optionally wherein the subject in said alternative treatment regimen is not administered said agent, and optionally is not administered any other treatment designed to treat CRS or neurotoxicity, following the administration of the cells and prior to said development of grade 2 or higher CRS or severe CRS. In some embodiments, the increase in or prolonging of expansion and/or persistence is by 2-fold, 3-fold, 4-fold, 5-fold, 6-fold, 7-fold, 8-fold, 9-fold or 10-fold.

In some embodiments, the therapy is a cell therapy comprising a dosage of cells and the cells exhibit increased or prolonged expansion and/or persistence in the subject as compared to the administration of the cell therapy (in the subject or a corresponding subject in an alternative cohort or treatment group) using alternative treatment regimen. In some cases, said alternative treatment regimen comprises administering the cell therapy and subsequently administering the agent or other treatment after severe CRS or neurotoxicity has developed or after grade 2 or higher CRS or neurotoxicity has developed. In some cases, the subject in said alternative treatment regimen is not administered said agent. In some instances, the subject in said alternative treatment regimen is not administered any other treatment designed to treat CRS or neurotoxicity, following the administration of the cells and prior to said development of grade 2 or higher CRS or severe CRS or grade 2 or higher neurotoxicity or severe neurotoxicity.

In some embodiments, the increase in or prolonging of expansion and/or persistence is by 2-fold, 3-fold, 4-fold, 5-fold, 6-fold, 7-fold, 8-fold, 9-fold or 10-fold.

In some embodiments, the therapy is a cell therapy, comprising engineered and/or CAR-expressing cells. In some cases, the concentration or number of the engineered and/or CAR-expressing cells in the blood of the subject at day 30, day 60, or day 90 following initiation of administration of the therapy is at least at or about 10 engineered or CAR-expressing cells per microliter, at least 50% of the total number of peripheral blood mononuclear cells (PBMCs), at least or at least about 1×10⁵ engineered or CAR-expressing cells, and/or at least 5,000 copies of CAR-encoding or engineered receptor-encoding DNA per micrograms DNA. In some embodiments, at day 30, 60, or 90 following the initiation of the administration of the therapy, the CAR-expressing and/or engineered cells are detectable in the blood or serum of the subject. In some instances, at day 30, 60, or 90 following the initiation of the administration of the therapy, the blood of the subject contains at least 20% CAR-expressing cells, at least 10 CAR-expressing cells per microliter or at least 1×10⁴ CAR-expressing cells. In some cases, at day 30, 60, or 90 following the initiation of the administration of the therapy, the blood of the subject contains at least 50%, 60%, 70%, 80%, or 90% of a biologically effective dose of the cells. In some embodiments, at day 30, 60, or 90 following the initiation of the administration of the therapy, the blood of the subject contains at least 20% engineered and/or CAR-expressing cells, at least 10 engineered and/or CAR-expressing cells per microliter and/or at least 1×10⁴ engineered and/or CAR-expressing cells. In some cases, at day 30, 60, or 90 following the initiation of the administration of the therapy, the subject exhibits a reduction or sustained reduction in burden of the disease or condition. In some cases, the reduction or sustained reduction in burden of the disease or condition is at or about or at least at or about 50, 60, 70, or 80% peak reduction following the therapy administration or reduction associated with effective dose.

In some embodiments, at day 30, 60 or 90 following the initiation of the administration of the therapy, the subject does not, and/or has not, following the cell therapy treatment, exhibited severe neurotoxicity, severe CRS, grade 2 or higher CRS, grade 2 or higher neurotoxicity, and/or has not exhibited seizures or other CNS outcome; or at day 30, 60, or 90 following the initiation of the administration of the therapy, less than or about less than 25%, less than or about less than 20%, less than or about less than 15%, or less than or about less than 10%) of the subjects so treated do not, and/or have not, following the cell therapy treatment, exhibited severe neurotoxicity, severe CRS, grade 2 or higher CRS, grade 2 or higher neurotoxicity, and/or have not exhibited seizures or other CNS outcome.

In some embodiments, the therapy is a cell therapy, comprising engineered and/or CAR-expressing cells; and the area under the curve (AUC) for blood concentration of engineered and/or CAR-expressing cells over time following the administration of the therapy is greater as compared to that achieved via a method comprising an alternative dosing regimen, such as where the subject is administered the therapy and is administered the agent or other treatment at a time at which the subject exhibits a severe or grade 2 or higher or grade 3 or higher CRS or neurotoxicity.

In some embodiments, also provided are agents, e.g., a TRAIL antagonist, e.g., DR5-Fc and/or MAB375-SP or other treatment for use in the treatment, prevention, delay or attenuation of the development of a toxicity in a subject that has been previously administered a therapy, which therapy comprises an immunotherapy and/or a cell therapy. In some embodiments, (a) the agents, e.g., TRAIL antagonists or other treatment are administered to a subject: (i) at a time that is less than or no more than ten, seven, six, five, four or three days after initiation of the subject having been administered the therapy; and/or (ii) at a time at which the subject does not exhibit a sign or symptom of severe cytokine release syndrome (CRS) and/or does not exhibit grade 2 or higher CRS; and/or (iii) at a time at which the subject does not exhibit a sign or symptom of severe neurotoxicity and/or does not exhibit grade 2 or higher neurotoxicity; and/or (b) between the time of initiation of the subject having been administered the therapy and the time of the administration of the agent or other treatment, (i) the subject has not exhibited severe CRS and/or has not exhibited grade 2 or higher CRS and/or (ii) the subject has not exhibited severe neurotoxicity and/or does not exhibit grade 2 or higher neurotoxicity.

In some embodiments, the agent, e.g., a TRAIL antagonist, e.g., DR5-Fc and/or MAB375-SP or other treatment is administered at a time at which the subject exhibits a sign or symptom of CRS and/or exhibits grade 1 CRS or is administered within 24 hours after the subject exhibits a first sign or symptom of grade 1 CRS following the administration of the therapy. In some embodiments, the sign or symptom of grade 1 CRS is a fever; and/or the agent or other treatment is administered within 24 hours after the first sign of a fever following administration of the therapy.

In some embodiments, also provided are agents, e.g., a TRAIL antagonist, e.g., DR5-Fc and/or MAB375-SP or other treatment use in the treatment, prevention, delay or attenuation of the development of a toxicity in a subject that has been previously administered a therapy, which therapy comprises an immunotherapy and/or a cell therapy, wherein the agent or other treatment is administered within 24 hours of the first sign of a fever following administration of the therapy.

In some embodiments, also provided are agents, e.g., a TRAIL antagonist, e.g., DR5-Fc and/or MAB375-SP or other treatment use in the treatment, prevention, delay or attenuation of the development of a toxicity in a subject that has been previously administered a therapy, which therapy comprises an immunotherapy and/or a cell therapy, wherein the agent or other treatment is administered within 24 hours of the first sign of a fever following administration of the therapy.

In some embodiments, also provided are agents, e.g., a TRAIL antagonist, e.g., DR5-Fc and/or MAB375-SP or other treatment for use as a medicament in treating, preventing, delaying, or attenuating the development of a toxicity in a subject that has been previously administered a therapy, which therapy comprises an immunotherapy and/or a cell therapy. In some embodiments, (a) the agent or other treatment is administered to a subject: (i) at a time that is less than or no more than ten, seven, six, five, four or three days after initiation of the subject having been administered the therapy; and/or (ii) at a time at which the subject does not exhibit a sign or symptom of severe cytokine release syndrome (CRS) and/or does not exhibit grade 2 or higher CRS; and/or (iii) at a time at which the subject does not exhibit a sign or symptom of severe neurotoxicity and/or does not exhibit grade 2 or higher neurotoxicity; and/or (b) between the time of initiation of the subject having been administered the therapy and the time of the administration of the agent or other treatment, (i) the subject has not exhibited severe CRS and/or has not exhibited grade 2 or higher CRS and/or (ii) the subject has not exhibited severe neurotoxicity and/or does not exhibit grade 2 or higher neurotoxicity.

In one embodiment, the subject can be administered an agent which alters the activity of cell therapy, e.g., an immune effector cell, e.g., CAR/TCR-expressing cell. In one embodiment, the subject can be administered an agent which enhances the activity of a bispecific antibody that binds to immune effector cell. For example, in one embodiment, the agent can be a TRAIL antagonist, e.g., a TRAIL antagonist described herein. In another embodiment, the agent can be an inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, and/or DR5. In another embodiment, the agent can be an agent which inhibits an inhibitory molecule. Inhibitory molecules, e.g., Programmed Death 1 (PD-1), can, in some embodiments, decrease the ability of a CAR/TCR-expressing cell to mount an immune effector response. Examples of inhibitory molecules include PD-1, PD-Ll, CTLA-4, TIM-3, CEACAM (e.g., CEACAM-1, CEACAM-3 and/or CEACAM-5), LAG-3, VISTA, BTLA, TIGIT, LAIR1, CD160, 2B4 and TGF beta. Inhibition of an inhibitory molecule, e.g., by inhibition at the DNA, RNA or protein level, can optimize a CAR-expressing cell performance. In embodiments, an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used to inhibit expression of an inhibitory molecule in the CAR-expressing cell. In an embodiment the inhibitor is an shRNA. In an embodiment, the inhibitory molecule is inhibited within a CAR/TCR-expressing cell. In these embodiments, a dsRNA molecule thatinhibits expression of the inhibitory molecule is linked to the nucleic acid that encodes a component, e.g., all of the components, of the CAR/TCR. In one embodiment, the inhibitor of an inhibitory signal can be, e.g., an antibody or antibody fragment that binds to an inhibitory molecule. For example, the agent can be an antibody or antibody fragment that binds to PD-1, PD-Ll, PD-L2 or CTLA4 (e.g., ipilimumab (also referred to as MDX-010 and MDX-101, and marketed as Yervoy@; Bristol-Myers Squibb; Tremelimumab (IgG2 monoclonal antibody available from Pfizer, formerly known as ticilimumab, CP-675,206).). In an embodiment, the agent is an antibody or antibody fragment that binds to TIM3. In an embodiment, the agent is an antibody or antibody fragment that binds to CEACAM (CEACAM-1, CEACAM-3, and/or CEACAM-5). In an embodiment, the agent is an antibody or antibody fragment that binds to LAG3.

The present disclosure also relates to methods for prevention and treatment of immune disorders, such as Rheumatoid Arthritis, Systemic-onsetjuvenile idiopathic arthritis, Still's disease, Macrophage activation syndrome, Hemophagocytic lymphohistiocytosis (HLH), systemic lupus erythematosus (SLE), Kawasaki disease, inflammatory bowel disease by interfering with and/or blocking the activity of TRAIL (TNFSF10) or its receptors, e.g., DR5. In particular, the present disclosure relates to methods for prevention and treatment of immune disorders caused by macrophage activation. In some embodiments, the present disclosure relates to methods for prevention and treatment of immune disorders caused by macrophage activation induced by T cells.

In an embodiment, the subject is administered a TRAIL antagonist, e.g. TRAIL antagonists described herein, to manage elevated levels of a soluble factor (e.g., cytokines) resulting from activation of macrophage and monocytes. In an embodiment, the subject is administered a TRAIL antagonist to manage elevated levels of a soluble factor (e.g., cytokines) resulting from activation of macrophages and monocytes by immune effector cells, e.g. T cells. An example of a TRAIL antagonist is a neutralizaing antibody against TRAIL e.g. MAB375-SP. In an embodiment, MAB375-SP is administered to a subject for the prevention or treatment of Rheumatoid Arthritis, Systemic-onset juvenile idiopathic arthritis, Still's disease, Macrophage activation syndrome, Hemophagocytic lymphohistiocytosis (HLH), systemic lupus erythematosus (SLE), Kawasaki disease, inflammatory bowel disease at a dose of about 5 mg/kg (e.g. 5 mg/kg, 10 mg/kg, 20 mg/kg, 50 mg/kg, 100/kg mg) by subcutaneous or intravenous injection. In some embodiments, MAB375-SP is administered by intra-thecal or intra-ventricular injection to prevent or treat neurotoxicity associated with immune disorders, e.g., macrophage activation syndrome. In some embodiment, the dose of MAB375-SP for intrathecal or intra-ventricular injection is about 20 mg (e.g., 20 mg, 50 mg, 100 mg) every week. In an embodiment, more than one dose of MAB375-SP is administered in case of no response to first dose. In an embodiment, MAB375-SP is administered about once every week, (e.g., once every week, twice every week, three times every week, once every two weeks, once every month etc.). In an embodiment, MAB375-SP is administered prophylactically, i.e., to prevent the flare of immune disorders. In other embodiment, MAB375-SP is administered to treat immune disorders, e.g., immune disorders described herein, e.g., Rheumatoid Arthritis, Systemic-onset juvenile idiopathic arthritis, Still's disease, Macrophage activation syndrome, Hemophagocytic lymphohistiocytosis (HLH), systemic lupus erythematosus (SLE), Kawasaki disease and inflammatory bowel disease. In yet other embodiment, MAB375-SP is administered at the earliest signs and symptoms of a flare of an immune disorder, such as fever >38.5° C., drop in systolic or diastolic blood pressure of more than 10 mm Hg, systolic blood pressure of <100 mm Hg or diastolic blood pressure of <70 mm Hg. In an embodiment, MAB375-SP is administered as a monotherapy. In other embodiments, MAB375-SP is administered in combination with other agents, e.g., corticosteroids, DR5-Fc, tocilizumab or anakinra. Another example of a TRAIL antagonist is an antibody or a protein that binds to DR5 and prevent its binding to TRAIL. An example of a TRAIL antagonist is an antibody or a protein that binds to TRAIL and prevent or compete with its binding to DR5. An example of a protein that binds to TRAIL and compete with its binding to DR5 is DR5-Fc. In an embodiment, DR5-Fc is administered to a subject for the prevention or treatment of an immune disorders, e.g., immune disorders described herein, e.g., Rheumatoid Arthritis, Systemic-onset juvenile idiopathic arthritis, Still's disease, Macrophage activation syndrome, Hemophagocytic lymphohistiocytosis (HLH), systemic lupus erythematosus (SLE), Kawasaki disease and inflammatory bowel disease at a dose of about 10 mg (e.g. 10 mg, 20 mg, 50 mg, 100 mg) by subcutaneous or intravenous injection. In an embodiment, DR5-Fc is administered about once weekly, (e.g., once weekly, twice weekly, or three times every week). In some embodiments, DR5-Fc is administered by intra-thecal or intra-ventricular injection to prevent or treat neurotoxicity associated with an immune disorder. In some embodiment, the dose of DR5-Fc for intrathecal or intra-ventricular injection is about 20 mg (e.g., 20 mg, 50 mg, 100 mg) every week. In some embodiments, DR5-Fc is administered by intra-articular injection. In an embodiment, more than one dose of DR5-Fc is administered in case of no response to first dose. In an embodiment, DR5-Fc is administered as a monotherapy. In other embodiments, DR5-Fc is administered in combination with other agents, e.g., MAB375-SP, corticosteroids, tocilizumab or anakinra. Other examples of proteins that bind to TRAIL and compete with its binding to DR5 are known in the art, e.g., DR4-Fc, DcR1-Fc, DcR2-Fc, and can be used in alternate embodiments of the disclosure. In some embodiments, a TRAIL antagonist is chosen from
i) A neutralizing antibody against TRAIL
ii) An antagonistic antibody against DR5 (TRAIL-R2)
iii) A competitive antagonist of DR5 (e.g., DR5-Fc)
iv) A protein capable of binding TRAIL and competes for its binding to DR5
v) A nucleic acid inhibitor of TRAIL and/or DR5
vi) A small molecule inhibitor of TRAIL and/or DR5
vii) A combination of the above

Pharmaceutical compositions of the disclosure may comprise a cell therapy product, e.g., CAR/TCR-expressing product, e.g., a plurality of CAR/TCR-expressing cells, as described herein, in combination with one or more pharmaceutically or physiologically acceptable carriers, diluents or excipients. Pharmaceutical compositions of the disclosure may comprise a small molecule inhibitor, a polypeptide inhibitor or a nucleic acid inhibitor of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL (TNFSF10) and Death Receptor 5 (DR5 or TNFRSF10B).

Pharmaceutical compositions of the disclosure may comprise a bispecific or multispecific engager. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure are in one aspect formulated for intravenous administration.

Pharmaceutical compositions of the disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus.

When “an immunologically effective amount,” “an anti-tumor effective amount,” “a tumor-inhibiting effective amount,” or “therapeutic amount” is indicated, the precise amount of the compositions of the disclosure to be administered can be determined by a physician with consideration of individual differences in age, weight, tumor size, extent of infection or metastasis, and condition of the patient (subject). It can generally be stated that a pharmaceutical composition comprising the immune effector cells (e.g., T cells, NK cells) described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally.

In one aspect, the T cell compositions of the disclosure are administered by i.v. injection. The compositions of immune effector cells (e.g., T cells, NK cells) may be injected directly into a tumor, lymph node, or site of infection.

In aparticular exemplary aspect, subjects may receive a mobilization agent (e.g., a mobilization agent described herein, e.g., a CXCR4 antagonist) and then undergo leukapheresis, wherein leukocytes are collected, enriched, or depleted ex vivo to select and/or isolate the cells of interest, e.g., T cells. These T cell isolates may be expanded by methods known in the art or the methods described in this disclosure and treated such that one or more CAR/TCR constructs and accessory modules (e.g., constitutive active mutants of JAK3, STAT5 and/or inhibitor of BRD9 etc.) of the disclosure may be introduced, thereby creating a cell therapy product of the disclosure. The CAR/TCR cells may be activated and/or expanded in vitro using bispecific/multispecific engagers. Subjects in need thereof may subsequently undergo standard treatment with high dose chemotherapy followed by peripheral blood stem cell transplantation. In certain aspects, prior to, following or concurrent with the transplant, subjects receive an infusion of the expanded cell therapy product (e.g. CAR/TCR T cells) of the disclosure. In an additional aspect, expanded cells are administered before or following surgery. The subject may receive bispecific/multispecific engagers with or without costimulatory agents (e.g., 41BBL or Utomilumab) following administration of T cells to allow in vivo expansion of the administered cells. The subject may further receive a TRAIL antagonist to prevent, delay, attenuate or treat toxicitiy of infused cell therapy products.

The dosage of the above treatments to be administered to a patient will vary with the precise nature of the condition being treated and the recipient of the treatment. The scaling of dosages for human administration can be performed according to art-accepted practices.

In one embodiment, the CAR/TCR is introduced into immune effector cells (e.g., T cells, NK cells), e.g., using in vitro transcription, and the subject (e.g., human) receives an initial administration of CAR/TCR immune effector cells (e.g., T cells, NK cells) of the disclosure, and one or more subsequent administrations of the CAR/TCR immune effector cells (e.g., T cells, NK cells) of the disclosure, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the CAR/TCR immune effector cells (e.g., T cells, NK cells) of the disclosure are administered to the subject (e.g., human) per week, e.g., 2, 3, or 4 administrations of the CAR/TCR immune effector cells (e.g., T cells, NK cells) of the disclosure are administered per week. In one embodiment, the subject (e.g., human subject) receives more than one administration of the CAR/TCR immune effector cells (e.g., T cells, NK cells) per week (e.g., 2, 3 or 4 administrations per week) (also referred to herein as a cycle), followed by a week of no CAR/TCR immune effector cells (e.g., T cells, NK cells) administrations, and then one or more additional administration of the CAR/TCR immune effector cells (e.g., T cells, NK cells) (e.g., more than one administration of the CAR/TCR immune effector cells (e.g., T cells, NK cells) per week) is administered to the subject. In another embodiment, the subject (e.g., human subject) receives more than one cycle of CAR/TCR immune effector cells (e.g., T cells, NK cells), and the time between each cycle is less than 10, 9, 8, 7, 6, 5, 4, or 3 days. In one embodiment, the CAR/TCR immune effector cells (e.g., T cells, NK cells) are administered every other day for 3 administrations per week. In one embodiment, the CAR/TCR immune effector cells (e.g., T cells, NK cells) of the disclosure are administered for at least two, three, four, five, six, seven, eight or more weeks.

In one aspect, CAR/TCR-expressing cells of the disclosures are generated using lentiviral viral vectors, such as lentivirus. Cells, e.g., CAR/TCR Ts, generated that way will have stable-expression.

In one aspect, CAR/TCR-expressing cells, e.g., CARTs, are generated using a viral vector such as a gammaretroviral vector, e.g., a gammaretroviral vector described herein. CARTs generated using these vectors can have stable CAR expression.

In one aspect, CARTs transiently express CAR vectors for 4, 5, 6, 7, 8, 9, 10, 11, 12, 13, 14, 15 days after transduction. Transient expression of CARs/TCRs can be effected by RNA CAR vector delivery. In one aspect, the CAR/TCR RNA is transduced into the T cell by electroporation.

A potential issue that can arise in patients being treated using transiently expressing CAR immune effector cells (e.g., T cells, NK cells) (particularly with murine scFv bearing CARTs) is anaphylaxis after multiple treatments.

Without being bound by this theory, it is believed that such an anaphylactic response might be caused by a patient developing humoral anti-CAR response, i.e., anti-CAR antibodies having an anti-IgE isotype. It is thought that a patient's antibody producing cells undergo a class switch from IgG isotype (that does not cause anaphylaxis) to IgE isotype when there is a ten to fourteen day break in exposure to antigen.

If a patient is at high risk of generating an anti-CAR antibody response during the course of transient CAR therapy (such as those generated by RNA transductions), CART infusion breaks should not last more than ten to fourteen days.

### EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Methods for generation and characterization of CAR-T cells, including generation of lentivirus and retroviruses, infection of T cells and PBMC, culture and expansion of T cells, in vitro assays for T cell function, such as ELISA, Flow cytometry, cell death assays (e.g., Matador assay), antigen detection assay (e.g., Topanga assay) and in vivo assays are known in the literature and have been described in WO 2018/102795 which is incorporated in its entirety by reference herein.

Generation of Lentiviruses and retroviruses. Lentiviruses were generated by calcium phosphate based transfection in 293FT cells essentially as described previously (Matta, Hozayev, Tomar, Chugh, & Chaudhary, 2003).

Infection of T cells and PBMC. Unless indicated otherwise, buffy coat cells were obtained from healthy de-identified adult donors from the Blood Bank at Children Hospital of Los Angeles and used to isolate peripheral blood mononuclear cells (PBMC) by Ficoll-Hypaque gradient centrifugation. PBMC were either used as such or used to isolate T cells using CD3 magnetic microbeads (Miltenyi Biotech) and following the manufacturer's instructions. PBMC or isolated T cells were re-suspended in XVIVO medium (Lonza) supplanted with 10 ng/ml CD3 antibody, 10 ng/ml CD28 antibody and 100 IU recombinant human-IL2. Alternatively, CD3/CD28 beads and 100 IU recombinant human-IL2 were used. Cells were cultured at 37° C., in a 5% CO₂ humidified incubator. Cells were activated in the above medium for 1 day prior to infection with lentiviral vectors. In general, primary cells (e.g. T cells) were infected in the morning using spin-infection (1800 rpm for 90 minutes at 37° C. with 300 μl of concentrated virus that had been re-suspended in XVIVO medium in the presence of 8 μg/ml of Polybrene® (Sigma, Catalog no. H9268). The media was changed in the evening and the infection was repeated on consecutive days for a total of 2-3 infections. After the last infection, the cells were pelleted and resuspended in fresh XVIVO media containing 10 ng/ml CD3 antibody, 10 ng/ml CD28 antibody and 100 IU recombinant human-IL2 and supplemented with respective antibiotics (if indicated) and placed in the cell culture flask for selection, unless indicated otherwise. Alternatively, CD3/CD28 beads and 100 IU recombinant human-IL2 were used. Cells were cultured in the above medium for 10-15 days in case no drug selection was used and for 20-30 days in case drug-selection was used. In cases, where cells were infected with a lentivirus expressing EGFP, they were expanded without drug-selection or flow-sorted to enrich for EGFP-expressing cells. For infection of cancer cell lines, approximately 500,000 cells were infected with 2 ml of the un-concentrated viral supernatant in a total volume of 3 ml with Polybrene® (Sigma, Catalog no. H9268). Then next morning, the cells were pelleted and resuspended in the media with respective antibiotics and place in the cell culture flask for selection.

Antibodies and drug. Digitonin was purchased from Sigma (Cat. no D141) and a stock solution of 100 mg/ml was made in DMSO. A diluted stock of 1 mg/ml was made in PBS. Final concentration of digitonin used for cell lysis was 30 μg/ml unless indicated otherwise.

Clinical Grade CAR-T Manufacturing and Administration

For clinical grade CAR-T manufacturing, cGMP grade lentiviruses encoding the CARs are generated using commercial sources (e.g., Lentigen, Lonza etc.). The T cell are collected from donors (autologous or allogeneic) using leukapheresis. CAR-T cells are manufactured using CLINIMAC Prodigy (Miltenyi Biotech) automated closed system as described (Zhu F, Shah N et al, Cytotherapy, 2017) and following the instructions of the manufacturer. The multiplicity of infection (MOI) of between 5 to 10 is used. Alternate methods for clinical grade CAR-T manufacturing, such as Cocoon (Lonza) and manual open systems, are known in the art and can be used in alternate embodiment of the invention. CAR-T cells are administered to the patient after lympho-depleting chemotherapy at escalating doses starting at approximately of 1×106 CD3 CAR-T cells/kg.

IL2 ELISA. Human IL2, IFNγ, IL6 and TNFα was measured in the cell culture supernatant of CAR-expressing Jurkat-NFAT-GFP effector cells or T cells that had been co-cultured with the specific target cell lines for 24 to 96 hours using ELISA kits from R&D systems (Minneapolis, Minn.) and following the recommendations of the manufacturer.

FACS analysis. Mouse Anti-Human c Myc APC conjugated Monoclonal Antibody (Catalog #IC3696A) was from R&D Systems (Minneapolis, Minn.). Biotinylated protein L was purchased from GeneScript (Piscataway, N.J.), reconstituted in phosphate buffered saline (PBS) at 1 mg/ml and stored at 4° C. Streptavidin-APC (SA1005) was purchased from ThermoFisher Scientific.

For detection of CARs using Myc staining, 1×10⁶ cells were harvested and washed three times with 3 ml of ice-cold 1×PBS containing 4% bovine serum albumin (BSA) wash buffer. After wash, cells were resuspended in 0.1 ml of the ice-cold wash buffer containing 10 μl of APC-conjugated Myc antibody and incubated in dark for 1 hour followed by two washings with ice cold wash buffer.

For detection of CARs using Protein L staining, 1×10⁶ cells were harvested and washed three times with 3 ml of ice-cold 1×PBS containing 4% bovine serum albumin (BSA) wash buffer. After wash, cells were resuspended in 0.1 ml of the ice-cold wash buffer containing 1 μg of protein L at 4° C. for 1 hour. Cells were washed three times with ice-cold wash buffer, and then incubated (in the dark) with 10 μl of APC-conjugated streptavidin in 0.1 ml of the wash buffer for 30 minutes followed by two washings with ice cold wash buffer. FACS was done using FACSVerse analyzer from BD Biosciences.

Cell death assay. To measure cell death, a novel assay based on ectopic cytosolic expression of Gluc, NLuc or LucPPe was utilized as described in PCT/US17/52344 “A Non-Radioactive Cytotoxicity Assay”.

Unless indicated otherwise, for measurement of LucPPe-146-1H2 activity, a 10× Luciferin stock solution was prepared consisting of 1 mM D-luciferin synthetic crystalline (Sigma), 25 mM glycylglycine, pH 7.8. A stock solution of luciferin assay buffer was prepared containing 25 mM glycylglycine, pH 7.8, 15 mM potassium phosphate, pH 7.8, 15 mM MgSO4, 4 mM EGTA, 2 mM ATP (Sigma). Working solution of Luciferase assay buffer for each 1.0 ml consisted of 885.5 μl assay buffer+1 μl DTT (1M stock)+100 μl of 10× Luciferin stock solution+13.5 μl ATP (100 mM stock). The assay buffer containing substrate was generally added to the media containing cells at 1:1 (v/v) ratio, unless indicated otherwise.

Cell lines engineered to express luciferases (e.g., GLuc, NLuc, LucPPe) for measuring cytotoxicity of different constructs targeting different cell surface and intracellular antigens using the Matador cytotoxicity assay are provided in Table A. Cell lines used in this experiments, target antigens on the cells lines and their growth media are shown in the following Table A. These cell lines can be also used as antigen presenting cells (APC) for activation/expansion of immune cells expressing CARs/TCRs targeting different antigens in the absence and presence of different bispecific/multispecific engagers of the disclosure. For example, CD19+ REC-1 cells can be used as APC for CARs targeting CD19. Alternatively, REC-1 cells can be used as APC for immune cells, e.g., T cells or Mesothelin CAR-T cells or NYESO-1-TCR-T cells, in the presence of Blinatumomab. Cells were cultured at 37° C., in a 5% CO₂ humidified incubator. The cell lines were obtained from ATCC, NIH AIDS reagent program or were available in the laboratory.

TABLE A

| Cell line | Culture Conditions | Exemplary CAR Target Antigens Expressed |
|---|---|---|
| BC-1 | RPMI, 20% FCS | BCMA, GPRC, CD138 |
| BC-3 | RPMI, 20% FCS | BCMA, GPRC, CD138 |
| BCBL-1 | RPMI, 20% FCS | GPRC, CD138 |
| JSC-1 | RPMI, 20% FCS | GPRC, CD138 |
| MM1S | RPMI, 10% FCS | CD38, GPRC, CD44, CD200R |
| U266 | RPMI, 10% FCS | BCMA, WT1/HLA-A2+, CS1, CLL1, CD138, |
| | | c-MET, IL6R, CD179b, NY-ESO/HLA-A2, |
| | | NYBR, LAMP1 |
| L363 | RPMI, 10% FCS | BCMA, GPRC, WT1/HLA-A2+, CS1, CLL1, |
| | | CD138, NY-ESO/HLA-A2, NYBR, LAMP1 |
| K562 | RPMI, 10% FCS | CD33, IL1Ra, TnAg |
| BV173 | RPMI, 10% FCS | CD123, CD179b, IL1Ra, WT1/HLA-A2+, |
| | | CXCR4, FLT3, CD179a |
| Nalm6 | RPMI, 10% FCS | CD19, CD20, CD22, CD179b, CD179a |
| HL60 | RPMI, 10% FCS | CD33, CD34, CLL1, IL6R, CD32, CD179 |
| U937 | RPMI, 10% FCS | CD4, CLL1 |
| RS: 411 | RPMI, 20% FCS | CD19, Folate Receptor beta (FRbeta), |
| | | TGFbeta, CD179b, NKG2D, FLT3, CD179a |
| MV: 411 | RPMI, 10% FCS | FLT3, CD123, FRbeta |
| Raji | RPMI, 10% FCS | CD19, CD20/MS4A1, CD22, BCMA, CD38, |
| | | CD70, CD79, Folate Receptor beta, CLL1 |
| HEL-92.1.7 | RPMI, 10% FCS | MPL, CD33, CD32, CD200R |
| (HEL) | | |
| Jurkat | RPMI, 10% FCS | TnAg, TSLRP, TSHR, CD4, CD38 |
| Daudi | RPMI, 10% FCS | BCMA, FRbeta |
| REC-1 | RPMI, 10% FCS | NKG2D, ROR1, CD19, CD20/MS4A1, CD22 |
| KG-1 | RPMI, 20% FCS | CD33, CD34, CD123, TSLRP |
| CEM | RPMI, 10% FCS | CD5, CD43 |
| U937 | RPMI, 10% FCS | CD4, CLL1 |
| LAMA5 | RPMI, 10% FCS | WT1/HLA-A2 |
| A549 | DMEM, 10% FCS | ROR1, CD22, TIM1, CDH17 |
| HT29 | DMEM, 10% FCS | EGFR, SLEA, c-MET |
| Molm-13 | RPMI, 20% FCS | FLT3, IL6R, LAMP1, TSLRP, CD4, |
| | | CSF2RA, CXCR4, IL6R, CSF2RA, GPC3 |
| A431 | DMEM, 10% FCS | EGFR, Folate Receptor Alpha, Her3 |
| P19 | DMEM, 10% FCS | SSEA |
| THP-1 | RPMI, 10% FCS | CD32, CD33, CXCR4, CD123, CD44, IL6R, |
| | | Folate Receptor beta, CD70, LAMP1, |
| | | FLT3, CSF2RA |
| U87MG | DMEM, 10% FCS | CD276, gpNMB, IL13RA2 |
| LoVo | DMEM, 10% FCS | Tissue Factor, CDH17, EGFR |
| SKOV-3 | DMEM, 10% FCS | Folate Receptor alpha (FR1), FSHR, |
| | | Her2, Her3, LHR, MSLN, TIM1, EPCAM |
| NCI-H1993 | DMEM, 10% FCS | EGFR |
| Kasumi-1 | RPMI, 20% FCS | CLEC5A, PR1/HLA-A2, TGFbeta, |
| Jeko-1 | RPMI, 20% FCS | BCMA, ROR1, CD20/MS4A1, CD22, CD19 |
| JVM-2 | RPMI, 20% FCS | BCMA, ROR1, CD20/MS4A1, CD22, CD19 |
| MINO | RPMI, 20% FCS | BCMA, CD20/MS4A1, CD19 |
| GRANTA-519 | RPMI, 20% FCS | CD20/MS4A1, CD22, CD19 |
| PC-3 | DMEM, 10% FCS | CGH, TROP2, PSCA, PSMA. EPCAM, FSHR, |
| | | CLD18A2 (CLDN18.2) |
| HeLa | DMEM, 10% FCS | EGFR, FR1, MSLN, TSHR |
| LnCap | DMEM, 10% FCS | EGFR, FSHR, PSCA, PSMA, CD22, Her3, |
| | | CD22, LHR, CLD18A2 (CLDN18.2) |
| OVCAR-3 | DMEM, 10% FCS | B7H4, CDH6, DLL3, FR1, FSH, LHR, MSLN, |
| | | PTK7, TnAg, TSHR, L1CAM |
| MEL-624 | DMEM, 10% FCS | CDH19, GD2, GD3, gp100/HLA-A2, gpNMB, |
| | | HMWMAA, NYESO/HLA-A2, MART1/HLA-A2 |
| LS174-T | DMEM, 10% FCS | CEA |
| MEL-526 | DMEM, 10% FCS | GD2 |
| MDA-MB231 | DMEM, 10% FCS | CD324, Muc1 |
| L1236 | RPMI, 20% FCS | CD30, CD23, PDL1 |
| L428 | RPMI, 20% FCS | CD30, CD123, CCR4, PDL1 |
| L540 | RPMI, 20% FCS | CD30, CCR4, PDL1 |
| Molt-16 | RPMI, 20% FCS | IL1ra, NKG2DNKG2D |
| CEM | RPMI, 10% FCS | CD5 |
| MG-63 | DMEM, 10% FCS | IL13RA2 |
| Karpass-299 | RPMI, 20% FCS | Alk, GPRC, PDL1 |
| MCF7 | DMEM, 10% FCS | B7D4, CD276, TROP2, Her3, Muc1, |
| | | LewisY, LHR |
| AA-2 | RPMI, 10% FCS | HIV1 env glycoprotein (gp120) |
| HL2/3 | DMEM, 10% FCS | HIV1 env glycoprotein (gpl20) |
| TF228.1.16 | DMEM, 10% FCS | HIV1 env glycoprotein (gp120), CCR4 |
| TT | DMEM, 10% FCS | TGF-Beta, TSHR, GFRalpha4 |
| DMS79 | RPMI, 10% FCS | Fucosyl-GM1, Slea (CA19.9; Sialyl |
| | | Lewis Antigen) |
| LAN-5 | DMEM, 10% FCS | ALK, DLL3, GFRalpha4, FUCOSYL-GM1 |
| PEER1 | RPMI, 10% FCS | TSHR |
| SK-MEL-37 | DMEM, 10% FCS | DLL3, GD2 |
| F9 | DMEM, 10% FCS | SSEA |
| HepG2 | DMEM, 10% FBS | GPC3, AFP/HLA-A2 |

Jurkat cell line (clone E6-1) engineered with a NFAT-dependent GFP reporter gene was a gift from Dr. Arthur Weiss at UCSF. Jurkat cells were maintained in RPMJ-1640 medium supplemented with 10% FBS, penicillin and streptomycin.

Guide RNA Molecules

gRNA molecules comprising the targeting sequences listed in Table 8 were used for the experiments described in this subexample. Unless otherwise indicated, all gRNA molecules were tested as dual gRNA molecules comprising the tracr and crRNA sequences described in this subexample.

TABLE 8

| | | gRNA Target |
|---|---|---|
| | Target | Sequence |
| TARGET GENE | Gene ID | SEQ ID NO: |
| BRD9 | 65980 | 1936-1939 |
| EZH2 | 2146 | 1940-1943 |
| KMT2D (MLL2) | 8085 | 1944-1947 |
| KMT2C (MLL3) | 58508 | 1948-1951 |
| KMT2B (MLL4) | 9757 | 1952-1955 |
| BRD8 | 10902 | 1956-1959 |
| CHEK2 | 11200 | 1960-1963 |
| TET1 | 80312 | 1964-1967 |
| TET2 | 54790 | 1968-1971 |
| TET3 | 200424 | 1972-1975 |
| ATM | 472 | 1976-1979 |
| FAT1 | 2195 | 1980-1983 |
| FBXW10 | 10517 | 1984-1987 |
| BCOR | 54880 | 1988-1991 |
| ASXL1 | 171023 | 1992-1995 |
| PHF6 | 84295 | 1996-1999 |
| KDM6A | 7403 | 2000-2003 |
| SF3B1 | 23451 | 2004-2007 |
| YY1 | 7528 | 2008-2011 |
| CBFB | 865 | 2012-2015 |
| Runx1 | 861 | 2016-2019 |
| PRDM1 (BLIMP1) | 639 | 2020-2023 |
| HDAC2 | 3066 | 2024-2027 |
| EHMT2 (G9A) | 10919 | 2028-2031 |
| SMARCA4 | 6597 | 2032-2035 |
| CREBBP | 1387 | 2036-2039 |
| TNFSF10 | 8743 | 2040-2043 |
| TNFRSF10A | 8797 | 2044-2047 |
| TNFRSF10B | 8795 | 2048-2051 |

Generation of CRISPR CAR T Cells

Isolated and frozen Pan T cells are thawed and activated with CD3/CD28 beads (CD3/CD28 CTS Dynabeads® 43205D) on day 0. Activated T cells are transduced with lentivirus encoding either a CD19 CAR (SEQ ID NO: 2822) or a CD20 CAR (SEQ ID NO: 2824) CAR) on day 1. On day 3, transduced CART cells are electroporated to introduce CRISPR/Cas systems in the form of pre-complexed gRNA/Cas9 ribonuclear protein (“RNP”). To form RNP, all RNA samples are heated at 95 C. S. pyogenes CAS9 Protein (NLS CAS9 iPROT106154, 37 μM) is diluted in buffer before tracrRNA (having the sequence AACAGCAUAGCAAGUUAAAAUAAGGCUAGUCCGUUAUCAACUUGAAAAAGUG GC ACCGAGUCGGUGUUUUUUU (SEQ ID NO: 2054); AXO Labs) is added to it. After mixing CAS9 Protein with tracrRNA, the CRISPR RNA is added (in each case, each crRNA comprised the sequence nnnnnnnnnnnnnnnnnnnnGUUUUAGAGCUAUGCUGUUUUG (SEQ ID NO: 2052), where the n residues represent the 20 ribonucleic acid residues of the indicated targeting sequence targeting the different genes shown in Table 8). The precomplexed RNPs are then added to a total of 1 million cells, RNP concentration is 3.2 μM. Electroporation is done by neon electroporator using Neon® Transfection System 100 μL Kit (MPKI0096) at 1600V, 10 ms, 3 pulses. The cells are kept in culture for 7 more days. Cells are then divided, with some used to perform flow cytometry: Staining for CAR (PE), and different markers, such as CD3 (PerCP-Cy5.5), CD4 (V450) and CD8 (APC), CD45RA, CCR7, CD62L. Remaining T cells are frozen and used for functional assays and next generation sequencing (NGS) sample generation. Other methods for gene editing of T cells using the CRISPR system are known in the art and can be used in alternate enablement of the disclosure (Osborn, M J et al, Molecular Therapy, 24; 3, 2016)

Alternatively, the double stranded oligonucleotides encoding the gRNAs targeting the different genes shown in Table 4a are cloned downstream of a U6 promoter in pLenti-CRISPR-v2 (SEQ ID NO: 359), which is available from Addgene and following the recommendations of the distributor (i.e. Addgene). The vector also encodes for S. pyogenes CAS9 Protein (Sp-NLS-Cas9). The lentiviruses are prepared by transfection of the gRNA encoding lentiviral vectors along with packaging vectors into 293FT cells as per the recommendations of the distributor (Addgene). Isolated and frozen Pan T cells were thawed and activated with CD3/CD28 beads (CD3/CD28 CTS Dynabeads@ 43205D) on day 0. Activated T cells were transduced with the lentiviruses encoding the different gRNA (or empty vector) and lentivirus encoding either a CD19 CAR (SEQ ID NO: 477) or a CD20 CAR (SEQ ID NO: 478) CAR) on day 1. The cells are kept in culture for 7 more days. Cells were then divided, with some used to perform flow cytometry: Staining for CAR (PE), and different markers, such as CD3 (PerCP-Cy5.5), CD4 (V450) and CD8 (APC), CD45RA, CCR7, CD62L. Remaining T cells are frozen and used for functional assays and next generation sequencing (NGS) sample generation.

Essentially a similar procedure is used to generate CRISPR T cells targeting the different genes and expressing TCR or next generation CARs (e.g., SIR, Ab-TCR, TFP etc.).

Generation of shRNA CAR T Cells

To generate shRNA encoding lentiviruses, the double stranded oligonucleotides encoding the shRNAs (SEQ ID NO: 274 to SEQ ID NO: 335) targeting the different genes shown in Table 5 are cloned downstream of U6 promoter in the lentivirus vector pLKO.1 (SEQ ID NO: 357) using standard molecular biology techniques. The lentiviruses are prepared after transfection of the shRNA encoding transfer vector into 293FT cells along with the packaging plasmids following the recommendations of the distributor (Addgene). Activated T cells are infected with the resulting viruses and expanded as describe above. In certain embodiments, the T cells are coinfected with shRNA encoding lentiviruses and CAR-encoding lentiviruses. In some experiments, T cells infected with shRNA encoded lentiviruses are selected with puromycin.

Generation of Lentiviral Expression Vectors Encoding Constitutive Active Mutants of JAK1, JAK3, Stat5b, Stat3, BRAF, IL2RG and CARD11.

cDNA encoding JAK1, JAK3, Stat5b, Stat3, BRAF, IL2RG and CARD11 are obtained from Sinobiological and are mutagenized using PCR-based Quick-Change protocol using custom primers. The mutant clones are confirmed by automated sequencing. The resulting clones serve as templates in PCR to amplify the coding sequence. The amplified PCR fragments are cloned in the lentiviral vector pLENTI-EFlu (SEQ ID NO: 337) or the retroviral vector MSCV-hygro (Clontech). The cDNA encoding the wild type and mutant forms of JAK1, JAK3, Stat5b, Stat3, BRAF, IL2RG and CARD11 are also cloned in lentiviral or retroviral vectors encoding CARs/TCRs and separated from them by 2A sequences.

T Cell Proliferation Assay

CART cell proliferation in response to target cells is evaluated. Target cell lines are listed in Table A. CAR-T cells are thawed incubated for 2 hours in T cell medium to recover. Cells are counted on a Cellometer. Target cells are irradiated at 10,000 rad. After irradiation, target cells are washed twice in complete T cell medium and counted. 30,000 Irradiated target cells are then co cultured with CART cells at 1:1 ratio. As a negative control, medium alone is added to CART cells.

The co-culture is incubated for 4 days at 37° C. On Day 4, coculture cells are stained for 20 mins on ice with CD3-percp cy5.5 (Ebioscience:45-0037), CD4-eflor450(Ebioscience:48-0047), and CD8-APC (Ebioscience 17-0087) and measured by flow cytometry relative to CountBright Absolute Counting Beads (Life Technologies) to determine relative cell counts.

CAR expression is measured by two step incubation of 20 mins each on ice: Biotinylated-Protein L+Streptavidin-PE (Jackson immuno research). Flow cytometry data is acquired using BD 5 laser Fortessa and analyzed by FlowJo software.

Bispecific Antibody Expression and Purification

DNA fragments encoding the bispecific antibiody sequences (SEQ ID NO: 2470-2645) are synthesized by IDT and clones in the pcDNA3.1 vector (ThermoFisher). The constructs are sequence confirmed using Automated Sanger Sequencing. Chinese hamster ovary (CHO) cells are transfected with the bispecific antibody expression vectors, and then cultured for 7 days for bispecific antibody production. CHO cell supernatants containing secreted bispecific antibody molecules are collected. Bispecific antibodies arre purified using HisTrap HP column (GE healthcare) by FPLC AKTA system. Briefly, CHO cell culture is clarified and loaded onto the column with low imidazole concentration (20 mM), and then an isocratic high imidazole concentration elution buffer (500 mM) is used to elute the bound bispecific proteins.

Generation and Characterization of CAR-T Cells from Perixafor Mobilized Cells

A patient with blood cancer was administered G-CSF (Neupogen) at dose of 10 μg/kg daily from days 1-5 by subcutaneous injection and Perixafor at a dose of 0.24 mg/kg subcutaneously daily on days 3-5. A leukapheresis product was collected from the patient 6 h after the last dose of Perixafor. The CD3+ T cells were isolated from the leukapheresis product using magnetic beads (Miltenyi Biotech) and following the manufacturer's instructions. T cells were re-suspended in XVIVO medium (Lonza) supplanted with 10 ng/ml CD3 antibody, 10 ng/ml CD28 antibody and 100 IU recombinant human-IL2. T cells were infected twice with three different lentiviral vectors encoding chimeric antigen receptors targeting CD19; a) FMC63-BBz-2A-PAC; b) FMC63-MYC-CD8TM-BBZ-T2A-eGFP and c) CD19-hu-mROO5-1-CD8TM-BBZ-T2A-eGFP using polybrene. The sequence of the CAR FMC63-BBz-2A-PAC is presented in SEQ ID NO: 478. This CAR construct is a 2nd generation CAR construct containing FMC63 scFv as the antigen binding domain, 4-1BB costimulatory domain and a CD3z activation domain. The construct also co-expresses a puromycin resistance gene (PAC) via a 2A cleavable linker. The CAR construct FMC63-MYC-CD8TM-BBZ-T2A-eGFP is similar in design to the construct FMC63-BBz-2A-PAC with the exception that the PAC gene is replace by EGFP (enhanced Green Fluorescent Protein). Finally, the construct CD19-hu-mROO5-1-CD8TM-BBZ-T2A-eGFP is similar in design to the construct FMC63-MYC-CD8TM-BBZ-T2A-eGFP with the exception that the FMC63 scFv is replaced by CD19-hu-mROO5 scFv, which also targets CD19. The CAR FMC63-BBz-2A-PAC was cloned in the pLENTI-EF1 vector (SEQ ID NO: 337), while the other two CARs were cloned in the pHAGE vector (Addgene Cat #24527). Following infection with the CAR constructs, the T cells were expanded in in XVIVO medium (Lonza) supplanted with 10 ng/ml CD3 antibody, 10 ng/ml CD28 antibody and 100 IU recombinant human-IL2. The cells transduced with the FMC63-BBz-2A-PAC CAR construct were also selected with puromycin for 12 days.

The T cells transduced with the CAR constructs FMC63-MYC-CD8TM-BBZ-T2A-eGFP and CD19-hu-mROO5-1-CD8TM-BBZ-T2A-eGFP were expanded without drug selection and monitored for transduction efficiency by flow cytometry. At day 8, approximately 37.2% and 42.8% of T cells infected with the the CAR constructs FMC63-MYC-CD8TM-BBZ-T2A-eGFP and CD19-hu-mROO5-1-CD8TM-BBZ-T2A-eGFP showed EGFP expression, respectively. At day 24, approximately 34.4% and 60.8% of T cells infected with the the CAR constructs FMC63-MYC-CD8TM-BBZ-T2A-eGFP and CD19-hu-mROO5-1-CD8TM-BBZ-T2A-eGFP showed EGFP expression, respectively. These results demonstrate that Perixafor mobilized cells can be used to generate CAR-T cells and these cells can be expanded in vitro without undergoing exhaustion.

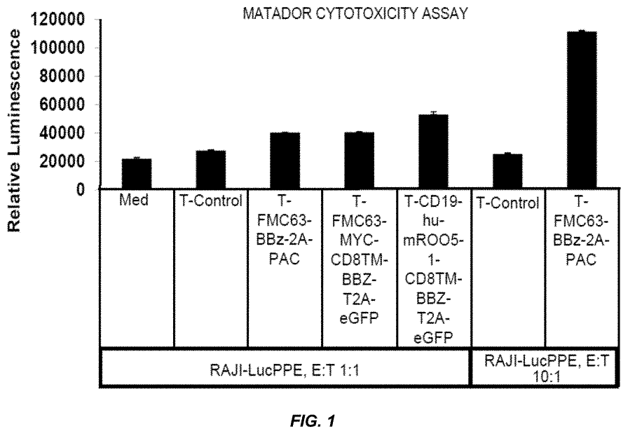

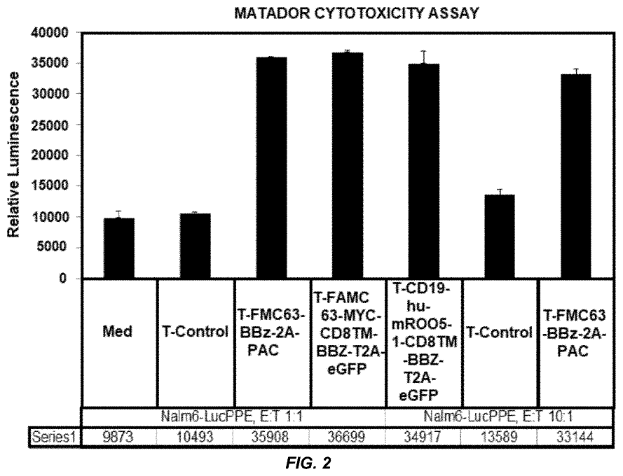

To examine whether CAR-T cells generated from Perixafor mobilized cells are functionally active, after 10 days in culture, approximately 3×104 CAR-T cells or control T cells were co-cultured with equal number of RAJI or NALM6 cells stably expressing LucPPe-146-1H2 (LucPPe) in a 384 well plate in 60 μl of XVIVO medium in triplicate for 4 hours. Alternatively, cells were cultured at an E;T ratio of 10:1. Cellular toxicity was measured by addition of D-luciferin as described in PCT/US17/52344 “A Non-Radioactive Cytotoxicity Assay”. Treatment with Digitonin was used to measure maximum cell death. FIG. 1 shows effective induction of death of RAJI cells, as measured by increase in luminescence, by all three CAR constructs. Similarly, FIG. 2 shows effective induction of death of NALM6 cells, as measured by increase in luminescence, by all three CAR constructs. These results demonstrate that CAR-T cells can be generated from Perixafor mobilized blood cells and such cells demonstrate cytotoxicity against cells expressing their target antigen.

In a separate experiment, the supernatant from the co-culture experiment is collected after 24 hours and assayed for induction of cytokines. It is observed that co-culture of CAR-T cells with the RAJI cells results in increased production of IFNγ, TNFα and IL2 as compared to co-culture with parental T cells.

The CAR-T cells are analyzed for the expression of different markers (e.g., CD4, CD8, CD62L, P-glycoprotein, CD25, CD127 and FoxP3 etc.) by flow cytometry. No major increase in the proportion of TREGs (CD3+, CD4+, CD25high, CD127low, FoxP3+) cells is observed, suggesting that CAR-T cells can be generated from Perixafor mobilized blood cells without over-growth of TREGs. In addition, the proportion of CD4 and CD8 cells in the CAR-T cell product generated from Perixafor mobilized blood is similar to the proportion of CD4 and CD8 cells in the CAR-T cell product generated from non-mobilized cells.

To test the in vivo efficacy of the different CAR-T cells prepared from Perixafor mobilized blood, NSG mice are injected via tail vein with 0.5×10⁶ RAJI cells stably expressing firefly luciferase (RAJI-Luc) and three days later injected with 4×10⁶ T cells expressing the different CAR constructs. Animals are imaged weekly by bioluminescence imaging following injection of D-luciferin. There is significant tumor growth in animals given no T cells or control T cells and they all died. In contrast, animals given the CAR-T cells generated from Perixafor mobilized blood show improved survival.

Essentially similar results are obtained when the CAR-T cells are generated from blood cells that have been mobilized with treatment with Perixafor alone without treatment with G-CSF.

Generation and Characterization of CAR-T Cells from BL-8040, G-CSF, GM-CSF-Mobilized Cells

Leukapheresis products are collected from healthy subjects or subjects with different cancers who have been administered BL-8040 at a dose of 1 mg/kg subcutaneously for two days. The resulting leukapheresis cell product is used to isolate T cells and then used to generate CD19 SIR-T cells as described in the preceding example. CD19 SIR-T cells generated from BL-8040-mobilized blood are shown to expand in vitro, demonstrate effective cytotoxicity and cytokine production when exposed to their target cells in vitro and effective anti-cancer activity when tested in vivo using a RAJI xenograft model in immunodeficient mice. Essentially similar results are obtained when CAR-T or SIR-T cells are generated from leukapheresis products collected from healthy subjects or subjects with different cancers who have been administered G-CSF (10 μg/kg subcuteneously for 5 days) or GM-CSF (250 μg/m²/day s.c for 5 days).

Effect of Perixafor, BL-8040, G-CSF, GM-CSF-Mobilization on the Generation of Cell Therapy Products

A study is conducted to compare the generation of cell therapy products from cancer patients who have received 2 or more cycles of prior chemotherapy. Patients with different types of different cancers (e.g., diffuse large B cell lymphoma, acute B-ALL, CLL, multiple myeloma, breast cancer, ovarian cancer, lung cancer etc.) are eligible. Leukapheresis products are collected from the subjects with and without mobilization with Perixafor (0.24 mg/kg subcutaneously daily on days 1-3). T cells are isolated and used for manufacturing of CAR-T and TCR-T cells using lentiviral mediated gene transduction as described in the preceding sections. It is observed that mobilization with Perixafor improves the yield of T cells in the initial leukapheresis product and has no major adverse effect on a) gene transduction; b) expression of CAR/TCR; c) expansion of CAR-T/TCR-T products in vitro; d) cytotoxicity and cytokine production by the CAR-T/TCR-T cell products in vitro; e) expansion of CAR-T/TCR-T cell products in vivo; and f) anti-tumor efficacy of CAR-T/TCR-T cell products in vivo. Generation of CAR-T/TCR-T product from Perixafor-mobilized leukapheresis products is found to improve the total number of CAR-T/TCR-T cells obtained upon manufacturing, reduce manufacturing time and cost and reduce manufacturing failure. Similar results are obtained when other CXCR4 antagonists (e.g., BL-8040) is used for mobilization prior to leukapheresis. Finally, mobilization with G-CSF, GM-CSF is also found to improve the yield of leukapheresis product for generation of CAR-T cells.

Treatment of a Patient with Diffuse Large B Cell Lymphoma Using Perixafor Mobilized Immune Cells

A patient diffuse large B cell lymphoma receives Perixafor at a dose of 0.24 mg/kg subcutaneously daily for 3 days. A leukapheresis product is collected from a patient 6 h after the last dose of Perixafor. The leukapheresis product undergoes selection of CD3-positive T lymphocytes using the CliniMACS Prodigy® System from Miltenyi Biotec and following the manufacturer's recommendations. Cells are transduced with clinical grade lentivirus encoding a CD19-CAR (e.g., SEQ ID NO: 2822] and then selection and expansion of the CAR-T cells occurs in a closed system using the CliniMACS Prodigy@ System. After the resulting cell products have undergone quality control testing (including sterility and tumor specific cytotoxicity tests), they are cryopreserved. Meanwhile, following leukapheresis, the patient commences with lymphodepletive chemotherapy (30 mg/m²/day fludarabine plus 500 mg/m²/day cyclophosphamide x 3 days). One day after completion of their lymphodepleting regimen, the previously stored CAR-T cell product is transported, thawed and infused at the patient's bedside. The patient receives CAR-transduced lymphocytes infused intravenously. The dose of CAR-T product varies from 1×10⁴ CAR+ve CD3 cells/kg to 5×10⁸ CAR+ve CD3 cells/kg as per the study protocol. The CAR-T product may be administered in a single infusion or split infusions. The patient can be pre-medicated at least 30 minutes prior to T cell infusion with 15 mg/kg of acetaminophen P.O. (max. 650 mg.) and diphenhydramine 0.5-1 mg/kg I.V. (max dose 50 mg). The patient may optionally receive dailiy injections of human IL-2. Clinical and laboratory correlative follow-up studies are then be performed at the physician's discretion, and may include quantitative RT-PCR studies for the presence of CD19-expressing ALL/lymphoma cells and/or the adoptively transferred T cells; FDG-PET and/or CT scans; bone marrow examination for disease specific pathologic evaluation; lymph node biopsy; and/or long-term follow up per the guidelines set forth by the FDA's Biologic Response Modifiers Advisory Committee that apply to gene transfer studies. Essentially a similar approach can be used to treat other diseases using Perixafor-mobilized immune cells (e.g., T cells) that have been engineered to express the CAR where the CAR targets an antigen or antigens expressed on the disease causing or disease-associated cells.

Treatment of a Patient with Ovarian Cancer Using Perixafor and Chemotherapy Mobilized Immune Cells

A patient ovarian cancer receives chemotherapy with cyclophosphamide (4 gm/m²) plus etoposide (600 mg/m²). One day after completion of chemotherapy, patient receives an injection of Pegfilgastrim (12 mg) subcutaneously. When the absolute neutrophil count reaches more than 500/mm³, patient receives Perixafor at a dose of 0.24 mg/kg subcutaneously daily for 3 days. A leukapheresis product is collected from a patient 6 h after the last dose of Perixafor. The leukapheresis product undergoes selection of CD3-positive T lymphocytes using the CliniMACS Prodigy® System from Miltenyi Biotec and following the manufacturer's recommendations. Optionally, T regulatory cells (CD3+, CD4+, CD25^{high}, CD127^{low}, FoxP3+) are removed by depletion of CD25-positive T cells. Cells are transduced with clinical grade lentivirus encoding a FR1-CAR (e.g., SEQ ID NO:

and then selection and expansion of the CAR-T cells occurs in a closed system. After the resulting cell products passes quality control testing (including sterility and tumor specific cytotoxicity tests), they are cryopreserved. Meanwhile, following leukapheresis, the patient commences with lymphodepletive chemotherapy (30 mg/m²/day fludarabine plus 500 mg/m²/day cyclophosphamide x 3 days). One day after completion of their lymphodepleting regimen, the previously stored CAR-T cell product is transported, thawed and infused at the patient's bedside. The patient receives CAR-transduced lymphocytes infused intravenously. The dose of CAR-T product varies from 1×10⁴ CAR+ve CD3 cells/kg to 5×10⁸ CAR+ve CD3 cells/kg as per the study protocol. The CAR-T product may be administered in a single infusion or split infusions. The patient can be pre-medicated at least 30 minutes prior to T cell infusion. The patient may optionally receive dailiy injections of human IL-2. Clinical and laboratory correlative follow-up studies are then be performed at the physician's discretion. Essentially a similar approach can be used to treat other diseases using chemotherapy and Perixafor-mobilized immune cells (e.g., T cells) that have been engineered to express the CAR or TCR where the CAR or the TCR targets an antigen or antigens expressed on the disease causing or disease-associated cells.

Use of G-CSF and Perixafor to Mobilize Immune Prior to Leukapheresis for Manufacturing of Cell Therapy Products.

A healthy donor receives G-CSF (Neupogen) at dose of 10 μg/kg daily from days 1-5 by subcutaneous injection and Perixafor at a dose of 0.24 mg/kg subcutaneously daily on days 3-5. A leukapheresis product is collected from a donor 6 h after the last dose of Perixafor. The CD3 cells are isolated from the leukapheresis product using magnetic beads. A Mesothelin-targeted chimeric antigen receptor (e.g. SEQ ID NO: 2832) is targeted to the TCR alpha (TRAC) locus according to methods known in the art. The Mesothelin-CAR-T cells expressing TCRa-deficient T cells are purified, expanded using CD3/CD28 beads using methods known in the art. An aliquote of Mesothelin-CAR-T cells is tested for cytotoxicity against SKOV3-GLuc cells using Matador asaay and is shown to exert robust cytotoxicity as compared to control CAR-T cells. Another aliquote Mesothelin-CAR-T cells (5×10⁵ CAR-T cells/mice) is tested for in vivo activity using a xenograft model of SKOV3 cells (1×10⁵ cells by subcutaneous injection) in NSG mice using methods known in the art and is shown to reduce tumor growth and prolong survival as compared to mice given control CAR-T cells. Another aliquote of Mesothelin-CAR-T cells (5×10⁸ CAR-T cells) is administered to a patient with Mesothelin-expressing mesothelioma by intravenous infusion using methods known in the art.

Use of Beta2 Adrenergic Agonists or Exercise to Mobilize Immune Prior to Leukapheresis for Manufacturing of Cell Therapy Products.

A leukapheresis product is collected from a patient with metastatic ovarian cancer expressing Mesothelin after the patient has received epinephrine intravenous infusion at 0.005 mg/kg/min for about 30 minutes. In an alternate embodiment, the patient performs moderate exercise on a treadmill for 30 minutes to increase the heart rate to above 150 beats/minute. Selection of CD3-positive T lymphocytes is performed using the CliniMACS Prodigy® System from Miltenyi Biotec and following the manufacturer's recommendations. Approximately 10⁸ to 10⁸ T cells are transduced with a lentiviral encoding a Mesothlin CAR (SEQ ID NO: 2831). The CAR-T cells are expanded for 14-21 days in a closed system using CD3/CD28 beads. The resulting cell products undergoes quality control testing (including sterility and tumor specific cytotoxicity tests). The CAR-T cell product is administered to the patient by intravenous infusion. The dose of CAR-T product varies from 1×10⁴ CAR+ve CD3 cells/kg to 5×10⁹ CAR+ve CD3 cells/kg.

Use of BL-8040 to Mobilize Immune Prior to Leukapheresis for Manufacturing of Cell Therapy Products.

A leukapheresis product is collected from ahealthy volunteer after administration of 1 mg/kg BL-8040 subcutaneoulsy for 2 days. Selection of CD3-positive T lymphocytes is performed using the CliniMACS Prodigy@ System from Miltenyi Biotec and following the manufacturer's recommendations. Approximately 10⁸ to 10⁸ T cells are transduced with a lentiviral encoding a CD19 CAR (SEQ ID NO:478). The CAR-T cells are expanded for 14-21 days in a closed system using CD3/CD28 beads. The resulting CAR-T cell product passes quality control testing (e.g., sterility, yield, tumor specific cytotoxicity tests, antigen induced cytokine production, immunophenotype etc.) and is cryopreserved in aliquotes. An aliquote of CAR-T cell product is thawed and administered to the patient with CD19+ ALL by intravenous infusion. The dose of CAR-T product varies from 1×10⁴ CAR+ve CD3 cells/kg to 5×10⁹ CAR+ve CD3 cells/kg. Essentially a similar approach is used to manufacture an autologous CAR-T cell product from BL-8040 mobilized immune cells.

Effect of EZH2 Inhibitors Tazemetostat (EPZ6438, Cat No S7128, Cayman Chemicals) and GSK343 (Cat No. S7164 Cayman Chemicals) to Generate T Stem Cells for Adoptive Immunotherapy Showing Surface Expression of CD45RA+CD62L+CCR7+

The use of P-glycoprotein (Pgp)-mediated efflux of DiOC2(3) to isolate stem like T cells for adoptive cellular therapy has been described in application no. PCT/US2017/042248. In the following examples, DiOC2(3) staining was used to study the effect of different inhibitors on generation of stem like T cells.

DAY 0: Blood was obtained from unidentified donor. After RBC lysis, PBMC were collected using Ficoll-Hypaque separation. 208 million T cells were isolated from 440 million PBMCs using CD3 microbeads (cat. No. 130-050-101) from Miltenyi. T cells were cultured in three different conditions:
1. XVIVO 15 medium (Lonza) with IL2 50 IU/ml, CD3 antibody (30 ng/ml) and CD28 antibody (30 ng/ml).
2. XVIVO 15 medium (Lonza) with CD3 antibody (30 ng/ml) and CD28 antibody (30 ng/ml).
3. XVIVO 15 medium (Lonza) with IL2 50 IU/ml
   DAY 1: Cells were plated in a 6-well plate at a density of 1 million cells in 2 ml/of media (as described above) and treated with different inhibitors

Cells were cultured for 6 days and an aliquot of cells with all the treatments listed above (1-21) was used to stain the cells with DiOC2(3) (60 ng/ml in RPMI medium) at 4° C. for 40 min, washed with RPMI medium, DiOC2(3) dye was effluxed at 37° C. for 90 min, washed with PBS+1% FBS, stained with CD62L-APC antibdoy (5 μl/100 μl/sample) at 4° C. for 1 h, washed twice with PBS+1% FBS and analyzed on flow cytometer to check if these drugs are affecting the dye-effluxing or Pgp+ T cell population which are enriched for T stem cells and are better suited for adoptive cellular therapy, e.g., for manufacturing of CAR-T or TCR-T cell therapy products.

The cell growth medium was changed in 6-well plates and fresh drugs were added at same concentration for one more week.

The effect of different tested inhibitors on the percentage of the Pgp+ and CD62L+ T cell population is shown below in Table 9 and Table 10. Most inhibitors increased the percentage of Pgp+ and CD62L+ stem like T cells when added to the culture containing treatment with CD3 antibody, CD28 antibdy and L2. For example, the perecentage ofPgp+ cells increased from 19.800 cells in culture containing treatment with CD3-CD28-IL2 to 27.17 in culture containing treatment with CD3, CD28, L2 and Tazemetostat (10 nM) and 30.62% in culture containing CD3, CD28, L2 and GSK343 (500 nM). Similarly, the perecentage of Pgp+CD62L+ cells increased from 6.57% cells in culture containing treatment with CD3-CD28-IL2 to 13.168 in culture containing treatment with CD3, CD28, L2 and Tazemetostat (100 nM) and 13.33% in culture containing CD3, CD28, L2 and GSK343 (500 nM).

TABLE 9
Table 9: Percentage of Pgp+ (DiOC2 (3)-dye effluxing cells)
T cells are increased after 6 days of treatment with EZH2-inhibitors.

| S. | | % of Pgp+ cells |
|---|---|---|
| No. | Treatment | out of live cells |
| 1 | CD3-CD28-IL2 | 19.89 |
| 2 | CD3-CD28 | 35.34 |
| 3 | CD3-IL2 | 18.41 |
| 4 | CD3-CD28-IL2-Tazemetostat 10 nM | 21.86 |
| 5 | CD3-CD28-IL2-Tazemetostat 100 nM | 27.17 |
| 6 | CD3-CD28-IL2-Tazemetostat 200 nM | 24.85 |
| 7 | CD3-CD28-IL2-GSK343 100 nM | 23.29 |
| 8 | CD3-CD28-IL2-GSK343 500 nM | 30.62 |
| 9 | CD3-CD28-IL2-GSK343 1 μM | 28.03 |
| 10 | CD3-CD28-Tazemetostat 10 nM | 39.28 |
| 11 | CD3-CD28-Tazemetostat 100 nM | 40.50 |
| 12 | CD3-CD28-Tazemetostat 200 nM | 43.11 |
| 13 | CD3-CD28-GSK343 100 nM | 48.43 |
| 14 | CD3-CD28-GSK343 500 nM | 47.09 |
| 15 | CD3-CD28-GSK343 1 μM | 46.26 |
| 16 | CD3-IL2-Tazemetostat 10 nM | 24.77 |
| 17 | CD3-IL2-Tazemetostat 100 nM | 25.31 |
| 18 | CD3-IL2-Tazemetostat 200 nM | 25.89 |
| 19 | CD3-IL2-GSK343 100 nM | 25.49 |
| 20 | CD3-IL2-GSK343 500 nM | 25.21 |
| 21 | CD3-IL2-GSK343 1 μM | 28.31 |

TABLE 10
Table 10: Percentage of Pgp+ (DiOC2(3)-dye effluxing
cells) and CD62L+ T cells are increased after 6
days treatment with EZH2-inhibitors

| S. | | % of Pgp+ CD62L+cells |
|---|---|---|
| No. | Treatment | out of live cells |
| 1 | CD3-CD28-IL2 | 6.57 |
| 2 | CD3-CD28 | 15.66 |
| 3 | CD3-IL2 | 4.29 |
| 4 | CD3-CD28-IL2-Tazemetostat 10 nM | 8.42 |
| 5 | CD3-CD28-IL2-Tazemetostat 100 nM | 13.16 |
| 6 | CD3-CD28-IL2-Tazemetostat 200 nM | 11.30 |
| 7 | CD3-CD28-IL2-GSK343 100 nM | 8.57 |
| 8 | CD3-CD28-IL2-GSK343 500 nM | 13.33 |
| 9 | CD3-CD28-IL2-GSK343 1 μM | 12.05 |
| 10 | CD3-CD28-Tazemetostat 10 nM | 19.36 |
| 11 | CD3-CD28-Tazemetostat 100 nM | 18.38 |
| 12 | CD3-CD28-Tazemetostat 200 nM | 20.94 |
| 13 | CD3-CD28-GSK343 100 nM | 23.35 |
| 14 | CD3-CD28-GSK343 500 nM | 24.79 |
| 15 | CD3-CD28-GSK343 1 μM | 23.41 |
| 16 | CD3-IL2-Tazemetostat 10 nM | 8.76 |
| 17 | CD3-IL2-Tazemetostat 100 nM | 10.11 |
| 18 | CD3-IL2-Tazemetostat 200 nM | 11.97 |
| 19 | CD3-IL2-GSK343 100 nM | 8.62 |
| 20 | CD3-IL2-GSK343 500 nM | 9.32 |
| 21 | CD3-IL2-GSK343 1 μM | 10.99 |

O11618 (DAY 11): Culture medium was changed and fresh drugs were added in 6-well plates.

O11818 (DAY 13): One aliquot of the cells that had been cultured for 13 days with all the treatments listed above (1-21) were used to stain the cells with DiOC2(3) (60 ng/ml in RPMI medium) at 4° C. for 40 min. Subsequently, cells were washed with RPMI medium and dye was effluxed at 37° C. for 90 min. Cells were washed with PBS+100 FBS, stained with CD62L-APC (5 μl/100 μl/sample) at 4° C. for 1 h, washed twice with PBS+1% FBS and analyzed on flow cytometer to check if these drugs treatment leads to increase in Pgp+/CD62L positive population.

TABLE 11
Table 11: Percentage of Pgp+ (DIOC2-dye effluxing cells) T
cells are increased after 13 days of treatment with EZH2-inhibitors.

| S. | | % of Pgp+ cells |
|---|---|---|
| No. | Treatment | out of live cells |
| 1 | CD3-CD28-IL2 | 25.55 |
| 2 | CD3-CD28 | 10.22 |
| 3 | CD3-IL2 | 17.85 |
| 4 | CD3-CD28-IL2-Tazemetostat 10 nM | 30.38 |
| 5 | CD3-CD28-IL2-Tazemetostat 100 nM | 38.45 |
| 6 | CD3-CD28-IL2-Tazemetostat 200 nM | 41.22 |
| 7 | CD3-CD28-IL2-GSK343 100 nM | 27.15 |
| 8 | CD3-CD28-IL2-GSK343 500 nM | 18.00 |
| 9 | CD3-CD28-IL2-GSK343 1 μM | 23.42 |
| 10 | CD3-CD28-Tazemetostat 10 nM | 12.41 |
| 11 | CD3-CD28-Tazemetostat 100 nM | 5.19 |
| 12 | CD3-CD28-Tazemetostat 200 nM | 11.82 |
| 13 | CD3-CD28-GSK343 100 nM | 11.66 |
| 14 | CD3-CD28-GSK343 500 nM | 10.34 |
| 15 | CD3-CD28-GSK343 1 μM | 9.43 |
| 16 | CD3-IL2-Tazemetostat 10 nM | 28.61 |
| 17 | CD3-IL2-Tazemetostat 100 nM | 16.74 |
| 18 | CD3-IL2-Tazemetostat 200 nM | 28.06 |
| 19 | CD3-IL2-GSK343 100 nM | 20.31 |
| 20 | CD3-IL2-GSK343 500 nM | 23.54 |
| 21 | CD3-IL2-GSK343 1 μM | 17.50 |

TABLE 12
Table 12: percentage of Pgp+ (DiOC2(3)-dye effluxing
cells) and CD62L+ T cells are increased with 13
days treatment with EZH2-inhibitors

| S. | | % of Pgp+ CD62L+cells |
|---|---|---|
| No. | Treatment | out of live cells |
| 1 | CD3-CD28-IL2 | 6.41 |
| 2 | CD3-CD28 | 2.09 |
| 3 | CD3-IL2 | 4.70 |
| 4 | CD3-CD28-IL2-Tazemetostat 10 nM | 8.03 |
| 5 | CD3-CD28-IL2-Tazemetostat 100 nM | 12.57 |
| 6 | CD3-CD28-IL2-Tazemetostat 200 nM | 14.25 |
| 7 | CD3-CD28-IL2-GSK343 100 nM | 7.96 |
| 8 | CD3-CD28-IL2-GSK343 500 nM | 4.79 |
| 9 | CD3-CD28-IL2-GSK343 1 μM | 5.75 |
| 10 | CD3-CD28-Tazemetostat 10 nM | 2.95 |
| 11 | CD3-CD28-Tazemetostat 100 nM | 1.18 |
| 12 | CD3-CD28-Tazemetostat 200 nM | 2.66 |
| 13 | CD3-CD28-GSK343 100 nM | 2.50 |
| 14 | CD3-CD28-GSK343 500 nM | 2.12 |
| 15 | CD3-CD28-GSK343 1 μM | 2.28 |
| 16 | CD3-IL2-Tazemetostat 10 nM | 9.87 |
| 17 | CD3-IL2-Tazemetostat 100 nM | 4.31 |
| 18 | CD3-IL2-Tazemetostat 200 nM | 9.08 |
| 19 | CD3-IL2-GSK343 100 nM | 5.24 |
| 20 | CD3-IL2-GSK343 500 nM | 6.82 |
| 21 | CD3-IL2-GSK343 1 μM | 5.17 |

A second aliquot of the cells cultured for 13 days with all the treatments listed above (1-21) was washed with PBS+1% FBS, stained with CD8-PerCP, CD45RA-FITC, CCR7-PE and CD62L-APC (5% Y/100 μl/sample for each of 4 antibodies) at 4° C. for 1 h, washed twice with PBS+100 FBS and analyzed on flow cytometer to check if these drugs are enriching T stem cells showing surface expression of CD8+CD45RA+CCR7+CD62L+.

TABLE 13
Table 13: percentage of CD8+CD45RA+CCR7+CD62L+ T cells are
increased with 13 days of treatment with EZH2-inhibitors

| | | % of CD62L+CCR7+ |
|---|---|---|
| | | double positive cells |
| S. | | out of CD45RA+ cells in |
| No. | Treatment | CD8+ T cell population |
| 1 | CD3-CD28-IL2 | 60.01 |
| 2 | CD3-CD28 | 65.86 |
| 3 | CD3-IL2 | 58.84 |
| 4 | CD3-CD28-IL2-Tazemetostat 10 nM | 62.73 |
| 5 | CD3-CD28-IL2-Tazemetostat 100 nM | 83.53 |
| 6 | CD3-CD28-IL2-Tazemetostat 200 nM | 80.78 |
| 7 | CD3-CD28-IL2-GSK343 100 nM | 82.96 |
| 8 | CD3-CD28-IL2-GSK343 500 nM | 82.42 |
| 9 | CD3-CD28-IL2-GSK343 1 μM | 85.00 |
| 10 | CD3-CD28-Tazemetostat 10 nM | 87.00 |
| 11 | CD3-CD28-Tazemetostat 100 nM | 88.26 |
| 12 | CD3-CD28-Tazemetostat 200 nM | 88.37 |
| 13 | CD3-CD28-GSK343 100 nM | 85.28 |
| 14 | CD3-CD28-GSK343 500 nM | 80.34 |
| 15 | CD3-CD28-GSK343 1 μM | 85.70 |
| 16 | CD3-IL2-Tazemetostat 10 nM | 82.23 |
| 17 | CD3-IL2-Tazemetostat 100 nM | 79.86 |
| 18 | CD3-IL2-Tazemetostat 200 nM | 77.35 |
| 19 | CD3-IL2-GSK343 100 nM | 73.95 |
| 20 | CD3-IL2-GSK343 500 nM | 77.29 |
| 21 | CD3-IL2-GSK343 1 μM | 81.47 |

TABLE 14
percentage of CD45RA+CCR7+CD62L+ T
cells (all T cells not only CD8+ or CD8−) are
increased after 13 days treatment with EZH2-inhibitors

| | | % of CD62L+CCR7+ |
|---|---|---|
| | | double positive cells |
| S. | | out of CD45RA+ cells in |
| No. | Treatment | live T cell population |
| 1 | CD3-CD28-IL2-UT | 51.86 |
| 2 | CD3-CD28-UT | 60.55 |
| 3 | CD3-IL2-UT | 50.51 |
| 4 | CD3-CD28-IL2-Tazemetostat 10 nM | 50.85 |
| 5 | CD3-CD28-IL2-Tazemetostat 100 nM | 67.51 |
| 6 | CD3-CD28-IL2-Tazemetostat 200 nM | 66.36 |
| 7 | CD3-CD28-IL2-GSK343 100 nM | 66.40 |
| 8 | CD3-CD28-IL2-GSK343 500 nM | 64.47 |
| 9 | CD3-CD28-IL2-GSK343 1 μM | 67.37 |
| 10 | CD3-CD28-Tazemetostat 10 nM | 77.81 |
| 11 | CD3-CD28-Tazemetostat 100 nM | 79.62 |
| 12 | CD3-CD28-Tazemetostat 200 nM | 78.45 |
| 13 | CD3-CD28-GSK343 100 nM | 75.26 |
| 14 | CD3-CD28-GSK343 500 nM | 67.97 |
| 15 | CD3-CD28-GSK343 1 μM | 76.18 |
| 16 | CD3-IL2-Tazemetostat 10 nM | 67.30 |
| 17 | CD3-IL2-Tazemetostat 100 nM | 63.89 |
| 18 | CD3-IL2-Tazemetostat 200 nM | 59.08 |
| 19 | CD3-IL2-GSK343 100 nM | 58.51 |
| 20 | CD3-IL2-GSK343 500 nM | 61.18 |
| 21 | CD3-IL2-GSK343 1 μM | 61.06 |

Effect of EZH2, BRD and Other Inhibitors to Generate Superior T Stem Cells for Adoptive Immunotherapy Showing Surface Expression of CD45RA+CD62L+CCR7+

Day 0: Blood was obtained from unidentified donor. After RBC lysis, PBMC were collected using Ficoll-Hypaque separation. 200 million T cells were isolated from PBMCs using CD3 microbeads. T cells were cultured overnight in XVIVO 15 medium (Lonza) with IL2 (50 IU/ml), 30 ng/ml of CD3 and CD28 antibodies

Day 1: Cells were plated in 24-well plates at a density of 0.3 million cells/ml in each well and treated with different drugs.

On day 7, culture medium was replaced with fresh medium with fresh drugs.

On day 13, culture medium was replaced with fresh medium and fresh drugs. Half of the cells were transferred to 6-well plates.

Day 14: An aliquot of cells with all the treatments listed above (for 13 days) was used to stain the cells with DiOC2(3) (60 ng/ml in RPMI medium) at 4° C. for 40 min, washed with RPMI medium, DiOC2(3) dye was effluxed at 37° C. for 90 min, washed with PBS+1% FBS, stained with CD62L-APC antibody. CD62L is a marker for resting T cells. (5 μl/100 μl/sample) at 4° C. for 1 h, washed twice with PBS+1% FBS and analyzed on flow cytometer to check if these drugs are affecting the dye-effluxing or Pgp+ (i.e., DiOC2(3)-dull) T-cell population.

Table 15 shows that treatment with inhibitors increased the Pgp+(DiOC2(3)-dull) and CD62L+ T cell population as shown below:

| | Treatment | |
|---|---|---|
| | | p5: double +ve |
| | p2: pgp+ | pgp+ and CD62L-APC+ |
| Name | from Live cells | from Live cells |
| Untreated (UT) | 21.12 | 3.78 |
| TAZE-100 nM | 42.9 | 14.5 |
| GSK334-500 nM | 15.65 | 3.15 |
| MECLO-100 nM | 22.22 | 3.74 |
| MECLO-500 nM | 20.11 | 3.72 |
| TAZE-100 + MECLO-100 | 38.06 | 9.76 |
| TAZE-100 + SC1-500 | 54.64 | 24.86 |
| GSK-500 + MECLO-100 | 19.11 | 4.15 |
| GSK-500 + SC1-500 | 53.03 | 24.84 |
| R05335-100 nM | 14.71 | 2.55 |
| R05335-500 nM | 16.22 | 2.3 |
| CBFB-500 nM | 16.01 | 2.85 |
| CBFB-1 μM | 20.93 | 3.56 |
| BI7273-100 nM | 16.69 | 2.73 |
| BI7273-500 nM | 16.34 | 2.38 |
| B9564-100 nM | 13.39 | 2.53 |
| B9564-500 nM | 15.56 | 3.03 |
| LP99-500 nM | 19.46 | 3.52 |
| LP99-1 μM | 20.66 | 4.18 |
| IBRD9-500 nM | 21.12 | 4.51 |
| IBRD9-1 μM | 31.69 | 7.26 |
| GSK503-1 μM | 47.87 | 15.79 |
| GSK503-2 μM | 56.13 | 19.99 |
| GSK126-100 nM | 36.37 | 8.83 |
| GSK126-500 nM | 26.61 | 8.08 |
| UNC1999-100 nM | 36.8 | 11.06 |
| UNC1999-500 nM | 31.14 | 8.9 |
| CPI169-100 nM | 25.52 | 4.68 |
| CPI169-200 nM | 30.65 | 6.4 |
| CPI360-100 nM | 25.15 | 5.82 |
| CPI360-500 | 35.55 | 11.11 |
| EI1-500 nM | 33.53 | 10.2 |
| EI1-1 μM | 59.77 | 19.21 |
| EPZ011989-500 nM | 64.39 | 18.77 |
| EPZ011989-1 μM | 62.54 | 16.8 |
| EPZ005687-10 nM | 23.99 | 5.21 |
| EPZ005687-100 nM | 16.94 | 3.87 |
| EED226-500 nM | 37.6 | 10.41 |
| EED226-1 μM | 43.53 | 11.82 |
| UNC0642-10 nM | 22.03 | 4.46 |
| UNC0642-100 nM | 32.51 | 4.81 |

A second aliquot of the cells cultured for 14 days with all the treatments listed above was washed with PBS+1% FBS, stained with CD8-PerCP, CD45RA-FITC, CCR7-PE and CD62L-APC (5 ul/100 ul/sample for each of 4 antibodies) at 4° C. for 1 h, washed twice with PBS+1% FBS and analyzed on flow cytometer to check if these drugs are enriching for T cells showing surface expression of CD8+CD45RA+CCR7+CD62L+.

The effect of various inhibitors on % of CD8+/CD45RA+/CCR7+/CD62L+(P4) and CD45RA+/CCR7+/CD62L+(P6) is shown in the following Table 16. The results show marked increase in the % of CD8+/CD45RA+/CCR7+/CD62L+ and CD45RA+/CCR7+/CD62L+ cells following treatment with different inhibitors. For example, the % of CD8+/CD45RA+/CCR7+/CD62L+ cells in the untreated sample was 56.3% and increased to >70% following treatment with R05335-500 nM, CBFB-500 nM, CBFB 1 pM, B17273 100 nM, B17273 500 nM, B9564 100 nM, B9564 500 nM, LP99 500 nM, LP99 1 μM, IBRD9 500 nM, IBRD9 1 μM, GSK503 1 μM, GSK503 2 μM, GSK126 100 nM, GSK126 500 nM, UNC1999 100 nM, UNC1999 500 nM, CP1169 100 nM, CP1169 200 nM, CPI360 100 nM, CPI360 500 nM, EI1 500 nM, EI1 1 μM, EPZ011989 500 nM, EPZ011989 1 μM, EPZ005687 10 nM, EPZ005687 100 nM, and EED226 500 nEED226 1 μM, UNC0642 10 nM and UNC0642 100 nM.

TABLE 16

| | | GATE P4 | |
|---|---|---|---|
| | | CD8(PerCP)+, | GATE P6 |
| | | CD45RA(FITC)+, | CD45RA(FITC)+, |
| | stat-020818 | CCR7 (PE)+, | CCR7 (PE)+, |
| Target Gene | Name of compound | CD62L(APC) | CD62L(APC) |
| | UTREATED | 56.3 | 51.92 |
| EZH2 | TAZE-100 nM | 59.72 | 52.24 |
| Tet2 | MECLO-100 nM | 57.65 | 53.02 |
| Tet2 | MECLO-500 nM | 75.78 | 66.64 |
| | TAZE-100 + MECLO-100 | 73.6 | 61.32 |
| | TAZE-100 + SC1-500 | 82.12 | 80.18 |
| | GSK-500 + MECLO-100 | 72.18 | 63.12 |
| | GSK-500 + SC1-500 | 85.56 | 78.74 |
| CBFb-Runx1 | R05335-500 nM | 79.9 | 69.95 |
| CBFb | CBFB-500 nM | 74.77 | 64.71 |
| CBFb | CBFB-1 μM | 74.12 | 65.61 |
| BRD9 | BI7273-100 nM | 70.33 | 57.15 |
| BRD9 | BI7273-500 nM | 77.81 | 61.86 |
| BRD9 | B9564-100 nM | 73.18 | 63.27 |
| BRD9 | B9564-500 nM | 83.31 | 73.75 |
| BRD9 | LP99-500 nM | 79.25 | 69.67 |
| BRD9 | LP99-1 μM | 79.77 | 67.58 |
| BRD9 | IBRD9-500 nM | 74.3 | 61.53 |
| BRD9 | IBRD9-1 μM | 79.18 | 67.77 |
| EZH2 | GSK503-1 μM | 75.24 | 63.54 |
| EZH2 | GSK503-2 μM | 75.8 | 62.62 |
| EZH2 | GSK126-100 nM | 76.44 | 67.75 |
| EZH2 | GSK126-500 nM | 79.6 | 67.65 |
| EZH2/EZH1 | UNC1999-100 nM | 71.75 | 59.08 |
| EZH2/EZH1 | UNC1999-500 nM | 74.57 | 60.97 |
| EZH2 | CPI169-100 nM | 81.07 | 70.48 |
| EZH2 | CPI169-200 nM | 75.83 | 66.23 |
| EZH2 | CPI360-100 nM | 81.01 | 70.85 |
| EZH2 | CPI360-500 nM | 81.26 | 69.62 |
| EZH2 | EI1-500 nM | 76.19 | 67.64 |
| EZH2 | EI1-1 μM | 78.47 | 66.83 |
| EZH2 | EPZ011989-500 nM | 70.63 | 57.62 |
| EZH2 | EPZ011989-1 μM | 72.83 | 58.31 |
| EZH2 | EPZ005687-10 nM | 74.86 | 64.01 |
| EZH2 | EPZ005687-100 nM | 79.65 | 69.92 |
| PCR2 (polycomb | EED226-500 nM | 70.21 | 60.45 |
| repressive complex 2) | | | |
| PCR2 (polycomb | EED226-1 μM | 74.83 | 60.45 |
| repressive complex 2) | | | |
| G9a histone | UNC0642-10 nM | 71.62 | 63.95 |
| methyltransferase | | | |
| G9a histone | UNC0642-100 nM | 82.37 | 75.75 |
| methyltransferase | | | |

Effect of Drug Treatments on Blincyto (Blinatumomab) Induced Cytotoxicity

After 20 days of treatment with the inhibitors, cells from the preceding experiment were tested for their ability to induce Blinatumomab induced cell death using the Matador cytotoxicity assay. For this purpose, untreated and inhibitors-treated T cells were plated in a 24 well plate at 2×10⁵ cells/well either alone or in the presence of 2×10⁵ Nalm6-GLuc cells in XVIVO medium without any additional growth factors. Blinatumomab (Blincyto) was added at final concentration of 100 ng/million T cells. Cells were cultured overnight and next day supernatant was collected and assayed using the Matador assay. For the Matador Assay, 15 μl of supernatant was added to 15 μl of XVIVO medium in each well of a 384-well plate and hGLuc assay was done by adding 15 μl of 1:100 CTZ per well in well mode. The results showed significant increase in cytotoxicity in cells treated with Eli (500 nM), EPZ005687 (100 nM), LP99 (1 μM), UNC1999 (100 nM), CPI169 (200 nM), CPI360 (100 nM) and CPI360 (500 nM) as compared to untreated T cells. Thus, in addition to their effect on T stem cells, the above inhibitors can be also used to enhance the cytotoxic potential of immune effector cells, such as CAR-T cells, TCR-T cells and Bispecific T cell engager, e.g, Blinatumomab-treated T cells.

Preventing and Reversing T-Cell Exhaustion in Disease

Unmodified Host: Infection

Patients suffering from chronic HIV, HCV, HBV infections are extensively reported to have T cells exhausted

In some embodiments, patients with chronic viral infections are treated with BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1 and HDAC2 inhibitor drugs either singly or in combination as follow: The population of patients suffering from chronic HIV, HCV, HBV infections is sampled to determine PD1 expression on their T cells obtained from peripheral blood mononuclear cells (PBMCs). The procedure of using PDl as a marker of exhaustion is applied to sample exhausted T cells present in chronic HIV and EBV infections and is, thus, an accepted criteria of exhaustion in chronic viral infections (Day et al., 2006, supra). This analysis is performed in comparison with healthy donors. Alternatively, virus-specific T cells are assayed for PD1 determined by tetramer co-staining. Subsequently, the CD4+ and CD8+ T cells are analyzed for PDl expression and co-stained for YY1, Ezh2 or cJun expression. This is followed by treatment with BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and DR5 inhibitor drugs either singly or in combination. The BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1 and HDAC2 inhibitor drugs are directly injected or orally administered, e.g., i.p, i.v routes into patients suffering from chronic viral infections such as HIV, HCV, HBV. The dose and frequency of administration for each inhibitor is determined by measuring the inhibition of the target protein in the peripheral blood lymphocytes. In an alternate embodiment, the dose and frequency of administration is determined by pharmacokinetic studies to maintain the plasma level above the IC50 for each of the inhibitor drug.

Therefore, a major effect of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1 and HDAC2 inhibitor drugs is expected to be on the virus-specific exhausted T cells that may reverse exhaustion by restoring IL2 production and T cell proliferation and BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1 and HDAC2 inhibitor drugs apart from IL2 rescue are expected to reduce exhaustion markers of PD1, Lag3 and Tim3.

Unmodified Host: Cancer

In some embodiments, patients with malignant cancers are treated with BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and DR5 inhibitor drugs either alone or in combination. In one embodiment, the inhibitors are administered orally. In another embodiment, the inhibitors are administered parentally. In another embodiment, the inhibitors are directly injected at the site of tumors. A major effect of the inhibitor drugs is expected to be on the exhausted T cells infiltrating the tumors to help reverse exhaustion by restoring IL2 production and T cell proliferation.

Modified Host: Cancer, TILs Treatment

In some embodiments, cancer patients are treated with TILs modified with BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and DR5 downregulation or inactivation as follows: Tumor infiltrating lymphocytes (TILs) are extracted from the tumor biopsies and grown in cultures. A standard protocol to grow TILs in tissue culture conditions is applied. In some embodiments, commercially available tumor dissociation kits (Miltenyi Biotech) are used. TILs are expanded in culture, activated and treated with BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 inhibitor or infected with BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 shRNA lentiviral particles to determine reversal of exhaustion phenotype. The chemical inhibitors used are listed in Table 6 are used at dose above their IC50 concentrations. The sequences of the shRNAs (e.g., SEQ ID NO: 274-335; 2226-2237) targeting the above genes is shown in Table 5. Once these in vitro results are confirmed from sample patients, a similar procedure is applied to the melanoma, ovarian cancer, synovial sarcoma, non-Small lung cancer patients. In such patients, TILs are extracted, expanded ex vivo and treated with BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1 and HDAC2 chemical inhibitors or infected with BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 shRNA encoding lentiviral vectors and infused back into subjects to generate antitumor responses. In another embodiment, TILs are extracted, expanded ex vivo and one or both alleles of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and/or DR5 are knocked out either singly or in combination using Cas9/CRISP approach using standard molecular biology techniques. In one embodiment, the knock-down of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 is achieved by infecting cells with a lentiviral vector encoding Streptococcus pyogenese Cas9/CRISP and gRNA sequences targeting BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5. For example, double stranded oligonucleotides corresponding to gRNA sequences targeting BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and DR5 are synthesized and cloned in the pLenti-CRISPR-v2 vector (SEQ ID NO: 359) obtained from Addgene. The sequence of the gRNAs (SEQ ID NO: 1-104; 1775-1786) targeting the above genes is provided in Table 4a. The lentiviruses are prepared following the recommendations available from Addgene and used to infect TILs. Subsequently, TILs are expanded in T cell culture media containing CD3/CD28 beads and IL2 (30 IU) and used for in vitro and in vivo studies and for human clinical trials.

In another embodiment, TILs are extracted, expanded ex vivo and genetically engineered to express constitutively active mutants of JAK1, JAK3, STAT5b, STAT3, IL2RG, CARD11 and BRAF either alone or in combination. The SEQ ID NO of the constitutive active mutants of JAK1(e.g., SEQ ID NO:375-376), JAK3 (e.g., SEQ ID NO:361-367), STAT5b (e.g., SEQ ID NO:369-373), STAT3 (e.g., SEQ ID NO:381-385), IL2RG (e.g., SEQ ID NO:387), CARD11 (e.g., SEQ ID NO:378-379) and BRAF (e.g., SEQ ID NO:389) are provided in Table 1. In another embodiment, TILs are extracted, expanded ex vivo and genetically engineered to express constitutively active mutants of JAK1, JAK3, STAT5b, STAT3, IL2RG, CARD11 and BRAF either alone or in combination along with lentiviral vectors encoding shRNAs targeting one or more of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and/or DR5. In another embodiment, TILs are extracted, expanded ex vivo and genetically engineered to express constitutively active mutants of JAK1, JAK3, STAT5b, STAT3, IL2RG, CARD11 and BRAF either alone or in combination along with lentiviral vectors encoding Streptococcus pyogenese Cas9/CRISP and gRNA sequences targeting BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and/or DR5. In another embodiment, TILs are extracted, expanded ex vivo in the presence of chemical inhibitors of one or more of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL and/or DR5 and genetically engineered to express constitutively active mutants of JAK1, JAK3, STAT5b, STAT3, IL2RG, CARD11 and BRAF either alone or in combination

Modified Host: Infection, CAR-T and TCR-T Treatment

CAR-T cells and TCR-T cells are modified by co-transduction or sequential transduction to express RNAi or shRNA or other inhibitory RNA to prevent BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 expression. In another embodiment, the CAR-T/TCR-T cells are modified by co-transduction or sequential transduction to express constitutively active form of JAK1, JAK3, STAT5b, STAT3, IL2RG, CARD11 and BRAF either alone or in combination. In another embodiment, the CAR-T and TCR-T cells are modified by co-transduction or sequential transduction to express constitutively active form of JAK1, JAK3, STAT5b, STAT3, IL2RG, CARD11 and BRAF either alone or in combination along with RNAi or Rz or other inhibitory RNA to prevent BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 expression.

For CAR/TCR vector with BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 knockdown, one may use two separate vector systems to express CARs and BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1 and HDAC2 siRNA, shRNA or miRNA. In some embodiments, a single vector system employing separate promoters, one specific for CAR/TCR transcription and the other promoter for BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 siRNA, shRNA or miRNA transcription is used.

For CAR/TCR vector with co-expression of constitutively active form of JAK1, JAK3, STAT5b, STAT3, IL2RG, CARD11 and BRAF, one may use two separate vector systems to express CARs and constitutively active mutant forms of JAK1, JAK3, STAT5b, STAT3, IL2RG, CARD11 and BRAF. In some embodiments, a single vector system employing separate promoters, one specific for CAR/TCR transcription and the other promoter for expression of constitutively active form of JAK1, JAK3, STAT5b, STAT3, IL2RG, CARD11 and BRAF transcription is used. In some embodiments, a single vector system employing a single promoter is used to express an expression cassette encoding both a CAR/TCR transcription and a constitutively active form of JAK1, JAK3, STAT5b, STAT3, IL2RG, CARD11 and BRAF in which the nucleic acids encoding the CAR/TCR and the mutants are separated by a 2A sequence. Several exemplary expression cassettes encoding a CAR/TCR and a a constitutively active form of JAK1, JAK3, STAT5b, STAT3, IL2RG, CARD11 and BRAF are represented by SEQ ID NO: 484-499. Other such expression constructs can be constructed by replacing the nucleic acids encoding the CAR/TCR with nucleic acids encoding different CAR/TCRs. Thus, the nucleic acid encoding CD8SP-FMC63-(vL-vH)-Myc-BBz cassette in SEQ ID NO: 484 can be replaced by nucleic acid encoding a different CAR or a TCR. Similarly, the nucleic acids encoding the JAK3-M511I mutant can be replaced by nucleic acid encoding a different mutant JAK1(e.g., SEQ ID NO:375-376), JAK3 (e.g., SEQ ID NO:362-367), STAT5b (e.g., SEQ ID NO:369-373), STAT3 (e.g., SEQ ID NO:381-385), IL2RG (e.g., SEQ ID NO:387), CARD11 (e.g., SEQ ID NO:378-379) and BRAF (e.g., SEQ ID NO:389). In some embodiments, the expression of constitutively active form of JAK1, JAK3, STAT5b, STAT3, IL2RG, CARD11 and BRAF in CAR-T/TCR-T cells is achieved by alteration of their genomic locus by homologous recombination. For example, one or more copies of JAK3 gene in the CAR-T cells can be modified by homologous recombination to mutate it to JAK3-M511I mutant form by using Cas9/CRISP system.

In another embodiment, the patients have CAR-T/TCR-t expanded ex vivo in the presence of chemical inhibitors of one or more of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, before administration to the patient. The ex-vivo expansion of CAR-T/TCR-T cells may also include inhibitors of AKT/PI3K pathway and other immunomodulatory drugs, such as Lenalidomide.

In another embodiment, the patients have CAR-T/TCR-T expanded and administered without ex vivo treatment or modification. The CAR-T/TCR-T cells are infused and the patient treated with BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 inhibitor for days, weeks, months or years as needed to maintain the anticancer or antiviral effect. The patient may also receive treatment with AKT/PI3K inhibitors and/or immunomodulatory drugs (IMiDs) such as lenalidomide.

Essentially a similar approach is used to expand other immune effector cells including but not limited to those expressing TCR, SIR, TFP, and/or AbTCR etc.

Co-Application with Other Anti-Exhaustion Measures

BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 inhibitor agents and/or constitutively active form of JAK1, JAK3, STAT5b, STAT3, IL2RG, CARD11 and BRAF described above are administered in combination with interventions against checkpoint receptors or their ligands (“dual treatment”). In some embodiments, BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 inhibitor agents and/or constitutively active form of JAK1, JAK3, STAT5b, STAT3, IL2RG, CARD11 and BRAF described above are administered in combination with bispecific and multispecific engagers described in this disclosure, such as Blinatumomab.

In some embodiments, cancer patients who are eligible for treatment with antibodies for checkpoint receptor blockade (e.g., non-small cell lung cancer, transitional cell cancer, melanoma, others) are treated with checkpoint axis antibody (e.g., nivolumab, atezolizumab) and BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, inhibitor drugs (dual treatment) as follows: The procedure of applying dual treatment in cancer patients follows the methods established for each of the checkpoint axis antibodies for determining eligibility with specific tumor types: some require testing tumor for PDL1 expression and others do not. If data support that reversal of exhaustion with checkpoint receptor antibodies induces tumor responses, this is considered adequate justification to improve the responses with the addition of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, inhibitor strategies.

In some embodiments, BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, inhibitor agents and/or constitutively active form of JAK1, JAK3, STAT5b, STAT3, IL2RG, CARD11 and BRAF described above are administered in combination with bispecific and multispecific engagers, such as Blinatumomab, and/or costimulatory agents (e.g., Utomilumab) and/or check point inhibitors (e.g., nivolumab, atezolizumab) and/or immunomodulatory drugs (e.g, Lenalidomide).

Knockout in CAR-T Cells to Resist Exhaustion and to Promote Long-Term Persistence

Exhaustion is reported in several different CAR-T cells (Long et al., 2015). Knocking out BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 is contemplated to allow the generation of a diverse immune response by altering the proliferation, survival, cytokine secretion, cytotoxicity, terminal differentiation, exhaustion and in vivo persistence of engineered T cells.

To generate a diverse pool of CAR-T cells, BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, is knocked-out using CRISPR/Cas9-mediated gene knockout in CAR-T cells. The knock out in CAR-T cells is achieved by electroporating Cas9 mRNA and guide RNA (gRNA) for BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5. The guide RNA (gRNA) are designed to target the exons of the above genes and are listed in Table 8. In some assays, cas-9 is Cas9 ribonucleoproteins (RNPs). In some assays, Cas9 RNP and in vitro transcribed gRNA targeting BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 are preassembled and then electroporated into T cells.

In another embodiment, the CAR-T cells are modified by co-transduction or sequential transduction to express constitutively active form of JAK1, JAK3, STAT5b, STAT3, IL2RG, CARD11 and BRAF either alone or in combination along with knock-out of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5. In some embodiments, constitutively active form of JAK1, JAK3, STAT5b, STAT3, IL2RG, CARD11 and/or BRAF are expressed in CAR-T cells using the same vector as that used to express CAR. In alternate embodiments, constitutively active form of JAK1, JAK3, STAT5b, STAT3, IL2RG, CARD11 and/or BRAF are expressed in CAR-T cells using a separate vector then the one used to express CAR. In some embodiment, constitutively active forms of JAK1, JAK3, STAT5b, STAT3, IL2RG, CARD11 and/or BRAF are expressed in immune effector cells by inducing mutation in their genomic allele(s) using homologous recombination, for example, using CAS9 system.

Analogous applications of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 knockout and/or expression of constitutively active forms of JAK1, JAK3, STAT5b, STAT3, IL2RG, CARD11 and/or BRAF with other immune effector cells including, but not limited to, TILs, transferred-TCR T cells or anti-viral CAR-T cells is also suitable.

Use of Blinatumomab and Other T Cell Bispecific Engagers for Activation and Expansion of T Cells In Vitro

Buffy coat cells are obtained from healthy de-identified adult donors from the Blood Bank and used to isolate peripheral blood mononuclear cells (PBMC) by Ficoll-Hypaque gradient centrifugation. PBMC are either used as such or used to isolate T cells using CD3 magnetic microbeads (Miltenyi Biotech) and following the manufacturer's instructions. PBMC or isolated T cells are re-suspended in XVIVO medium (Lonza) supplanted with 10 ng/ml CD3 antibody, 10 ng/ml CD28 antibody and 100 IU recombinant human-IL2. In an alternate embodiment, PBCM or T cells are activated by resuspending them at concentration of 5×10⁵ cells/ml in fresh XVIVO media containing Blinatumomab (10 ng/ml) plus irradiated (100 Gy) REC-1 cells (Mantle Cell lymphoma; ATCC Catalog #CRL-3004) at concentration of 5×10⁶ cells/ml in the presence of 50 IU recombinant human-IL2. In another alternate embodiment, the cell culture medium further contains a purified CD19×CD28 bispecific antibody (SEQ ID NO: 2708, 2705 or 2706) and/or a CD19×41BB bispecific antibody (SEQ ID NO: 2761, 2762, 2758 or 2764) each at concentration of about 10 ng/ml. Cells are cultured at 37° C., in a 5% CO₂ humidified incubator. Cells are activated in the above medium for 1 day prior to infection with lentiviral vectors encoding a CAR or a TCR (e.g., SEQ ID NO: 2822 to 2835) or a TCR (SEQ ID NO: 2836). In general, primary cells (e.g. T cells) are infected in the morning using spin-infection (1800 rpm for 90 minutes at 37° C. with 300 μl of concentrated virus that has been re-suspended in XVIVO medium in the presence of 8 μg/ml of Polybrene® (Sigma, Catalog no. H9268). After the infection, the cells are pelleted and resuspended at concentration of 5×10⁵ cells/ml in fresh XVIVO media containing Blinatumomab (10 ng/ml) plus irradiated (100 Gy) REC-1 (Mantle Cell lymphoma; ATCC Catalog #CRL-3004) cells (5×10⁶ cells/ml) and 50 IU recombinant human-IL2. Cells are cultured in the above medium for 10-15 days. Culture media is supplemented with fresh REC-1 cells, blinatumomab and IL2 every 2-4 days. Robust expansion of cells is achieved upon culture in the presence of REC-1 cells and blinatumomab as measured by CFSE stainng. The expanded CAR-T cells are subsequently used for in vitro cytotoxicity studies, in vivo efficacy studies in NSG mice and clinical trial of patients with cancers expressing the antigens targeted by the CAR/TCR using methods known in the art.

The above experiment is also repeated using CD19+ RAJI and NALM6 cells. Although expansion is seen upon co-culturing of CAR or TCR expressing T cells with RAJI and NALM6 cells in the presence of Blinatumomb, the degree of expansion is less than that observed with REC-1 cells. Thus, mantle cell lymphoma cells and, in particular, REC-1 cells are the preferred antigen presenting cells for expansion of T cells (e.g., CAR-T cells, TCR-T cells and TILs) when used in conjuction with bispecific T cell engagers that has at least one binding domain that binds to an antigen expressed on REC-1 (e.g, CD19, CD20, CD22 and BCMA). REC-1 cells also are the preferred antigen presenting cells for expansion of CAR-T cells targeting CD19, CD20, CD22 and BCMA.

The experiment is repeated by replacing REC-1 cells by CD19-ectodomain coated magnetic beads (CD19-ECD-beads). CAR-T cells are incubated with CD19-ECD-beads at ratio of 1:1, 1:5 and 1:10 in in fresh XVIVO media containing Blinatumomab (10 ng/ml) and a CD28 antibody (10 ng/ml) and IL2 (50 IU). In an alternate embodiment, the cell culture medium further contains a purified CD19×CD28 bispecific antibody (SEQ ID NO: 2708, 2705 or 2706) and/or a CD19×41BB bispecific antibody (SEQ ID NO: 2761, 2762, 2758 or 2764) each at concentration of about 10 ng/ml. Cells are cultured at 37° C., in a 5% CO₂ humidified incubator for 10-25 days. Robust expansion of CAR-T cells is seen upon incubation with CD19-ECD beads in the presence of Blinatumomab.

Essentially a similar procedure as described above is used for infection with CAR-encoding retroviruses. Essentially a similar procedure is used for expansion of T cells transduced with CARs and TCR encoded by sleeping beauty or piggybac transposons. Transduction of T cells with CAR-encoding sleeping beauty and piggyback transposons is carried out essentially as described previously (Nakazawa et al, J Immunother 32, 8, October 2009 and Kebriaei et al, Clin Invest. 2016; 126(9):3363-3376). Cells are expanded with Blinatumomab and REC-1 cells as described above.

The above experiments are repeated with the exception that CD28 antibody is replaced by a CD19×CD28 bispecific antibody (e.g., SEQ ID NO: 2708, 2705 or 2706). The CAR expressing PBMC and/or T cells in the presence of Blinatumomab and a CD19×CD28 bispecific antibody (SEQ ID NO: 2708, 2705 or 2706) and REC-1 cells or JEKO cells and IL2 (50 IU). The CD19×CD28 bispecific antibody is used at concentrations between 100 μg/ml and 100 ng/ml. Robust expansion of CAR-expressing PBMC and/or T cells is seen when expanded in the presence of REC-1 cells and Blinatumomab, a CD19×CD28 bispecific antibody and IL2. The expanded CAR-T cells are subsequently used for in vitro cytotoxicity studies, in vivo efficacy studies in NSG mice and clinical trial of patients with cancers expressing the antigens targeted by the CAR/TCR using methods known in the art.

Essentially a similar procedure is used to expand the CAR-T expressing PBMC and/or T cells in the presence of Blinatumomab and a CD19×41BB bispecific antibody (SEQ ID NO: 2761, 2762, 2758 or 2764) and REC-1 cells. The CD19×41BB bispecific antibody is used at concentrations between 100 μg/ml and 100 ng/ml.

Essentially a similar procedure is used to expand the CAR-T expressing PBMC and/or T cells in the presence of Blinatumomab and REC-1 cells plus Utomilumab. The Blinatumomab and Utomilumab are each used at concentrations between 100 μg/ml and 100 ng/ml.

Essentially a similar procedure is used to expand the CAR-T expressing PBMC and/or T cells in the presence of Blinatumomab and REC-1 cells plus a CD28 agonist antibody. The Blinatumomab and Utomilumab are each used at concentrations between 100 μg/ml and 100 ng/ml.

Essentially a similar procedure is used to expand the CAR-T expressing PBMC and/or T cells in the presence of a BCMA x CD3 DART and a BCMA x CD28 bispecific antibody (SEQ ID NO: 2709, 2711 or 2712) and U266 cells. The BCMA x CD3 DART and BCMA x CD28 bispecific antibodies are used at concentrations between 100 μg/ml and 100 ng/ml.

Essentially a similar procedure is used to expand the CAR-T expressing PBMC and/or T cells in the presence of a BCMA x CD3 DART bispecific anitbody and a BCMA x 41BB bispecific antibody (SEQ ID NO: 2765, 2766 or 2767) and U266 cells. The BCMA x CD3 DART and BCMA x 41BB bispecific antibodies are used at concentrations between 100 μg/ml and 100 ng/ml.

Essentially a similar procedure is used to expand the CAR-T expressing PBMC and/or T cells in the presence of a CD22×CD3 bispecific antibody (SEQ ID NO: 2668, 2667, or 2663) and a CD19×CD28 bispecific antibody ((SEQ ID NO: 2708, 2705 or 2706) and REC-1 or JEKO cells. The CD22×CD3 bispecific and CD19×CD28 bispecific antibodies are used at concentrations between 100 μg/ml and 100 ng/ml. The experiment is also repeated using RAJI and NALM6 cells. Although expansion is seen upon co-culturing of CAR or TCR expressing T cells with RAJI and NALM6 cells in the presence of Blinatumomb, the degree of expansion is less than that observed with REC-1 cells.

Essentially a similar procedure is used to expand the CAR-T expressing PBMC and/or T cells in the presence of Blinatumomab and a CD22×41BB bispecific antibody (SEQ ID NO: 2775, 2778 or 2780) and REC-1 cells. The Blinatumomab and CD22×41BB bispecific antibody are used at concentrations between 100 μg/ml and 100 ng/ml.

In the preceding examples, bispecific antibodies are used to expand CAR-T expressing cells.

Essentially a similar procedure can be used to expand any T cell (e.g., TILs, recombinant TCR expressing T cell etc.) by using bispecific antibodies that bind to and activate T cells through one of their antigen binding domains and bind to an antigen expressed on an antigen presenting cell. For example, Blinatumomab, with or without a CD19×CD28 bispecific antibody (e.g., SEQ ID NO; 2708, 2705 or 2706) can be used to expand TILs or to expand T cells expressing a recombinant NY-ESO1 TCR or to expand T cells expressing a Mesothelin SIR when grown in the presence of CD19-expressing cells, such as REC-1 or K562-CD19 or Jeko-1 cells.

Use of Bispecific T Cell Engagers Targeting Different Antigens in the Presence of APC and APS

Buffy coat cells are obtained from healthy de-identified adult donors from the Blood Bank and used to isolate peripheral blood mononuclear cells (PBMC) by Ficoll-Hypaque gradient centrifugation. PBMC are either used as such or used to isolate T cells using CD3 magnetic microbeads (Miltenyi Biotech) and following the manufacturer's instructions. PBMC or isolated T cells are re-suspended in XVIVO medium (Lonza) supplanted with 10 ng/ml CD3 antibody, 10 ng/ml CD28 antibody and 100 IU recombinant human-IL2. Cells are cultured at 37° C., in a 5% CO2 humidified incubator. Cells are activated in the above medium for 1 day prior to infection with lentiviral vectors encoding a CAR or a TCR (e.g., SEQ ID NO: 2822 to 2835) or a TCR (SEQ ID NO: 2836). After the infection, the cells are pelleted and resuspended at concentration of 5×10⁵ cells/ml in fresh XVIVO media containing the Antigen presenting cells (APC) or antigen presenting substrate (APS), primary activation stimuli, co-stimulation agents and additional cytokines as shown in Table 17. All antibodies, inlcuding bispecific atnibodies, CD3, CD28 and Utomilumab are added at concentration of 10 ng/ml. The cytokines (IL2, IL7 or IL15) are added at concentration of 30-50 IU each. Cells are cultured in the above medium for 10-25 days. Culture media is exchanged with fresh media every 2-4 days. The expanded CAR-T cells are subsequently used for in vitro cytotoxicity studies, in vivo efficacy studies in NSG mice and clinical trial of patients with cancers expressing the antigens targeted by the CAR/TCR using methods known in the art.

Table 17

TABLE 17

| Exp. | | Activation | SEQ ID | | SEQ ID | Others |
|---|---|---|---|---|---|---|
| # | APC/APS | Stimulus | (PRT) | Costimulation | (PRT) | Additives |
| 1 | CD19-ECD- | Blinatumomab | | CD28 antibody | | IL2 |
| | Beads | | | beads | | |
| 2 | REC-1 | Blinatumomab | | Soluble CD28 | | IL2 |
| | | | | antibody | | |
| 3 | REC-1 | Blinatumomab | | Utomilumab | | IL2 + IL7 |
| 4 | REC-1 | Blinatumomab | | CD19 × CD28 | 2702 | IL15 + IL7 |
| 5 | REC-1 | Blinatumomab | | CD19 × 41BB | 2758 | IL2 |
| 6 | JEKO-1 | Blinatumomab | | CD22 × CD28 | 2724 | IL2 |
| 7 | REC-1 | Blinatumomab | | CD22 × 41BB | 2780 | IL2 + IL7 |
| 8 | REC-1 | Blinatumomab | | CD22 × CD28 | 2719 | IL15 + IL7 |
| 9 | REC-1 | Blinatumomab | | CD22 × 41BB | 2775 | IL2 |
| 10 | JEKO-1 | Blinatumomab | | CD20 × CD28 | 2713 | IL2 |
| 11 | REC-1 | Blinatumomab | | CD20 × 41BB | 2774 | IL2 |
| 12 | CD22-ECD- | CD22 × CD3 | 2663 | CD28 antibody | | IL2 |
| | Beads | | | beads | | |
| 13 | REC-1 | CD22 × CD3 | 2663 | CD28 antibody | | IL2 |
| | beads | | | | | |
| 14 | REC-1 | CD22 × CD3 | 2666 | CD28 antibody | | IL2 + IL7 |
| 15 | REC-1 | CD22 × CD3 | 2668 | Utomilumab | | IL15 + IL7 |
| 16 | REC-1 | CD22 × CD3 | 2663 | CD19 × CD28 | 2702 | IL2 |
| 17 | REC-1 | CD22 × CD3 | 2490 | CD19 × 41BB | 2758 | IL2 |
| 18 | JEKO-1 | CD22 × CD3 | 2668 | CD22 × CD28 | 2724 | IL2 + IL7 |
| 19 | REC-1 | CD22 × CD3 | 2663 | CD22 × 41BB | 2780 | IL15 + IL7 |
| 20 | REC-1 | CD22 × CD3 | 2666 | CD22 × CD28 | 2719 | IL2 |
| 21 | REC-1 | CD22 × CD3 | 2668 | CD22 × 41BB | 2775 | IL2 |
| 22 | JEKO-1 | CD22 × CD3 | 2663 | CD20 × CD28 | 2713 | IL2 |
| 23 | REC-1 | CD22 × CD3 | 2668 | CD20 × 41BB | 2774 | IL2 |
| 24 | CD19-ECD- | CD20 × CD3 | 2657 | CD28 antibody | | IL2 |
| | Beads | | | beads | | |
| 25 | REC-1 | CD20 × CD3 | 2658 | CD28 antibody | | IL2 |
| 26 | GRANTA-519 | CD20 × CD3 | 2660 | Utomilumab | | IL2 + IL7 |
| 27 | REC-1 | CD20 × CD3 | 2657 | CD19 × CD28 | 2702 | IL15 + IL7 |
| 28 | REC-1 | CD20 × CD3 | 2658 | CD19 × 41BB | 2758 | IL2 |
| 29 | JEKO-1 | CD20 × CD3 | 2660 | CD22 × CD28 | 2724 | IL2 |
| 30 | REC-1 | CD20 × CD3 | 2657 | CD22 × 41BB | 2780 | IL2 + IL7 |
| 31 | REC-1 | CD20 × CD3 | 2658 | CD22 × CD28 | 2719 | IL15 + IL7 |
| 32 | REC-1 | CD20 × CD3 | 2660 | CD22 × 41BB | 2775 | IL2 |
| 33 | JEKO-1 | CD20 × CD3 | 2657 | CD20 × CD28 | 2713 | IL2 |
| 34 | REC-1 | CD20 × CD3 | 2658 | CD20 × 41BB | 2774 | IL2 |
| 35 | BCMA-ECD- | BCMA × CD3 | 2653 | CD28 antibody | | IL2 |
| | Beads | | | beads | | |
| 36 | REC-1 | BCMA × CD3 | 2654 | CD28 antibody | | IL2 |
| 37 | U266 | BCMA × CD3 | 2655 | Utomilumab | | IL2 + IL7 |
| 38 | REC-1 | BCMA × CD3 | 2653 | CD19 × CD28 | 2702 | IL15 + IL7 |
| 39 | REC-1 | BCMA × CD3 | 2654 | CD20 × 41BB | 2774 | IL2 |
| 40 | U266 | BCMA × CD3 | 2655 | BCMA × CD28 | 2710 | IL2 |
| 41 | U266 | BCMA × CD3 | 2653 | BCMA × 41BB | 2766 | IL2 + IL7 |
| 42 | REC-1 | BCMA × CD3 | 2654 | BCMA × CD28 | 2709 | IL15 + IL7 |
| 43 | REC-1 | BCMA × CD3 | 2655 | BCMA × 41BB | 2765 | IL2 |
| 44 | REC-1 | BCMA × CD3 | | BCMA × CD28 | 2709 | IL2 |
| | | DART | | | | |
| 45 | REC-1 | BCMA × CD3 | | BCMA × 41BB | 2765 | IL2 |
| | | DART | | | | |
| 46 | EGFR-ECD- | EGFR × CD3 | 2814 | CD28 antibody | | IL2 |
| | Beads | | | beads | | |
| 47 | Hela | EGFR × CD3 | 2814 | CD28 antibody | | IL2 |
| 48 | Hela | EGFR × CD3 | 2814 | Utomilumab | | IL2 + IL7 |
| 49 | Hela | EGFR × CD3 | 2814 | EGFR × CD28 | 2815 | IL15 + IL7 |
| 50 | Hela | EGFR × CD3 | 2814 | EGFR × 41BB | 2816 | IL2 |
| 51 | A431 | EGFR × CD3 | 2814 | EGFR × CD28 | 2710 | IL2 |
| 52 | FITC-Beads | FITC × CD3 | 2817 | CD28 beads | | IL2 |
| 53 | FITC-Beads | FITC × CD3 | 2817 | CD28 antibody | | IL2 |
| 54 | FITC-Beads | FITC × CD3 | 2817 | Utomilumab | | IL2 + IL7 |
| 55 | FITC-Beads | FITC × CD3 | 2817 | FITC × CD28 | 2818 | IL15 + IL7 |
| 56 | FITC-Beads | FITC × CD3 | 2817 | FITC × 41BB | 2819 | IL2 |
| 57 | STREPTA | STREPTAG × | 2820 | CD28 beads | | IL2 |
| | G-Beads | CD3 | | | | |
| 58 | STREPTA | STREPTAG × | 2820 | CD28 antibody | | IL2 |
| | G-Beads | CD3 | | | | |
| 59 | STREPTA | STREPTAG × | 2820 | Utomilumab | | IL2 + IL7 |
| | G-Beads | CD3 | | | | |
| 60 | STREPTA | STREPTAG × | 2820 | STREPTAG × | 2821 | IL15 + IL7 |
| | G-Beads | CD3 | | CD28 | | |
| 61 | Hela- | STREPTAG × | 2820 | STREPTAG × | 2852 | IL2 |
| | STREPTA | CD3 | | 41BB | | |
| | G-EGFR | | | | | |

Use of Blinatumomab and/or Other T Cell Bispecific Antibodies and/or Costimulatory Molecules (e.g., 41BB Ligand or Utomilumab) for Activation and Expansion of T Cells In Vivo

A leukapheresis product is collected from a patient with metastatic ovarian cancer expressing Mesothelin. Selection of CD3-positive T lymphocytes is performed using the CliniMACS Prodigy® System from Miltenyi Biotec and following the manufacturer's recommendations. Approximately 10⁸ to 10⁸ T cells are transduced with a lentiviral encoding a Mesothlin CAR (e.g., SEQ ID NO: 2832). The CAR-T cells are expanded for 14-21 days in a closed system using CD3/CD28 beads. The resulting cell products undergoes quality control testing (including sterility and tumor specific cytotoxicity tests). Patient optionally receives lymphodelpeting chemotherapy prior to receving the infusion of CAR-T cells. An exemplary lymphodepleting chemotherapy regimens includes 30 mg/m²/day i.v. fludarabine plus 500 mg/m²/day cyclophosphamide i.v. x 3 days. An alternate regimen includes Cyclophosphamide at a dose of 60 mg/kg/day IV on Day −7 and Day −6 followed by Fludarabine 25 mg/m²/day IVPB on Day −5 through Day −1. Another alternate regimen is Cyclophosphamide 1000 mg/m². If the lymphodepleting chemotherapy is administered, then the CAR-T cells are infused 1 day after completion of lymphodepleting chemotherapy. Otherwise, CAR-T cells are infused without prior chemotherapy. The CAR-T cell product is administered to the patient by intravenous infusion. The dose of CAR-T product varies from 1×10⁴ CAR+ve CD3 cells/kg to 5×10¹ CAR+ve CD3 cells/kg. Within 3 hours of infusion of CAR-T cells, the patient is administered Blinatumomab. The initial dose of Blinatunomab is 9 μg/day by continuous intravenous infusion for the first 7 day s of treatment and then escalated to 28 μg/day by continuous intravenous infusion starting on Day 8 (Week 2) through Day 29 (Week 41 Optionally, Interleukin (IL)-2 is administered subcutaneously at a dose of 250000 IU/kg once daily for a maximum of 21 doses, with the first dose administered within 3 hours of infusion of CAR-T cell product. Optionally, utomilumab is administered at dose of 0.6 to 10 mg/kg every 4 weeks intravenously. Infusion of Blinatumomab is stopped in case patient shows any signs of toxicity, including symptoms and signs suggestive of cytokine release syndrome or neurotoxicity

Example: Use of Perixafor Mobilized T Cells for Manufacturing of TCR/CAR-T Cell Product and Use of Blinatumomab for In Vivo Expansion of the TCR/CAR T Cell Product.

A patient (HLA-A2) with multiple myeloma receives Perixafor at a dose of 0.24 mg/kg subcutaneously daily for 3 days. A leukapheresis product is collected from a patient 6 h after the last dose of Perixafor. Selection of CD3-positive T lymphocytes is performed using the CliniMACS Prodigy® System from Miltenyi Biotec and following the manufacturer's recommendations. Approximately 10⁸ to 10⁸ T cells are transduced with a lentiviral encoding a NY-ESO-1 T Cell Receptor (SEQ ID NO: 2836) and engineered T Cell product are expanded using CD3/CD28 beads according to methods known in the art. The resulting cell products undergoes quality control testing (including sterility and tumor specific cytotoxicity tests). The TCR-T cell product is administered to the patient by intravenous infusion. The dose of TCR-T product varies from 1×10⁴ NY-ESO-1 TCR+ve CD3 cells/kg to NY-ESO-1 TCR 5×10⁹ CAR+ve CD3 cells/kg. Blinatumomab (100 ng/10⁶ cells) is added to the bag containing the NY-ESO T Cell Receptor Engineered T Cell product, the products are mixed on a rocking platform for 15 minutes and then infused into the patient. The patient also receives an infusion of Blinatumomab within 5 minutes after completion of infusion of NY-ESO TCR engineered T cell product. The initial dose of Blinatumomab is 9 μg/day by continuous intravenous infusion for the first 7 days of treatment and then escalated to 28 μg/day by continuous intravenous infusion starting on Day 8 (Week 2) through Day 29 (Week 4). Essentially a similar procedure is used to treat patients with synovial sarcoma, myxoid round cell liposarcoma, melanoma, NSCLC and ovarian cancer using the NY-ESO TCR product.

Essentially a similar procedure is used to treat patients with acute myeloid leukemia with a WT1-expressing AML using a recombinant WT1 targeted TCR engineered T cell product. Essentially a similar approach can be used to treat other diseases using Perixafor-mobilized immune cells (e.g., T cells) that have been engineered to express a CAR (e.g., a Mesothelin CAR, SEQ ID NO: 2832; a IL13Ra2 CAR, SEQ ID NO:2829; Her2 CAR, SEQ ID NO: 2831; AFP/MHC CAR, SEQ ID NO: 2835) or a TCR where the CAR or the TCR targets an antigen or antigens expressed on the disease causing or disease-associated cells. The method can be also used for adoptive cellular therapy involving Tumor Infiltrating lymphocytes (TIL) and/or with T cell vaccination approaches.

Use of TRAIL Antagonists and Bispecific T Cell Engagers for Manufacturing of CAR-T Cell Products

Buffy coat cells are obtained from healthy de-identified adult donors from the Blood Bank and used to isolate peripheral blood mononuclear cells (PBMC) by Ficoll-Hypaque gradient centrifugation. PBMC are either used as such or used to isolate T cells using CD3 magnetic microbeads (Miltenyi Biotech) and following the manufacturer's instructions. PBMC or isolated T cells are re-suspended in XVIVO medium (Lonza) supplanted with 10 ng/ml CD3 antibody, 10 ng/ml CD28 antibody and 100 IU recombinant human-IL2. Optionally, TRAIL antagonists MAB375-SP, DR5-Fc or DR4-Fc are added at concentration of 50 ng/ml each, either singly or in combination. Cells are cultured at 37° C., in a 5% CO₂ humidified incubator. Cells are activated in the above medium for 1 day prior to infection with a lentiviral vector encoding a CAR targeting CD19 (e.g., SEQ ID NO: 2822). In general, primary cells (e.g. T cells) are infected in the morning using spin-infection (1800 rpm for 90 minutes at 37° C. with 300 μl of concentrated virus that has been re-suspended in XVIVO medium in the presence of 8 μg/ml of Polybrene® (Sigma, Catalog no. H9268). The media is changed in the evening and the infection is repeated for two more days for a total of 3 infections. After the 3rd infection, the cells are pelleted and resuspended at concentration of 5×10⁵ cells/ml in fresh XVIVO media containing BCMA x CD3 bispecific antibody (SEQ ID NO: 2653) at 10 ng/ml plus irradiated (100 Gy) REC-1 cells (5×10⁶ cells/ml) and 50 IU recombinant human-IL2. Cells are cultured in the above medium for 10-15 days. Optionally, TRAIL antagonists MAB375-SP, DR5-Fc or DR4-Fc are added at concentration of 50 ng/ml each, either singly or in combination. Culture media is supplemented with fresh REC-1 cells, BCMA x CD3 BiTE and IL2 every 3-4 days. Robust expansion of cells is achieved upon culture in the presence of REC-1 cells and bispecific antibodies. Essentially a similar procedure as described above is used for infection with CAR-encoding retroviruses. In an alternate embodiment, the BCMA x CD3 BiTE is replaced by Blinatumomab at 10 ng/ml

TRAIL Neutralizing Antibody Blocks IL1α Production by CAR-T Cells when Cultured with THP-1 Cells.

T cells isolated from mononuclear cells using CD3 magnetic microbeads (Miltenyi Biotech) are stimulated with CD3 antibody (10 ng/ml) and CD28 antibody (10 ng/ml) in the presence of IL2 (30 IU) for 24 hours and then infected with a lentiviral vector encoding a chimeric antigen receptor targeting CD19 (GV8) and co-expressing EGFP (Enhanced Green Fluorescent Protein) or a control vector encoding EGFP alone. Cells were expanded in XVIVO medium (Lonza) supplanted with 10 ng/ml CD3 antibody, 10 ng/ml CD28 antibody and 30 IU recombinant human-IL2 and used in the following experiments:

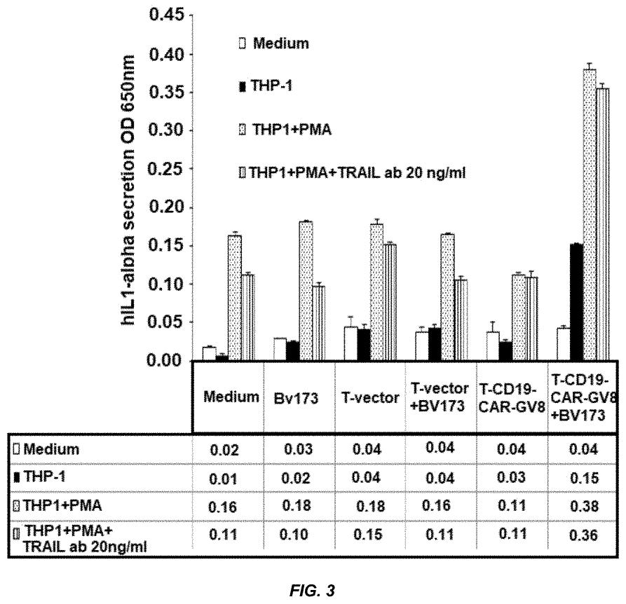

THP-1 cells (Monocytic Leukemia) were plated in 24-well plate at a density of 10⁶ cells/well and were either left untreated, or treated with PMA 50 ng/ml (to induced macrophage differentiation) or PMA 50 ng/ml+TRAIL antibody (MAB375-SP 20 ng/ml). Plates were incubated overnight for THP1 cells to differentiate. In another 24-well plate Bv173-hGLuc (3 million cells/well/500 μl) were co-cultured with either T-vector (3×10⁶ cells/well/500 μl) cells or T-CD19-CAR cells (3×10⁶ cells/well/500 μl) at an E:T ratio of 1:10 for 24 h. Next day, supernatant was collected from Bv173+ T cells and added on top of THP-1 cells that had been plated in a separate 24-well plate one day earlier. THP1 cells were cultured in the presence of supernatant for 72 h and cells were collected, centrifuged and supernatant was tested for IL1α and IL6 production by ELISA. The FIG. 3 shows inhibition of PMA-induced macrophage differentiation of THP1 cells is accompanied by increase in IL1α production which is blocked by TRAIL antibody. The TRAIL antibody also has a modest inhibitory effect on IL1α production when supernatant from T cells, including CAR-T cells, is incubated with TPA-differentiated THP1 cells.

AIL Antibody Blocks IL1-Alpha Production by THP1 Cells when Co-Cultured with CAR-T Cells

T cells isolated from mononuclear cells using CD3 magnetic microbeads (Miltenyi Biotech) are stimulated with CD3 antibody (10 ng/ml) and CD28 antibody (10 ng/ml) in the presence of IL2 (30 IU) for 24 hours and then infected with a lentiviral vector encoding a chimeric antigen receptor targeting CD19 (GV8) and co-expressing EGFP (Enhanced Green Fluorescent Protein) or a control vector encoding EGFP alone. Cells are expanded in XVIVO medium (Lonza) supplanted with 10 ng/ml CD3 antibody, 10 ng/ml CD28 antibody and 30 IU recombinant human-IL2.

THP1 cells were plated in a 24-well plate at 1.5×10⁵ cells per well either alone or with CD19+ Bv173 (at 2.5×10⁵ cells per well) and/or T-vector cells or T-cells expressing a CAR (GV8) directed against CD19 (25K cells per well) with or without a TRAIL antibody MAB375-SP (R&D Systems) added at concentrations of 12 ng/ml or 20 ng/ml. The E:T ratio of T cells to BV173 cells was 1:10. Supernatant was collected after 48 hours of co-culture and 25 μl of supernatant was assayed per well in triplicate for IL1-alpha and IL6-using a Duel-Set ELISA kit (R&D systems).

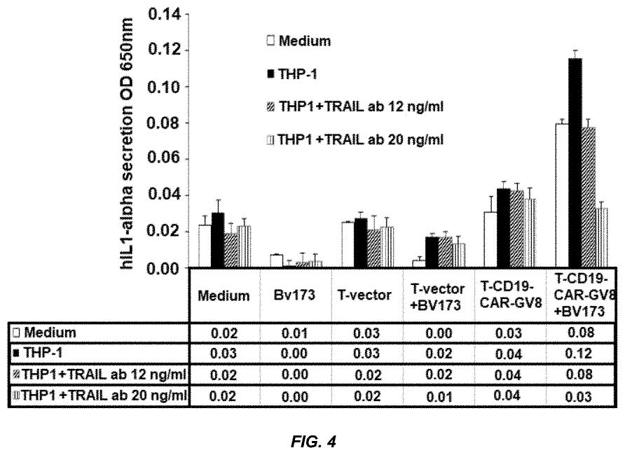

FIG. 4 shows that treatment with the TRAIL antibody significantly blocks IL1α production by THP1 cells when co-cultured with T cells expressing a CD19 CAR (GV8) plus BV173 target cells.

TRAIL Antibody Blocks IL1-Alpha Production by PMA-Differentiated THP1 Cells when Co-Cultured with CAR-T Cells

T cells isolated from mononuclear cells using CD3 magnetic microbeads (Miltenyi Biotech) are stimulated with CD3 antibody (10 ng/ml) and CD28 antibody (10 ng/ml) in the presence of IL2 (30 IU) for 24 hours and then infected with a lentiviral vector encoding a chimeric antigen receptor targeting CD19 (GV8) and co-expressing EGFP (Enhanced Green Fluorescent Protein) or a control vector encoding EGFP alone. Cells are expanded in XVIVO medium (Lonza) supplanted with 10 ng/ml CD3 antibody, 10 ng/ml CD28 antibody and 30 IU recombinant human-IL2.

THP1 (6 million) cells were plated in a 6-well plate and treated with 50 ng/ml PMA for 24 hours. Next day, cells were treated with PBS-EDTA to detach the cells, given a wash to remove PMA and washed cells were plated in a 24-well plate at 1.5×10⁵ cells per well either alone or with CD19+ Bv173 (2.5×10⁵ cells per well) and/or T-Parental cells or T-cells expressing a CAR (GV8) directed against CD19 (2.5×10⁴ cells per well) with or without a TRAIL antibody MAB375-SP (R&D Systems) added at concentrations of 12 ng/ml or 20 ng/ml. The E:T ratio of T cells to BV173 cells was 1:10. Supernatant was collected after 48 hours of co-culture and 25 μl of supernatant was assayed per well in triplicate for IL1-alpha and IL6-using a Duel-Set ELISA kit (R&D systems).

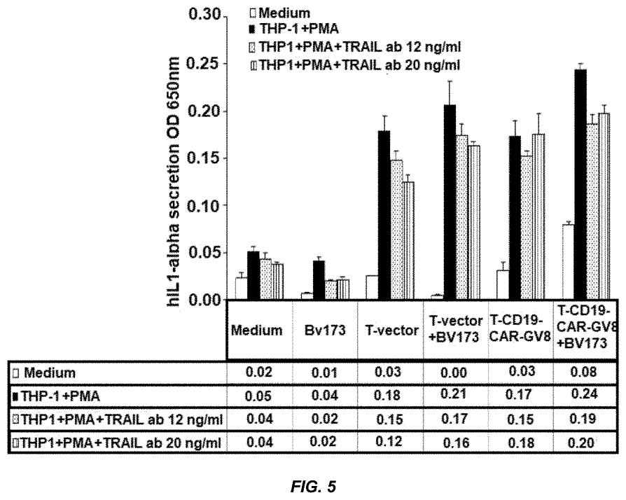

The FIG. 5 shows that treatment with TRAIL antibody blocks IL1α production by PMA-differentiated THP cells (mTHP1), mTHP1 cells co-cultured with BV173 cells, mTHP1 cells cocultured with T-vector cells and mTHP1 cells cocultered with CD19-CAR-T cells plus BV173 cells. Thus, TRAIL is a major mediator of T cells-induced (including CAR-T cells-induced) IL1α production by monocytes/macrophages cells. Since IL1α production by monocytes/macrophage lineage cells is believed to be a major mediator of cytokine release syndrome and neurotoxicity associated with administration of adoptively transferred T cells, including CAR-T and TCR-T cells, these results demonstrate that TRAIL inhibitors (e.g., TRAIL antibody, DR5-Fc, DR4-Fc, DcR1-Fc, DcR2-Fc, antagonist DR5 antibodies, small molecule and nucleic acid inhibitors of TRAIL/DR5 signaling) can be used for the prevention and treatment of CRS and neurotoxicity associated with administration of adoptively transferred T cells.

Use of a Neutralizing TRAIL Antibody to Prevent Cytokine Release Syndrome and Neurotoxicity

A patient with Acute Lymphocytic Leukemia receives a CD19-CAR-T cell product (5×10⁸ CAR+ve CD3 cells/kg). Patient is at high risk of developing CRS and neurotoxicity due to high leukemia burden. On day after administration of CAR-T cells, patient is given a prophylactic dose of clinical-grade neutralizing antibody against TRAIL (MAB375-SP) at dose of 5 mg/kg by intravenous infusion following pre-medication with Benadryl (50 mg i.v.) and Paracetamol (750 mg PO). The dose is repeated after 1 week. In an alternate example, a patient with multiple myeloma receives a BCMA-CAR-T cell product (5×10⁸ CAR+ve CD3 cells/kg). Patient is given a prophylactic dose of clinical-grade neutralizing antibody against TRAIL (MAB375-SP) at dose of 5 mg/kg by intravenous infusion following pre-medication with Benadryl (50 mg i.v.) and Paracetamol (750 mg PO).

Use of a Neutralizing TRAIL Antibody to Treat Cytokine Release Syndrome and Neurotoxicity

A patient with Acute Lymphocytic Leukemia receives a CD19-CAR-T cell product. Three days after infusion, patient develops signs and symptoms suggestive of Cytokine Release syndrome and neurotoxicity, including high fever, hypotension, respiratory distress and altered mental status. Patient is administered a clinical grade neutralizing antibody against TRAIL (MAB375-SP) at dose of 5 mg/kg by intravenous infusion after pre-medication with Benadryl (50 mg i.v.) and Paracetamol (750 mg PO). A second dose of the neutralizing antibody against TRAIL is administered after 12 hours.

Use of DR5-Fc to Prevent Cytokine Release Syndrome and Neurotoxicity

A patient with Acute Lymphocytic Leukemia receives a CD19-CAR-T cell product. Patient is at high risk of developing CRS and neurotoxicity due to high leukemia burden. On day after administration of CAR-T cells, patient is given a prophylactic dose of clinical-grade DR5-Fc at dose of 5 mg/kg by intravenous infusion following pre-medication with Benadryl (50 mg i.v.) and Paracetamol (750 mg PO). The dose is repeated after 1 week. In an alternate example, a with multiple myeloma receives a BCMA-CAR-T cell product. Patient is given a prophylactic dose of clinical-grade neutralizing antibody against TRAIL (MAB375-SP) at dose of 5 mg/kg by intravenous infusion following pre-medication with Benadryl (50 mg i.v.) and Paracetamol (750 mg PO). In an alternate embodiment, the patient is administered other TRAIL antagonists (e.g, DR4-Fc, DcR1-Fc and DcR2-Fc) instead of or in combination with DR5-Fc.

Use of DR5-Fc to Treat Cytokine Release Syndrome and Neurotoxicity

A patient with Acute Lymphocytic Leukemia receives a CD19-CAR-T cell product. Three days after infusion, patient develops signs and symptoms suggestive of Cytokine Release syndrome and neurotoxicity, including high fever, hypotension, respiratory distress and altered mental status. Patient is administered a clinical grade DR5-Fc at dose of 5 mg/kg by intravenous infusion after pre-medication with Benadryl (50 mg i.v.) and Paracetamol (750 mg PO). A second dose of DR5-Fc is administered after 12 hours. In an alternate embodiment, the patient is administered other TRAIL antagonists (e.g, DR4-Fc, DcR1-Fc and DcR2-Fc) instead of or in combination with DR5-Fc.

Use of Intra-Thecal MAB375-SP to Treat Neurotoxicity

A patient with Acute Lymphocytic Leukemia receives a CD22-CAR-T cell product. Ten days after infusion of CAR-T cells, patient develops signs and symptoms suggestive of neurotoxicity, including confusion, aphasia, altered mental status and seizures. Patient is administered a clinical grade neutralizing antibody against TRAIL (MAB375-SP) at dose of 20 mg (resuspended in 2 ml of normal saline) by intra-thecal injection. A second dose of intra-thecal MAB375 is administered after 3 days.

Use of Intra-Thecal DR5-Fc to Treat Neurotoxicity

A patient with multiple myeloma receives a BCMA-CAR-T cell product. Ten days after infusion of CAR-T cells, patient develops signs and symptoms suggestive of neurotoxicity, including confusion, aphasia, altered mental status and seizures. Patient is administered a clinical grade neutralizing DR5-Fc at dose of 20 mg (resuspended in 2 ml of normal saline) by intra-thecal injection. A second dose of intra-thecal DR5-Fc is administered after 3 days. In an alternate embodiment, the patient is administered other TRAIL antagonists (e.g, DR4-Fc, DcRT-Fc and DcR2-Fc) instead of or in combination with DR5-Fc.

Use of TRAIL Antagonist in the Manufacturing of Cellular Therapy Products

Buffy coat cells are obtained from healthy de-identified adult donors from the Blood Bank and used to isolate peripheral blood mononuclear cells (PBMC) by Ficoll-Hypaque gradient centrifugation. PBMC are either used as such or used to isolate T cells using CD3 magnetic microbeads (Miltenyi Biotech) and following the manufacturer's instructions. PBMC or isolated T cells are re-suspended in XVIVO medium (Lonza) supplanted with 10 ng/ml CD3 antibody, 10 ng/ml CD28 antibody and 100 IU recombinant human-IL2. Cells are cultured at 37° C., in a 5% CO2 humidified incubator. Cells are activated in the above medium for 1 day prior to infection with a lentiviral vector encoding a CAR or a TCR (e.g., SEQ ID NO: 2822 to 2836). After the infection, the cells are pelleted and resuspended at concentration of 5×10⁵ cells/ml in fresh XVIVO media containing 10 ng/ml CD3 antibody, 10 ng/ml CD28 antibody, 50 IU recombinant human-IL2. The following TRAIL antagonists are added either singly or in various combinations i) MAB375-SP at 50 ng/ml, ii) recombinant DR5-Fc (Sigma-Aldrich; D9563) at 50 ng/ml; iii) recombinant DR4-Fc (Sigma-Aldrich; D9438) at 50 ng/ml; iv) Recombinant Human TRAIL R3/TNFRSF10C Fc Chimera Protein (R&D Systems) at 50 ng/ml; and v) DcR2-Fc at 50 ng/ml. Cells are cultured at 37° C., in a 5% CO2 humidified incubator. Cells are cultured in the above medium for 10-27 days with change of medium and addition of fresh TRAIL antagonists every 2-3 days. At the end of culture medium, the T cells are collected, washed with normal saline and resuspended in normal saline and used for in vivo studies in mice. T cells cultured in the presence of TRAIL antagonists are shown to possess robust anti-tumor activity and long term persistence in vivo when tested in appropriate model in NSG mice consisting of xenograft of human tumor cells expressing the antigen targeted by the CAR or TCR constructs. Essentially a similar procedure is used to manufacture CAR-T cells for human clinical trials.

Expression of TRAIL Antagonists in the T Cells

Buffy coat cells are obtained from healthy de-identified adult donors from the Blood Bank and used to isolate peripheral blood mononuclear cells (PBMC) by Ficoll-Hypaque gradient centrifugation. PBMC are either used as such or used to isolate T cells using CD3 magnetic microbeads (Miltenyi Biotech) and following the manufacturer's instructions. PBMC or isolated T cells are re-suspended in XVIVO medium (Lonza) supplanted with 10 ng/ml CD3 antibody, 10 ng/ml CD28 antibody and 100 IU recombinant human-IL2. Cells are cultured at 37° C., in a 5% CO2 humidified incubator. Cells are activated in the above medium for 1 day prior to infection with a lentiviral vector expressing a cassette encoding a CAR or a TCR either alone or co-expressing a TRAIL antagonist (e.g, DR5-Fc, DR4-Fc, DcR1-Fc, DcR2-Fc and chimeric fusion proteins encoding the extracellular TRAIL binding domain linked to the cytosolic domain of a different signaling receptor via a transmembrane domain). The SEQ ID NO: of the exemplary expression constructs targeting CD19 (using FMC63 antigen binding domain) and NYESO-1/MHC I or II complex and co-expressing TRAIL antagonists are provided in Table 7a in SEQ ID NO: 2853-2863; 2865-2875; 2877-2887; 2889-2899; 2901-2911; 2913-2923; 2925-2935. After the infection, the cells are pelleted and resuspended at concentration of 5×10⁵ cells/ml in fresh XVIVO media containing 10 ng/ml CD3 antibody, 10 ng/ml CD28 antibody, 50 IU recombinant human-IL2. Cells are used expanded in the above medium for 14 days and then used for in vitro studies to determine their phenotype (e.g., expression of exhaustion and activation markers), cytokine production (e.g., IL1α, IL1P, TNFα, IFNγ and IL6), proliferative potential, cytotoxicity (e.g. using Matador assay) using appropriate cell line models. NALM6 and REC-1 cells are used as the target cells for FMC63 based constructs, while MEL624 cell lines is used for NYESO-1/HLA-A2 complex targeting constructs. The in vivo activity of the T cells expressing the CAR/TCR and co-expressing the TRAIL antagonist is confirmed using xenograft of appropriate cell lines (e.g., NALM6 and MEL624) expressing their target antigens in immunodeficient mice (e.g., NSG mice). In addition, effect of the CAR/TCR coexpressing the TRAIL antagonists on development of cytokine release syndrome is tested using the SCID-Biege mouse model of CRS as decribed by Giavridis, T et al (Nat Med. 2018 June; 24(6):731-738. doi: 10.1038/s41591-018-0041-7; PMID: 29808005). Finally, the CAR-T and NYESO/HLA-A2 TCR-T cell products are administered to patients enrolled in a clinical trial to test the safety and efficacy of CAR/TCR products coexpressing TRAIL antagonists for the treatment of CD19+ B cell malignancies (for FMC63 based constructs) and NYESO-1/HLA-A2 expressing melanomas (for NYESO-1/HLA-A2 targeting constructs), respectively.

Use of T Cells Expressing the Costimulatory Molecules (e.g., DcR1, DcR2, CD27, CD28, 41BB, OX40 and GITR)

A clinical trial is conducted to test the safety and efficacy of T cells expressing different costimulatory molecules in subjects with cancer. The cancer tissue (e.g., a biopsy of lung cancer, ovarian, colon cancer or lymphoma etc.) is obtained from the subject and then subjected to gene expression analysis by RNA-SEQ and/or microarray and protein analysis by histochemistry and/or flow cytometry to analyze the expression of different ligands (e.g., TRAIL, CD70, CD80, CD84, 41BBL, OX40L and GITRL) of the costimulatory receptors (e.g., DcR1, DcR2, CD27, CD28, 4TBB1, OX40 and GITR). Subsequently, CAR-T cells are generated targeting an antigen expressed on the tumor cells and co-expressing a co-stimulatory receptor whose ligand is expressed or overexpressed in the cancer tissue. For example, for a patient with CD20-expressing lymphoma overexpressing TRAIL, a CD19-CAR-T cell product is manufactured that also co-expresses DcRT or DcR2. In another exemplary embodiment, for a patient with Mesothelin-expres sing ovarian cancer overexpressing 4TBB1L, a mesothelin CAR-T cell product is generated that co-expresses 4TBB1. Table 18 provides an example where the expression of different ligands in the diseased tissue, e.g., cancer cell or tissue, is matched by expressing the corresponding co-stimulatory receptor in the immune cells, e.g, T cells, e.g., CAR-T cells or TCR-T cells or TILs.

TABLE 18

| | | Costimualtory | Costimualtory |
|---|---|---|---|
| | | Molecule | Molecule |
| Antigen Expressed in | Costimulatory molecule to | SEQ ID NO | SEQ ID NO |
| Disease Cell or Tissue | be expressed in Immune Cell | (DNA) | (PRT) |
| TRAIL | DcR1 | 2250 | 2360 |
| TRAIL | DcR2 | 2251 | 2361 |
| TRAIL | DR5-SP-ECD-TM-CD27-CP | 2319 | 2429 |
| TRAIL | DR5-SP-ECD-TM-CD28-CP | 2320 | 2430 |
| TRAIL | DR5-SP-ECD-TM-41BB-CP | 2321 | 2431 |
| TRAIL | DR5-SP-ECD-TM-OX40-CP | 2322 | 2432 |
| TRAIL | DR5-SP-ECD-TM-GITR-CP | 2323 | 2433 |
| TRAIL | DR5-SP-ECD-TM-BCMA-CP | 2324 | 2434 |
| TRAIL | DR4-SP-ECD-CD27-TM-CP | 2332 | 2442 |
| TRAIL | DR4-SP-ECD-CD28-TM-CP | 2333 | 2443 |
| TRAIL | DR4-SP-ECD-41BB-TM-CP | 2334 | 2444 |
| TRAIL | DR4-SP-ECD-OX40-TM-CP | 2335 | 2445 |
| TRAIL | DR4-SP-ECD-GITR-TM-CP | 2336 | 2446 |
| TRAIL | DR4-SP-ECD-BCMA-TM-CP | 2337 | 2447 |
| TRAIL | DcR1-SP-ECD-CD27-TM-CP | 2339 | 2449 |
| TRAIL | DcR1-SP-ECD-CD28-TM-CP | 2340 | 2450 |
| TRAIL | DcR1-SP-ECD-41BB-TM-CP | 2341 | 2451 |
| TRAIL | DcR1-SP-ECD-OX40-TM-CP | 2342 | 2452 |
| TRAIL | DcR1-SP-ECD-GITR-TM-CP | 2343 | 2453 |
| TRAIL | DcR1-SP-ECD-BCMA-TM-CP | 2344 | 2454 |
| TRAIL | DcR2-SP-ECD-CD27-TM-CP | 2346 | 2456 |
| TRAIL | DcR2-SP-ECD-CD28-TM-CP | 2347 | 2457 |
| TRAIL | DcR2-SP-ECD-41BB-TM-CP | 2348 | 2458 |
| TRAIL | DcR2-SP-ECD-OX40-TM-CP | 2349 | 2459 |
| TRAIL | DcR2-SP-ECD-GITR-TM-CP | 2350 | 2460 |
| TRAIL | DcR2-SP-ECD-BCMA-TM-CP | 2351 | 2461 |
| CD70 | CD27 | 2254 | 2364 |
| 41BBL | 41BB | 2256 | 2366 |
| CD80 | CD28 | 2255 | 2365 |
| CD86 | CD28 | 2255 | 2365 |
| OX40L(CD252) | OX40 | 2257 | 2367 |
| GITR Ligand/TNFSF18 | GITR/TNFRSF18 | 2258 | 2368 |
| TNFSF13B/TALL-1/BAFF | BCMA | 2259 | 2369 |

The SEQ ID NO: of the nucleic acids and polypeptides encoding exemplary costimulatory molecules are provided in Table 1 (SEQ ID NO: 2250-2259 and SEQ ID NO: 2360-2368). Other co-stimulatory molecules are known in the art and can be used in alternate embodiment of the disclosure. The co-stimulatory molecules are expressed in the T cell product either using the same vector as being used to express the CAR/TCR module or using a separate vector. In certain embodiments, the costimulatory molecule is expressed in T cells (e.g, TILs) without expression of a CAR or an exogenous TCR. Essentially a similar approach is used to match any immune therapy product (e.g. T cell, NK cell, Macrophage) to a diseased cell or tissue (e.g., cancer) by ectopically expressing a receptor in the immune cell whose ligand is expressed or overexpressed in the diseased cell or tissue. It is expected that the immune cells that are engineered to ectopically express or overexpress one or more of the co-stimulatory receptors show better infiltration into the diseased tissue, e.g., cancer, and long-term persistence as compared to the control immune cells, e.g., immune cells that have not been engineered to ectopically express or overexpress the co-stimulatory receptors. Furthermore, it is expected that immune cells that are engineered to ectopically express or overexpress one or more of the co-stimulatory receptors whose ligands are expressed or overexpressed in the diseased tissue/cells, e.g., cancer or cancer cells, show better infiltration into the diseased tissue, e.g., cancer, and long-term persistence as compared to the control immune cells, e.g., immune cells that have not been engineered to ectopically express or overexpress the co-stimulatory receptors.

Use of Immune Effector Cell Therapy in Combination with Intratumoral Injection of K13, Nemo-K277A and IKK2-S177E-S181E

A patient with mesothelin expressing ovarian cancer, receives intratumoral delivery of a mammalian expression vector encoding nucleic acid encoding vFLIP K13, NEMO-K277A and/or IKK2-S177E-S181E by electroporation using methods known in the art followed 2 day later by infusion of 5×10⁸ Mesothelin-targeted CAR-T cells. Intratumoral expression of vFLIP K13, NEMO-K277A and/or IKK2-S177E-S181E is shown to stimulate immune cell infiltration into the tumor, including CAR-T cells. Essentially a similar procedure is used to enhance infiltration into the tumor tissue of immune effector cells engineered to express a NY-ESO-1 TCR.

Use of a Neutralizing TRAIL Antibody and DR5-Fc to Treat Immune Disorders, Such as Rheumatoid Arthritis, Systemic-Onset Juvenile Idiopathic Arthritis, Still's Disease, Macrophage Activation Syndrome, Hemophagocytic Lymphohistiocytosis (HLH), Systemic Lupus Erythematosus (SLE), Kawasaki Disease, and Inflammatory Bowel Disease.

A clinical trial is conducted to test a neutralizing TRAIL antibody (MAB375-SP) or DR5-Fc in patients with immune disorders, such as Rheumatoid Arthritis, Systemic-onset juvenile idiopathic arthritis, Still's disease, Macrophage activation syndrome, Hemophagocytic lymphohistiocytosis (HLH), systemic lupus erythematosus (SLE), Kawasaki disease, and inflammatory bowel disease Eligibility criteria for the trial include prior diagnosis of the above disorders and laboratory and clinical evidence of disease activity at study entry. Baseline clinical and laboratory measures of disease activity are obtained. Patients are randomized to receive a placebo, MAB375-SP or DR5-Fc. Patiens randomized to the MAB375-SP or DR5-Fc arms are administered a clinical grade neutralizing antibody against TRAIL (MAB375-SP) or DR5-Fc at dose of 5 mg/kg by intravenous infusion every week after pre-medication with Benadryl (50 mg i.v.) and Paracetamol (750 mg PO). Response to treatment is evaluated after 4 doses based on established clinical and laboratory measures of activity of the different diseases.

Use of a Neutralizing TRAIL Antibody and DR5-Fc to Prevent Flare of Immune Disorders, Such as Rheumatoid Arthritis, Systemic-Onset Juvenile Idiopathic Arthritis, Still's Disease, Macrophage Activation Syndrome, Hemophagocytic Lymphohistiocytosis (HLH), Systemic Lupus Erythematosus (SLE), Kawasaki Disease, and Inflammatory Bowel Disease.

A clinical trial is conducted to test a clinical-grade neutralizing TRAIL antibody (MAB375-SP) and DR5-Fc for prevention of flare or disease reactivation in patients with immune disorders, such as Rheumatoid Arthritis, Systemic-onset juvenile idiopathic arthritis, Still's disease, Macrophage activation syndrome, Hemophagocytic lymphohistiocytosis (HLH), systemic lupus erythematosus (SLE), Kawasaki disease, and inflammatory bowel disease Eligibility criteria for the trial include prior diagnosis of the above disorders and no active laboratory and/or clinical evidence of ongoing disease activity at study entry. Baseline clinical and laboratory measures of disease activity are obtained. Patients are randomized to receive a placebo, MAB375-SP or DR5-Fc. Patiens randomized to the MAB375-SP or DR5-Fc arms are administered a clinical grade neutralizing antibody against TRAIL (MAB375-SP) or DR5-Fc at dose of 5 mg/kg by intravenous infusion every week after pre-medication with Benadryl (50 mg i.v.) and Paracetamol (750 mg PO). Prevention of disease recurrence is evaluated after 3 and 6 months based on established clinical and laboratory measures of activity of the different diseases.

All publications and patents mentioned in the above specification are herein incorporated by reference. Various modifications and variations of the described method and system of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled relevant fields are intended to be within the scope of the following claims.

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

1. A method of manufacturing an immune effector cell therapy product comprising treating a donor subject with:
(a) a CXCR4 antagonist;
(b) a cytokine;
(c) a chemotherapeutic agent;
(d) a beta2 adrenergic agonist;
(e) a Src kinase inhibitor;
(f) exercise so as to reach a heart rate at least >25% higher than the heart rate at rest; or
(g) a combination of one or more of (a), (b), (c), (d), (e) or (f);
isolating blood from the donor subject;
isolating an immune effector cell or population thereof from the blood; and
optionally transforming the immune effector cell or population thereof with a heterologous polynucleotide to express or over express an endogenous or exogenous immune receptor, a chimeric antigen receptor (CAR) or a T cell receptor, wherein the immune receptor, CAR or T cell receptor comprises at least one antigen-binding domains and at least one transmembrane domain; or
optionally expanding the immune effector cell or population thereof in culture for a period of 1 to 30 days.

2. The method of claim 1, wherein the immune effector cell or population thereof has reduced, eliminated or altered expression and/or function of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1 HDAC2, JAK1, JAK3, STAT5b, STAT3, IL2RG, CARD11, BRAF, CD27, CD28, 41BB, DcR1, DcR2, OX40, GITR, BCMA, TRAIL, DR4 and/or DR5.

3. The method of claim 1, wherein the antigen-binding domain binds to a tumor antigen selected from a group consisting of: CD5, CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant Ill (EGFRviii); ganglioside G2 (GD2); ganglioside GD3 (aNeu5Ac(2-8)aNeu5Ac(2-3)bDGalp(I-4)bDGlcp(I-I)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); FmsLike Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors; a glycosylated CD43 epitope expressed on non-hematopoietic cancers; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-IIRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20/MS4A1; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDClalp(I-4)bDGlcp(I-1)Cer); transglutaminase 5 (TGS5); high molecular weight-melanomaassociated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51 E2 (OR51 E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member IA (XAGEI); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8); melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin BI; v-myc avian myelocytomatosis viral oncogene neuroblastoma derived homolog (MYCN); Ras Homolog Family Member C (RhoC); Tyrosinase-related protein 2 (TRP-2); Cytochrome P450 IB 1 (CYPIB 1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TESI); lymphocyte-specific protein tyrosine kinase (LCK); A kinase anchor protein 4 (AKAP-4); synovial sarcoma, X breakpoint 2 (SSX2); Receptor for Advanced Glycation End products (RAGE-1); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRI); Fc fragment of IgA receptor (FCAR or CD89); Leukocyte immunoglobulin-like receptor subfamily A member 2 (LILRA2); CD300 molecule-like family memberf (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); immunoglobulin lambda-like polypeptide 1 (IGLLI), MPL; Biotin; c-MYC epitope Tag; CD34; LAMP1 TROP2; GFRalpha4; CDH17; CDH6; NYBR1; CDH19; CD200R; Slea (CA19.9; Sialyl Lewis Antigen) Fucosyl-GM1; PTK7; gpNMB; CDH1-CD324; DLL3; CD276/B7H3; IL11Ra; IL13Ra2; CD179b-IGLI1; ALK TCRgamma-delta; NKG2D; CD32 (FCGR2A); CSPG4-HMW-MAA; Tim1-/HVCR1; CSF2RA (GM-CSFR-alpha); TGFbetaR2; VEGFR2/KDR; Lews Ag; TCR-beta1 chain; TCR-beta2 chain; TCR-gamma chain; TCR-delta chain; FITC; Leutenizing hormone receptor (LHR); Follicle stimulating hormone receptor (FSHR); Chorionic Gonadotropin Hormone receptor (CGHR); CCR4; SLAMF6; SLAMF4; HIV1 envelope glycoprotein; HTLV1-Tax; CMV pp65; EBV-EBNA3c; influenza A hemagglutinin (HA); GAD; PDL1; Guanylyl cyclase C (GCC); KSHV-K8.1 protein; KSHV-gH protein; auto-antibody to desmoglein 3 (Dsg3); autoantibody to desmoglein 1 (Dsg1); HLA; HLA-A; HLA-A2; HLA-B; HLA-C; HLA-DP; HLA-DM; HLA-DOA; HLA-DOB; HLA-DQ; HLA-DR; HLA-G; IGE; CD99; RAS G12V; Tissue Factor 1 (TF1); AFP; GPRC5D; claudin18.2 (CLD18A2 OR CLDN18A.2)); P-glycoprotein; STEAP1; LIV1; NECTIN-4; CRIPTO; MPL; GPA33; BST1/CD157; low conductance chloride channel; Integrin B7; Muc17; C16ORF54; VISTA; Muc5Ac; FCRH5; CLDN6; MMP16, UPK1B; BMPR1 B; Ly6E, WISP1 and SLC34A2.

4-9. (canceled)

10. The method of claim 1, wherein the immune effector cell or population thereof is engineered to express a constitutive active mutant of JAK1, JAK3, STAT5b, STAT3, IL2RG, CARD11 and/or BRAF.

11. The method of claim 1, wherein the expression and/or function of BRD9, EZH2, MLL2, MLL3, MLL4, methylcytosine dioxygenase genes (e.g., Tet1, Tet2, Tet3), ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5, is reduced or eliminated and expression and function of JAK1, JAK3, STAT5b, STAT3, IL2RG, CARD11 and/or BRAF is enhanced in the immune effector cell or population thereof.

12. The method of claim 1, wherein the immune effector cell or population thereof expresses a CAR, a next generation CAR or a TCR.

13-15. (canceled)

16. The method of claim 1, wherein the CXCR4 antagonist is selected from the group consisting of Plerixafor or BL-80400.

17-28. (canceled)

29. A method of manufacturing an immune effector cell therapy product of claim 11, the method comprising introducing polynucleotide encoding a CAR/TCR into an immune effector cell such that said polynucleotide integrates into the genome of the immune effector cell within a BRD9, EZH2, MLL2, MLL3, MLL4, methylcytosine dioxygenase genes, ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 gene or intron or exon thereof of wherein expression and/or function is reduced or eliminated.

30. A method of manufacturing an immune effector cell therapy product of claim 11, the method comprising contacting said immune effector cell ex vivo with a BRD9, EZH2, MLL2, MLL3, MLL4, methylcytosine dioxygenase genes, ATM, CHEK, FBXW10, BCOR, FAT1, ASXL1, PHF6, SF3B1, YY1, CBFb, Runx1, EHMT2 (G9A), SMARCA4, CREBBP, PRDM1/BLIMP1, HDAC2, TRAIL, and/or DR5 inhibitor.

31-51. (canceled)

52. A method of manufacturing an immune effector cell therapy product of claim 1 comprising exposing the cell to a bispecific or multispecific engager in the presence of an antigen presenting cell or an anti-presenting substrate.

53. The method of claim 52, where the bispecific or multi-specific engager comprises at least one antigen binding domain capable of engaging the immune effector cell and at least one antigen binding domain capable of engaging an antigen presenting cell (APC) or antigen presenting substrate (APS).

54. The method of claim 53, where the APC is a hematopoietic cell, a B cell lineage cell or wherein the cell comprises a CD19 antigen.

55-56. (canceled)

57. The method of claim 53, where the APC is selected from the group consisting of a REC-1, JEKO-1, MINO, GRANTA-519, NALM6 and RAJI cell.

58-59. (canceled)

60. A method of claim 52, where the method further involves exposing the immune effector cells to an agonist that activates a costimulatory receptor on immune cells

61. The method of claim 52, wherein the bispecific or multispecific engager comprises at least one binding domain capable of binding to and activating the T cell receptor (TCR) complex of T cells.

62. (canceled)

63. The method of claim 52, wherein the bispecific or multispecific engager comprises at least one binding domain capable of binding to and activating a receptor on the T cells that provides co-stimulation.

64. The method of claim 63, where the receptor bound by the bispecific or multispecific engager is selected from the group consisting of CD28, CD137 (4-1BB), CD134 (OX40), Dap10, CD27, CD2, CD5, ICAM-1, LFA-1, TNFR-I, TNFR-II, Fas, CD30 and CD40.

65-83. (canceled)

84. A method of expanding and/or activating an immune effector cell comprising administering to a subject a bispecific or multispecific T cell engager.

85. A method of claim 84, where the bispecific or multi-specific engager contains at least one antigen binding domain capable of engaging the immune cells and at least one antigen binding domain capable of engaging an antigen presenting cell (APC).

86. The method of claim 85, wherein the APC is a hematopoietic cell, a B cell lineage cell or a cell expressing the CD19 antigen.

87-153. (canceled)
